SEQUENCE LISTING (35 sequences)

SEQ ID NO: 1 - DNA, 778 bp, Artificial Sequence
  miR223 promoter sequence
acttgtacagcttcacagggctccatgcttagaaggaccccacacttagtttaatgttctgctgtcatcatcttgatattcttaatttttaaataaagggcctatcgttttcattttttactgggccttgcaaattatgtagctggttctgtatgccaggagagaagttggaagtaaaatggtattccaggaccaggaggcattctggcagagtgaaagaacatgtgatttggagtccatggggatgggtttaaatttcagctttccactaatttgctttgtgatactgagtatttccttttatccctcagaggctctgtttctcaattttgactacgggttttttcattagataatgtctcagttctggtattccaggtttccctcaattattctgggaaaacctccttgacccacaggcagagcctagggcagccaggtgctttctactctctctctctctgcagcttggaaagttagtgtctgttgaaggtcagctgggagttggtggaggcagggcagtggcctgctactattgctgcagtagcagaccctttcacaacagcattgttttgtcattttgcatccagatttccgttggctaacctcagtcttatcttcctcatttctgtttcctgttgaagacaccaagggcccttcaaaacacagaagcttcttgctcacggcagaaagcccaattccatctggcccctgcaggttggctcagcactggggaatcagagtcccctccatgaccaaggcaccactccactgacag

SEQ ID NO: 2 - DNA, 1424 bp, Artificial Sequence
  P2Y12 promoter sequence
ggtgttggagaggatgtggagaaataggaacacttttacactgttggtgggactataaactagttcaaccattgtggaagtcagtgtggtgattcctcagtgatctagaactagaaataccatttaacccagccatcccattactgggtatatacccaaaggattataagtcatgctgctataaagacacatgcacacgtatgtttattgcggcactattcataatagcaaagacttggaaccaacccaaaagtccaacaatgatagactggattaagaaaatgtggcacatatacaccatggaatactatgcagccataaaaaatgatgagttcatgtcctttgtagggacatggatgaaattagaaatcatcattctcagtaaactatcgcaagaacaaaaaaccaaacaccacatattctcactcataggtgggaactgaacaatgagaacacatggacacaggaagggaaacactacactctggggactgttgtggggtggggggatgggggagggatagctttaggagatatacctaatgctaaatgacgagttaatgggtgtagcacaccagcatggcacatgtatacatatgtaactaacctgcacattgtgcacatgtaccctaaaacttaacgtataataataataaaattaaaaaaaaaaagttaaagcagcaaaacactttgcccttcaatctcacccctaacatattttttgcccttctggtttcaaagttaaacaactgtaaataattgtgatacaaggatgccttaatttaatgttatattttcccaaaaactcaaagttaggtaaagaaacaaaaaaaaattgtttatatttaaattctattcaagaaaagcatgaacgacacagtatataataagcctggcaatggatacaatcacttctctaatgtaattttggaatctgctaatttataatagaaggaagctgtttcacctacaaaggagttaatcaaacacaggtttaaaataatgacattattaaccaagggaaaaacaaagggccagagacttaacatccctagccagcacgcattttgagttaacataattacttgttagaagaaaatacatcacccagtgttgtacacaatatatttcagataaattaaccacccaagaaagcaagcttaaaatcttctccaggaagcagacttcgaaggcttgatctcaacttggatttatcatttgcatagaaaataaccataactcgaagttataaatcatcaactctatagcaggtttcagtaaaaagccgcaagattttaaattgctttttaaaagatgacttctcagccatcctcatcccacatttcctgggaaataaaagcagaagtcctaaaagaggacagatagaaattcagtgtctgcatagctttgagtccagtgtttga

SEQ ID NO: 3 - DNA, 1308 bp, Artificial Sequence
  TMEM119 promoter sequence
gttcctacccagagagcacgcactcatccttcatgcactcccctgttccaaaccctcactggctccgtactgcctccgaccttccgagactttagcctggctcctgtcaacatctctgacccttactacatgatcctctctttggtccatgctccagcctaatctaattgcggtggcttgtgcgtggtggcattcccagccaccatacctttacccacgctggtccttccatgcggaatgcctttccagggcctgctttgcccgcttctgctcatacacaggcatgccctccaggatggcttcctacctctttcccttgggggattgatctctctgtcttggggttctcggagcccttgacctgacccctttctgtttggcaaaaaagtaatttacctcggtgtccttctccctggtagtctgtgagctccccaaggctgggctgtgcctgattcacctctggaacttgcttagcacagtgcgtggcctgctgcaggtgttcattgagcacttgccgaatgaatgcatgaatgaatgaatgaatgaatgaatgcaaggggctgctaatccacaggactcctcaggtcagccagacgtcccggttccaaggcctgccactgactcacctcaggaccctgcttgaaccattagaactcaccctgcctcactttccccctctgtgaaatggggctccaactcctattcaagctactatcatttgggggcattgtgaggccacagatcccagaacatcagagtcagaggtagcccagaaagcttcccacccatccctacaaatgggaaactgaggtctggagagggaagggcagagttgggctccctgtctcaggctcggacccaccatcaggcctgtctctaaaacgaatcccagctcccacgctgcaccctgagcctggaagcctgagccacacaaggacggggaattttccttcccacttccagaggcctctgaacctccctgagcttgtcccctttggagggtattgggcagcagcgtgggcagaaccccagctcactgtctgggggagcgctgcaggacagccttgtctgtctgtctcagcctgccctggggacccgaggtcagggaggaagtgccgcatctggtcttccccagagcgagagtgtgagcaagggtgggattgcgtgtggcccgagagtagcccctcccctccccctgtccccaccccaaaccctcttaatgaaatcaagctggccctgcggcccagccggggagggaggaaggaggagggacgggaggagggacgggaggagggagggcgggcaggc

SEQ ID NO: 4 - DNA, 1417 bp, Artificial Sequence
  OLFML3 promoter sequence
gcagtgtccagggtccttacttcacatcatctggatctgaccctttgaaagaggtagaagacttctgagaccggctaattaagctttgtttcctcatatgttttgccagatagcagtagcagaatgaaaagatgagtaaccacaggaagctgctatttttcccctcctttcaaactgtactgttagagtcatggtcctttttacagaaggaacctctcatcagattctgttgattctaaagtgaatagaatttctcccgataaagaaataggggtttgtttcgattaatgactgcaggtctctgagtaaatgctctatttgattttttttttcggcccgtgtgtctaccttatggcccaagtctaccttatggtggccattaattcattttgggctcctgcagccttagttgggatatagaaatgagaaacactcagaaatacccttttggaccacaaccaagagaaataaccaatagtcttttctcccagtggtaaggaagtcagaatacattgatctagactgcaacaacatatatatatatatcagattccgcccccccgcaatacatgaatgtatagtaaattagtgtgaactcactgaacactcctcagttttggtgagagactatatctggcctctttcaagcaaaggaaagccatgtaaaacagcgctgctgtcagccttaacttccagacgatcgagttaatttactaacttctcagtgacctgttttttttttttttttaatctcagttatattttcttccttgggctaaatcagatatttgcatagcccccaaagtagtaattggatagtcttgggggaaatatgcatttcagtggtgaaaacccctgtaaattcaatatatttggcttttgtggaaaattttcctcatggggtgaagtctaagccttagtttctgtattatcatgagagatgacaccagctgcttagcacaaggtggcgccaatgagctttagaataagttgggcttgaccacttgggccattgttttcctgcttcctcccttcaagcctcacctccccagctcccagcttctactgaacaaggctgaaaacccactctattgcagggaaagggaaagattaatgaaaaatgtcagtttcttaagtcagcactggtgaaactttcctaaaacaggaatggcgtttgctgagttttctctggggtctctgctttctgcagctagcttccctgcttgactgcctagaaggcctctgctttcgggtttccatctctttcccctccagaggaccctacagcctaggcgggaggtggttaaggcttctggctgctgtgcaatggggccatctgtgtttgatcaatcctggcggaaaggagggggtgggggttgtaaagagaactgaaagcattccagagtagtgagagaga

SEQ ID NO: 5 - DNA, 1247 bp, Artificial Sequence
  AIF1 promoter sequence
cgcctgtagtcccagctactcaggaggctgaggtaggagaattgcttgaacccaggaggcagtggttgcagtgagccgagattgcaccattgcactcccgcctgggcgacagagcaagactccgactcaaaaaaaaaaaaaaaagcagcagcagcagccagaggccactccagcatctcccctacctggcttgggtcagggagagggcagtgagaagtgaaaactcccagctacagaaaaggaaatatgttggggggaagggagaaggaaaggtgtcttcatcaatgccggggcagggtagatggagccctgggcagggagtttggaccaggaaatctcaatgagggaaatgtgctgtcctcacctctccaagaagcgactggccaaacagagtgacagaggggataaaggttatgcctagggaggcatgtgtcagaggctatcatccactctgttgaacccacagtgaccagcaccaccatcacacaaacatgcctgcatgtgtgcacgcacgtgcagtgtgcaaacctgatgtcagcctcactccctggctcttctgtccacaaacgctgtttctttaagtaccactttcagttcctccaaagaatctacttaaactcttaaattcctgatctctatagattttactaaagatttcaaaggagataagatgagagggttacgttgcacattctaaagcaaacaaattaaaatgttttgttagacatttccatatttttaagggcctccttggagctgccaggctgggagtgaggtttctctccctttctaaaccctgtgcccatcttgtcaccctcctggagctgccagcagacttcagattcttctccgatctacagagcagaaaaattcagccagcccttccttgtcttcctatccacagctgcctgcccagactcatgaaacctgacaaaatgcaaggtcttatcattacctgaaccttggacctgttcaaaaatactagttcctgagaataaatatccctggtgtcttcctgcccttcctgcacacctccagtggcttatcaaaatatttgtttcatgcgcacactgggctctcatttaagaggaatttgggagaatgttattttctaatctgcatttcacaccaggctccccctccttcctggggtgctagtgtcagcagaacctgatggggaagtgaggtctgggaggcagaggaggaaggaatgaggggaaaggggaagtttgggaggaaggcttctg

SEQ ID NO: 6 - DNA, 1402 bp, Artificial Sequence
  ITGAM promoter sequence
cgcacccagccaagtttgtacatatatttttgactacacttcttaactattcttaggataaattactagaagtgaaaattcttgggtgaagagcttgaggcctttacacacacacacacacacacacacaaaaataggctggatgcagtggctcacacctgtaatctcagcagtttgggaggctgaggaaggaggatcacttgagtccaggaggttgagaatagcctgaacaacatagcaagatcttgtctctacaaaaaatttaaaaaaaattagctggccatggcagcatgtgcctgtagtaccagctactcggaaggctgaggtaggaggatcgcttgagcccaggaggttgattgaagctgcagtgagctgtgattacaccactgcactccagcctgggcaacagagctagactctgtctctaaaaaaagcacaaaataatatttaaaaagcaccaggtatgcctgtacttgagttgtctttgttgatggctacaaatgaggacagctctggctgaagggcgcttccatttccatgggctgaaggagggacattttgcaaagtgtgttttcaggaagacacagagttttacctcctacacttgtttgatctgtattaatgtttgcttatttatttatttaattttttttttgagacagagtctcactctgtcacctgggctggagtgcagtggcattattgaggctcattgcagtctcagactcctgagctcaaacaatcctcctgcctcagcctctggagtagctaggactacaggcatgtgccaccatgcctggctaattttttaaatgtatttttttgtagagtcggggtctccctatgttgcccaggctggagtgcagtggtgtgatcctagctcactgcagcctggacctcgggctcaagtaattctcacacctcagcctgtccagtagcaggggctacaggcgcgcaccaccatgcccagctaattaaaaatatttttttgtagagacagggtctctctatgttgcccaggctggtttcaaactcccaggctcaagcaatcctcctgccttggcctcccaaagtgctggcattacaggcgtgagccactgcgcctggcccgtattaatgtttagaacacgaattccaggaggcaggctaagtctgttcagcttgttcatatgcttgggccaacccaagaaacaagtgggtgacaaatggcaccttttggatagtggtattgactttgaaagtttgggtcaggaagctggggaggaagggtgggcaggctgtgggcagtcctgggcggaagaccaggcagggctatgtgctcactgagcctccgccctcttcctttgaatctctgatagacttctgcctcctacttctccttttctgcccttctttgctttgg

SEQ ID NO: 7 - protein, 593 aa, Artificial Sequence
  Progranulin isoform 1
MWTLVSWVALTAGLVAGTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 8 - protein, 438 aa, Artificial Sequence
  Progranulin isoform 2
MWTLVSWVALTAGLVAGTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCRDNRQGWACCPYRQGVCCADRRHCCPAGFRCAARGTKCLRREAPRWDAPLRDPALRQLL

SEQ ID NO: 9 - protein, 413 aa, Artificial Sequence
  Progranulin isoform 3
MAITAAHGASTAVQTGDPASKDQVTTPWVPSSALIVSSNARTSPRAVLWSMAPGGAAPCPRLPAVKTGCTAVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 10 - protein, 166 aa, Artificial Sequence
  Human interferon-gamma
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 11 - protein, 219 aa, Artificial Sequence
  Human IL-12 subunit alpha
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 12 - protein, 328 aa, Artificial Sequence
  Human IL-12 subunit beta
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 13 - protein, 144 aa, Artificial Sequence
  Human GM-CSF
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 14 - protein, 207 aa, Artificial Sequence
  Human G-CSF
MAGPATQSPMKLMALQLLLWHSALWTVQEATPLGPASSLPQSFLLKCLEQVRKIQGDGAALQEKLVSECATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 15 - DNA, 1532 bp, Artificial Sequence
  GM-CSF - INF-gamma coexpression construct
tccttccagccatgtttaaatatacaagttatatcttggcttttcagctctgcatcgttttgggttctcttggctgttactgccaggacccatatgtaaaagaagcagaaaaccttaagaaatattttaatgccggtcattcagatgtagcggataatggaactcttttcttaggcattttgaagaattggaaagaggagagtgacagaaaaataatgcagagccaaattgtctccttttacttcaaactttttaagaactttaaggatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagcgaaaacagggaagcgaaaaaggagtcagatgctgtttcgaggtcgaagagcatcccagtaagatatcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaagataccccattgtatgggatctgatctggggcctcggtgcacatgctttacatgtgtttagtcgaggttaaaaaaacgtctaggccccccgaaccacggggacgtggttttcctttgaaaaacacgatgataaatgtggccactgcaaagtctgcttttgctgggcaccgtagcttgtagcatatcagcgcctgctcggagtccctctccatcaacgcaaccctgggaacacgtgaacgcaattcaggaggcaagaaggttgctgaacctgagccgggacaccgccgctgaaatgaatgaaaccgtagaagtgatttccgagatgtttgacctccaagaaccaacttgtctgcaaacaagacttgagctttataaacagggactccgaggcagcctgacaaaactcaaggggcccctcacaatgatggcaagccattataaacaacactgtcctccgaccccggagacttcttgcgccacacagatcatcacttttgagagcttcaaagagaaccttaaagactttctgctggtcattccgttcgattgctgggaacccgtgcaggagtga

SEQ ID NO: 16 - DNA, 1718 bp, Artificial Sequence
  G-CSF - INF-gamma coexpression construct
tccttccagccatgtttaaatatacaagttatatcttggcttttcagctctgcatcgttttgggttctcttggctgttactgccaggacccatatgtaaaagaagcagaaaaccttaagaaatattttaatgccggtcattcagatgtagcggataatggaactcttttcttaggcattttgaagaattggaaagaggagagtgacagaaaaataatgcagagccaaattgtctccttttacttcaaactttttaagaactttaaggatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagcgaaaacagggaagcgaaaaaggagtcagatgctgtttcgaggtcgaagagcatcccagtaagatatcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaagataccccattgtatgggatctgatctggggcctcggtgcacatgctttacatgtgtttagtcgaggttaaaaaaacgtctaggccccccgaaccacggggacgtggttttcctttgaaaaacacgatgataaatggccggccccgccacccagagccccatgaagctgatggccctgcagctgctgctgtggcacagcgccctgtggaccgtgcaggaggccacccccctcggccccgccagcagcctgccccagagcttcctgctgaagtgcctcgaacaagtgcgcaagatacaaggcgacggcgccgccctgcaggagaagctcgtgagcgagtgcgccacctacaagctgtgccaccccgaggagctggtgctgctgggccacagcctcggcatcccctgggcccccctgagcagctgccccagccaagccctgcagctggccggctgcctgagccagctgcacagcggcctgttcctgtaccaaggcttactacaggccctcgaaggcatcagccccgagctgggccccaccctcgacaccctgcagctggacgtggccgacttcgccaccaccatctggcagcagatggaggagctgggcatggcccccgccctgcagcccacccaaggcgccatgcccgccttcgccagcgccttccagcgccgcgccgggggcgtgctggtggccagccacctgcagagcttcctcgaagtgagctaccgcgtgctgcgccacctcgcccagccctga

SEQ ID NO: 17 - protein, 153 aa, Artificial Sequence
  Human IL-2
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 18 - protein, 162 aa, Artificial Sequence
  Human IL-15
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 19 - protein, 162 aa, Artificial Sequence
  Human IL-21
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 20 - protein, 188 aa, Artificial Sequence [sequence omitted]

SEQ ID NO: 21 - DNA, 760 bp, Artificial Sequence
  P2Y12 promoter sequence 2 (760 bp)
ttgcccttcaatctcacccctaacatattttttgcccttctggtttcaaagttaaacaactgtaaataattgtgatacaaggatgccttaatttaatgttatattttcccaaaaactcaaagttaggtaaagaaacaaaaaaaaattgtttatatttaaattctattcaagaaaagcatgaacgacacagtatataataagcctggcaatggatacaatcacttctctaatgtaattttggaatctgctaatttataatagaaggaagctgtttcacctacaaaggagttaatcaaacacaggtttaaaataatgacattattaaccaagggaaaaacaaagggccagagacttaacatccctagccagcacgcattttgagttaacataattacttgttagaagaaaatacatcacccagtgttgtacacaatatatttcagataaattaaccacccaagaaagcaagcttaaaatcttctccaggaagcagacttcgaaggcttgatctcaacttggatttatcatttgcatagaaaataaccataactcgaagttataaatcatcaactctatagcaggtttcagtaaaaagccgcaagattttaaattgctttttaaaagatgacttctcagccatcctcatcccacatttcctgggaaataaaagcagaagtcctaaaagaggacagatagaaattcagtgtctgcatagctttgagtccagtgtttgaagacaatctctgattgtgaagccctc

SEQ ID NO: 22 - DNA, 438 bp, Artificial Sequence
  P2Y12 promoter sequence 3 (438 bp)
taaccaagggaaaaacaaagggccagagacttaacatccctagccagcacgcattttgagttaacataattacttgttagaagaaaatacatcacccagtgttgtacacaatatatttcagataaattaaccacccaagaaagcaagcttaaaatcttctccaggaagcagacttcgaaggcttgatctcaacttggatttatcatttgcatagaaaataaccataactcgaagttataaatcatcaactctatagcaggtttcagtaaaaagccgcaagattttaaattgctttttaaaagatgacttctcagccatcctcatcccacatttcctgggaaataaaagcagaagtcctaaaagaggacagatagaaattcagtgtctgcatagctttgagtccagtgtttgaagacaatctctgattgtgaagccctc

SEQ ID NO: 23 - DNA, 1005 bp, Artificial Sequence
  TMEM119 promoter sequence 2 (1005 bp)
cccttgacctgacccctttctgtttggcaaaaaagtaatttacctcggtgtccttctccctggtagtctgtgagctccccaaggctgggctgtgcctgattcacctctggaacttgcttagcacagtgcgtggcctgctgcaggtgttcattgagcacttgccgaatgaatgcatgaatgaatgaatgaatgaatgaatgcaaggggctgctaatccacaggactcctcaggtcagccagacgtcccggttccaaggcctgccactgactcacctcaggaccctgcttgaaccattagaactcaccctgcctcactttccccctctgtgaaatggggctccaactcctattcaagctactatcatttgggggcattgtgaggccacagatcccagaacatcagagtcagaggtagcccagaaagcttcccacccatccctacaaatgggaaactgaggtctggagagggaagggcagagttgggctccctgtctcaggctcggacccaccatcaggcctgtctctaaaacgaatcccagctcccacgctgcaccctgagcctggaagcctgagccacacaaggacggggaattttccttcccacttccagaggcctctgaacctccctgagcttgtcccctttggagggtattgggcagcagcgtgggcagaaccccagctcactgtctgggggagcgctgcaggacagccttgtctgtctgtctcagcctgccctggggacccgaggtcagggaggaagtgccgcatctggtcttccccagagcgagagtgtgagcaagggtgggattgcgtgtggcccgagagtagcccctcccctccccctgtccccaccccaaaccctcttaatgaaatcaagctggccctgcggcccagccggggagggaggaaggaggagggacgggaggagggacgggaggagggagggcgggcaggcgccagcccagagcagccccgggcaccagcacggactctctcttccatcta

SEQ ID NO: 24 - DNA, 590 bp, Artificial Sequence
  TMEM119 promoter sequence 3 (590 bp)
cccagaaagcttcccacccatccctacaaatgggaaactgaggtctggagagggaagggcagagttgggctccctgtctcaggctcggacccaccatcaggcctgtctctaaaacgaatcccagctcccacgctgcaccctgagcctggaagcctgagccacacaaggacggggaattttccttcccacttccagaggcctctgaacctccctgagcttgtcccctttggagggtattgggcagcagcgtgggcagaaccccagctcactgtctgggggagcgctgcaggacagccttgtctgtctgtctcagcctgccctggggacccgaggtcagggaggaagtgccgcatctggtcttccccagagcgagagtgtgagcaagggtgggattgcgtgtggcccgagagtagcccctcccctccccctgtccccaccccaaaccctcttaatgaaatcaagctggccctgcggcccagccggggagggaggaaggaggagggacgggaggagggacgggaggagggagggcgggcaggcgccagcccagagcagccccgggcaccagcacggactctctcttccatcta

SEQ ID NO: 25 - DNA, 990 bp, Artificial Sequence
  OLFML3 promoter sequence 2 (990 bp)
tacccttttggaccacaaccaagagaaataaccaatagtcttttctcccagtggtaaggaagtcagaatacattgatctagactgcaacaacatatatatatatatcagattccgcccccccgcaatacatgaatgtatagtaaattagtgtgaactcactgaacactcctcagttttggtgagagactatatctggcctctttcaagcaaaggaaagccatgtaaaacagcgctgctgtcagccttaacttccagacgatcgagttaatttactaacttctcagtgacctgttttttttttttttttaatctcagttatattttcttccttgggctaaatcagatatttgcatagcccccaaagtagtaattggatagtcttgggggaaatatgcatttcagtggtgaaaacccctgtaaattcaatatatttggcttttgtggaaaattttcctcatggggtgaagtctaagccttagtttctgtattatcatgagagatgacaccagctgcttagcacaaggtggcgccaatgagctttagaataagttgggcttgaccacttgggccattgttttcctgcttcctcccttcaagcctcacctccccagctcccagcttctactgaacaaggctgaaaacccactctattgcagggaaagggaaagattaatgaaaaatgtcagtttcttaagtcagcactggtgaaactttcctaaaacaggaatggcgtttgctgagttttctctggggtctctgctttctgcagctagcttccctgcttgactgcctagaaggcctctgctttcgggtttccatctctttcccctccagaggaccctacagcctaggcgggaggtggttaaggcttctggctgctgtgcaatggggccatctgtgtttgatcaatcctggcggaaaggagggggtgggggttgtaaagagaactgaaagcattccagagtagtgagagagacccagagatcagg

SEQ ID NO: 26 - DNA, 1544 bp, Artificial Sequence
  miR223_P2RY12 (760 bp) fusion promoter
acttgtacagcttcacagggctccatgcttagaaggaccccacacttagtttaatgttctgctgtcatcatcttgatattcttaatttttaaataaagggcctatcgttttcattttttactgggccttgcaaattatgtagctggttctgtatgccaggagagaagttggaagtaaaatggtattccaggaccaggaggcattctggcagagtgaaagaacatgtgatttggagtccatggggatgggtttaaatttcagctttccactaatttgctttgtgatactgagtatttccttttatccctcagaggctctgtttctcaattttgactacgggttttttcattagataatgtctcagttctggtattccaggtttccctcaattattctgggaaaacctccttgacccacaggcagagcctagggcagccaggtgctttctactctctctctctctgcagcttggaaagttagtgtctgttgaaggtcagctgggagttggtggaggcagggcagtggcctgctactattgctgcagtagcagaccctttcacaacagcattgttttgtcattttgcatccagatttccgttggctaacctcagtcttatcttcctcatttctgtttcctgttgaagacaccaagggcccttcaaaacacagaagcttcttgctcacggcagaaagcccaattccatctggcccctgcaggttggctcagcactggggaatcagagtcccctccatgaccaaggcaccactccactgacagggatccttgcccttcaatctcacccctaacatattttttgcccttctggtttcaaagttaaacaactgtaaataattgtgatacaaggatgccttaatttaatgttatattttcccaaaaactcaaagttaggtaaagaaacaaaaaaaaattgtttatatttaaattctattcaagaaaagcatgaacgacacagtatataataagcctggcaatggatacaatcacttctctaatgtaattttggaatctgctaatttataatagaaggaagctgtttcacctacaaaggagttaatcaaacacaggtttaaaataatgacattattaaccaagggaaaaacaaagggccagagacttaacatccctagccagcacgcattttgagttaacataattacttgttagaagaaaatacatcacccagtgttgtacacaatatatttcagataaattaaccacccaagaaagcaagcttaaaatcttctccaggaagcagacttcgaaggcttgatctcaacttggatttatcatttgcatagaaaataaccataactcgaagttataaatcatcaactctatagcaggtttcagtaaaaagccgcaagattttaaattgctttttaaaagatgacttctcagccatcctcatcccacatttcctgggaaataaaagcagaagtcctaaaagaggacagatagaaattcagtgtctgcatagctttgagtccagtgtttgaagacaatctctgattgtgaagccctc

SEQ ID NO: 27 - DNA, 1222 bp, Artificial Sequence
  miR223_P2RY12 (438 bp) fusion promoter
acttgtacagcttcacagggctccatgcttagaaggaccccacacttagtttaatgttctgctgtcatcatcttgatattcttaatttttaaataaagggcctatcgttttcattttttactgggccttgcaaattatgtagctggttctgtatgccaggagagaagttggaagtaaaatggtattccaggaccaggaggcattctggcagagtgaaagaacatgtgatttggagtccatggggatgggtttaaatttcagctttccactaatttgctttgtgatactgagtatttccttttatccctcagaggctctgtttctcaattttgactacgggttttttcattagataatgtctcagttctggtattccaggtttccctcaattattctgggaaaacctccttgacccacaggcagagcctagggcagccaggtgctttctactctctctctctctgcagcttggaaagttagtgtctgttgaaggtcagctgggagttggtggaggcagggcagtggcctgctactattgctgcagtagcagaccctttcacaacagcattgttttgtcattttgcatccagatttccgttggctaacctcagtcttatcttcctcatttctgtttcctgttgaagacaccaagggcccttcaaaacacagaagcttcttgctcacggcagaaagcccaattccatctggcccctgcaggttggctcagcactggggaatcagagtcccctccatgaccaaggcaccactccactgacagggatcctaaccaagggaaaaacaaagggccagagacttaacatccctagccagcacgcattttgagttaacataattacttgttagaagaaaatacatcacccagtgttgtacacaatatatttcagataaattaaccacccaagaaagcaagcttaaaatcttctccaggaagcagacttcgaaggcttgatctcaacttggatttatcatttgcatagaaaataaccataactcgaagttataaatcatcaactctatagcaggtttcagtaaaaagccgcaagattttaaattgctttttaaaagatgacttctcagccatcctcatcccacatttcctgggaaataaaagcagaagtcctaaaagaggacagatagaaattcagtgtctgcatagctttgagtccagtgtttgaagacaatctctgattgtgaagccctc

SEQ ID NO: 28 - DNA, 1789 bp, Artificial Sequence
  miR223_TMEM119 fusion promoter
acttgtacagcttcacagggctccatgcttagaaggaccccacacttagtttaatgttctgctgtcatcatcttgatattcttaatttttaaataaagggcctatcgttttcattttttactgggccttgcaaattatgtagctggttctgtatgccaggagagaagttggaagtaaaatggtattccaggaccaggaggcattctggcagagtgaaagaacatgtgatttggagtccatggggatgggtttaaatttcagctttccactaatttgctttgtgatactgagtatttccttttatccctcagaggctctgtttctcaattttgactacgggttttttcattagataatgtctcagttctggtattccaggtttccctcaattattctgggaaaacctccttgacccacaggcagagcctagggcagccaggtgctttctactctctctctctctgcagcttggaaagttagtgtctgttgaaggtcagctgggagttggtggaggcagggcagtggcctgctactattgctgcagtagcagaccctttcacaacagcattgttttgtcattttgcatccagatttccgttggctaacctcagtcttatcttcctcatttctgtttcctgttgaagacaccaagggcccttcaaaacacagaagcttcttgctcacggcagaaagcccaattccatctggcccctgcaggttggctcagcactggggaatcagagtcccctccatgaccaaggcaccactccactgacagggatcccccttgacctgacccctttctgtttggcaaaaaagtaatttacctcggtgtccttctccctggtagtctgtgagctccccaaggctgggctgtgcctgattcacctctggaacttgcttagcacagtgcgtggcctgctgcaggtgttcattgagcacttgccgaatgaatgcatgaatgaatgaatgaatgaatgaatgcaaggggctgctaatccacaggactcctcaggtcagccagacgtcccggttccaaggcctgccactgactcacctcaggaccctgcttgaaccattagaactcaccctgcctcactttccccctctgtgaaatggggctccaactcctattcaagctactatcatttgggggcattgtgaggccacagatcccagaacatcagagtcagaggtagcccagaaagcttcccacccatccctacaaatgggaaactgaggtctggagagggaagggcagagttgggctccctgtctcaggctcggacccaccatcaggcctgtctctaaaacgaatcccagctcccacgctgcaccctgagcctggaagcctgagccacacaaggacggggaattttccttcccacttccagaggcctctgaacctccctgagcttgtcccctttggagggtattgggcagcagcgtgggcagaaccccagctcactgtctgggggagcgctgcaggacagccttgtctgtctgtctcagcctgccctggggacccgaggtcagggaggaagtgccgcatctggtcttccccagagcgagagtgtgagcaagggtgggattgcgtgtggcccgagagtagcccctcccctccccctgtccccaccccaaaccctcttaatgaaatcaagctggccctgcggcccagccggggagggaggaaggaggagggacgggaggagggacgggaggagggagggcgggcaggcgccagcccagagcagccccgggcaccagcacggactctctcttccatcta

SEQ ID NO: 29 - DNA, 1774 bp, Artificial Sequence
  miR223_OLFML3 fusion promoter
acttgtacagcttcacagggctccatgcttagaaggaccccacacttagtttaatgttctgctgtcatcatcttgatattcttaatttttaaataaagggcctatcgttttcattttttactgggccttgcaaattatgtagctggttctgtatgccaggagagaagttggaagtaaaatggtattccaggaccaggaggcattctggcagagtgaaagaacatgtgatttggagtccatggggatgggtttaaatttcagctttccactaatttgctttgtgatactgagtatttccttttatccctcagaggctctgtttctcaattttgactacgggttttttcattagataatgtctcagttctggtattccaggtttccctcaattattctgggaaaacctccttgacccacaggcagagcctagggcagccaggtgctttctactctctctctctctgcagcttggaaagttagtgtctgttgaaggtcagctgggagttggtggaggcagggcagtggcctgctactattgctgcagtagcagaccctttcacaacagcattgttttgtcattttgcatccagatttccgttggctaacctcagtcttatcttcctcatttctgtttcctgttgaagacaccaagggcccttcaaaacacagaagcttcttgctcacggcagaaagcccaattccatctggcccctgcaggttggctcagcactggggaatcagagtcccctccatgaccaaggcaccactccactgacagggatcctacccttttggaccacaaccaagagaaataaccaatagtcttttctcccagtggtaaggaagtcagaatacattgatctagactgcaacaacatatatatatatatcagattccgcccccccgcaatacatgaatgtatagtaaattagtgtgaactcactgaacactcctcagttttggtgagagactatatctggcctctttcaagcaaaggaaagccatgtaaaacagcgctgctgtcagccttaacttccagacgatcgagttaatttactaacttctcagtgacctgttttttttttttttttaatctcagttatattttcttccttgggctaaatcagatatttgcatagcccccaaagtagtaattggatagtcttgggggaaatatgcatttcagtggtgaaaacccctgtaaattcaatatatttggcttttgtggaaaattttcctcatggggtgaagtctaagccttagtttctgtattatcatgagagatgacaccagctgcttagcacaaggtggcgccaatgagctttagaataagttgggcttgaccacttgggccattgttttcctgcttcctcccttcaagcctcacctccccagctcccagcttctactgaacaaggctgaaaacccactctattgcagggaaagggaaagattaatgaaaaatgtcagtttcttaagtcagcactggtgaaactttcctaaaacaggaatggcgtttgctgagttttctctggggtctctgctttctgcagctagcttccctgcttgactgcctagaaggcctctgctttcgggtttccatctctttcccctccagaggaccctacagcctaggcgggaggtggttaaggcttctggctgctgtgcaatggggccatctgtgtttgatcaatcctggcggaaaggagggggtgggggttgtaaagagaactgaaagcattccagagtagtgagagagacccagagatcagg

SEQ ID NO: 30 - DNA, 1779 bp, Artificial Sequence
  Progranulin isoform 1
atgtggacactggtgagttgggtggcattgacagcaggtctggtagcaggcacaagatgtccagatggtcagttttgtccagtggcttgttgtcttgaccctgggggtgcctcatatagttgctgccgcccactgctcgataagtggcctaccacactgagtcgccacttgggtggtccttgtcaagttgatgcccattgttcagcaggacattcatgcatctttaccgtaagcggaaccagttcctgttgcccatttccagaggcagtagcgtgtggggacggtcaccactgttgcccaaggggattccactgctcagccgacggcaggagttgtttccagaggagcggcaataactctgtcggggcaatccagtgtccagacagccagtttgagtgccctgacttttctacttgctgcgtgatggttgacggctcctggggttgctgtccaatgccccaggcaagttgctgtgaggatagagtgcactgctgcccccacggcgcattttgcgacctggttcatactaggtgtatcacccccactggtacacaccccctcgctaaaaagctcccagctcagagaactaacagagccgttgcgttgtcatcctccgtcatgtgtcctgatgcacggagcagatgtccagatggaagcacatgctgtgagctgccaagcggcaagtacggctgttgtccgatgcccaacgccacctgttgttcagaccaccttcattgttgtccacaggacaccgtgtgtgacctcattcagagcaaatgcctgagtaaagaaaacgcgactacagacctcctcacaaagttgcccgcgcacacagtgggggatgtgaaatgtgacatggaagtgtcctgtcctgacgggtacacttgctgtagacttcagtctggcgcgtgggggtgctgtccttttacccaggcagtgtgctgtgaagaccacattcattgttgccccgcagggttcacatgtgacacacaaaagggaacctgcgaacagggtcctcaccaggtgccttggatggagaaagccccagcacatctgtcactgcctgaccctcaagctttgaaacgggacgtgccttgcgacaatgtgtcttcctgtccttcatctgacacgtgctgccaattgacaagtggagagtggggatgctgccccattccagaggccgtctgttgcagcgaccatcagcactgttgtccccagggatacacgtgtgtggccgagggacagtgtcagcgagggagtgagattgtggctggtctggaaaagatgccagcaagaagagcgtccctttctcatcccagggatattggctgtgaccaacacactagctgtcctgtgggtcagacatgttgccccagtctgggtggttcatgggcctgctgccagctccctcatgccgtttgttgcgaagaccgccagcattgctgtccagcgggatacacatgcaacgtgaaggcacggagctgtgaaaaggaagtcgtatcagcacagcccgcaacttttctcgctcgctccccccatgtgggggtaaaggacgtggagtgtggtgagggccatttctgccacgataaccagacatgttgccgcgataatcgccagggttgggcctgctgtccctacagacagggagtctgctgtgctgataggcgacattgttgtccagcaggatttaggtgtgctgcgagaggcacgaaatgcctgaggcgggaggctccaagatgggatgcacctcttcgggacccagctctcaggcaactgctg

SEQ ID NO: 31 - DNA, 1314 bp, Artificial Sequence
  Progranulin isoform 2
atgtggaccctcgtatcttgggttgctcttacagcaggtctcgttgcagggactagatgtcctgacggacagttttgccctgtggcatgttgtctggacccaggcggagcatcctactcatgttgtcgccccctcctggacaagtggcctactactctgtcaagacatttgggaggaccctgtcaagttgacgcccattgttccgcaggacactcttgcatctttactgtctctgggacaagctcctgttgcccatttcctgaagccgtggcttgcggcgacggacatcactgttgtccacgagggttccattgctcagcagatggcagaagctgttttcaaaggagtgggaacaattccgtaggcgcaattcagtgcccagattcacagttcgagtgccctgatttcagcacctgctgtgtgatggtcgatgggtcatggggatgctgccctatgccacaagcctcttgttgtgaggaccgagtccactgttgccctcatggcgcattttgtgacctggtgcatacaaggtgcatcacgcccacaggaacccacccccttgctaaaaaacttccagcacaaaggacaaacagagccgttgctctctcttcttccgtgatgtgccccgacgccagaagcagatgcccagatggctcaacttgttgcgagctgcctagtgggaagtacgggtgctgcccaatgccaaatgccacgtgttgctctgaccaccttcactgctgcccgcaagatactgtgtgcgacctgattcagtccaaatgtttgtctaaggaaaatgccaccaccgacctgctgacaaaacttcccgctcacacagtgggggacgtgaaatgtgatatggaggtttcatgccccgacgggtatacatgttgtagattgcagagcggcgcatggggatgttgtccattcacccaggctgtttgttgcgaggaccacatccactgttgtcctgctgggttcacatgcgacacacaaaaaggcacttgcgagcaaggaccacaccaggttccttggatggagaaagccccagcccacctgtctttgcctgaccctcaggctttgaagcgcgatgtaccctgcgacaacgtttcttcctgtccctcctccgatacatgttgcagagacaatagacagggatgggcttgttgtccttatcgacagggcgtttgctgcgctgataggaggcattgttgtcccgcaggttttcggtgcgctgctaggggaacaaaatgtctgagacgggaggctccaagatgggatgctcctttgcgcgaccccgctctgcgacaactgctc

SEQ ID NO: 32 - DNA, 1239 bp, Artificial Sequence
  Progranulin isoform 3
atggcaataactgctgcacatggggcctctacagccgtgcaaactggagacccagcaagtaaggaccaagttacaaccccctgggtgcccagctctgctcttatcgtatccagtaatgctcgaaccagtccccgagctgtattgtggtctatggccccaggaggggcagctccatgtcctagattgcctgccgtaaagacaggatgcaccgctgtatgcgacctcatccaatctaagtgtctgtctaaagaaaacgcaactacagacctgcttacgaagctgcccgctcataccgtaggggacgtcaaatgtgatatggaggtatcctgtcctgacggctatacatgttgtcgcttgcagagtggtgcttggggatgttgtcccttcacacaggccgtgtgttgtgaggaccacatacactgctgccctgctgggttcacatgtgacacgcagaaggggacatgtgagcagggaccccatcaagtaccttggatggagaaggccccagctcatctgagtctgcctgacccccaggcactgaagagagatgtgccctgtgacaacgtgtcctcctgcccctcatctgacacttgctgccagttgacatctggcgaatggggctgctgccctatacccgaagctgtgtgttgtagtgaccaccaacactgctgcccccagggatacacctgtgtggctgagggacagtgccagaggggttccgaaattgttgctggcttggagaagatgcctgctcggagagcctcacttagccatccacgcgatattgggtgtgaccagcacacctcctgtcctgtcggacagacatgttgcccgtcccttggtggaagttgggcttgttgtcagctcccccatgccgtttgttgcgaggaccgacagcactgttgtccagcaggctatacctgcaatgtgaaagctaggagctgcgagaaggaggttgttagcgctcaaccagcaacatttctggcccgctccccccatgtcggtgtgaaagatgttgaatgtggcgagggacacttctgccacgacaaccagacttgctgtagggacaaccgccagggttgggcttgctgtccatatagacagggggtgtgctgtgctgataggagacattgttgtcctgctggatttagatgtgcggctagaggcactaaatgtttgcgaagggaagctcctagatgggatgctccacttagagacccagccctgcggcaattgctc

SEQ ID NO: 33 - protein, 540 aa, Artificial Sequence
  IL-12 beta and alpha subunit connected with a linker
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 34 - protein, 15 aa, Artificial Sequence
  Linker sequence
GGGGSGGGGSGGGGS

SEQ ID NO: 35 - DNA, 498 bp, Artificial Sequence
  IFN-gamma
atgaagtacacctcctacatcctcgcttttcaactgtgcattgtccttgggtctcttggatgttactgtcaagacccatacgtgaaagaggcagagaacctcaaaaagtatttcaatgctggacatagcgacgtggccgataatggcactctcttcctgggcatcctgaagaactggaaggaagaatctgaccgcaagattatgcagtcccagattgtgtccttttatttcaaactcttcaagaatttcaaagatgaccagagcattcagaaaagcgtggaaacaatcaaagaggatatgaacgtgaagtttttcaattcaaataagaagaagcgcgatgactttgagaaacttaccaactattccgtgaccgacttgaatgtgcagaggaaggccatacatgagttgatacaagttatggctgaactgagccccgccgctaaaactggtaaaaggaagcgcagccaaatgctgtttcgagggaggcgcgccagtcag

(19) United States
(12) Patent Application Publication
(10) Pub. No.: US 2023/0227847 A1
(43) Pub. Date: Jul. 20, 2023

(54) VIRAL VECTORS EXPRESSING THERAPEUTIC PROTEINS SPECIFICALLY IN MYELOID CELLS AND MICROGLIA

(71) Applicant: UNIVERSITÄT ZÜRICH, Zürich (CH)

(72) Inventors: Ulrich SILER, Leymen (FR); Janine REICHENBACH, Zürich (CH); Martina NUBIE, Zürich (CH)

(21) Appl. No.: 17/998,587

(22) PCT Filed: Apr. 7, 2021
(86) PCT No.: PCT/EP2021/059070

§ 371 Date: Nov. 11, 2022

(30) Foreign Application Priority Data
May 27, 2020 (EP) 20176939.5

(51) Int. Cl. C12N 15/86; C07K 14/54; C07K 14/57; C07K 14/535; C07K 14/55; C07K 14/56; A61P 25/28

(52) U.S. Cl. CPC C12N 15/86; C07K 14/5434; C07K 14/57; C07K 14/535; C07K 14/55; C07K 14/5443; C07K 14/56; A61P 25/28

(57) ABSTRACT

The present invention provides novel viral vectors for use in human therapy, particularly for use in in the treatment of a disease or disorder which has its origin in the brain or is brain based, particularly a PGRN-associated neurodegenerative disease or disorder including frontotemporal degenerative disease or disorder such as Alzheimer's disease, amyotrophic lateral sclerosis, and Parkinson's disease. The invention also provides viral vectors for use in the treatment of brain tumors, particularly brain tumors selected from the group consisting of glioblastoma, glioma, ganglioneuroblastoma, astrocytoma, oligodendroglioma, PNET (primitive neuroectodermal), medulloblastoma, CNS lymphoma, and neuroblastoma, or any other CNS tumor and further in the treatment of brain metastasis, originating from any forms of breast, lung, colon, testicular, renal carcinomas and melanoma, or any other solid tumor, and any hematologic tumor, comprising all forms of leukemia and lymphomas. Further, the viral vectors may be used in the treatment of autoimmune diseases, inflammatory diseases and/or allergic diseases.

### RELATED APPLICATIONS

The instant application is a 35 U.S.C. § filing of International Patent Application No. PCT/EP2021/059070, filed Apr. 7, 2021, which claims priority to European Patent Application No. 20176939.5, filed May 27, 2020, the entire contents of which are incorporated herein by reference for all purposes.

### SEQUENCE LISTING

The instant application contains a Sequence Listing which has been submitted electronically in ASCII format and is hereby incorporated by reference in its entirety. Said ASCII copy, created on Nov. 11, 2022, is named 732949_VSO9-001US_ST25.txt and is 68,870 bytes in size.

The present invention provides novel viral vectors for use in human gene therapy, particularly for use in the treatment of a disease or disorder which has its origin in the brain or is brain-based, particularly a PGRN-associated neurodegenerative disease or disorder including frontotemporal degenerative disease or disorders such as Alzheimer's disease, amyotrophic lateral sclerosis, and Parkinson's disease. The invention also provides viral vectors for use in the treatment of brain tumors, particularly brain tumors selected from the group consisting of glioblastoma, glioma, ganglioneuroblastoma, astrocytoma, oligodendroglioma, PNET (primitive neuroectodermal), medulloblastoma, CNS lymphoma, and neuroblastoma, or any other CNS tumor and further in the treatment of brain metastasis, originating from any forms of breast, lung, colon, testicular, renal carcinomas and melanoma, or any other solid tumor, and/or any hematologic tumor, comprising all forms of leukemias and lymphomas.

### BACKGROUND OF THE INVENTION

Gene therapy for treatment of human diseases comprises all methods of genetic manipulation of isolated cells ex vivo, or of cells and tissues in vivo. First clinically successful gene therapy studies were published in 2000, addressing hematopoietic stem cells (HSCs) to treat children with a life-threatening inborn defect of the immune system (Cavazzana-Calvo et al. (2000) Science 288: 669-72). These studies were based on ex vivo manipulation of HSCs within a CD34+ bone marrow cell population, using gammaretroviral gene therapy vectors.

Retroviral gene therapy vectors are viral vectors in which single stranded RNA, comprising viral vector RNA sequences and the RNA sequence, encoding the therapeutic protein sequence (i.e. healthy copy of the patient's diseased gene), are incorporated in and transported by retroviral particles. Within one gene therapy retroviral particle, the two RNA molecules, as well as viral proteins required for reverse transcription to double stranded DNA, are enclosed by a capsid structure that consists of viral proteins. The viral capsid is enclosed in a viral envelope, which has the capacity to fuse with the cellular membrane of the target cell during the transduction process. The retroviral proteins enable the reverse transcription of the transported therapeutic RNA sequence to double stranded DNA, which is then transported into the nucleus of transduced cells and integrated into the genome of the transduced target cell.

Neurodegenerative dementia is an important cause of disability in middle aged and elderly patients, leading to loss of physical and social independence. Not only the treatment, but also the daily care at home or in nursery home, are a great challenge to families, the medical staff, and society. The prevalence of dementia in people aged over 60 years is estimated to be 5-7%, with more than 35 million people affected worldwide in 2010.

Overall, up to 20% of all patients with dementia onset under 65 years of age are affected by frontotemporal dementia (FTD). A study estimated the prevalence to range between 15 and 22/100,000 (Onyike & Diehl-Schmid (2013) Int Rev Psychiatry 25: 130-137), with an overall incidence of 2.7-4.1 new cases per 100,000 (Onyike & Diehl-Schmid (2013) Int Rev Psychiatry 25: 130-137). In two UK counties, the prevalence showed a peak of 42.6/100,000 between 65 and 69 years of age. Curative treatment options for neurodegenerative dementia including FTD currently do not exist.

Various estimations exist on the proportion of mutations in the GRN gene, encoding the granulin precursor protein, or progranulin (PGRN), to all FTD cases. These estimations range roughly from 5% (Gass et al. (2006) Hum Mol Genet. 15: 2988-3001; Le Ber et al. (2007) Hum Mutat. 28: 846-55) to 30% (Bunessi et al. (2009) Neurobiology of Disease 33: 379-385) with a penetrance of ⅓ in individuals below the age of 65, and ⅔ in individuals above 65 years of age.

All GRN mutations identified in patients have been associated with loss-of-function and haploinsufficiency, with the consequence of lower levels of PGRN. This fact makes PGRN-deficient FTD a suitable target for therapeutic approaches that aim at restoring physiological levels of PGRN.

PGRN is mainly expressed in microglia, the brain resident counterpart of tissue-resident macrophages. In none of the three reported animal studies (Arrant et al. (2018) J Neurosci. 38: 2341-58; Arrant et al. (2017) Brain 140: 1447-65; Amado et al. (2019) Mol Ther. 27: 465-478) using gene therapeutic approaches, PGRN expression in microglia could be restored: In the studies reported in the prior art, AAV viral gene therapy vectors were injected into mouse brain, leading to PGRN expression in neurons, but not in microglia. In addition, in the latest animal study, strong PGRN overexpression was associated with signs of neuronal toxicity.

There is, therefore, a need for alternative treatment strategies, aiming for physiological PGRN expression in brain microglia. This targeting strategy is different to above mentioned earlier attempts using AAV viral vectors, which led to neuronal PGRN over-expression and neuronal toxicity.

Further, there is a need in the art for safer strategies to express transgenes in myeloid cells, in particular after transduction of HSCs, in the peripheral blood, peripheral tissues, and in the brain/CNS.

### SUMMARY OF THE INVENTION

The present invention provides such alternative and improved strategies, which are defined in the various embodiments described herein and in the claims.

In a particular embodiment, the invention relates to a viral vector comprising a nucleic acid molecule encoding a therapeutic polypeptide or a combination of therapeutic polypeptides under control of a promoter or promoter fragment, wherein the promoter or promoter fragment drives expression of the therapeutic protein or the combination of therapeutic proteins in myeloid cells and microglia, and wherein the promoter or promoter fragment is inactive in hematopoietic progenitor and/or stem cells.

That is, the invention is based on the surprising identification of promoters that can drive expression of a transgene in myeloid cells and microglia, but are silent in stem cells, in particular in hematopoietic stem cells and hematopoietic stem and progenitor cells. Such cell-specific promoters are advantageous in cell and gene therapy applications, since they restrict vector activity with concomitant transgene expression to differentiated target cells, i.e. myeloid cells and microglia. This is of particular importance, since promoter/enhancer activity in undifferentiated stem cells may lead to complications, such as oncogene transactivation, clonal dominance, chromosomal instability, monosomy 7 or leukemic transformation, and transgene expression in undifferentiated stem cells may lead to impaired cellular function or immune reactions. Accordingly, the promoters of the invention are advantageous to ubiquitous promoters, since they can significantly increase the precision and safety of cell and gene therapy applications.

It has been shown that gene therapy in haematopoietic stem cells accompanied by busulfan mediated bone marrow conditioning results in at least partial reconstitution of the myeloid compartment in brain by cells derived from gene modified haematopoietic stem cells graft (Biffi et al. (2013) Science 341:1233158). Hence there is a need in the art of promoters facilitating and restricting transgene expression to haematopoietic phagocytes and to brain myeloid cells i.e. to microglia. The inventors surprisingly identified promoters that drive expression of transgenes in myeloid cells and microglia. The term “myeloid cells” as used herein refers to a series of bone marrow-derived cell lineages including granulocytes (neutrophils, eosinophils, and basophils), monocytes, macrophages, Kupffer cells and mast cells. Furthermore, peripheral blood dendritic cells of myeloid origin, and dendritic cells and macrophages derived in vitro from monocytes in the presence of appropriate culture conditions, are also included.

The term “microglial cell” or “microglia”, as used herein, refers to a class of glial cells involved in the mediation of an immune response within the central nervous system by acting as macrophages. Microglial cells are capable of producing exosomes, cytokines, chemokines, and neurotrophic factors, and further include different forms of microglial cells, including amoeboid microglial cells, ramified microglial cells and reactive microglial cells. Microglial cells include reactive microglia, which are defined as quiescent ramified microglia that transform into a reactive, macrophage-like state and accumulate at sites of brain injury and inflammation to assist in tissue repair and neural regeneration. It is known in the art that hematopoietic stem cells can migrate to the brain and differentiate into macrophages having many characteristics of microglia. Since the promoters of the invention have been demonstrated to be active in macrophages and microglia, it is at least plausible that these promoters will also be active in hematopoietic stem cell (HSC)-derived microglia-like cells.

While myeloid cells in peripheral blood exclusively derive from HSCs, tissue-resident macrophages and microglia are believed to arise solely from yolk sac erythromyeloid precursors under normal conditions. Based on this different origin of peripheral blood myeloid cells and microglia, it can be considered surprising that the promoters of the invention can drive expression in both cell types.

Importantly, the promoters of the invention do not drive expression in stem cells or in progenitor cells. In particular, the promoters of the invention do not drive expression in hematopoietic stem cells and hematopoietic stem and progenitor cells (HSPCs) (see FIG. 21).

As used herein, the term “hematopoietic stem and progenitor cell” or “HSPC” refers to a cell identified by the presence of the antigenic marker CD34 (CD34+) and are therefore characterized as CD34+ cells, and populations of such cells. In particular embodiments, the term “HSPC” refers to a cell identified by the presence of the antigenic marker CD34 (CD34+) and the absence of lineage (lin) markers and are therefore characterized as CD34+/Lin(−) cells, and populations of such cells. It is recognized that the population of cells comprising CD34+ and/or Lin(−) cells also includes hematopoietic progenitor cells, and so for the purposes of this application the term “HSPC” includes hematopoietic stem cells and hematopoietic progenitor cells.

The skilled person is aware of methods to determine whether a promoter is active in a specific cell type or not. For example, to determine whether a promoter is active in a specific cell type, cells of the respective cell type may be transduced with a viral vector comprising a fluorescence marker under control of the promoter of interest. Whether a promoter drives expression of the fluorescence marker can, for example, be detected by flow cytometry. That is, if a fluorescence marker can be detected in transduced cells in sufficient amounts, a promoter is said to drive expression of a transgene in this cell type. If however, none or only minuscule amounts of the fluorescence marker can be detected in a transduced cell, a promoter is said not to drive expression in this cell type. The skilled person is further aware that cells may differentiate into other cell types during the transduction procedure. However, the skilled person is aware of specific combinations of cell surface markers to determine the cell type before and after the transduction procedure. The person skilled in the art is aware that the statement of no promoter activity is limited by the sensitivity of promoter driven transgene product detection and that fluorescent proteins especially e.g. EGFP with high quantum yield as transgene products can be detected with high sensitivity. Hence, the absence of fluorescent protein detection in these kinds of expression experiments is accepted as indication for a promoter below limit of detection and most likely without biological relevance.

The promoter of the invention can drive expression of a transgene encoding a therapeutic protein or a combination of therapeutic proteins in myeloid cells and microglia. That is, the promoter of the invention is operably linked to the transgene. As used herein, the term “operably linked” refers to a functional relationship between two or more nucleic acid (e.g., DNA) segments. Typically, it refers to the functional relationship of a transcriptional regulatory sequence to a transcribed sequence. For example, a promoter sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting.

The transgene may be any nucleic acid that encodes a protein or a functional RNA. Preferred examples of transgenes are discussed below.

In a particular embodiment, the invention related to the viral vector according to the invention, wherein the promoter is
a) a miR223 promoter or a promoter having at least 95%, 96%, 97%, 98%, 99% or 100% sequence identity to the sequence shown in SEQ ID NO: 1, or a functional fragment thereof; or
b) a TMEM119 promoter, or a promoter having at least 95%, 96%, 97%, 98%, 99% or 100% sequence identity to the sequence shown in SEQ ID NO: 3, SEQ ID NO:23 or SEQ ID NO:24, or a functional fragment thereof; or
c) a P2RY12 promoter, or a promoter having at least 95%, 96%, 97%, 98%, 99% or 100% sequence identity to the sequence shown in SEQ ID NO: 2, SEQ ID NO: 21 or SEQ ID NO: 22, or a functional fragment thereof; or
d) an OLFML3 promoter, or a promoter having at least 95%, 96%, 97%, 98%, 99% or 100% sequence identity to the sequence shown in SEQ ID NO: 4 or SEQ ID NO:25, or a functional fragment thereof; or
e) an ITGAM promoter or a promoter having at least 95%, 96%, 97%, 98%, 99% or 100% sequence identity to the sequence shown in SEQ ID NO: 6, or a functional fragment thereof; or
f) an AIF1 promoter or a promoter having at least 95%, 96%, 97%, 98%, 99% or 100% sequence identity to the sequence shown in SEQ ID NO: 5, or a functional fragment thereof; or
g) a fusion promoter comprising a miR233 promoter, or a promoter having at least 95%, 96%, 97%, 98%, 99% or 100% sequence identity to the sequence shown in SEQ ID NO: 1, or a functional fragment thereof; operably linked to
   i) a TMEM119 promoter, or a promoter having at least 95%, 96%, 97%, 98%, 99% or 100% sequence identity to the sequence shown in SEQ ID NO: 3, SEQ ID NO:23 or SEQ ID NO:24, or a functional fragment thereof; and/or
   ii) a P2RY12 promoter, or a promoter having at least 95%, 96%, 97%, 98%, 99% or 100% sequence identity to the sequence shown in SEQ ID NO: 2, SEQ ID NO: 21 or SEQ ID NO:22, or a functional fragment thereof; and/or
   iii) an OLFML3 promoter, or a promoter having at least 95%, 96%, 97%, 98%, 99% or 100% sequence identity to the sequence shown in SEQ ID NO: 4 or SEQ ID NO:25, or a functional fragment thereof; and/or
   iv) an ITGAM promoter, or a promoter having at least 95%, 96%, 97%, 98%, 99% or 100% sequence identity to the sequence shown in SEQ ID NO:6, or a functional fragment thereof; and/or
   v) an AIF1 promoter or a promoter having at least 95%, 96%, 97%, 98%, 99% or 100% sequence identity to the sequence shown in SEQ ID NO:5, or a functional fragment thereof.

That is, in certain embodiments, the promoter is the promoter miR223 or a functional fragment thereof. The term “miR223 promoter” refers to the sequence of SEQ ID NO: 1 and/or any fragment thereof of at least 200 nucleotides, and/or to any sequence or fragment thereof of at least 200 nucleotides with a sequence identity of >95%.

(SEQ ID NO: 1)
ACTTGTACAGCTTCACAGGGCTCCATGCTTAGAAG
GACCCCACACTTAGTTTAATGTTCTGCTGTCATCA
TCTTGATATTCTTAATTTTTAAATAAAGGGCCTAT
CGTTTTCATTTTTTACTGGGCCTTGCAAATTATGT
AGCTGGTTCTGTATGCCAGGAGAGAAGTTGGAAGT
AAAATGGTATTCCAGGACCAGGAGGCATTCTGGCA
GAGTGAAAGAACATGTGATTTGGAGTCCATGGGGA
TGGGTTTAAATTTCAGCTTTCCACTAATTTGCTTT
GTGATACTGAGTATTTCCTTTTATCCCTCAGAGGC
TCTGTTTCTCAATTTTGACTACGGGTTTTTTCATT
AGATAATGTCTCAGTTCTGGTATTCCAGGTTTCCC
TCAATTATTCTGGGAAAACCTCCTTGACCCACAGG
CAGAGCCTAGGGCAGCCAGGTGCTTTCTACTCTCT
CTCTCTCTGCAGCTTGGAAAGTTAGTGTCTGTTGA
AGGTCAGCTGGGAGTTGGTGGAGGCAGGGCAGTGG
CCTGCTACTATTGCTGCAGTAGCAGACCCTTTCAC
AACAGCATTGTTTTGTCATTTTGCATCCAGATTTC
CGTTGGCTAACCTCAGTCTTATCTTCCTCATTTCT
GTTTCCTGTTGAAGACACCAAGGGCCCTTCAAAAC
ACAGAAGCTTCTTGCTCACGGCAGAAAGCCCAATT
CCATCTGGCCCCTGCAGGTTGGCTCAGCACTGGGG
AATCAGAGTCCCCTCCATGACCAAGGCACCACTCC
ACTGACAG

It has been shown herein that the promoter miR223 drives expression in various myeloid cell types (FIGS. 11A-11B and FIG. 18), but according to literature not in microglia. The inventors surprisingly detected miR223 promoter activity in an immortalized microglia cell line (FIGS. 13A-13B).

The miR223 promoter may have the sequence of SEQ ID NO:1. However, the skilled person is aware that fragments and/or sequence variants of SEQ ID NO:1 may have the same characteristics as the miR223 promoter.

Accordingly, the term “miR223 promoter” also extends to functional fragments of the miR223 promoter. A functional fragment of the miR223 promoter is a nucleotide sequence comprising at least 20, at least 30, at least 40, at least 50, at least 60, at least 70, at least 80, at least 90, at least 100, at least 150, at least 200, at least 300, at least 400, at least 500, at least 600 or at least 700 consecutive nucleotides of SEQ ID NO:1. Functional fragments of the miR223 promoter are defined to drive expression in the same cell types and at comparable levels as the promoter shown in SEQ ID NO:1.

It is further to be understood that the invention encompasses promoters comprising two or more functional fragments of the miR223 promoter. That is, in certain embodiments, a promoter may comprise two different nucleotide sequences comprising at least 20, at least 30, at least 40, at least 50, at least 60, at least 70, at least 80, at least 90, at least 100, at least 150, at least 200 or at least 300 consecutive nucleotides of SEQ ID NO:1. In certain embodiments, a promoter may comprise three different nucleotide sequences comprising at least 20, at least 30, at least 40, at least 50, at least 60, at least 70, at least 80, at least 90, at least 100, at least 150 or at least 200 consecutive nucleotides of SEQ ID NO:1. In certain embodiments, a promoter may comprise four different nucleotide sequences comprising at least 20, at least 30, at least 40, at least 50, at least 60, at least 70, at least 80, at least 90 or at least 100 consecutive nucleotides of SEQ ID NO:1.

The term “miR223 promoter” also extends to promoters having the promoter functionality of the miR223 promoter. A promoter is said to have the functionality of the miR223 promoter, if it drives expression in the same cell types and at comparable levels and if it comprises at least a certain degree of sequence similarity to the miR223 promoter.

A promoter is said to have a certain degree of similarity to the miR223 promoter, if the promoter comprises a consecutive stretch of at least 20, at least 30, at least 40, at least 50, at least 60, at least 70, at least 80, at least 90, at least 100, at least 150, at least 200, at least 300, at least 400, at least 500, at least 600 or at least 700 nucleotides of SEQ ID NO:1.

Alternatively, a promoter is said to have a certain degree of similarity to the miR223 promoter, if the promoter has at least 80%, at least 85%, at least 90%, at least 95% sequence identity to the sequence shown in SEQ ID NO:1.

Further, a promoter may be determined to have a certain degree of similarity to the miR223 promoter, if the promoter comprises a consecutive stretch of at least 100, at least 150, at least 200, at least 300, at least 400, at least 500, at least 600 or at least 700 nucleotides of SEQ ID NO:1, wherein said consecutive stretch has at least 95% sequence identity to the corresponding fragment of SEQ ID NO:1.

That is, in certain embodiments, a functional fragment of the miR223 promoter is a nucleic acid sequence of at least 100, 150, 200, 300, 400, 500, 600 or 700 base pairs having at least 95% identity with SEQ ID NO:1, wherein the nucleic acid sequence has miR223 promoter activity.

The term “sequence identity” as used herein is determined by comparing two optimally aligned sequences over a comparison window, where the fragment of the polynucleotide in the comparison window may comprise additions or deletions (e.g., gaps or overhangs) as compared to the reference sequence, which does not comprise additions or deletions, for optimal alignment of the two sequences. The percentage of sequence identity is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (USA) 85: 2444 (1988), by computerized implementations of these algorithms (GAP, BESTFIT, BLAST, PASTA, and TFASTA in the Wisconsin Genetics Software Package, Genetics Computer Group (GCG), 575 Science Dr., Madison, Wis.), or by inspection.

In certain embodiments, the promoter is the promoter ITGAM or a functional fragment thereof. The term “ITGAM promoter” refers to the sequence of SEQ ID NO: 6 and/or any fragment thereof of at least 200 nucleotides, and/or to any sequence or fragment thereof of at least 200 nucleotides with a sequence identity of >95%.

(SEQ ID NO: 6)
CGCACCCAGCCAAGTTTGTACATATATTTTTGACT
ACACTTCTTAACTATTCTTAGGATAAATTACTAGA
AGTGAAAATTCTTGGGTGAAGAGCTTGAGGCCTTT
ACACACACACACACACACACACACAAAAATAGGCT
GGATGCAGTGGCTCACACCTGTAATCTCAGCAGTT
TGGGAGGCTGAGGAAGGAGGATCACTTGAGTCCAG
GAGGTTGAGAATAGCCTGAACAACATAGCAAGATC
TTGTCTCTACAAAAAATTTAAAAAAAATTAGCTGG
CCATGGCAGCATGTGCCTGTAGTACCAGCTACTCG
GAAGGCTGAGGTAGGAGGATCGCTTGAGCCCAGGA
GGTTGATTGAAGCTGCAGTGAGCTGTGATTACACC
ACTGCACTCCAGCCTGGGCAACAGAGCTAGACTCT
GTCTCTAAAAAAAGCACAAAATAATATTTAAAAAG
CACCAGGTATGCCTGTACTTGAGTTGTCTTTGTTG
ATGGCTACAAATGAGGACAGCTCTGGCTGAAGGGC
GCTTCCATTTCCATGGGCTGAAGGAGGGACATTTT
GCAAAGTGTGTTTTCAGGAAGACACAGAGTTTTAC
CTCCTACACTTGTTTGATCTGTATTAATGTTTGCT
TATTTATTTATTTAATTTTTTTTTTGAGACAGAGT
CTCACTCTGTCACCTGGGCTGGAGTGCAGTGGCAT
TATTGAGGCTCATTGCAGTCTCAGACTCCTGAGCT
CAAACAATCCTCCTGCCTCAGCCTCTGGAGTAGCT
AGGACTACAGGCATGTGCCACCATGCCTGGCTAAT
TTTTTAAATGTATTTTTTTGTAGAGTCGGGGTCTC
CCTATGTTGCCCAGGCTGGAGTGCAGTGGTGTGAT
CCTAGCTCACTGCAGCCTGGACCTCGGGCTCAAGT
AATTCTCACACCTCAGCCTGTCCAGTAGCAGGGGC
TACAGGCGCGCACCACCATGCCCAGCTAATTAAAA
ATATTTTTTTGTAGAGACAGGGTCTCTCTATGTTG
CCCAGGCTGGTTTCAAACTCCCAGGCTCAAGCAAT
CCTCCTGCCTTGGCCTCCCAAAGTGCTGGCATTAC
AGGCGTGAGCCACTGCGCCTGGCCCGTATTAATGT
TTAGAACACGAATTCCAGGAGGCAGGCTAAGTCTG
TTCAGCTTGTTCATATGCTTGGGCCAACCCAAGAA
ACAAGTGGGTGACAAATGGCACCTTTTGGATAGTG
GTATTGACTTTGAAAGTTTGGGTCAGGAAGCTGGG
GAGGAAGGGTGGGCAGGCTGTGGGCAGTCCTGGGC
GGAAGACCAGGCAGGGCTATGTGCTCACTGAGCCT
CCGCCCTCTTCCTTTGAATCTCTGATAGACTTCTG
CCTCCTACTTCTCCTTTTCTGCCCTTCTTTGCTTT
GG

It has been shown herein that the promoter ITGAM drives expression is various myeloid cell types (FIGS. 11A-11B and FIG. 18) and in microglia (FIGS. 13A-13B).

The ITGAM promoter may have the sequence of SEQ ID NO:6. However, the skilled person is aware that fragments and/or sequence variants of SEQ ID NO:6 may have the same characteristics as the ITGAM promoter.

Accordingly, the term “ITGAM promoter” also extends to functional fragments of the ITGAM promoter. A functional fragment of the ITGAM promoter is a nucleotide sequence comprising at least 20, at least 30, at least 40, at least 50, at least 60, at least 70, at least 80, at least 90, at least 100, at least 150, at least 200, at least 300, at least 400, at least 500, at least 600 or at least 700 consecutive nucleotides of SEQ ID NO:6. Functional fragments of the ITGAM promoter drive expression in the same cell types and at comparable levels as the promoter shown in SEQ ID NO:6.

It is further to be understood that the invention encompasses promoters comprising two or more functional fragments of the ITGAM promoter. That is, in certain embodiments, a promoter may comprise two different nucleotide sequences comprising at least 20, at least 30, at least 40, at least 50, at least 60, at least 70, at least 80, at least 90, at least 100, at least 150, at least 200 or at least 300 consecutive nucleotides of SEQ ID NO:6. In certain embodiments, a promoter may comprise three different nucleotide sequences comprising at least 20, at least 30, at least 40, at least 50, at least 60, at least 70, at least 80, at least 90, at least 100, at least 150 or at least 200 consecutive nucleotides of SEQ ID NO:6. In certain embodiments, a promoter may comprise four different nucleotide sequences comprising at least 20, at least 30, at least 40, at least 50, at least 60, at least 70, at least 80, at least 90 or at least 100 consecutive nucleotides of SEQ ID NO:6.

The term “ITGAM promoter” also extends to promoters having the promoter functionality of the ITGAM promoter. A promoter is said to have the functionality of the ITGAM promoter, if it drives expression in the same cell types and at comparable levels and if it comprises at least a certain degree of sequence similarity to the ITGAM promoter.

A promoter is said to have a certain degree of similarity to the ITGAM promoter, if the promoter comprises a consecutive stretch of at least 20, at least 30, at least 40, at least 50, at least 60, at least 70, at least 80, at least 90, at least 100, at least 150, at least 200, at least 300, at least 400, at least 500, at least 600 or at least 700 nucleotides of SEQ ID NO:6.

Alternatively, a promoter is said to have a certain degree of similarity to the ITGAM promoter, if the promoter has at least 80%, at least 85%, at least 90%, at least 95% sequence identity to the sequence shown in SEQ ID NO:6.

Further, a promoter may be determined to have a certain degree of similarity to the ITGAM promoter, if the promoter comprises a consecutive stretch of at least 100, at least 150, at least 200, at least 300, at least 400, at least 500, at least 600 or at least 700 nucleotides of SEQ ID NO:6, wherein said consecutive stretch has at least 95% sequence identity to the corresponding fragment of SEQ ID NO:6.

That is, in certain embodiments, a functional fragment of the ITGAM promoter is a nucleic acid sequence of at least 100, 150, 200, 300, 400, 500, 600 or 700 base pairs having at least 95% identity with SEQ ID NO:6, wherein the nucleic acid sequence has ITGAM promoter activity.

In certain embodiments, the promoter is the promoter AIF1 or a functional fragment thereof. The term “AIF1 promoter” refers to the sequence of SEQ ID NO: 5 and/or any fragment thereof of at least 200 nucleotides, and/or to any sequence or fragment thereof of at least 200 nucleotides with a sequence identity of >95%.

(SEQ ID NO: 5)
CGCCTGTAGTCCCAGCTACTCAGGAGGCTGAGGTA
GGAGAATTGCTTGAACCCAGGAGGCAGTGGTTGCA
GTGAGCCGAGATTGCACCATTGCACTCCCGCCTGG
GCGACAGAGCAAGACTCCGACTCAAAAAAAAAAAA
AAAAGCAGCAGCAGCAGCCAGAGGCCACTCCAGCA
TCTCCCCTACCTGGCTTGGGTCAGGGAGAGGGCAG
TGAGAAGTGAAAACTCCCAGCTACAGAAAAGGAAA
TATGTTGGGGGGAAGGGAGAAGGAAAGGTGTCTTC
ATCAATGCCGGGGCAGGGTAGATGGAGCCCTGGGC
AGGGAGTTTGGACCAGGAAATCTCAATGAGGGAAA
TGTGCTGTCCTCACCTCTCCAAGAAGCGACTGGCC
AAACAGAGTGACAGAGGGGATAAAGGTTATGCCTA
GGGAGGCATGTGTCAGAGGCTATCATCCACTCTGT
TGAACCCACAGTGACCAGCACCACCATCACACAAA
CATGCCTGCATGTGTGCACGCACGTGCAGTGTGCA
AACCTGATGTCAGCCTCACTCCCTGGCTCTTCTGT
CCACAAACGCTGTTTCTTTAAGTACCACTTTCAGT
TCCTCCAAAGAATCTACTTAAACTCTTAAATTCCT
GATCTCTATAGATTTTACTAAAGATTTCAAAGGAG
ATAAGATGAGAGGGTTACGTTGCACATTCTAAAGC
AAACAAATTAAAATGTTTTGTTAGACATTTCCATA
TTTTTAAGGGCCTCCTTGGAGCTGCCAGGCTGGGA
GTGAGGTTTCTCTCCCTTTCTAAACCCTGTGCCCA
TCTTGTCACCCTCCTGGAGCTGCCAGCAGACTTCA
GATTCTTCTCCGATCTACAGAGCAGAAAAATTCAG
CCAGCCCTTCCTTGTCTTCCTATCCACAGCTGCCT
GCCCAGACTCATGAAACCTGACAAAATGCAAGGTC
TTATCATTACCTGAACCTTGGACCTGTTCAAAAAT
ACTAGTTCCTGAGAATAAATATCCCTGGTGTCTTC
CTGCCCTTCCTGCACACCTCCAGTGGCTTATCAAA
ATATTTGTTTCATGCGCACACTGGGCTCTCATTTA
AGAGGAATTTGGGAGAATGTTATTTTCTAATCTGC
ATTTCACACCAGGCTCCCCCTCCTTCCTGGGGTGC
TAGTGTCAGCAGAACCTGATGGGGAAGTGAGGTCT
GGGAGGCAGAGGAGGAAGGAATGAGGGGAAAGGGG
AAGTTTGGGAGGAAGGCTTCTG

The AIF1 promoter may have the sequence of SEQ ID NO:5. However, the skilled person is aware that fragments and/or sequence variants of SEQ ID NO:5 may have the same characteristics as the AIF1 promoter.

Accordingly, the term “AIF1 promoter” also extends to functional fragments of the AIF1 promoter. A functional fragment of the AIF1 promoter is a nucleotide sequence comprising at least 20, at least 30, at least 40, at least 50, at least 60, at least 70, at least 80, at least 90, at least 100, at least 150, at least 200, at least 300, at least 400, at least 500, at least 600 or at least 700 consecutive nucleotides of SEQ ID NO:5. Functional fragments of the AIF1 promoter drive expression in the same cell types and at comparable levels as the promoter shown in SEQ ID NO:5.

It is further to be understood that the invention encompasses promoters comprising two or more functional fragments of the AIF1 promoter. That is, in certain embodiments, a promoter may comprise two different nucleotide sequences comprising at least 20, at least 30, at least 40, at least 50, at least 60, at least 70, at least 80, at least 90, at least 100, at least 150, at least 200 or at least 300 consecutive nucleotides of SEQ ID NO:5. In certain embodiments, a promoter may comprise three different nucleotide sequences comprising at least 20, at least 30, at least 40, at least 50, at least 60, at least 70, at least 80, at least 90, at least 100, at least 150 or at least 200 consecutive nucleotides of SEQ ID NO:5. In certain embodiments, a promoter may comprise four different nucleotide sequences comprising at least 20, at least 30, at least 40, at least 50, at least 60, at least 70, at least 80, at least 90 or at least 100 consecutive nucleotides of SEQ ID NO:5.

The term “AIF1 promoter” also extends to promoters having the promoter functionality of the AIF1 promoter. A promoter is said to have the functionality of the AIF1 promoter, if it drives expression in the same cell types and at comparable levels and if it comprises at least a certain degree of sequence similarity to the AIF1 promoter.

A promoter is said to have a certain degree of similarity to the AIF1 promoter, if the promoter comprises a consecutive stretch of at least 20, at least 30, at least 40, at least 50, at least 60, at least 70, at least 80, at least 90, at least 100, at least 150, at least 200, at least 300, at least 400, at least 500, at least 600 or at least 700 nucleotides of SEQ ID NO:5.

Alternatively, a promoter is said to have a certain degree of similarity to the AIF1 promoter, if the promoter has at least 80%, at least 85%, at least 90%, at least 95% sequence identity to the sequence shown in SEQ ID NO:5.

Further, a promoter may be determined to have a certain degree of similarity to the AIF1 promoter, if the promoter comprises a consecutive stretch of at least 100, at least 150, at least 200, at least 300, at least 400, at least 500, at least 600 or at least 700 nucleotides of SEQ ID NO:5, wherein said consecutive stretch has at least 95% sequence identity to the corresponding fragment of SEQ ID NO:5.

That is, in certain embodiments, a functional fragment of the AIF1 promoter is a nucleic acid sequence of at least 100, 150, 200, 300, 400, 500, 600 or 700 base pairs having at least 95% identity with SEQ ID NO:5, wherein the nucleic acid sequence has AIF1 promoter activity.

In certain embodiments, the promoter is the promoter P2RY12 (alias also P2Y12, see https://www.genenames.org/data/gene-symbol-report/#!/hgnc_id/18124) or a functional fragment thereof. The term P2RY12 promoter refers to the sequence of SEQ ID NO: 2 and/or any fragment thereof of at least 200 nucleotides, and/or to any sequence or fragment thereof of at least 200 nucleotides with a sequence identity of >95%.

(SEQ ID NO: 2)
GGTGTTGGAGAGGATGTGGAGAAATAGGAACACTTTTACACTGTTGGTGG
GACTATAAACTAGTTCAACCATTGTGGAAGTCAGTGTGGTGATTCCTCAG
TGATCTAGAACTAGAAATACCATTTAACCCAGCCATCCCATTACTGGGTA
TATACCCAAAGGATTATAAGTCATGCTGCTATAAAGACACATGCACACGT
ATGTTTATTGCGGCACTATTCATAATAGCAAAGACTTGGAACCAACCCAA
AAGTCCAACAATGATAGACTGGATTAAGAAAATGTGGCACATATACACCA
TGGAATACTATGCAGCCATAAAAAATGATGAGTTCATGTCCTTTGTAGGG
ACATGGATGAAATTAGAAATCATCATTCTCAGTAAACTATCGCAAGAACA
AAAAACCAAACACCACATATTCTCACTCATAGGTGGGAACTGAACAATGA
GAACACATGGACACAGGAAGGGAAACACTACACTCTGGGGACTGTTGTGG
GGTGGGGGGATGGGGGAGGGATAGCTTTAGGAGATATACCTAATGCTAAA
TGACGAGTTAATGGGTGTAGCACACCAGCATGGCACATGTATACATATGT
AACTAACCTGCACATTGTGCACATGTACCCTAAAACTTAACGTATAATAA
TAATAAAATTAAAAAAAAAAAGTTAAAGCAGCAAAACACTTTGCCCTTCA
ATCTCACCCCTAACATATTTTTTGCCCTTCTGGTTTCAAAGTTAAACAAC
TGTAAATAATTGTGATACAAGGATGCCTTAATTTAATGTTATATTTTCCC
AAAAACTCAAAGTTAGGTAAAGAAACAAAAAAAAATTGTTTATATTTAAA
TTCTATTCAAGAAAAGCATGAACGACACAGTATATAATAAGCCTGGCAAT
GGATACAATCACTTCTCTAATGTAATTTTGGAATCTGCTAATTTATAATA
GAAGGAAGCTGTTTCACCTACAAAGGAGTTAATCAAACACAGGTTTAAAA
TAATGACATTATTAACCAAGGGAAAAACAAAGGGCCAGAGACTTAACATC
CCTAGCCAGCACGCATTTTGAGTTAACATAATTACTTGTTAGAAGAAAAT
ACATCACCCAGTGTTGTACACAATATATTTCAGATAAATTAACCACCCAA
GAAAGCAAGCTTAAAATCTTCTCCAGGAAGCAGACTTCGAAGGCTTGATC
TCAACTTGGATTTATCATTTGCATAGAAAATAACCATAACTCGAAGTTAT
AAATCATCAACTCTATAGCAGGTTTCAGTAAAAAGCCGCAAGATTTTAAA
TTGCTTTTTAAAAGATGACTTCTCAGCCATCCTCATCCCACATTTCCTGG
GAAATAAAAGCAGAAGTCCTAAAAGAGGACAGATAGAAATTCAGTGTCTG
CATAGCTTTGAGTCCAGTGTTTGA

The P2RY12 promoter may have the sequence of SEQ ID NO:2. However, the skilled person is aware that fragments and/or sequence variants of SEQ ID NO:2 may have the same characteristics as the P2RY12 promoter.

Accordingly, the term “P2RY12 promoter” also extends to functional fragments of the P2RY12 promoter. A functional fragment of the P2RY12 promoter is a nucleotide sequence comprising at least 20, at least 30, at least 40, at least 50, at least 60, at least 70, at least 80, at least 90, at least 100, at least 150, at least 200, at least 300, at least 400, at least 500, at least 600 or at least 700 consecutive nucleotides of SEQ ID NO:2. Functional fragments of the P2RY12 promoter drive expression in the same cell types and at comparable levels as the promoter shown in SEQ ID NO:2. In certain embodiments, the functional fragment of the P2RY12 promoter has the sequence of SEQ ID NO: 21. In certain embodiments, the functional fragment of the P2RY12 promoter has the sequence of SEQ ID NO:22.

It is further to be understood that the invention encompasses promoters comprising two or more functional fragments of the P2RY12 promoter. That is, in certain embodiments, a promoter may comprise two different nucleotide sequences comprising at least 20, at least 30, at least 40, at least 50, at least 60, at least 70, at least 80, at least 90, at least 100, at least 150, at least 200 or at least 300 consecutive nucleotides of SEQ ID NO:2. In certain embodiments, a promoter may comprise three different nucleotide sequences comprising at least 20, at least 30, at least 40, at least 50, at least 60, at least 70, at least 80, at least 90, at least 100, at least 150 or at least 200 consecutive nucleotides of SEQ ID NO:2. In certain embodiments, a promoter may comprise four different nucleotide sequences comprising at least 20, at least 30, at least 40, at least 50, at least 60, at least 70, at least 80, at least 90 or at least 100 consecutive nucleotides of SEQ ID NO:2.

The term “P2RY12 promoter” also extends to promoters having the promoter functionality of the P2RY12 promoter. A promoter is said to have the functionality of the P2RY12 promoter, if it drives expression in the same cell types and at comparable levels and if it comprises at least a certain degree of sequence similarity to the P2RY12 promoter.

A promoter is said to have a certain degree of similarity to the P2RY12 promoter, if the promoter comprises a consecutive stretch of at least 20, at least 30, at least 40, at least 50, at least 60, at least 70, at least 80, at least 90, at least 100, at least 150, at least 200, at least 300, at least 400, at least 500, at least 600 or at least 700 nucleotides from SEQ ID NO:2.

Alternatively, a promoter is said to have a certain degree of similarity to the P2RY12 promoter, if the promoter has at least 80%, at least 85%, at least 90%, at least 95% sequence identity to the sequence shown in SEQ ID NO:2, SEQ ID NO:21 or SEQ ID NO:22.

Further, a promoter may be determined to have a certain degree of similarity to the P2RY12 promoter, if the promoter comprises a consecutive stretch of at least 100, at least 150, at least 200, at least 300, at least 400, at least 500, at least 600 or at least 700 nucleotides from SEQ ID NO:2, wherein said consecutive stretch has at least 95% sequence identity to the corresponding fragment of SEQ ID NO:2.

That is, in certain embodiments, a functional fragment of the P2RY12 promoter is a nucleic acid sequence of at least 100, 150, 200, 300, 400, 500, 600 or 700 base pairs having at least 95% identity with SEQ ID NO:2, wherein the nucleic acid sequence has P2RY12 promoter activity.

In certain embodiments, the promoter is the promoter TMEM119 or a functional fragment thereof. The term “TMEM119 promoter” refers to the sequence of SEQ ID NO: 3 and/or any fragment thereof of at least 200 nucleotides, and/or to any sequence or fragment thereof of at least 200 nucleotides with a sequence identity of >95%.

(SEQ ID NO: 3)
GTTCCTACCCAGAGAGCACGCACTCATCCTTCATGCACTCCCCTGTTCCA
AACCCTCACTGGCTCCGTACTGCCTCCGACCTTCCGAGACTTTAGCCTGG
CTCCTGTCAACATCTCTGACCCTTACTACATGATCCTCTCTTTGGTCCAT
GCTCCAGCCTAATCTAATTGCGGTGGCTTGTGCGTGGTGGCATTCCCAGC
CACCATACCTTTACCCACGCTGGTCCTTCCATGCGGAATGCCTTTCCAGG
GCCTGCTTTGCCCGCTTCTGCTCATACACAGGCATGCCCTCCAGGATGGC
TTCCTACCTCTTTCCCTTGGGGGATTGATCTCTCTGTCTTGGGGTTCTCG
GAGCCCTTGACCTGACCCCTTTCTGTTTGGCAAAAAAGTAATTTACCTCG
GTGTCCTTCTCCCTGGTAGTCTGTGAGCTCCCCAAGGCTGGGCTGTGCCT
GATTCACCTCTGGAACTTGCTTAGCACAGTGCGTGGCCTGCTGCAGGTGT
TCATTGAGCACTTGCCGAATGAATGCATGAATGAATGAATGAATGAATGA
ATGCAAGGGGCTGCTAATCCACAGGACTCCTCAGGTCAGCCAGACGTCCC
GGTTCCAAGGCCTGCCACTGACTCACCTCAGGACCCTGCTTGAACCATTA
GAACTCACCCTGCCTCACTTTCCCCCTCTGTGAAATGGGGCTCCAACTCC
TATTCAAGCTACTATCATTTGGGGGCATTGTGAGGCCACAGATCCCAGAA
CATCAGAGTCAGAGGTAGCCCAGAAAGCTTCCCACCCATCCCTACAAATG
GGAAACTGAGGTCTGGAGAGGGAAGGGCAGAGTTGGGCTCCCTGTCTCAG
GCTCGGACCCACCATCAGGCCTGTCTCTAAAACGAATCCCAGCTCCCACG
CTGCACCCTGAGCCTGGAAGCCTGAGCCACACAAGGACGGGGAATTTTCC
TTCCCACTTCCAGAGGCCTCTGAACCTCCCTGAGCTTGTCCCCTTTGGAG
GGTATTGGGCAGCAGCGTGGGCAGAACCCCAGCTCACTGTCTGGGGGAGC
GCTGCAGGACAGCCTTGTCTGTCTGTCTCAGCCTGCCCTGGGGACCCGAG
GTCAGGGAGGAAGTGCCGCATCTGGTCTTCCCCAGAGCGAGAGTGTGAGC
AAGGGTGGGATTGCGTGTGGCCCGAGAGTAGCCCCTCCCCTCCCCCTGTC
CCCACCCCAAACCCTCTTAATGAAATCAAGCTGGCCCTGCGGCCCAGCCG
GGGAGGGAGGAAGGAGGAGGGACGGGAGGAGGGACGGGAGGAGGGAGGGC
GGGCAGGC

The TMEM119 promoter may have the sequence of SEQ ID NO:3. However, the skilled person is aware that fragments and/or sequence variants of SEQ ID NO:3 may have the same characteristics as the TMEM119 promoter.

Accordingly, the term “TMEM119 promoter” also extends to functional fragments of the TMEM119 promoter. A functional fragment of the TMEM119 promoter is a nucleotide sequence comprising at least 20, at least 30, at least 40, at least 50, at least 60, at least 70, at least 80, at least 90, at least 100, at least 150, at least 200, at least 300, at least 400, at least 500, at least 600 or at least 700 consecutive nucleotides of SEQ ID NO:3. Functional fragments of the TMEM119 promoter drive expression in the same cell types and at comparable levels as the promoter shown in SEQ ID NO:3. In certain embodiments, a functional fragment of the TMEM119 promoter has the sequence of SEQ ID NO:23. In certain embodiments, a functional fragment of the TMEM119 promoter has the sequence of SEQ ID NO:24.

It is further to be understood that the invention encompasses promoters comprising two or more functional fragments of the TMEM119 promoter. That is, in certain embodiments, a promoter may comprise two different nucleotide sequences comprising at least 20, at least 30, at least 40, at least 50, at least 60, at least 70, at least 80, at least 90, at least 100, at least 150, at least 200 or at least 300 consecutive nucleotides of SEQ ID NO:3. In certain embodiments, a promoter may comprise three different nucleotide sequences comprising at least 20, at least 30, at least 40, at least 50, at least 60, at least 70, at least 80, at least 90, at least 100, at least 150 or at least 200 consecutive nucleotides of SEQ ID NO:3. In certain embodiments, a promoter may comprise four different nucleotide sequences comprising at least 20, at least 30, at least 40, at least 50, at least 60, at least 70, at least 80, at least 90 or at least 100 consecutive nucleotides of SEQ ID NO:3.

The term “TMEM119 promoter” also extends to promoters having the promoter functionality of the TMEM119 promoter. A promoter is said to have the functionality of the TMEM119 promoter, if it drives expression in the same cell types and at comparable levels and if it comprises at least a certain degree of sequence similarity to the TMEM119 promoter.

A promoter is said to have a certain degree of similarity to the TMEM119 promoter, if the promoter comprises a consecutive stretch of at least 20, at least 30, at least 40, at least 50, at least 60, at least 70, at least 80, at least 90, at least 100, at least 150, at least 200, at least 300, at least 400, at least 500, at least 600 or at least 700 nucleotides of SEQ ID NO:3.

Alternatively, a promoter is said to have a certain degree of similarity to the TMEM119 promoter, if the promoter has at least 80%, at least 85%, at least 90%, at least 95% sequence identity to the sequence shown in SEQ ID NO:3, SEQ ID NO:23 or SEQ ID NO:24.

Further, a promoter may be determined to have a certain degree of similarity to the TMEM119 promoter, if the promoter comprises a consecutive stretch of at least 100, at least 150, at least 200, at least 300, at least 400, at least 500, at least 600 or at least 700 nucleotides from SEQ ID NO:3, wherein said consecutive stretch has at least 95% sequence identity to the corresponding fragment of SEQ ID NO:3.

That is, in certain embodiments, a functional fragment of the TMEM119 promoter is a nucleic acid sequence of at least 100, 150, 200, 300, 400, 500, 600 or 700 base pairs having at least 95% identity with SEQ ID NO:3, wherein the nucleic acid sequence has TMEM119 promoter activity.

In certain embodiments, the promoter is the promoter OLFML3 or a functional fragment thereof. The term “OLFML3 promoter” refers to the sequence of SEQ ID NO: 4 and/or any fragment thereof of at least 200 nucleotides, and/or to any sequence or fragment thereof of at least 200 nucleotides with a sequence identity of >95%.

(SEQ ID NO: 4)
GCAGTGTCCAGGGTCCTTACTTCACATCATCTGGATCTGACCCTTTGAAA
GAGGTAGAAGACTTCTGAGACCGGCTAATTAAGCTTTGTTTCCTCATATG
TTTTGCCAGATAGCAGTAGCAGAATGAAAAGATGAGTAACCACAGGAAGC
TGCTATTTTTCCCCTCCTTTCAAACTGTACTGTTAGAGTCATGGTCCTTT
TTACAGAAGGAACCTCTCATCAGATTCTGTTGATTCTAAAGTGAATAGAA
TTTCTCCCGATAAAGAAATAGGGGTTTGTTTCGATTAATGACTGCAGGTC
TCTGAGTAAATGCTCTATTTGATTTTTTTTTTCGGCCCGTGTGTCTACCT
TATGGCCCAAGTCTACCTTATGGTGGCCATTAATTCATTTTGGGCTCCTG
CAGCCTTAGTTGGGATATAGAAATGAGAAACACTCAGAAATACCCTTTTG
GACCACAACCAAGAGAAATAACCAATAGTCTTTTCTCCCAGTGGTAAGGA
AGTCAGAATACATTGATCTAGACTGCAACAACATATATATATATATCAGA
TTCCGCCCCCCCGCAATACATGAATGTATAGTAAATTAGTGTGAACTCAC
TGAACACTCCTCAGTTTTGGTGAGAGACTATATCTGGCCTCTTTCAAGCA
AAGGAAAGCCATGTAAAACAGCGCTGCTGTCAGCCTTAACTTCCAGACGA
TCGAGTTAATTTACTAACTTCTCAGTGACCTGTTTTTTTTTTTTTTTTAA
TCTCAGTTATATTTTCTTCCTTGGGCTAAATCAGATATTTGCATAGCCCC
CAAAGTAGTAATTGGATAGTCTTGGGGGAAATATGCATTTCAGTGGTGAA
AACCCCTGTAAATTCAATATATTTGGCTTTTGTGGAAAATTTTCCTCATG
GGGTGAAGTCTAAGCCTTAGTTTCTGTATTATCATGAGAGATGACACCAG
CTGCTTAGCACAAGGTGGCGCCAATGAGCTTTAGAATAAGTTGGGCTTGA
CCACTTGGGCCATTGTTTTCCTGCTTCCTCCCTTCAAGCCTCACCTCCCC
AGCTCCCAGCTTCTACTGAACAAGGCTGAAAACCCACTCTATTGCAGGGA
AAGGGAAAGATTAATGAAAAATGTCAGTTTCTTAAGTCAGCACTGGTGAA
ACTTTCCTAAAACAGGAATGGCGTTTGCTGAGTTTTCTCTGGGGTCTCTG
CTTTCTGCAGCTAGCTTCCCTGCTTGACTGCCTAGAAGGCCTCTGCTTTC
GGGTTTCCATCTCTTTCCCCTCCAGAGGACCCTACAGCCTAGGCGGGAGG
TGGTTAAGGCTTCTGGCTGCTGTGCAATGGGGCCATCTGTGTTTGATCAA
TCCTGGCGGAAAGGAGGGGGTGGGGGTTGTAAAGAGAACTGAAAGCATTC
CAGAGTAGTGAGAGAGA

The OLFML3 promoter may have the sequence of SEQ ID NO:4. However, the skilled person is aware that fragments and/or sequence variants of SEQ ID NO:4 may have the same characteristics as the OLFML3 promoter.

Accordingly, the term “OLFML3 promoter” also extends to functional fragments of the OLFML3 promoter. A functional fragment of the OLFML3 promoter is a nucleotide sequence comprising at least 20, at least 30, at least 40, at least 50, at least 60, at least 70, at least 80, at least 90, at least 100, at least 150, at least 200, at least 300, at least 400, at least 500, at least 600 or at least 700 consecutive nucleotides of SEQ ID NO:4. Functional fragments of the OLFML3 promoter drive expression in the same cell types and at comparable levels as the promoter shown in SEQ ID NO:4. In certain embodiments, a functional fragment of the OLFML3 promoter has the sequence of SEQ ID NO:25.

It is further to be understood that the invention encompasses promoters comprising two or more functional fragments of the OLFML3 promoter. That is, in certain embodiments, a promoter may comprise two different nucleotide sequences comprising at least 20, at least 30, at least 40, at least 50, at least 60, at least 70, at least 80, at least 90, at least 100, at least 150, at least 200 or at least 300 consecutive nucleotides of SEQ ID NO:4. In certain embodiments, a promoter may comprise three different nucleotide sequences comprising at least 20, at least 30, at least 40, at least 50, at least 60, at least 70, at least 80, at least 90, at least 100, at least 150 or at least 200 consecutive nucleotides of SEQ ID NO:4. In certain embodiments, a promoter may comprise four different nucleotide sequences comprising at least 20, at least 30, at least 40, at least 50, at least 60, at least 70, at least 80, at least 90 or at least 100 consecutive nucleotides of SEQ ID NO:4.

The term “OLFML3 promoter” also extends to promoters having the promoter functionality of the OLFML3 promoter. A promoter is said to have the functionality of the OLFML3 promoter, if it drives expression in the same cell types and at comparable levels and if it comprises at least a certain degree of sequence similarity to the OLFML3 promoter.

A promoter is said to have a certain degree of similarity to the OLFML3 promoter, if the promoter comprises a consecutive stretch of at least 20, at least 30, at least 40, at least 50, at least 60, at least 70, at least 80, at least 90, at least 100, at least 150, at least 200, at least 300, at least 400, at least 500, at least 600 or at least 700 nucleotides of SEQ ID NO:4.

Alternatively, a promoter is said to have a certain degree of similarity to the OLFML3 promoter, if the promoter has at least 80%, at least 85%, at least 90%, at least 95% sequence identity to the sequence shown in SEQ ID NO:4 or SEQ ID NO:25.

Further, a promoter may be determined to have a certain degree of similarity to the OLFML3 promoter, if the promoter comprises a consecutive stretch of at least 100, at least 150, at least 200, at least 300, at least 400, at least 500, at least 600 or at least 700 nucleotides of SEQ ID NO:4, wherein said consecutive stretch has at least 95% sequence identity to the corresponding fragment of SEQ ID NO:4.

That is, in certain embodiments, a functional fragment of the OLFML3 promoter is a nucleic acid sequence of at least 100, 150, 200, 300, 400, 500, 600 or 700 base pairs having at least 95% identity with SEQ ID NO:4, wherein the nucleic acid sequence has OLFML3 promoter activity.

In certain embodiments, the promoter is a fusion promoter comprising (a) the miR223 promoter, a fragment thereof or a promoter with miR223 functionality and (b) a second promoter. Besides its specific activity in myeloid cells and microglia, the promoter miR223 is attractive for use in cell and gene therapy applications due to its resistance to DNA methylation. This is important since differentiation of stem cells into myeloid cells or microglia-like cells is known to result in extensive methylation of promoter sequences, which typically leads to the silencing of transgenes in differentiated cells. Accordingly, the promoter miR223 has the advantage that it enables stable transgene expression in differentiated cells that originate from HSCs.

Preferably, the fusion promoter comprises the miR223 promoter, a fragment thereof or a promoter with miR223 functionality and

(a) a TMEM119 promoter, a functional fragment thereof or a promoter with TMEM119 functionality; (b) a P2RY12 promoter, a functional fragment thereof or a promoter with P2RY12 functionality; (c) an OLFML3 promoter, a functional fragment thereof or a promoter with OLFML3 functionality; (d) an ITGAM promoter, a functional fragment thereof or a promoter with ITGAM functionality; or (e) an AIF1 promoter, a functional fragment thereof or a promoter with AIF1 functionality.

The term “miR223 fusion construct” or “miR223 fusion promoter” refers to a promoter construct, consisting of the miR223 promoter,

(i) fused to a P2Y12 promoter or a promoter fragment, derived from the P2Y12 promoter, consisting at least 200 nucleotides of the sequence of the P2Y12 promoter, or;

(ii) fused to a TMEM119 promoter or a promoter fragment, derived from the TMEM119 promoter, consisting at least 200 nucleotides of the sequence of the TMEM119 promoter, or;

(iii) fused to a OLFML3 promoter or a promoter fragment, derived from the OLFML3 promoter, consisting at least 200 nucleotides of the sequence of the OLFML3 promoter, or;

(iv) fused to a AIF1 promoter or a promoter fragment, derived from the AIF1 promoter, consisting at least 200 nucleotides of the sequence of the AIF1 promoter, or;

(v) fused to an ITGAM promoter or a promoter fragment, derived from the ITGAM promoter, consisting at least 200 nucleotides of the sequence of the ITGAM promoter.

That is, in certain embodiments, the fusion promoter comprises a miR223 promoter, or a promoter having at least 95%, 96%, 97%, 98%, 99% or 100% sequence identity to the sequence shown in SEQ ID NO: 1, or a functional fragment thereof; and a P2RY12 promoter, or a promoter having at least 95%, 96%, 97%, 98%, 99% or 100% sequence identity to the sequence shown in SEQ ID NO: 2, SEQ ID NO:21 or SEQ ID NO:22, or functional fragments thereof. It is to be understood that the functional fragments are preferably promoters with miR223 and/or P2RY12 functionality as defined above.

In certain embodiments, the fusion promoter comprising a miR223 promoter and a P2RY12 promoter may comprise the nucleotide sequence SEQ ID NO:26 or SEQ ID NO:27 or a nucleotide sequence having at least 90%, at least 95%, at least 96%, at least 97%, at least 98% or at least 99% sequence identity with SEQ ID NO:26 or SEQ ID NO:27.

In certain embodiments, the fusion promoter comprises a miR223 promoter, or a promoter having at least 95%, 96%, 97%, 98%, 99% or 100% sequence identity to the sequence shown in SEQ ID NO: 1, or a functional fragment thereof, and a TMEM119 promoter, or a promoter having at least 95%, 96%, 97%, 98%, 99% or 100% sequence identity to the sequence shown in SEQ ID NO: 3, SEQ ID NO:23 or SEQ ID NO:24, or functional fragments thereof. It is to be understood that the functional fragments are preferably promoters with miR223 and/or TMEM119 functionality as defined above.

In certain embodiments, the fusion promoter comprising a miR223 promoter and a TMEM119 promoter may comprise the nucleotide sequence SEQ ID NO:28 or a nucleotide sequence having at least 90%, at least 95%, at least 96%, at least 97%, at least 98% or at least 99% sequence identity with SEQ ID NO:28.

In certain embodiments, the fusion promoter comprises a miR223 promoter, or a promoter having at least 95%, 96%, 97%, 98%, 99% or 100% sequence identity to the sequence shown in SEQ ID NO: 1, or a functional fragment thereof; and an OLFML3 promoter, or a promoter having at least 95%, 96%, 97%, 98%, 99% or 100% sequence identity to the sequence shown in SEQ ID NO: 4 or SEQ ID NO:25, or a functional fragment thereof. It is to be understood that the functional fragments are preferably promoters with miR223 and/or OLFML3 functionality as defined above.

In certain embodiments, the fusion promoter comprising a miR223 promoter and an OLFML3 promoter may comprise the nucleotide sequence SEQ ID NO:29 or a nucleotide sequence having at least 90%, at least 95%, at least 96%, at least 97%, at least 98% or at least 99% sequence identity with SEQ ID NO:29.

In certain embodiments, the fusion promoter comprises a miR223 promoter, or a promoter having at least 95%, 96%, 97%, 98%, 99% or 100% sequence identity to the sequence shown in SEQ ID NO: 1, or a functional fragment thereof; and an ITGAM promoter, or a promoter having at least 95%, 96%, 97%, 98%, 99% or 100% sequence identity to the sequence shown in SEQ ID NO: 6, or a functional fragment thereof. It is to be understood that the functional fragments are preferably promoters with miR223 and/or ITGAM functionality as defined above.

In certain embodiments, the fusion promoter comprises a miR223 promoter, or a promoter having at least 95%, 96%, 97%, 98%, 99% or 100% sequence identity to the sequence shown in SEQ ID NO: 1, or a functional fragment thereof; and an AIF1 promoter, or a promoter having at least 95%, 96%, 97%, 98%, 99% or 100% sequence identity to the sequence shown in SEQ ID NO: 5, or a functional fragment thereof. It is to be understood that the functional fragments are preferably promoters with miR223 and/or AIF1 functionality as defined above.

In certain embodiments, the invention relates to the vector according to the invention, wherein the promoter is
a) a miR223 promoter or a promoter having at least 95%, 96%, 97%, 98%, 99% or 100% sequence identity to the sequence shown in SEQ ID NO: 1, or a functional fragment thereof; or
b) an ITGAM promoter or a promoter having at least 95%, 96%, 97%, 98%, 99% or 100% sequence identity to the sequence shown in SEQ ID NO: 6, or a functional fragment thereof; or
c) a fusion promoter comprising a miR233 promoter, or a promoter having at least 95%, 96%, 97%, 98%, 99% or 100% sequence identity to the sequence shown in SEQ ID NO: 1, or a functional fragment thereof; operably linked to a TMEM119 promoter, or a promoter having at least 95%, 96%, 97%, 98%, 99% or 100% sequence identity to the sequence shown in SEQ ID NO: 3, SEQ ID NO:23 or SEQ ID NO:24, or a functional fragment thereof.

In certain embodiments, the invention relates to the vector according to the invention, wherein the promoter is
a) a miR223 promoter or a promoter having at least 95%, 96%, 97%, 98%, 99% or 100% sequence identity to the sequence shown in SEQ ID NO: 1, or a functional fragment thereof; or
b) a fusion promoter comprising a miR233 promoter, or a promoter having at least 95%, 96%, 97%, 98%, 99% or 100% sequence identity to the sequence shown in SEQ ID NO: 1, or a functional fragment thereof.

In certain embodiments, the invention relates to the vector according to the invention, wherein the promoter is
a) a miR223 promoter or a promoter having at least 95%, 96%, 97%, 98%, 99% or 100% sequence identity to the sequence shown in SEQ ID NO: 1, or a functional fragment thereof; or
b) a fusion promoter comprising a miR233 promoter, or a promoter having at least 95%, 96%, 97%, 98%, 99% or 100% sequence identity to the sequence shown in SEQ ID NO: 1, or a functional fragment thereof; operably linked to a TMEM119 promoter, or a promoter having at least 95%, 96%, 97%, 98%, 99% or 100% sequence identity to the sequence shown in SEQ ID NO: 3, SEQ ID NO:23 or SEQ ID NO:24; or a functional fragment thereof.

In certain embodiments, the invention relates to the vector according to the invention, wherein the promoter is
a) a miR223 promoter or a promoter having at least 95%, 96%, 97%, 98%, 99% or 100% sequence identity to the sequence shown in SEQ ID NO: 1, or a functional fragment thereof; or
b) a fusion promoter comprising a miR233 promoter, or a promoter having at least 95%, 96%, 97%, 98%, 99% or 100% sequence identity to the sequence shown in SEQ ID NO: 1, or a functional fragment thereof; operably linked to a TMEM119 promoter, or a promoter having at least 95%, 96%, 97%, 98%, 99% or 100% sequence identity to the sequence shown in SEQ ID NO:23; or a functional fragment thereof.

In certain embodiments, the invention relates to the vector according to the invention, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof; operably linked to (b) a TMEM119 promoter, or a promoter having at least 95%, 96%, 97%, 98%, 99% or 100% sequence identity to the sequence shown in SEQ ID NO:23; or a functional fragment thereof.

In certain embodiments, the invention relates to the vector according to the invention, wherein the promoter is a fusion promoter comprising (a) a miR233 promoter, or a promoter having at least 95%, 96%, 97%, 98%, 99% or 100% sequence identity to the sequence shown in SEQ ID NO: 1; operably linked to (b) a TMEM119 promoter, or a promoter having at least 95%, 96%, 97%, 98%, 99% or 100% sequence identity to the sequence shown in SEQ ID NO:23.

Within the present invention, a promoter is said to have the functionality of a particular promoter (a reference promoter, e.g. miR223 according to SEQ ID NO:1), if, besides the sequence similarities disclosed above, it drives expression in the same cell types and at comparable levels. A promoter is said to drive expression at comparable levels, if the expression level of a reporter gene from said promoter is at least 50%, at least 60%, at least 70%, at least 80% or at least 90% of the expression level of the same reporter gene from the reference promoter under comparable conditions. Numerous reporter genes are known in the art that are suitable to determine whether two promoters have comparable activities and cell specificities.

In a particular embodiment, the invention relates to the viral vector according to the invention, wherein the viral vector comprises at least one transcriptional regulatory element, wherein said at least one transcriptional regulatory element is arranged such that it inhibits or activates a transcriptional activity of the promoter.

That is, the viral vector may further comprise a regulatory element that allows controlling expression of the transgene more precisely. The term ‘transcriptional regulatory element’, as used herein, refers to a nucleic acid fragment capable of regulating the expression of one or more genes, preferably, the transgene. The transcriptional regulatory element may activate or inhibit expression of a transgene. Thus, the transcriptional regulatory element, the transgene and the promoter are operably linked to each other.

It is to be understood that the transcriptional regulatory element is a nucleic acid sequence in close proximity to the promoter of the invention. Preferably, the transcriptional regulatory element constitutes a binding site for a transcriptional activator or repressor. A transcriptional activator is a protein that activates expression of the transgene when bound to the transcriptional regulatory element. A transcriptional repressor is a protein that prevents expression of the transgene when bound to the transcriptional regulatory element.

In certain embodiments, the transcriptional activator or repressor can undergo structural changes that determine the binding potential to the transcriptional regulatory element. For example, a transcriptional activator may only bind to a transcriptional regulatory element and thereby activate expression of the transgene when the activator is specifically bound by an inducer molecule. Alternatively, a transcriptional repressor may only bind to a transcriptional regulatory element and thereby inactivate expression of the transgene when the activator is specifically bound by a repressor molecule.

The skilled person is aware of various systems that may be used for controlling the expression of transgenes from the promoters of the present invention. In a particular embodiment, the invention relates to the viral vector according to the invention, wherein the at least one transcriptional regulatory element comprises a binding site for a transcriptional activator or repressor, in particular wherein the transcriptional activator or repressor comprises:
i) an antibiotic-binding domain, in particular a tetracycline/doxycycline-binding domain, a macrolide-binding domain or a pristinamycin-binding domain;
ii) a hormone-binding domain, in particular a RU486-binding domain or an abscisic acid-binding domain;
iii) a steroid-binding domain, in particular an ecdysone-binding domain; or
iv) a dimerizer system, in particular a rapamycin-based or rapalog-based dimerizer system.

That is, in certain embodiments, the inducer or repressor molecule is an antibiotic or an antibiotic derivative. Specific binding of the antibiotic or the antibiotic derivative to a transcriptional activator or repressor protein may induce or repress expression of a transgene, respectively. Well known examples of regulatory proteins that function as transcriptional activators or repressors are proteins comprising a tetracycline/doxycycline-binding domain, a macrolide-binding domain or a pristinamycin-binding domain.

Alternatively, transcriptional activators or repressors may comprise a binding site for a hormone. In this case, binding of the transcriptional activator or repressor to the transcriptional regulatory element comprised in the viral vector is controlled by the binding of a hormone to the transcriptional activator or repressor. Well known examples of regulatory proteins that function as transcriptional activators or repressors are proteins comprising a RU486-binding domain or an abscisic acid-binding domain.

Alternatively, transcriptional activators or repressors may comprise a binding site for a steroid. In this case, binding of the transcriptional activator or repressor to the transcriptional regulatory element comprised in the viral vector is controlled by the binding of a steroid to the transcriptional activator or repressor. Well known examples of regulatory proteins that function as transcriptional activators or repressors are proteins comprising an ecdysone-binding domain.

In certain embodiments, expression of the transgene may be controlled by a dimerizer system. A dimerizer system is a transcriptional activator that consists of two separate proteins. A first protein comprises a binding site for the transcriptional regulatory element comprised in the viral vector and additionally a drug-binding domain. The second protein comprises another drug-binding domain and an activator or repressor domain that can induce or repress expression of a transgene, respectively. Activation or repression with a dimerizer system only works in the presence of a dimerizer molecule, which can be specifically bound by the drug-binding domains of both proteins and thereby bring the two proteins into close proximity, such that they can induce or repress expression of a transgene. Well known examples of dimerizer systems are rapamycin-based or rapalog-based dimerizer systems.

In a particular embodiment, the invention relates to the viral vector according to the invention, wherein the viral vector encodes a riboswitch, wherein the riboswitch controls translation of an mRNA encoding the therapeutic protein or the combination of therapeutic proteins.

Alternatively or in addition to the transcriptional regulatory element, the viral vector according to the invention may encode a riboswitch that controls translation of the mRNA encoded by the transgene.

The term “riboswitch” as used herein refers to a regulatory segment of an RNA polynucleotide (or the DNA encoding the riboswitch). A riboswitch in the context of the present invention contains a sensor region (e.g., an aptamer) and an effector stem-loop that together are responsible for sensing the presence of a ligand (e.g., a small molecule) and modulating the accessibility of a polyadenylation sequence located in the effector stem-loop.

In a particular embodiment, the invention relates to the viral vector according to the invention, wherein the therapeutic polypeptide is
i) a polypeptide that restores a cellular function and/or elicits a cellular response in a cell or tissue; or
ii) a polypeptide that enables and/or increases target specificity of a cell.

The transgene preferably encodes one or more therapeutic protein(s). Within the present invention, two main types of therapeutic proteins are envisioned.

The first type of therapeutic proteins is a protein that restores a cellular function of a target cell or elicits a cellular response in a target cell. For example, certain diseases are known to be caused by unnaturally low levels of a specific protein or by an inactive mutant variant of a specific protein. Normal protein function in such cells can be restored by delivering a transgene encoding a functional variant of such proteins to these cells. Alternatively, the transgene may encode a protein that elicits a cellular response in the cell that is expressing the transgene or in the surrounding tissues. For example, the transgene may encode a cytokine which elicits a specific response in the target cell. In addition, the cytokine may be secreted out of the target cell, such that it cannot only elicit a response in the target cell, but also in the surrounding tissue.

That is, in a particular embodiments, the invention relates to the viral vector according to the invention, wherein the polypeptide that restores a cellular function and/or elicits a cellular response in a cell comprises at least a fragment of one or more polypeptides selected from the group consisting of: PGRN, Presenilin1, Presenilin 2, IL-2, IL-12, IL-15, IL-21, IFN-alpha, IFN-alpha Receptor, IFN-gamma, IFN-gamma Receptor, FasL/Fas, CD11b, selectins, such as L-Selectin or P-Selectin, PSGL (P-Selectin Ligand), TRAIL, TRAIL-R, Lymphotoxin beta (LT-β), LT-βR, decoyreceptors 1-3, TNF-alpha, TNF-alphaR, MSH, G-CSF, GM-CSF, IL-1, IL-6, IL-7, IL-8, IL31, IL1R, IL31R, IL-10, IL-23, CXCR3 ligands such as CXCL9 and CXCL-10, PD-1, PD-1L, PD-2 (PDC2), PD-2L, Granzyme B, Granulysine, CD11b, TIGIT, CD 112, CD 155, nitric oxide synthase, DNA methyltransferase 3b (DNMT3b), Jumonji domain-containing protein 1A (JMJD1A), somatostatine, histone deacetylases (HDAC) such as HDAC3 or HDAC 9, CSF1 receptor (CSF1R), IL-34, TAM, all chemokines and chemokine receptors, all cytokines and cytokine receptors.

In certain embodiments, the polypeptide that restores a cellular function and/or elicits a cellular response in a cell comprises at least a fragment of one or more polypeptides encoded by the genes MAPT, C9orf72, TDP-43, FUS, CHMP2B, VCP, SQSTM1, UBQLN2, TBK1, OPTN, SOD1, SYT11, FGF20, PM20D1, BST1, GPNMB, APP, PSEN1, and/or PSEN2.

Alternatively, the therapeutic protein may be a protein that directs a target cell to a specific location. For example, the therapeutic protein may be an antigen-binding molecule that directs the transduced cell to a specific cell type or tissue. For example, expressing a protein that specifically binds to a tumor antigen may direct a transduced cell, such as an immune cell, to a tumor. In certain embodiments, the antigen-binding molecule may be or may comprise an antibody. In certain embodiments, the antigen-binding molecule may be or may comprise a fragment of an antibody. In certain embodiments, the antigen-binding molecule may be a chimeric antigen receptor (CAR).

That is, in a particular embodiment, the invention relates to the viral vector according to the invention, wherein the polypeptide that enables and/or increases target specificity of a cell enables and/or increases specificity to a tumor antigen, in particular wherein the tumor antigen is VEGF, a VEGF-Receptor, an antagonist to a metalloproteinase (e.g. MMP-9), CD40/CD40L, EGFR, Annexin1, FGFR-1, Her2, St6galnac5, MMP1-28, TIMPS1-4, Melanotransferrin, alpha4-beta1 Integrin, VCAM-1, E-cadherin, Alpha-v-beta3 integrin, Alpha-v-beta5 integrin, Alpha-v-beta6 integrin, Alpha-v-beta8 integrin, CCND1, BRCA, CEA, cancer-related antigen 72-4 (CA 72-4), cancer-related antigen 19-9 (CA 19-9), WT1, CD11b, L-Selectin, NY-ESO-1, or a fragment thereof.

In a particular embodiment, the invention relates to a viral vector comprising a transgene under control of one or more promoters, wherein the transgene encodes
a) PGRN, or a functional fragment thereof; or
b) a polypeptide having at least 95%, 96%, 97%, 98%, 99% or 100% sequence identity to the sequence shown in SEQ ID NO: 7, SEQ ID NO: 8 or SEQ ID NO: 9, or a functional fragment thereof.

That is, in certain embodiments, the invention relates to a viral vector encoding progranulin (PGRN). The term “Progranulin”, “PGRN”, “Granulin”, “GRN” refers to a protein comprising the protein sequence of SEQ ID NO: 7 and/or the protein sequence of SEQ ID NO: 8, and/or the protein sequence of SEQ ID NO: 9, or any protein fragment derived from protein sequences of SEQ ID NO: 7, of SEQ ID NO: 8 or of SEQ ID NO: 9 with a length of at least 50 amino acids, or to any protein sequence with more than 95% homology thereof. Also provided herein are nucleic acid sequences encoding said proteins.

| Progranulin Isoform 1 (SEQ ID NO: 7) | |
|---|---|
| MWTLVSWVALTAGLVAGTRCPDGQFCPVACCLDPGGASYSCCRPLLDKWPTTLSRHLGGPCQ | |
| VDAHCSAGHSCIFTVSGTSSCCPFPEAVACGDGHHCCPRGFHCSADGRSCFQRSGNNSVGAI | |
| QCPDSQFECPDFSTCCVMVDGSWGCCPMPQASCCEDRVHCCPHGAFCDLVHTRCITPTGTHP | |
| LAKKLPAQRTNRAVALSSSVMCPDARSRCPDGSTCCELPSGKYGCCPMPNATCCSDHLHCCP | |
| QDTVCDLIQSKCLSKENATTDLLTKLPAHTVGDVKCDMEVSCPDGYTCCRLQSGAWGCCPFT | |
| QAVCCEDHIHCCPAGFTCDTQKGTCEQGPHQVPWMEKAPAHLSLPDPQALKRDVPCDNVSSC | |
| PSSDTCCQLTSGEWGCCPIPEAVCCSDHQHCCPQGYTCVAEGQCQRGSEIVAGLEKMPARRA | |
| SLSHPRDIGCDQHTSCPVGQTCCPSLGGSWACCQLPHAVCCEDRQHCCPAGYTCNVKARSCE | |
| KEVVSAQPATFLARSPHVGVKDVECGEGHFCHDNQTCCRDNRQGWACCPYRQGVCCADRRHC | |
| CPAGFRCAARGTKCLRREAPRWDAPLRDPALRQLL | |
| As encoded by the DNA sequence (SEQ ID NO: 30): | |
| 1 | ATGTGGACAC TGGTGAGTTG GGTGGCATTG ACAGCAGGTC TGGTAGCAGG CACAAGATGT CCAGATGGTC |
| 71 | AGTTTTGTCC AGTGGCTTGT TGTCTTGACC CTGGGGGTGC CTCATATAGT TGCTGCCGCC CACTGCTCGA |
| 141 | TAAGTGGCCT ACCACACTGA GTCGCCACTT GGGTGGTCCT TGTCAAGTTG ATGCCCATTG TTCAGCAGGA |
| 211 | CATTCATGCA TCTTTACCGT AAGCGGAACC AGTTCCTGTT GCCCATTTCC AGAGGCAGTA GCGTGTGGGG |
| 281 | ACGGTCACCA CTGTTGCCCA AGGGGATTCC ACTGCTCAGC CGACGGCAGG AGTTGTTTCC AGAGGAGCGG |
| 351 | CAATAACTCT GTCGGGGCAA TCCAGTGTCC AGACAGCCAG TTTGAGTGCC CTGACTTTTC TACTTGCTGC |
| 421 | GTGATGGTTG ACGGCTCCTG GGGTTGCTGT CCAATGCCCC AGGCAAGTTG CTGTGAGGAT AGAGTGCACT |
| 491 | GCTGCCCCCA CGGCGCATTT TGCGACCTGG TTCATACTAG GTGTATCACC CCCACTGGTA CACACCCCCT |
| 561 | CGCTAAAAAG CTCCCAGCTC AGAGAACTAA CAGAGCCGTT GCGTTGTCAT CCTCCGTCAT GTGTCCTGAT |
| 631 | GCACGGAGCA GATGTCCAGA TGGAAGCACA TGCTGTGAGC TGCCAAGCGG CAAGTACGGC TGTTGTCCGA |
| 701 | TGCCCAACGC CACCTGTTGT TCAGACCACC TTCATTGTTG TCCACAGGAC ACCGTGTGTG ACCTCATTCA |
| 771 | GAGCAAATGC CTGAGTAAAG AAAACGCGAC TACAGACCTC CTCACAAAGT TGCCCGCGCA CACAGTGGGG |
| 841 | GATGTGAAAT GTGACATGGA AGTGTCCTGT CCTGACGGGT ACACTTGCTG TAGACTTCAG TCTGGCGCGT |
| 911 | GGGGGTGCTG TCCTTTTACC CAGGCAGTGT GCTGTGAAGA CCACATTCAT TGTTGCCCCG CAGGGTTCAC |
| 981 | ATGTGACACA CAAAAGGGAA CCTGCGAACA GGGTCCTCAC CAGGTGCCTT GGATGGAGAA AGCCCCAGCA |
| 1051 | CATCTGTCAC TGCCTGACCC TCAAGCTTTG AAACGGGACG TGCCTTGCGA CAATGTGTCT TCCTGTCCTT |
| 1121 | CATCTGACAC GTGCTGCCAA TTGACAAGTG GAGAGTGGGG ATGCTGCCCC ATTCCAGAGG CCGTCTGTTG |
| 1191 | CAGCGACCAT CAGCACTGTT GTCCCCAGGG ATACACGTGT GTGGCCGAGG GACAGTGTCA GCGAGGGAGT |
| 1261 | GAGATTGTGG CTGGTCTGGA AAAGATGCCA GCAAGAAGAG CGTCCCTTTC TCATCCCAGG GATATTGGCT |
| 1331 | GTGACCAACA CACTAGCTGT CCTGTGGGTC AGACATGTTG CCCCAGTCTG GGTGGTTCAT GGGCCTGCTG |
| 1401 | CCAGCTCCCT CATGCCGTTT GTTGCGAAGA CCGCCAGCAT TGCTGTCCAG CGGGATACAC ATGCAACGTG |
| 1471 | AAGGCACGGA GCTGTGAAAA GGAAGTCGTA TCAGCACAGC CCGCAACTTT TCTCGCTCGC TCCCCCCATG |
| 1541 | TGGGGGTAAA GGACGTGGAG TGTGGTGAGG GCCATTTCTG CCACGATAAC CAGACATGTT GCCGCGATAA |
| 1611 | TCGCCAGGGT TGGGCCTGCT GTCCCTACAG ACAGGGAGTC TGCTGTGCTG ATAGGCGACA TTGTTGTCCA |
| 1681 | GCAGGATTTA GGTGTGCTGC GAGAGGCACG AAATGCCTGA GGCGGGAGGC TCCAAGATGG GATGCACCTC |
| 1751 | TTCGGGACCC AGCTCTCAGG CAACTGCTG |
| Progranulin Isoform 2 (SEQ ID NO: 8) | |
| MWTLVSWVALTAGLVAGTRCPDGQFCPVACCLDPGGASYSCCRPLLDKWPTTLSRHLGGPCQ | |
| VDAHCSAGHSCIFTVSGTSSCCPFPEAVACGDGHHCCPRGFHCSADGRSCFQRSGNNSVGAI | |
| QCPDSQFECPDFSTCCVMVDGSWGCCPMPQASCCEDRVHCCPHGAFCDLVHTRCITPTGTHP | |
| LAKKLPAQRTNRAVALSSSVMCPDARSRCPDGSTCCELPSGKYGCCPMPNATCCSDHLHCCP | |
| QDTVCDLIQSKCLSKENATTDLLTKLPAHTVGDVKCDMEVSCPDGYTCCRLQSGAWGCCPFT | |
| QAVCCEDHIHCCPAGFTCDTQKGTCEQGPHQVPWMEKAPAHLSLPDPQALKRDVPCDNVSSC | |
| PSSDTCCRDNRQGWACCPYRQGVCCADRRHCCPAGFRCAARGTKCLRREAPRWDAPLRDPAL | |
| RQLL | |
| As encoded by the DNA sequence (SEQ ID NO: 31): | |
| 1 | ATGTGGACCC TCGTATCTTG GGTTGCTCTT ACAGCAGGTC TCGTTGCAGG GACTAGATGT CCTGACGGAC |
| 71 | AGTTTTGCCC TGTGGCATGT TGTCTGGACC CAGGCGGAGC ATCCTACTCA TGTTGTCGCC CCCTCCTGGA |
| 141 | CAAGTGGCCT ACTACTCTGT CAAGACATTT GGGAGGACCC TGTCAAGTTG ACGCCCATTG TTCCGCAGGA |
| 211 | CACTCTTGCA TCTTTACTGT CTCTGGGACA AGCTCCTGTT GCCCATTTCC TGAAGCCGTG GCTTGCGGCG |
| 281 | ACGGACATCA CTGTTGTCCA CGAGGGTTCC ATTGCTCAGC AGATGGCAGA AGCTGTTTTC AAAGGAGTGG |
| 351 | GAACAATTCC GTAGGCGCAA TTCAGTGCCC AGATTCACAG TTCGAGTGCC CTGATTTCAG CACCTGCTGT |
| 421 | GTGATGGTCG ATGGGTCATG GGGATGCTGC CCTATGCCAC AAGCCTCTTG TTGTGAGGAC CGAGTCCACT |
| 491 | GTTGCCCTCA TGGCGCATTT TGTGACCTGG TGCATACAAG GTGCATCACG CCCACAGGAA CCCACCCCCT |
| 561 | TGCTAAAAAA CTTCCAGCAC AAAGGACAAA CAGAGCCGTT GCTCTCTCTT CTTCCGTGAT GTGCCCCGAC |
| 631 | GCCAGAAGCA GATGCCCAGA TGGCTCAACT TGTTGCGAGC TGCCTAGTGG GAAGTACGGG TGCTGCCCAA |
| 701 | TGCCAAATGC CACGTGTTGC TCTGACCACC TTCACTGCTG CCCGCAAGAT ACTGTGTGCG ACCTGATTCA |
| 771 | GTCCAAATGT TTGTCTAAGG AAAATGCCAC CACCGACCTG CTGACAAAAC TTCCCGCTCA CACAGTGGGG |
| 841 | GACGTGAAAT GTGATATGGA GGTTTCATGC CCCGACGGGT ATACATGTTG TAGATTGCAG AGCGGCGCAT |
| 911 | GGGGATGTTG TCCATTCACC CAGGCTGTTT GTTGCGAGGA CCACATCCAC TGTTGTCCTG CTGGGTTCAC |
| 981 | ATGCGACACA CAAAAAGGCA CTTGCGAGCA AGGACCACAC CAGGTTCCTT GGATGGAGAA AGCCCCAGCC |
| 1051 | CACCTGTCTT TGCCTGACCC TCAGGCTTTG AAGCGCGATG TACCCTGCGA CAACGTTTCT TCCTGTCCCT |
| 1121 | CCTCCGATAC ATGTTGCAGA GACAATAGAC AGGGATGGGC TTGTTGTCCT TATCGACAGG GCGTTTGCTG |
| 1191 | CGCTGATAGG AGGCATTGTT GTCCCGCAGG TTTTCGGTGC GCTGCTAGGG GAACAAAATG TCTGAGACGG |
| 1261 | GAGGCTCCAA GATGGGATGC TCCTTTGCGC GACCCCGCTC TGCGACAACT GCTC |
| Progranulin Isoform 3 (SEQ ID NO: 9) | |
| MAITAAHGASTAVQTGDPASKDQVTTPWVPSSALIVSSNARTSPRAVLWSMAPGGAAPCPRL | |
| PAVKTGCTAVCDLIQSKCLSKENATTDLLTKLPAHTVGDVKCDMEVSCPDGYTCCRLQSGAW | |
| GCCPFTQAVCCEDHIHCCPAGFTCDTQKGTCEQGPHQVPWMEKAPAHLSLPDPQALKRDVPC | |
| DNVSSCPSSDTCCQLTSGEWGCCPIPEAVCCSDHQHCCPQGYTCVAEGQCQRGSEIVAGLEK | |
| MPARRASLSHPRDIGCDQHTSCPVGQTCCPSLGGSWACCQLPHAVCCEDRQHCCPAGYTCNV | |
| KARSCEKEVVSAQPATFLARSPHVGVKDVECGEGHFCHDNQTCCRDNRQGWACCPYRQGVCC | |
| ADRRHCCPAGFRCAARGTKCLRREAPRWDAPLRDPALRQLL | |
| As encoded by the DNA sequence (SEQ ID NO: 32): | |
| 1 | ATGGCAATAA CTGCTGCACA TGGGGCCTCT ACAGCCGTGC AAACTGGAGA CCCAGCAAGT AAGGACCAAG |
| 71 | TTACAACCCC CTGGGTGCCC AGCTCTGCTC TTATCGTATC CAGTAATGCT CGAACCAGTC CCCGAGCTGT |
| 141 | ATTGTGGTCT ATGGCCCCAG GAGGGGCAGC TCCATGTCCT AGATTGCCTG CCGTAAAGAC AGGATGCACC |
| 211 | GCTGTATGCG ACCTCATCCA ATCTAAGTGT CTGTCTAAAG AAAACGCAAC TACAGACCTG CTTACGAAGC |
| 281 | TGCCCGCTCA TACCGTAGGG GACGTCAAAT GTGATATGGA GGTATCCTGT CCTGACGGCT ATACATGTTG |
| 351 | TCGCTTGCAG AGTGGTGCTT GGGGATGTTG TCCCTTCACA CAGGCCGTGT GTTGTGAGGA CCACATACAC |
| 421 | TGCTGCCCTG CTGGGTTCAC ATGTGACACG CAGAAGGGGA CATGTGAGCA GGGACCCCAT CAAGTACCTT |
| 491 | GGATGGAGAA GGCCCCAGCT CATCTGAGTC TGCCTGACCC CCAGGCACTG AAGAGAGATG TGCCCTGTGA |
| 561 | CAACGTGTCC TCCTGCCCCT CATCTGACAC TTGCTGCCAG TTGACATCTG GCGAATGGGG CTGCTGCCCT |
| 631 | ATACCCGAAG CTGTGTGTTG TAGTGACCAC CAACACTGCT GCCCCCAGGG ATACACCTGT GTGGCTGAGG |
| 701 | GACAGTGCCA GAGGGGTTCC GAAATTGTTG CTGGCTTGGA GAAGATGCCT GCTCGGAGAG CCTCACTTAG |
| 771 | CCATCCACGC GATATTGGGT GTGACCAGCA CACCTCCTGT CCTGTCGGAC AGACATGTTG CCCGTCCCTT |
| 841 | GGTGGAAGTT GGGCTTGTTG TCAGCTCCCC CATGCCGTTT GTTGCGAGGA CCGACAGCAC TGTTGTCCAG |
| 911 | CAGGCTATAC CTGCAATGTG AAAGCTAGGA GCTGCGAGAA GGAGGTTGTT AGCGCTCAAC CAGCAACATT |
| 981 | TCTGGCCCGC TCCCCCCATG TCGGTGTGAA AGATGTTGAA TGTGGCGAGG GACACTTCTG CCACGACAAC |
| 1051 | CAGACTTGCT GTAGGGACAA CCGCCAGGGT TGGGCTTGCT GTCCATATAG ACAGGGGGTG TGCTGTGCTG |
| 1121 | ATAGGAGACA TTGTTGTCCT GCTGGATTTA GATGTGCGGC TAGAGGCACT AAATGTTTGC GAAGGGAAGC |
| 1191 | TCCTAGATGG GATGCTCCAC TTAGAGACCC AGCCCTGCGG CAATTGCTC |

Progranulin is the precursor protein for granulin. Cleavage of progranulin produces a variety of active 6 kDa granulin peptides. These smaller cleavage products are named granulin A, granulin B, granulin C, etc. Epithelins 1 and 2 are synonymous with granulins A and B, respectively. Cleavage of progranulin into granulin occurs either in the extracellular matrix or the lysosome. Elastase, proteinase 3 and matrix metalloproteinase are proteases capable of cleaving progranulin into individual granulin peptides. Progranulin and granulin can be further differentiated by their hypothesized opposing roles in the cell. While progranulin is associated with anti-inflammation, cleaved granulin peptides have been implicated in pro-inflammatory behavior. Mutations in the progranulin (GRN) gene are a major cause of familial frontotemporal dementia. They result in a haploinsufficiency and therefore in a reduction of progranulin levels and in GRN-related brain degenerative changes that unfold over years if not decades. In such cases, progranulin levels may be restored with the viral vectors of the invention.

A functional fragment of progranulin is a fragment of at least 50, at least 100, at least 150, at least 200, at least 250, at least 300, at least 350, at least 400, at least 450 or at least 500 amino acids having at least 95% sequence identity with SEQ ID NO:7, SEQ ID NO:8 and/or SEQ ID NO:9, wherein the fragment has progranulin activity. A protein is said to have progranulin activity, if it can be cleaved into at least one granulin. In certain embodiments, a protein is said to have progranulin activity if it can be cleaved into at least one of granulin A, granulin B and/or granulin C. In certain embodiments, a protein is said to have progranulin activity, if it can be cleaved into granulin A, granulin B and granulin C.

In a particular embodiment, the invention relates to a viral vector encoding PGRN, or a functional fragment thereof; or a polypeptide having at least 95%, 96%, 97%, 98% or 99% sequence identity to the sequence shown in SEQ ID NO: 7, SEQ ID NO: 8 or SEQ ID NO: 9, or a functional fragment thereof, wherein the one or more promoters comprises:
a) a myelo-specific promoter, or a functional fragment thereof; and/or
b) a microglia-specific promoter, or a functional fragment thereof; and/or
c) a fusion promoter comprising or consisting of
   i) a first promoter, wherein said first promoter is a myelo-specific promoter or a microglia-specific promoter, or a functional fragment thereof; and
   ii) a second promoter.

That is, in certain embodiments, PGRN or a functional fragment or mutant variant thereof as disclosed above may be expressed from a myelo-specific promoter or from a functional fragment thereof. In other embodiments, PGRN or a functional fragment or mutant variant thereof as disclosed above may be expressed from a microglia-specific promoter or from a functional fragment thereof. In other embodiments, PGRN or a functional fragment or mutant variant thereof as disclosed above may be expressed from a fusion promoter, preferably wherein the fusion promoter comprises a myelo-specific or a microglia-specific promoter or functional fragments thereof.

The term “myelo-specific promoter” as used herein refers to any promoter that can drive expression in a myeloid cell. The skilled person is aware of methods to identify whether a promoter can drive expression in a myeloid cell. For example, a myeloid cell, such as the monocytic cell line THP-1, may be transduced with a viral vector encoding a fluorescent marker under control of the promoter in question. If expression of the fluorescent marker can be detected in the myeloid cell upon integration of the viral vector into the genome of the myeloid cell, the promoter is determined to be a myelo-specific promoter. Myelo-specific promoters within the meaning of the present invention include, without limitation, the miR223 promoter, the AIF1 promoter and the ITGAM promoter.

That is, in a particular embodiment, the invention relates to a viral vector encoding PGRN, or a functional fragment thereof; or a polypeptide having at least 95%, 96%, 97%, 98% or 99% sequence identity to the sequence shown in SEQ ID NO: 7, SEQ ID NO: 8 or SEQ ID NO: 9, or a functional fragment thereof, wherein the myelo-specific promoter is
a) a miR233 promoter, or a functional fragment thereof; or a promoter having at least 95%, 96%, 97%, 98%, 99% or 100% sequence identity to the sequence shown in SEQ ID NO: 1, or a functional fragment thereof; or
b) an ITGAM promoter, or a functional fragment thereof; or a promoter having at least 95%, 96%, 97%, 98%, 99% or 100% sequence identity to the sequence shown in SEQ ID NO:6, or a functional fragment thereof; or
c) an AIF1 promoter, or a functional fragment thereof; or a promoter having at least 95%, 96%, 97%, 98%, 99% or 100% sequence identity to the sequence shown in SEQ ID NO:5, or a functional fragment thereof.

The term “microglia-specific promoter” as used herein refers to any promoter that can drive expression in microglia. The skilled person is aware of methods to identify whether a promoter can drive expression in microglia. For example, microglia, such as an immortalized microglia cell line, may be transduced with a viral vector encoding a fluorescent marker under control of the promoter in question. If expression of the fluorescent marker can be detected in microglia upon integration of the viral vector into the genome of the microglia, the promoter is determined to be a microglia-specific promoter. Microglia-specific promoters within the meaning of the present invention include, without limitation, the P2RY12 promoter, the TMEM119 promoter, the OLFML3 promoter, the ITGAM promoter and the AIF1 promoter.

That is, in a particular embodiment, the invention relates to a viral vector encoding PGRN, or a functional fragment thereof or a polypeptide having at least 95%, 96%, 97%, 98% or 99% sequence identity to the sequence shown in SEQ ID NO: 7, SEQ ID NO: 8 or SEQ ID NO: 9, or a functional fragment thereof, wherein the microglia-specific promoter is
a) a TMEM119 promoter or a promoter having at least 95%, 96%, 97%, 98%, 99% or 100% sequence identity to the sequence shown in SEQ ID NO: 3, SEQ ID NO:23 or SEQ ID NO:24, or a functional fragment thereof; or
b) a P2RY12 promoter or a promoter having at least 95%, 96%, 97%, 98%, 99% or 100% sequence identity to the sequence shown in SEQ ID NO: 2, SEQ ID NO: 21 or SEQ ID NO: 22, or a functional fragment thereof; or
c) an OLFML3 promoter or a promoter having at least 95%, 96%, 97%, 98%, 99% or 100% sequence identity to the sequence shown in SEQ ID NO: 4 or SEQ ID NO:25, or a functional fragment thereof.

In a particular embodiment, the invention relates to a viral vector encoding PGRN, or a functional fragment thereof or a polypeptide having at least 95%, 96%, 97%, 98% or 99% sequence identity to the sequence shown in SEQ ID NO: 7, SEQ ID NO: 8 or SEQ ID NO: 9, or a functional fragment thereof, wherein the first promoter is a myelo-specific promoter and wherein the second promoter is a microglia-specific promoter, or vice versa.

The second promoter may be any promoter known in the art. However, in certain embodiments, PGRN or a functional fragment or mutant variant thereof as disclosed above may be expressed from a fusion promoter comprising a myelo-specific promoter and a microglia-specific promoter. That is, any of the myelo-specific promoters disclosed above may be combined with any of the microglia-specific promoters disclosed above, in any order.

In certain embodiments, PGRN or a functional fragment or mutant variant thereof as disclosed above may be expressed from a fusion promoter comprising miR223, a functional fragment thereof or a promoter with miR223 functionality, and a microglia-specific promoter.

That is, in a particular embodiment, the invention relates to a viral vector encoding PGRN, or a functional fragment thereof; or a polypeptide having at least 95%, 96%, 97%, 98% or 99% sequence identity to the sequence shown in SEQ ID NO:7, SEQ ID NO:8 or SEQ ID NO:9, or a functional fragment thereof, wherein the first promoter is a miR233 promoter, or a promoter having at least 95%, 96%, 97%, 98%, 99% or 100% sequence identity to the sequence shown in SEQ ID NO:1, or a functional fragment thereof; and wherein the first promoter is operably linked to
i) a TMEM119 promoter, or a promoter having at least 95%, 96%, 97%, 98%, 99% or 100% sequence identity to the sequence shown in SEQ ID NO:3, SEQ ID NO:23 or SEQ ID NO:24, or a functional fragment thereof
ii) a P2RY12 promoter, or a promoter having at least 95%, 96%, 97%, 98%, 99% or 100% sequence identity to the sequence shown in SEQ ID NO:2, SEQ ID NO:21 or SEQ ID NO:22, or a functional fragment thereof;
iii) an OLFML3 promoter, or a promoter having at least 95%, 96%, 97%, 98%, 99% or 100% sequence identity to the sequence shown in SEQ ID NO:4 or SEQ ID NO:25, or a functional fragment thereof;
iv) an ITGAM promoter, or a promoter having at least 95%, 96%, 97%, 98%, 99% or 100% sequence identity to the sequence shown in SEQ ID NO:6, or a functional fragment thereof; and/or
v) an AIF1 promoter or a promoter having at least 95%, 96%, 97%, 98%, 99% or 100% sequence identity to the sequence shown in SEQ ID NO:5, or a functional fragment thereof.

In certain embodiments, the invention relates to a viral vector encoding PGRN, or a functional fragment thereof; or a polypeptide having at least 95%, 96%, 97%, 98% or 99% sequence identity to the sequence shown in SEQ ID NO: 7, SEQ ID NO: 8 or SEQ ID NO: 9, or a functional fragment thereof, wherein the promoter is a miR233 promoter, or a promoter having at least 95%, 96%, 97%, 98%, 99% or 100% sequence identity to the sequence shown in SEQ ID NO: 1, or a functional fragment thereof.

In certain embodiments, the invention relates to a viral vector encoding PGRN, or a functional fragment thereof; or a polypeptide having at least 95%, 96%, 97%, 98% or 99% sequence identity to the sequence shown in SEQ ID NO: 7, SEQ ID NO: 8 or SEQ ID NO: 9, or a functional fragment thereof, wherein the first promoter is an ITGAM promoter, or a promoter having at least 95%, 96%, 97%, 98%, 99% or 100% sequence identity to the sequence shown in SEQ ID NO6, or a functional fragment thereof.

In certain embodiments, the invention relates to a viral vector encoding PGRN, or a functional fragment thereof; or a polypeptide having at least 95%, 96%, 97%, 98% or 99% sequence identity to the sequence shown in SEQ ID NO: 7, SEQ ID NO: 8 or SEQ ID NO: 9, or a functional fragment thereof, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof; and (b) a P2RY12 promoter, or a promoter having at least 95%, 96%, 97%, 98%, 99% or 100% sequence identity to the sequence shown in SEQ ID NO:2, SEQ ID NO:21 or SEQ ID NO:22, or a functional fragment thereof. In certain embodiments, the fusion promoter comprises a nucleotide sequence having at least 90%, at least 95%, at least 96%, at least 97%, at least 98%, at least 99% or 100% sequence identity with SEQ ID NO:26 or SEQ ID NO:27.

In certain embodiments, the invention relates to a viral vector encoding PGRN, or a functional fragment thereof; or a polypeptide having at least 95%, 96%, 97%, 98% or 99% sequence identity to the sequence shown in SEQ ID NO: 7, SEQ ID NO: 8 or SEQ ID NO: 9, or a functional fragment thereof, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof; and (b) a TMEM119 promoter, or a promoter having at least 95%, 96%, 97%, 98%, 99% or 100% sequence identity to the sequence shown in SEQ ID NO:3, SEQ ID NO:23 or SEQ ID NO:24, or a functional fragment thereof. In certain embodiments, the fusion promoter comprises a nucleotide sequence having at least 90%, at least 95%, at least 96%, at least 97%, at least 98%, at least 99% or 100% sequence identity with SEQ ID NO:28.

In certain embodiments, the invention relates to a viral vector encoding PGRN, or a functional fragment thereof; or a polypeptide having at least 95%, 96%, 97%, 98% or 99% sequence identity to the sequence shown in SEQ ID NO: 7, SEQ ID NO: 8 or SEQ ID NO: 9, or a functional fragment thereof, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof; and (b) an OLFML3 promoter, or a promoter having at least 95%, 96%, 97%, 98%, 99% or 100% sequence identity to the sequence shown in SEQ ID NO:4 or SEQ ID NO:25, or a functional fragment thereof. In certain embodiments, the fusion promoter comprises a nucleotide sequence having at least 90%, at least 95%, at least 96%, at least 97%, at least 98%, at least 99% or 100% sequence identity with SEQ ID NO:29.

In a particular embodiment, the invention relates to a viral vector comprising a transgene under control of one or more promoters, wherein the transgene encodes
a) IL-12, or a functional fragment thereof, or
b) a polypeptide having at least 95%, 96%, 97%, 98%, 99% or 100% sequence identity to the sequence shown in SEQ ID NO: 11, or a functional fragment thereof, and/or a polypeptide having at least 95%, 96%, 97%, 98%, 99% or 100% sequence identity to the sequence shown in SEQ ID NO: 12, or a functional fragment thereof.

That is, in certain embodiments, the invention relates to a viral vector encoding interleukin-12 (IL-12). The term “Interleukin-12” or “IL-12” refers to a protein comprising the protein sequence of SEQ ID NO: 11 and the protein sequence of SEQ ID NO: 12, or any protein fragment derived from protein sequences SEQ ID NO: 11 and/or SEQ ID NO: 12 with a length of at least 50 amino acids, or to any protein sequence with more than 95% homology thereof. Also provided herein are nucleic acid sequences encoding said proteins.

IL-12 subunit alpha
(SEQ ID NO: 11)
MCPARSLLLVATLVLLDHLSLARNLPVATPDPGMFPCLHHSQNLLRAVSN
MLQKARQTLEFYPCTSEEIDHEDITKDKTSTVEACLPLELTKNESCLNSR
ETSFITNGSCLASRKTSFMMALCLSSIYEDLKMYQVEFKTMNAKLLMDPK
RQIFLDQNMLAVIDELMQALNFNSETVPQKSSLEEPDFYKTKIKLCILLH
AFRIRAVTIDRVMSYLNAS
IL-12 subunit beta
(SEQ ID NO: 12)
MCHQQLVISWFSLVFLASPLVAIWELKKDVYVVELDWYPDAPGEMVVLTC
DTPEEDGITWTLDQSSEVLGSGKTLTIQVKEFGDAGQYTCHKGGEVLSHS
LLLLHKKEDGIWSTDILKDQKEPKNKTFLRCEAKNYSGRFTCWWLTTIST
DLTFSVKSSRGSSDPQGVTCGAATLSAERVRGDNKEYEYSVECQEDSACP
AAEESLPIEVMVDAVHKLKYENYTSSFFIRDIIKPDPPKNLQLKPLKNSR
QVEVSWEYPDTWSTPHSYFSLTFCVQVQGKSKREKKDRVFTDKTSATVIC
RKNASISVRAQDRYYSSSWSEWASVPCS
IL-12 beta and alpha subunit connected with a
linker
(SEQ ID NO: 33)
MCHQQLVISWFSLVFLASPLVAIWELKKDVYVVELDWYPDAPGEMVVLTC
DTPEEDGITWTLDQSSEVLGSGKTLTIQVKEFGDAGQYTCHKGGEVLSHS
LLLLHKKEDGIWSTDILKDQKEPKNKTFLRCEAKNYSGRFTCWWLTTIST
DLTFSVKSSRGSSDPQGVTCGAATLSAERVRGDNKEYEYSVECQEDSACP
AAEESLPIEVMVDAVHKLKYENYTSSFFIRDIIKPDPPKNLQLKPLKNSR
QVEVSWEYPDTWSTPHSYFSLTFCVQVQGKSKREKKDRVFTDKTSATVIC
RKNASISVRAQDRYYSSSWSEWASVPCSGGGGSGGGGSGGGGSRNLPVAT
PDPGMFPCLHHSQNLLRAVSNMLQKARQTLEFYPCTSEEIDHEDITKDKT
STVEACLPLELTKNESCLNSRETSFITNGSCLASRKTSFMMALCLSSIYE
DLKMYQVEFKTMNAKLLMDPKRQIFLDQNMLAVIDELMQALNFNSETVPQ
KSSLEEPDFYKTKIKLCILLHAFRIRAVTIDRVMSYLNAS

Preferably, the viral vector of the invention encodes both the polypeptide according to SEQ ID NO:11 and the polypeptide according to SEQ ID NO:12. In certain embodiments, the two IL-12 subunits alpha and beta may be connected via a linker. In certain embodiments the linker has the amino acid sequence GGGGSGGGGSGGGGS (SEQ ID NO:34). In certain embodiments, the IL-12 encoded in the viral vector according to the invention is a single-chain IL-12 variant. Single-chain IL-12 variants have been disclosed in the art.

Interleukin-12 (IL-12) is an interleukin that is naturally produced by dendritic cells, macrophages, neutrophils, and human B-lymphoblastoid cells (NC-37) in response to antigenic stimulation. IL-12 is composed of a bundle of four alpha helices. It is a heterodimeric cytokine encoded by two separate genes, IL-12A (p35) and IL-12B (p40). The active heterodimer (referred to as ‘p70’), and a homodimer of p40 are formed following protein synthesis. Accordingly, the viral vector of the invention preferably encodes both the alpha subunit (SEQ ID NO:11) and the beta subunit (SEQ ID NO:12) of IL-12. Interleukin-12 (IL-12) has emerged as one of the most potent agents for anti-tumor immunotherapy. However, potentially lethal toxicity associated with systemic administration of IL-12 precludes its clinical application in form of the pure cytokine.

A functional fragment of IL-12 is a fragment of at least 50, at least 100, at least 150 or at least 200 amino acids having at least 95% sequence identity with SEQ ID NO. 11 or SEQ ID NO:12, wherein the fragment has IL-12 activity. Assays to determine whether a protein has IL-12 activity have been described in the art, for example by Peng et al., A single-chain IL-12 IgG3 antibody fusion protein retains antibody specificity and IL-12 bioactivity and demonstrates antitumor activity; J Immunol. 1999 Jul. 1; 163(1):250-8.

In a particular embodiment, the invention relates to a viral vector encoding IL-12, including single-chain variants thereof, or a functional fragment thereof; or a polypeptide having at least 95%, 96%, 97%, 98% or 99% sequence identity to the sequence shown in SEQ ID NO: 11, and/or SEQ ID NO: 12, or functional fragments thereof, wherein the one or more promoters comprises:
a) a myelo-specific promoter, or a functional fragment thereof, and/or
b) a microglia-specific promoter, or a functional fragment thereof, and/or
c) a fusion promoter comprising or consisting of
   i) a first promoter, wherein said first promoter is a myelo-specific promoter or a microglia-specific promoter, or a functional fragment thereof; and
   ii) a second promoter.

That is, in certain embodiments, IL-12 or a functional fragment or mutant variant thereof as disclosed above may be expressed from a myelo-specific promoter or from a functional fragment thereof. In other embodiments, IL-12 or a functional fragment or mutant variant thereof as disclosed above may be expressed from a microglia-specific promoter or from a functional fragment thereof. In other embodiments, IL-12 or a functional fragment or mutant variant thereof as disclosed above may be expressed from a fusion promoter, preferably wherein the fusion promoter comprises a myelo-specific or a microglia-specific promoter or functional fragments thereof.

That is, in a particular embodiment, the invention relates to a viral vector encoding IL-12, or a functional fragment thereof, or a polypeptide having at least 95%, 96%, 97%, 98% or 99% sequence identity to the sequence shown in SEQ ID NO: 11 and/or SEQ ID NO: 12, or a functional fragment thereof, wherein the myelo-specific promoter is
a) a miR233 promoter, or a functional fragment thereof, or a promoter having at least 95%, 96%, 97%, 98%, 99% or 100% sequence identity to the sequence shown in SEQ ID NO: 1, or a functional fragment thereof;
b) an ITGAM promoter, or a functional fragment thereof, or a promoter having at least 95%, 96%, 97%, 98%, 99% or 100% sequence identity to the sequence shown in SEQ ID NO: 6, or a functional fragment thereof
c) an AIF1 promoter, or a functional fragment thereof; or a promoter having at least 95%, 96%, 97%, 98%, 99% or 100% sequence identity to the sequence shown in SEQ ID NO: 5, or a functional fragment thereof; or

In a particular embodiment, the invention relates to a viral vector encoding IL-12, or a functional fragment thereof, or a polypeptide having at least 95%, 96%, 97%, 98% or 99% sequence identity to the sequence shown in SEQ ID NO: 11 and/or SEQ ID NO: 12, or a functional fragment thereof, wherein the microglia-specific promoter is
a) a TMEM119 promoter or a promoter having at least 95%, 96%, 97%, 98%, 99% or 100% sequence identity to the sequence shown in SEQ ID NO:3, SEQ ID NO:23 or SEQ ID NO:24, or a functional fragment thereof, or
b) a P2RY12 promoter or a promoter having at least 95%, 96%, 97%, 98%, 99% or 100% sequence identity to the sequence shown in SEQ ID NO: 2, SEQ ID NO:21 or SEQ ID NO:22, or a functional fragment thereof;
c) an OLFML3 promoter or a promoter having at least 95%, 96%, 97%, 98%, 99% or 100% sequence identity to the sequence shown in SEQ ID NO:4 or SEQ ID NO:25, or a functional fragment thereof.

In a particular embodiment, the invention relates to a viral vector encoding IL-12, or a functional fragment thereof, or a polypeptide having at least 95%, 96%, 97%, 98% or 99% sequence identity to the sequence shown in SEQ ID NO: 11 and/or SEQ ID NO: 12, or a functional fragment thereof, wherein the first promoter is a myelo-specific promoter and wherein the second promoter is a microglia-specific promoter, or vice versa.

The second promoter may be any promoter known in the art. However, in certain embodiments, IL-12 or a functional fragment or mutant variant thereof as disclosed above may be expressed from a fusion promoter comprising a myelo-specific promoter and a microglia-specific promoter. That is, any of the myelo-specific promoters disclosed above may be combined with any of the microglia-specific promoters disclosed above, in any order.

In certain embodiments, IL-12 or a functional fragment or mutant variant thereof as disclosed above may be expressed from a fusion promoter comprising miR223, a functional fragment thereof or a promoter with miR223 functionality, and a microglia-specific promoter.

That is, in a particular embodiment, the invention relates to a viral vector encoding IL-12, or a functional fragment thereof, or a polypeptide having at least 95%, 96%, 97%, 98% or 99% sequence identity to the sequence shown in SEQ ID NO: 11 and/or SEQ ID NO: 12, or a functional fragment thereof, wherein the first promoter is a miR233 promoter, or a promoter having at least 95%, 96%, 97%, 98%, 99% or 100% sequence identity to the sequence shown in SEQ ID NO: 1, or a functional fragment thereof, and wherein the first promoter is operably linked to
i) a TMEM119 promoter, or a promoter having at least 95%, 96%, 97%, 98%, 99% or 100% sequence identity to the sequence shown in SEQ ID NO:3, SEQ ID NO:23 or SEQ ID NO:24, or a functional fragment thereof,
ii) a P2RY12 promoter, or a promoter having at least 95%, 96%, 97%, 98%, 99% or 100% sequence identity to the sequence shown in SEQ ID NO:2, SEQ ID NO:21 or SEQ ID NO:22, or a functional fragment thereof;
iii) an OLFML3 promoter, or a promoter having at least 95%, 96%, 97%, 98%, 99% or 100% sequence identity to the sequence shown in SEQ ID NO:4 or SEQ ID NO:25, or a functional fragment thereof;
iv) an ITGAM promoter, or a promoter having at least 95%, 96%, 97%, 98%, 99% or 100% sequence identity to the sequence shown in SEQ ID NO:6, or a functional fragment thereof; and/or
v) an AIF1 promoter or a promoter having at least 95%, 96%, 97%, 98%, 99% or 100% sequence identity to the sequence shown in SEQ ID NO:5, or a functional fragment thereof.

In certain embodiments, the invention relates to a viral vector encoding IL-12, or a functional fragment thereof; or a polypeptide having at least 95%, 96%, 97%, 98% or 99% sequence identity to the sequence shown in SEQ ID NO:11 and/or SEQ ID NO:12, or a functional fragment thereof, wherein the promoter is a miR233 promoter, or a promoter having at least 95%, 96%, 97%, 98%, 99% or 100% sequence identity to the sequence shown in SEQ ID NO:1, or a functional fragment thereof.

In certain embodiments, the invention relates to a viral vector encoding IL-12, or a functional fragment thereof; or a polypeptide having at least 95%, 96%, 97%, 98% or 99% sequence identity to the sequence shown in SEQ ID NO: 11 and/or SEQ ID NO: 12, or a functional fragment thereof, wherein the first promoter is an ITGAM promoter, or a promoter having at least 95%, 96%, 97%, 98%, 99% or 100% sequence identity to the sequence shown in SEQ ID NO:6, or a functional fragment thereof.

In certain embodiments, the invention relates to a viral vector encoding IL-12, or a functional fragment thereof; or a polypeptide having at least 95%, 96%, 97%, 98% or 99% sequence identity to the sequence shown in SEQ ID NO: 11 and/or SEQ ID NO: 12, or a functional fragment thereof, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof; and (b) a P2RY12 promoter, or a promoter having at least 95%, 96%, 97%, 98%, 99% or 100% sequence identity to the sequence shown in SEQ ID NO: 2, SEQ ID NO:21 or SEQ ID NO:22, or a functional fragment thereof. In certain embodiments, the fusion promoter comprises a nucleotide sequence having at least 90%, at least 95%, at least 96%, at least 97%, at least 98%, at least 99% or 100% sequence identity with SEQ ID NO:26 or SEQ ID NO:27.

In certain embodiments, the invention relates to a viral vector encoding IL-12, or a functional fragment thereof; or a polypeptide having at least 95%, 96%, 97%, 98% or 99% sequence identity to the sequence shown in SEQ ID NO: 11 and/or SEQ ID NO: 12, or a functional fragment thereof, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof; and (b) a TMEM119 promoter, or a promoter having at least 95%, 96%, 97%, 98%, 99% or 100% sequence identity to the sequence shown in SEQ ID NO:3, SEQ ID NO:23 or SEQ ID NO:24, or a functional fragment thereof. In certain embodiments, the fusion promoter comprises a nucleotide sequence having at least 90%, at least 95%, at least 96%, at least 97%, at least 98%, at least 99% or 100% sequence identity with SEQ ID NO:28.

In certain embodiments, the invention relates to a viral vector encoding IL-12, or a functional fragment thereof; or a polypeptide having at least 95%, 96%, 97%, 98% or 99% sequence identity to the sequence shown in SEQ ID NO: 11 and/or SEQ ID NO: 12, or a functional fragment thereof, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof; and (b) an OLFML3 promoter, or a promoter having at least 95%, 96%, 97%, 98%, 99% or 100% sequence identity to the sequence shown in SEQ ID NO:4 or SEQ ID NO:25, or a functional fragment thereof. In certain embodiments, the fusion promoter comprises a nucleotide sequence having at least 90%, at least 95%, at least 96%, at least 97%, at least 98%, at least 99% or 100% sequence identity with SEQ ID NO:29.

In a particular embodiment, the invention relates to a viral vector comprising a transgene under control of one or more promoters, wherein the transgene encodes
a) Interferon-gamma (IFN-gamma), or a functional fragment thereof; or
b) a polypeptide having at least 95%, 96%, 97%, 98%, 99% or 100% sequence identity to the sequence shown in SEQ ID NO: 10, or a functional fragment thereof.

That is, in certain embodiments, the invention relates to a viral vector encoding interferon gamma (IFN-gamma). The term “Interferon-gamma” or “IFN-gamma” or “IFN-γ” refers to the protein sequence of SEQ ID NO: 10, and/or to any sequence with a sequence identity of >95% homology thereof. Also provided herein are nucleic acid sequences encoding said proteins.

(SEQ ID NO: 10)
MKYTSYILAFQLCIVLGSLGCYCQDPYVKEAENLKKYFNAGHSDVADNGT
LFLGILKNWKEESDRKIMQSQIVSFYFKLFKNFKDDQSIQKSVETIKEDM
NVKFFNSNKKKRDDFEKLTNYSVTDLNVQRKAIHELIQVMAELSPAAKTG
KRKRSQMLFRGRRASQ

as encoded by the DNA sequence (SEQ ID NO:35):

| | |
|---|---|
| 1 | ATGAAGTACA CCTCCTACAT CCTCGCTTTT CAACTGTGCA |
| | TTGTCCTTGG GTCTCTTGGA TGTTACTGTC |
| 71 | AAGACCCATA CGTGAAAGAG GCAGAGAACC TCAAAAAGTA |
| | TTTCAATGCT GGACATAGCG ACGTGGCCGA |
| 141 | TAATGGCACT CTCTTCCTGG GCATCCTGAA GAACTGGAAG |
| | GAAGAATCTG ACCGCAAGAT TATGCAGTCC |
| 211 | CAGATTGTGT CCTTTTATTT CAAACTCTTC AAGAATTTCA |
| | AAGATGACCA GAGCATTCAG AAAAGCGTGG |
| 281 | AAACAATCAA AGAGGATATG AACGTGAAGT TTTTCAATTC |
| | AAATAAGAAG AAGCGCGATG ACTTTGAGAA |
| 351 | ACTTACCAAC TATTCCGTGA CCGACTTGAA TGTGCAGAGG |
| | AAGGCCATAC ATGAGTTGAT ACAAGTTATG |
| 421 | GCTGAACTGA GCCCCGCCGC TAAAACTGGT AAAAGGAAGC |
| | GCAGCCAAAT GCTGTTTCGA GGGAGGCGCG |
| 491 | CCAGTCAG |

IFN-gamma is a dimerized soluble cytokine that is the only member of the type II class of interferons. In humans, the IFN-gamma protein is encoded by the IFNG gene. IFN-gamma, or type II interferon, is a cytokine that is critical for innate and adaptive immunity against viral, some bacterial and protozoan infections. IFN-gamma is an important activator of macrophages and inducer of major histocompatibility complex class II molecule expression. Aberrant IFN-gamma expression is associated with a number of autoinflammatory and autoimmune diseases. The importance of IFN-gamma in the immune system stems in part from its ability to inhibit viral replication directly, and most importantly from its immunostimulatory and immunomodulatory effects. IFN-gamma is produced predominantly by natural killer cells (NK) and natural killer T cells (NKT) as part of the innate immune response, and by CD4 Th1 and CD8 cytotoxic T lymphocyte (CTL) effector T cells once antigen-specific immunity develops as part of the adaptive immune response. IFN-gamma is also produced by non-cytotoxic innate lymphoid cells (ILC), a family of immune cells first discovered in the early 2010s.

IFN-gamma 1b is approved by the U.S. Food and Drug Administration to treat chronic granulomatous disease and osteopetrosis. It is being studied for the treatment of Friedreich's ataxia. Although not officially approved, IFN-gamma has also been shown to be effective in treating patients with moderate to severe atopic dermatitis. IFN-gamma is not approved yet for the treatment in any cancer immunotherapy. However, improved survival was observed when IFN-gamma was administrated to patients with bladder carcinoma and melanoma cancers. The most promising result was achieved in patients with stage 2 and 3 of ovarian carcinoma.

A functional fragment of IFN-gamma is a fragment of at least 50, at least 100 or at least 150 amino acids having at least 95% sequence identity with SEQ ID NO:10, wherein the fragment has IFN-gamma activity. Assays to determine whether a protein has IFN-gamma activity have been described in the art, for example by Corstjens et al., A user-friendly, highly sensitive assay to detect the IFN-gamma secretion by T cells; Clin Biochem. 2008 April; 41(6): 440-444.

In a particular embodiment, the invention relates to a viral vector encoding IFN-gamma, or a functional fragment thereof, or a polypeptide having at least 95%, 96%, 97%, 98% or 99% sequence identity to the sequence shown in SEQ ID NO: 10, or a functional fragment thereof, wherein the one or more promoters comprises:
a) a myelo-specific promoter, or a functional fragment thereof, and/or
b) a microglia-specific promoter, or a functional fragment thereof, and/or
c) a fusion promoter comprising or consisting of
   i) a first promoter, wherein said first promoter is a myelo-specific promoter or a microglia-specific promoter, or a functional fragment thereof; and
   ii) a second promoter.

That is, in certain embodiments, IFN-gamma or a functional fragment or mutant variant thereof as disclosed above may be expressed from a myelo-specific promoter or from a functional fragment thereof. In other embodiments, IFN-gamma or a functional fragment or mutant variant thereof as disclosed above may be expressed from a microglia-specific promoter or from a functional fragment thereof. In other embodiments, IFN-gamma or a functional fragment or mutant variant thereof as disclosed above may be expressed from a fusion promoter, preferably wherein the fusion promoter comprises a myelo-specific or a microglia-specific promoter or functional fragments thereof.

That is, in a particular embodiment, the invention relates to a viral vector encoding IFN-gamma, or a functional fragment thereof, or a polypeptide having at least 95%, 96%, 97%, 98% or 99% sequence identity to the sequence shown in SEQ ID NO: 10, or a functional fragment thereof, wherein the myelo-specific promoter is
a) a miR233 promoter, or a functional fragment thereof, or a promoter having at least 95%, 96%, 97%, 98%, 99% or 100% sequence identity to the sequence shown in SEQ ID NO: 1, or a functional fragment thereof;
b) an ITGAM promoter, or a functional fragment thereof, or a promoter having at least 95%, 96%, 97%, 98%, 99% or 100% sequence identity to the sequence shown in SEQ ID NO:6, or a functional fragment thereof,
c) an AIF1 promoter, or a functional fragment thereof; or a promoter having at least 95%, 96%, 97%, 98%, 99% or 100% sequence identity to the sequence shown in SEQ ID NO:5, or a functional fragment thereof.

In a particular embodiment, the invention relates to a viral vector encoding IFN-gamma, or a functional fragment thereof, or a polypeptide having at least 95%, 96%, 97%, 98% or 99% sequence identity to the sequence shown in SEQ ID NO: 10, or a functional fragment thereof, wherein the microglia-specific promoter is
a) a TMEM119 promoter or a promoter having at least 95%, 96%, 97%, 98%, 99% or 100% sequence identity to the sequence shown in SEQ ID NO:3, SEQ ID NO:23 or SEQ ID NO:24, or a functional fragment thereof; or
b) a P2RY12 promoter or a promoter having at least 95%, 96%, 97%, 98%, 99% or 100% sequence identity to the sequence shown in SEQ ID NO: 2, SEQ ID NO:21 or SEQ ID NO:22, or a functional fragment thereof; or
c) an OLFML3 promoter or a promoter having at least 95%, 96%, 97%, 98%, 99% or 100% sequence identity to the sequence shown in SEQ ID NO:4 or SEQ ID NO:25, or a functional fragment thereof.

In a particular embodiment, the invention relates to a viral vector encoding IFN-gamma, or a functional fragment thereof; or a polypeptide having at least 95%, 96%, 97%, 98% or 99% sequence identity to the sequence shown in SEQ ID NO: 10, or a functional fragment thereof, wherein the first promoter is a myelo-specific promoter and wherein the second promoter is a microglia-specific promoter, or vice versa.

The second promoter may be any promoter known in the art. However, in certain embodiments, IFN-gamma or a functional fragment or mutant variant thereof as disclosed above may be expressed from a fusion promoter comprising a myelo-specific promoter and a microglia-specific promoter. That is, any of the myelo-specific promoters disclosed above may be combined with any of the microglia-specific promoters disclosed above, in any order.

In certain embodiments, IFN-gamma or a functional fragment or mutant variant thereof as disclosed above may be expressed from a fusion promoter comprising miR223, a functional fragment thereof or a promoter with miR223 functionality, and a microglia-specific promoter.

That is, in a particular embodiment, the invention relates to a viral vector encoding IFN-gamma, or a functional fragment thereof; or a polypeptide having at least 95%, 96%, 97%, 98% or 99% sequence identity to the sequence shown in SEQ ID NO:10, or a functional fragment thereof, wherein the first promoter is a miR233 promoter, or a promoter having at least 95%, 96%, 97%, 98%, 99% or 100% sequence identity to the sequence shown in SEQ ID NO: 1, or a functional fragment thereof; and wherein the first promoter is operably linked to
ii) a TMEM119 promoter, or a promoter having at least 95%, 96%, 97%, 98%, 99% or 100% sequence identity to the sequence shown in SEQ ID NO:3, SEQ ID NO:23 or SEQ ID NO:24, or a functional fragment thereof;
i) a P2RY12 promoter, or a promoter having at least 95%, 96%, 97%, 98%, 99% or 100% sequence identity to the sequence shown in SEQ ID NO:2, SEQ ID NO:21 or SEQ ID NO:22, or a functional fragment thereof;
iii) an OLFML3 promoter, or a promoter having at least 95%, 96%, 97%, 98%, 99% or 100% sequence identity to the sequence shown in SEQ ID NO:4 or SEQ ID NO:25, or a functional fragment thereof;
iv) an ITGAM promoter, or a promoter having at least 95%, 96%, 97%, 98%, 99% or 100% sequence identity to the sequence shown in SEQ ID NO:6, or a functional fragment thereof; and/or
v) an AIF1 promoter or a promoter having at least 95%, 96%, 97%, 98%, 99% or 100% sequence identity to the sequence shown in SEQ ID NO:5, or a functional fragment thereof.

In certain embodiments, the invention relates to a viral vector encoding IFN-gamma, or a functional fragment thereof; or a polypeptide having at least 95%, 96%, 97%, 98% or 99% sequence identity to the sequence shown in SEQ ID NO: 10, or a functional fragment thereof, wherein the promoter is a miR233 promoter, or a promoter having at least 95%, 96%, 97%, 98%, 99% or 100% sequence identity to the sequence shown in SEQ ID NO: 1, or a functional fragment thereof.

In certain embodiments, the invention relates to a viral vector encoding IFN-gamma, or a functional fragment thereof; or a polypeptide having at least 95%, 96%, 97%, 98% or 99% sequence identity to the sequence shown in SEQ ID NO: 10, or a functional fragment thereof, wherein the first promoter is an ITGAM promoter, or a promoter having at least 95%, 96%, 97%, 98%, 99% or 100% sequence identity to the sequence shown in SEQ ID NO:6, or a functional fragment thereof.

In certain embodiments, the invention relates to a viral vector encoding IFN-gamma, or a functional fragment thereof; or a polypeptide having at least 95%, 96%, 97%, 98% or 99% sequence identity to the sequence shown in SEQ ID NO: 10, or a functional fragment thereof, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof; and (b) a P2RY12 promoter, or a promoter having at least 95%, 96%, 97%, 98%, 99% or 100% sequence identity to the sequence shown in SEQ ID NO: 2, SEQ ID NO:21 or SEQ ID NO:22, or a functional fragment thereof. In certain embodiments, the fusion promoter comprises a nucleotide sequence having at least 90%, at least 95%, at least 96%, at least 97%, at least 98%, at least 99% or 100% sequence identity with SEQ ID NO:26 or SEQ ID NO:27.

In certain embodiments, the invention relates to a viral vector encoding IFN-gamma, or a functional fragment thereof; or a polypeptide having at least 95%, 96%, 97%, 98% or 99% sequence identity to the sequence shown in SEQ ID NO: 10, or a functional fragment thereof, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof; and (b) a TMEM119 promoter, or a promoter having at least 95%, 96%, 97%, 98%, 99% or 100% sequence identity to the sequence shown in SEQ ID NO:3, SEQ ID NO:23 or SEQ ID NO:24, or a functional fragment thereof. In certain embodiments, the fusion promoter comprises a nucleotide sequence having at least 90%, at least 95%, at least 96%, at least 97%, at least 98%, at least 99% or 100% sequence identity with SEQ ID NO:28.

In certain embodiments, the invention relates to a viral vector encoding IFN-gamma, or a functional fragment thereof; or a polypeptide having at least 95%, 96%, 97%, 98% or 99% sequence identity to the sequence shown in SEQ ID NO: 10, or a functional fragment thereof, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof; and (b) an OLFML3 promoter, or a promoter having at least 95%, 96%, 97%, 98%, 99% or 100% sequence identity to the sequence shown in SEQ ID NO: 4 or SEQ ID NO:25, or a functional fragment thereof. In certain embodiments, the fusion promoter comprises a nucleotide sequence having at least 90%, at least 95%, at least 96%, at least 97%, at least 98%, at least 99% or 100% sequence identity with SEQ ID NO:29.

In a particular embodiment, the invention relates to a viral vector comprising a transgene under control of one or more promoters, wherein the transgene encodes
a) GM-CSF, or a functional fragment thereof; or
b) a polypeptide having at least 95%, 96%, 97%, 98%, 99% or 100% sequence identity to the sequence shown in SEQ ID NO: 13, or a functional fragment thereof.

That is, in certain embodiments, the invention relates to a viral vector encoding Granulocyte-macrophage colony-stimulating factor (GM-CSF). The term “GM-CSF” refers to the protein sequence of SEQ ID NO: 13, and/or to any sequence with a sequence identity of >95% homology thereof. Also provided herein are nucleic acid sequences encoding said proteins.

(SEQ ID NO: 13)
MWLQSLLLLGTVACSISAPARSPSPSTQPWEHVNAIQEARRLLNLSRDTA
AEMNETVEVISEMFDLQEPTCLQTRLELYKQGLRGSLTKLKGPLTMMASH
YKQHCPPTPETSCATQIITFESFKENLKDFLLVIPFDCWEPVQE

Granulocyte-macrophage colony-stimulating factor (GM-CSF), also known as colony-stimulating factor 2 (CSF2), is a monomeric glycoprotein secreted by macrophages, T cells, mast cells, natural killer cells, endothelial cells and fibroblasts that functions as a cytokine. The pharmaceutical analogs of naturally occurring GM-CSF are called sargramostim and molgramostim. Unlike granulocyte colony-stimulating factor, which specifically promotes neutrophil proliferation and maturation, GM-CSF affects more cell types, especially macrophages and eosinophils. GM-CSF is a monomeric glycoprotein that functions as a cytokine—it is a white blood cell growth factor. GM-CSF stimulates stem cells to produce granulocytes (neutrophils, eosinophils, and basophils) and monocytes. Monocytes exit the circulation and migrate into tissue, whereupon they mature into macrophages and dendritic cells. Thus, it is part of the immune/inflammatory cascade, by which activation of a small number of macrophages can rapidly lead to an increase in their numbers, a process crucial for fighting infection.

A functional fragment of GM-CSF is a fragment of at least 50, at least 100 amino acids, at least 110, at least 120, at least 130 or at least 140 amino acids having at least 95% sequence identity with SEQ ID NO:13, wherein the fragment has GM-CSF activity. Assays to determine whether a protein has GM-CSF activity have been described in the art, for example by Singh et al, GM-CSF Enhances Macrophage Glycolytic Activity In Vitro and Improves Detection of Inflammation In Vivo; J Nucl Med. 2016 September; 57(9):1428-35. doi: 10.2967/jnumed.115.167387. Epub 2016 Apr. 14.

In a particular embodiment, the invention relates to a viral vector encoding GM-CSF, or a functional fragment thereof or a polypeptide having at least 95%, 96%, 97%, 98% or 99% sequence identity to the sequence shown in SEQ ID NO: 13, or a functional fragment thereof, wherein the one or more promoters comprises:
a) a myelo-specific promoter, or a functional fragment thereof and/or
b) a microglia-specific promoter, or a functional fragment thereof and/or
c) a fusion promoter comprising or consisting of
   i) a first promoter, wherein said first promoter is a myelo-specific promoter or a microglia-specific promoter, or a functional fragment thereof; and
   ii) a second promoter.

That is, in certain embodiments, GM-CSF or a functional fragment or mutant variant thereof as disclosed above may be expressed from a myelo-specific promoter or from a functional fragment thereof. In other embodiments, GM-CSF or a functional fragment or mutant variant thereof as disclosed above may be expressed from a microglia-specific promoter or from a functional fragment thereof. In other embodiments, GM-CSF or a functional fragment or mutant variant thereof as disclosed above may be expressed from a fusion promoter, preferably wherein the fusion promoter comprises a myelo-specific or a microglia-specific promoter or functional fragments thereof.

That is, in a particular embodiment, the invention relates to a viral vector encoding GM-CSF a, or a functional fragment thereof, or a polypeptide having at least 95%, 96%, 97%, 98% or 99% sequence identity to the sequence shown in SEQ ID NO: 13, or a functional fragment thereof, wherein the myelo-specific promoter is
a) a miR233 promoter, or a functional fragment thereof, or a promoter having at least 95%, 96%, 97%, 98%, 99% or 100% sequence identity to the sequence shown in SEQ ID NO: 1, or a functional fragment thereof;
b) an ITGAM promoter, or a functional fragment thereof, or a promoter having at least 95%, 96%, 97%, 98%, 99% or 100% sequence identity to the sequence shown in SEQ ID NO: 5, or a functional fragment thereof
b) an AIF1 promoter, or a functional fragment thereof, or a promoter having at least 95%, 96%, 97%, 98%, 99% or 100% sequence identity to the sequence shown in SEQ ID NO: 6, or a functional fragment thereof.

In a particular embodiment, the invention relates to a viral vector encoding GM-CSF, or a functional fragment thereof, or a polypeptide having at least 95%, 96%, 97%, 98% or 99% sequence identity to the sequence shown in SEQ ID NO: 13, or a functional fragment thereof, wherein the microglia-specific promoter is
a) a TMEM119 promoter or a promoter having at least 95%, 96%, 97%, 98%, 99% or 100% sequence identity to the sequence shown in SEQ ID NO:3, SEQ ID NO:23 or SEQ ID NO:24, or a functional fragment thereof, or
b) a P2RY12 promoter or a promoter having at least 95%, 96%, 97%, 98%, 99% or 100% sequence identity to the sequence shown in SEQ ID NO: 2, SEQ ID NO:21 or SEQ ID NO:22, or a functional fragment thereof;
c) an OLFML3 promoter or a promoter having at least 95%, 96%, 97%, 98%, 99% or 100% sequence identity to the sequence shown in SEQ ID NO:4 or SEQ ID NO:25, or a functional fragment thereof.

In a particular embodiment, the invention relates to a viral vector encoding GM-CSF, or a functional fragment thereof; or a polypeptide having at least 95%, 96%, 97%, 98% or 99% sequence identity to the sequence shown in SEQ ID NO: 13, or a functional fragment thereof, wherein the first promoter is a myelo-specific promoter and wherein the second promoter is a microglia-specific promoter, or vice versa.

The second promoter may be any promoter known in the art. However, in certain embodiments, GM-CSF or a functional fragment or mutant variant thereof as disclosed above may be expressed from a fusion promoter comprising a myelo-specific promoter and a microglia-specific promoter. That is, any of the myelo-specific promoters disclosed above may be combined with any of the microglia-specific promoters disclosed above, in any order.

In certain embodiments, GM-CSF or a functional fragment or mutant variant thereof as disclosed above may be expressed from a fusion promoter comprising miR223, a functional fragment thereof or a promoter with miR223 functionality, and a microglia-specific promoter.

That is, in a particular embodiment, the invention relates to a viral vector encoding GM-CSF, or a functional fragment thereof; or a polypeptide having at least 95%, 96%, 97%, 98% or 99% sequence identity to the sequence shown in SEQ ID NO: 13, or a functional fragment thereof, wherein the first promoter is a miR233 promoter, or a promoter having at least 95%, 96%, 97%, 98%, 99% or 100% sequence identity to the sequence shown in SEQ ID NO: 1, or a functional fragment thereof; and wherein the first promoter is operably linked to
i) a TMEM119 promoter, or a promoter having at least 95%, 96%, 97%, 98%, 99% or 100% sequence identity to the sequence shown in SEQ ID NO:3, SEQ ID NO:23 or SEQ ID NO:24, or a functional fragment thereof;
ii) a P2RY12 promoter, or a promoter having at least 95%, 96%, 97%, 98%, 99% or 100% sequence identity to the sequence shown in SEQ ID NO: 2, SEQ ID NO:21 or SEQ ID NO:22, or a functional fragment thereof;
iii) an OLFML3 promoter, or a promoter having at least 95%, 96%, 97%, 98%, 99% or 100% sequence identity to the sequence shown in SEQ ID NO:4 or SEQ ID NO:25, or a functional fragment thereof;
iv) an ITGAM promoter, or a promoter having at least 95%, 96%, 97%, 98%, 99% or 100% sequence identity to the sequence shown in SEQ ID NO:6, or a functional fragment thereof; and/or
v) an AIF1 promoter or a promoter having at least 95%, 96%, 97%, 98%, 99% or 100% sequence identity to the sequence shown in SEQ ID NO:5, or a functional fragment thereof.

In certain embodiments, the invention relates to a viral vector encoding GM-CSF, or a functional fragment thereof; or a polypeptide having at least 95%, 96%, 97%, 98% or 99% sequence identity to the sequence shown in SEQ ID NO: 13, or a functional fragment thereof, wherein the promoter is a miR233 promoter, or a promoter having at least 95%, 96%, 97%, 98%, 99% or 100% sequence identity to the sequence shown in SEQ ID NO: 1, or a functional fragment thereof.

In certain embodiments, the invention relates to a viral vector encoding GM-CSF, or a functional fragment thereof; or a polypeptide having at least 95%, 96%, 97%, 98% or 99% sequence identity to the sequence shown in SEQ ID NO: 13, or a functional fragment thereof, wherein the first promoter is an ITGAM promoter, or a promoter having at least 95%, 96%, 97%, 98%, 99% or 100% sequence identity to the sequence shown in SEQ ID NO:6, or a functional fragment thereof.

In certain embodiments, the invention relates to a viral vector encoding GM-CSF, or a functional fragment thereof; or a polypeptide having at least 95%, 96%, 97%, 98% or 99% sequence identity to the sequence shown in SEQ ID NO: 13, or a functional fragment thereof, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof; and (b) a P2RY12 promoter, or a promoter having at least 95%, 96%, 97%, 98%, 99% or 100% sequence identity to the sequence shown in SEQ ID NO: 2, SEQ ID NO:21 or SEQ ID NO:22, or a functional fragment thereof. In certain embodiments, the fusion promoter comprises a nucleotide sequence having at least 90%, at least 95%, at least 96%, at least 97%, at least 98%, at least 99% or 100% sequence identity with SEQ ID NO:26 or SEQ ID NO:27.

In certain embodiments, the invention relates to a viral vector encoding GM-CSF, or a functional fragment thereof; or a polypeptide having at least 95%, 96%, 97%, 98% or 99% sequence identity to the sequence shown in SEQ ID NO: 13, or a functional fragment thereof, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof; and (b) a TMEM119 promoter, or a promoter having at least 95%, 96%, 97%, 98%, 99% or 100% sequence identity to the sequence shown in SEQ ID NO:3, SEQ ID NO:23 or SEQ ID NO:24, or a functional fragment thereof. In certain embodiments, the fusion promoter comprises a nucleotide sequence having at least 90%, at least 95%, at least 96%, at least 97%, at least 98%, at least 99% or 100% sequence identity with SEQ ID NO:28.

In certain embodiments, the invention relates to a viral vector encoding GM-CSF, or a functional fragment thereof; or a polypeptide having at least 95%, 96%, 97%, 98% or 99% sequence identity to the sequence shown in SEQ ID NO: 13, or a functional fragment thereof, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof; and (b) an OLFML3 promoter, or a promoter having at least 95%, 96%, 97%, 98%, 99% or 100% sequence identity to the sequence shown in SEQ ID NO:4 or SEQ ID NO:25, or a functional fragment thereof. In certain embodiments, the fusion promoter comprises a nucleotide sequence having at least 90%, at least 95%, at least 96%, at least 97%, at least 98%, at least 99% or 100% sequence identity with SEQ ID NO:29.

In a particular embodiment, the invention relates to a viral vector comprising a transgene under control of one or more promoters, wherein the transgene encodes
a) G-CSF, or a functional fragment thereof; or
b) a polypeptide having at least 95%, 96%, 97%, 98%, 99% or 100% sequence identity to the sequence shown in SEQ ID NO: 14, or a functional fragment thereof.

That is, in certain embodiments, the invention relates to a viral vector encoding Granulocyte colony-stimulating factor (G-CSF). The term “G-CSF” refers to the protein sequence of SEQ ID NO: 14, and/or to any sequence with a sequence identity of >95% homology thereof. Also provided herein are nucleic acid sequences encoding said proteins.

(SEQ ID NO: 14)
MAGPATQSPMKLMALQLLLWHSALWTVQEATPLGPASSLPQSFLLKCLEQ
VRKIQGDGAALQEKLVSECATYKLCHPEELVLLGHSLGIPWAPLSSCPSQ
ALQLAGCLSQLHSGLFLYQGLLQALEGISPELGPTLDTLQLDVADFATTI
WQQMEELGMAPALQPTQGAMPAFASAFQRRAGGVLVASHLQSFLEVSYRV
LRHLAQP

Granulocyte colony-stimulating factor (G-CSF or GCSF), also known as colony-stimulating factor 3 (CSF 3), is a glycoprotein that stimulates the bone marrow to produce granulocytes and stem cells and release them into the bloodstream. Functionally, it is a cytokine and hormone, a type of colony-stimulating factor, and is produced by a number of different tissues. The pharmaceutical analogs of naturally occurring G-CSF are called filgrastim and lenograstim. G-CSF also stimulates the survival, proliferation, differentiation, and function of neutrophil precursors and mature neutrophils.

Chemotherapy can cause myelosuppression and unacceptably low levels of white blood cells (leukopenia), making patients susceptible to infections and sepsis. G-CSF stimulates the production of granulocytes, a type of white blood cell. In oncology and hematology, a recombinant form of G-CSF is used with certain cancer patients to accelerate recovery and reduce mortality from neutropenia after chemotherapy, allowing higher-intensity treatment regimens. G-CSF has been shown to reduce inflammation, reduce amyloid beta burden, and reverse cognitive impairment in a mouse model of Alzheimer's disease. Due to its neuroprotective properties, G-CSF is currently under investigation for cerebral ischemia in a clinical phase IIb and several clinical pilot studies are published for other neurological disease such as amyotrophic lateral sclerosis.

A functional fragment of G-CSF is a fragment of at least 50, at least 100 amino acids, at least 120, at least 140, at least 160 or at least 180 amino acids having at least 95% sequence identity with SEQ ID NO:14, wherein the fragment has G-CSF activity. Assays to determine whether a protein has G-CSF activity have been described in the art, for example by Mickiene et al., Human granulocyte-colony stimulating factor (G-CSF)/stem cell factor (SCF) fusion proteins: design, characterization and activity; PeerJ. 2020; 8: e9788.

In a particular embodiment, the invention relates to a viral vector encoding G-CSF, or a functional fragment thereof; or a polypeptide having at least 95%, 96%, 97%, 98% or 99% sequence identity to the sequence shown in SEQ ID NO: 14, or a functional fragment thereof, wherein the one or more promoters comprises:
a) a myelo-specific promoter, or a functional fragment thereof; and/or
b) a microglia-specific promoter, or a functional fragment thereof; and/or
c) a fusion promoter comprising or consisting of
   i) a first promoter, wherein said first promoter is a myelo-specific promoter or a microglia-specific promoter, or a functional fragment thereof; and
   ii) a second promoter.

That is, in certain embodiments, G-CSF or a functional fragment or mutant variant thereof as disclosed above may be expressed from a myelo-specific promoter or from a functional fragment thereof. In other embodiments, G-CSF or a functional fragment or mutant variant thereof as disclosed above may be expressed from a microglia-specific promoter or from a functional fragment thereof. In other embodiments, G-CSF or a functional fragment or mutant variant thereof as disclosed above may be expressed from a fusion promoter, preferably wherein the fusion promoter comprises a myelo-specific or a microglia-specific promoter or functional fragments thereof.

That is, in a particular embodiment, the invention relates to a viral vector encoding G-CSF a, or a functional fragment thereof; or a polypeptide having at least 95%, 96%, 97%, 98% or 99% sequence identity to the sequence shown in SEQ ID NO: 14, or a functional fragment thereof, wherein the myelo-specific promoter is
a) a miR233 promoter, or a functional fragment thereof; or a promoter having at least 95%, 96%, 97%, 98%, 99% or 100% sequence identity to the sequence shown in SEQ ID NO: 1, or a functional fragment thereof;
c) an ITGAM promoter, or a functional fragment thereof; or a promoter having at least 95%, 96%, 97%, 98%, 99% or 100% sequence identity to the sequence shown in SEQ ID NO: 6, or a functional fragment thereof; or
b) an AIF1 promoter, or a functional fragment thereof; or a promoter having at least 95%, 96%, 97%, 98%, 99% or 100% sequence identity to the sequence shown in SEQ ID NO: 5, or a functional fragment thereof.

In a particular embodiment, the invention relates to a viral vector encoding G-CSF, or a functional fragment thereof; or a polypeptide having at least 95%, 96%, 97%, 98% or 99% sequence identity to the sequence shown in SEQ ID NO: 14, or a functional fragment thereof, wherein the microglia-specific promoter is
a) a TMEM119 promoter or a promoter having at least 95%, 96%, 97%, 98%, 99% or 100% sequence identity to the sequence shown in SEQ ID NO:3, SEQ ID NO:23 or SEQ ID NO:24, or a functional fragment thereof;
b) a P2RY12 promoter or a promoter having at least 95%, 96%, 97%, 98%, 99% or 100% sequence identity to the sequence shown in SEQ ID NO: 2, SEQ ID NO:21 or SEQ ID NO:22, or a functional fragment thereof; or
c) an OLFML3 promoter or a promoter having at least 95%, 96%, 97%, 98%, 99% or 100% sequence identity to the sequence shown in SEQ ID NO:4 or SEQ ID NO:25, or a functional fragment thereof.

In a particular embodiment, the invention relates to a viral vector encoding G-CSF, or a functional fragment thereof; or a polypeptide having at least 95%, 96%, 97%, 98% or 99% sequence identity to the sequence shown in SEQ ID NO: 14, or a functional fragment thereof, wherein the first promoter is a myelo-specific promoter and wherein the second promoter is a microglia-specific promoter, or vice versa.

The second promoter may be any promoter known in the art. However, in certain embodiments, G-CSF or a functional fragment or mutant variant thereof as disclosed above may be expressed from a fusion promoter comprising a myelo-specific promoter and a microglia-specific promoter. That is, any of the myelo-specific promoters disclosed above may be combined with any of the microglia-specific promoters disclosed above, in any order.

In certain embodiments, G-CSF or a functional fragment or mutant variant thereof as disclosed above may be expressed from a fusion promoter comprising miR223, a functional fragment thereof or a promoter with miR223 functionality, and a microglia-specific promoter.

That is, in a particular embodiment, the invention relates to a viral vector encoding G-CSF, or a functional fragment thereof; or a polypeptide having at least 95%, 96%, 97%, 98% or 99% sequence identity to the sequence shown in SEQ ID NO: 14, or a functional fragment thereof, wherein the first promoter is a miR233 promoter, or a promoter having at least 95%, 96%, 97%, 98%, 99% or 100% sequence identity to the sequence shown in SEQ ID NO: 1, or a functional fragment thereof; and wherein the first promoter is operably linked to
i) a TMEM119 promoter, or a promoter having at least 95%, 96%, 97%, 98%, 99% or 100% sequence identity to the sequence shown in SEQ ID NO:3, SEQ ID NO:23 or SEQ ID NO:24, or a functional fragment thereof;
ii) a P2RY12 promoter, or a promoter having at least 95%, 96%, 97%, 98%, 99% or 100% sequence identity to the sequence shown in SEQ ID NO: 2, SEQ ID NO:21 or SEQ ID NO:22, or a functional fragment thereof;
iii) an OLFML3 promoter, or a promoter having at least 95%, 96%, 97%, 98%, 99% or 100% sequence identity to the sequence shown in SEQ ID NO:4 or SEQ ID NO:25, or a functional fragment thereof;
iv) an ITGAM promoter, or a promoter having at least 95%, 96%, 97%, 98%, 99% or 100% sequence identity to the sequence shown in SEQ ID NO:6, or a functional fragment thereof; and/or
v) an AIF1 promoter or a promoter having at least 95%, 96%, 97%, 98%, 99% or 100% sequence identity to the sequence shown in SEQ ID NO:5, or a functional fragment thereof.

In certain embodiments, the invention relates to a viral vector encoding G-CSF, or a functional fragment thereof; or a polypeptide having at least 95%, 96%, 97%, 98% or 99% sequence identity to the sequence shown in SEQ ID NO: 14, or a functional fragment thereof, wherein the promoter is a miR233 promoter, or a promoter having at least 95%, 96%, 97%, 98%, 99% or 100% sequence identity to the sequence shown in SEQ ID NO: 1, or a functional fragment thereof.

In certain embodiments, the invention relates to a viral vector encoding G-CSF, or a functional fragment thereof; or a polypeptide having at least 95%, 96%, 97%, 98% or 99% sequence identity to the sequence shown in SEQ ID NO: 14, or a functional fragment thereof, wherein the first promoter is an ITGAM promoter, or a promoter having at least 95%, 96%, 97%, 98%, 99% or 100% sequence identity to the sequence shown in SEQ ID NO:6, or a functional fragment thereof.

In certain embodiments, the invention relates to a viral vector encoding G-CSF, or a functional fragment thereof; or a polypeptide having at least 95%, 96%, 97%, 98% or 99% sequence identity to the sequence shown in SEQ ID NO: 14, or a functional fragment thereof, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof; and (b) a P2RY12 promoter, or a promoter having at least 95%, 96%, 97%, 98%, 99% or 100% sequence identity to the sequence shown in SEQ ID NO: 2, SEQ ID NO:21 or SEQ ID NO:22, or a functional fragment thereof. In certain embodiments, the fusion promoter comprises a nucleotide sequence having at least 90%, at least 95%, at least 96%, at least 97%, at least 98%, at least 99% or 100% sequence identity with SEQ ID NO:26 or SEQ ID NO:27.

In certain embodiments, the invention relates to a viral vector encoding G-CSF, or a functional fragment thereof; or a polypeptide having at least 95%, 96%, 97%, 98% or 99% sequence identity to the sequence shown in SEQ ID NO: 14, or a functional fragment thereof, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof; and (b) a TMEM119 promoter, or a promoter having at least 95%, 96%, 97%, 98%, 99% or 100% sequence identity to the sequence shown in SEQ ID NO:3, SEQ ID NO:23 or SEQ ID NO:24, or a functional fragment thereof. In certain embodiments, the fusion promoter comprises a nucleotide sequence having at least 90%, at least 95%, at least 96%, at least 97%, at least 98%, at least 99% or 100% sequence identity with SEQ ID NO:28.

In certain embodiments, the invention relates to a viral vector encoding G-CSF, or a functional fragment thereof; or a polypeptide having at least 95%, 96%, 97%, 98% or 99% sequence identity to the sequence shown in SEQ ID NO: 14, or a functional fragment thereof, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof; and (b) an OLFML3 promoter, or a promoter having at least 95%, 96%, 97%, 98%, 99% or 100% sequence identity to the sequence shown in SEQ ID NO:4 or SEQ ID NO:25, or a functional fragment thereof. In certain embodiments, the fusion promoter comprises a nucleotide sequence having at least 90%, at least 95%, at least 96%, at least 97%, at least 98%, at least 99% or 100% sequence identity with SEQ ID NO:29.

In a particular embodiment, the invention relates to a viral vector comprising a transgene under control of one or more promoters, wherein the transgene encodes
a) GM-CSF and IFN-gamma, or functional fragments thereof; or
b) a first polypeptide having at least 95%, 96%, 97%, 98% or 99% sequence identity to the sequence shown in SEQ ID NO: 10 or a functional fragment thereof, and a second polypeptide having at least 95%, 96%, 97%, 98% or 99% sequence identity to the sequence shown in SEQ ID NO: 13, or a functional fragment thereof; or
c) a nucleic acid sequence having at least 95%, 96%, 97%, 98% or 99% sequence identity to the sequence shown in SEQ ID NO: 15.

That is, in certain embodiments, the invention relates to a viral vector encoding a GM-CSF-INF-gamma co-expression construct. The co-expression construct may encode GM-CSF or any functional fragment or variant thereof as defined above. The co-expression construct may further encode INF-gamma or any functional fragment or variant thereof as defined above. GM-CSF and INF-gamma, and the functional fragments or variants thereof, may be expressed as separate polypeptides from the viral vector of the invention. In certain embodiments, GM-CSF and INF-gamma may be expressed as a fusion protein.

An exemplary nucleic acid sequence for GM-CSF-INF-gamma co-expression may comprise the following nucleic acid sequence:

(SEQ ID NO: 15)
TCCTTCCAGCCATGTTTAAATATACAAGTTATATCTTGGCTTTTCAGCTC
TGCATCGTTTTGGGTTCTCTTGGCTGTTACTGCCAGGACCCATATGTAAA
AGAAGCAGAAAACCTTAAGAAATATTTTAATGCCGGTCATTCAGATGTAG
CGGATAATGGAACTCTTTTCTTAGGCATTTTGAAGAATTGGAAAGAGGAG
AGTGACAGAAAAATAATGCAGAGCCAAATTGTCTCCTTTTACTTCAAACT
TTTTAAGAACTTTAAGGATGACCAGAGCATCCAAAAGAGTGTGGAGACCA
TCAAGGAAGACATGAATGTCAAGTTTTTCAATAGCAACAAAAAGAAACGA
GATGACTTCGAAAAGCTGACTAATTATTCGGTAACTGACTTGAATGTCCA
ACGCAAAGCAATACATGAACTCATCCAAGTGATGGCTGAACTGTCGCCAG
CAGCGAAAACAGGGAAGCGAAAAAGGAGTCAGATGCTGTTTCGAGGTCGA
AGAGCATCCCAGTAAGATATCCCCTCTCCCTCCCCCCCCCCTAACGTTAC
TGGCCGAAGCCGCTTGGAATAAGGCCGGTGTGCGTTTGTCTATATGTTAT
TTTCCACCATATTGCCGTCTTTTGGCAATGTGAGGGCCCGGAAACCTGGC
CCTGTCTTCTTGACGAGCATTCCTAGGGGTCTTTCCCCTCTCGCCAAAGG
AATGCAAGGTCTGTTGAATGTCGTGAAGGAAGCAGTTCCTCTGGAAGCTT
CTTGAAGACAAACAACGTCTGTAGCGACCCTTTGCAGGCAGCGGAACCCC
CCACCTGGCGACAGGTGCCTCTGCGGCCAAAAGCCACGTGTATAAGATAC
ACCTGCAAAGGCGGCACAACCCCAGTGCCACGTTGTGAGTTGGATAGTTG
TGGAAAGAGTCAAATGGCTCTCCTCAAGCGTATTCAACAAGGGGCTGAAG
GATGCCCAGAAGATACCCCATTGTATGGGATCTGATCTGGGGCCTCGGTG
CACATGCTTTACATGTGTTTAGTCGAGGTTAAAAAAACGTCTAGGCCCCC
CGAACCACGGGGACGTGGTTTTCCTTTGAAAAACACGATGATAAATGTGG
CCACTGCAAAGTCTGCTTTTGCTGGGCACCGTAGCTTGTAGCATATCAGC
GCCTGCTCGGAGTCCCTCTCCATCAACGCAACCCTGGGAACACGTGAACG
CAATTCAGGAGGCAAGAAGGTTGCTGAACCTGAGCCGGGACACCGCCGCT
GAAATGAATGAAACCGTAGAAGTGATTTCCGAGATGTTTGACCTCCAAGA
ACCAACTTGTCTGCAAACAAGACTTGAGCTTTATAAACAGGGACTCCGAG
GCAGCCTGACAAAACTCAAGGGGCCCCTCACAATGATGGCAAGCCATTAT
AAACAACACTGTCCTCCGACCCCGGAGACTTCTTGCGCCACACAGATCAT
CACTTTTGAGAGCTTCAAAGAGAACCTTAAAGACTTTCTGCTGGTCATTC
CGTTCGATTGCTGGGAACCCGTGCAGGAGTGA

In a particular embodiment, the invention relates to a viral vector encoding a GM-CSF-INF-gamma co-expression construct, or a functional fragment thereof; or a sequence having at least 95%, 96%, 97%, 98% or 99% sequence identity to the sequence shown in SEQ ID NO: 15; or encode a first polypeptide having at least 95%, 96%, 97%, 98% or 99% sequence identity to the sequence shown in SEQ ID NO: 10, or a functional fragment thereof, and a second polypeptide having at least 95%, 96%, 97%, 98% or 99% sequence identity to the sequence shown in SEQ ID NO: 13, or a functional fragment thereof, wherein the one or more promoters comprises:
a) a myelo-specific promoter, or a functional fragment thereof; and/or
b) a microglia-specific promoter, or a functional fragment thereof; and/or
c) a fusion promoter comprising or consisting of
   i) a first promoter, wherein said first promoter is a myelo-specific promoter or a microglia-specific promoter, or a functional fragment thereof; and
   ii) a second promoter.

That is, in certain embodiments, a GM-CSF-INF-gamma co-expression construct or a functional fragment or mutant variant thereof as disclosed above may be expressed from a myelo-specific promoter or from a functional fragment thereof. In other embodiments, a GM-CSF-INF-gamma co-expression construct or a functional fragment or mutant variant thereof as disclosed above may be expressed from a microglia-specific promoter or from a functional fragment thereof. In other embodiments, a GM-CSF-INF-gamma co-expression construct or a functional fragment or mutant variant thereof as disclosed above may be expressed from a fusion promoter, preferably wherein the fusion promoter comprises a myelo-specific or a microglia-specific promoter or functional fragments thereof.

That is, in a particular embodiment, the invention relates to a viral vector encoding a GM-CSF-INF-gamma co-expression construct, or a functional fragment thereof; or a nucleic acid sequence having at least 95%, 96%, 97%, 98% or 99% sequence identity to the sequence shown in SEQ ID NO: 15; or encoding a first polypeptide having at least 95%, 96%, 97%, 98% or 99% sequence identity to the sequence shown in SEQ ID NO: 10, or encoding a functional fragment thereof, and a second polypeptide having at least 95%, 96%, 97%, 98% or 99% sequence identity to the sequence shown in SEQ ID NO: 13, or a functional fragment thereof, wherein the myelo-specific promoter is
a) a miR233 promoter, or a functional fragment thereof; or a promoter having at least 95%, 96%, 97%, 98%, 99% or 100% sequence identity to the sequence shown in SEQ ID NO: 1, or a functional fragment thereof;
b) an ITGAM promoter, or a functional fragment thereof; or a promoter having at least 95%, 96%, 97%, 98%, 99% or 100% sequence identity to the sequence shown in SEQ ID NO:6, or a functional fragment thereof;
c) an AIF1 promoter, or a functional fragment thereof; or a promoter having at least 95%, 96%, 97%, 98%, 99% or 100% sequence identity to the sequence shown in SEQ ID NO:5, or a functional fragment thereof; or

In a particular embodiment, the invention relates to a viral vector encoding a GM-CSF-INF-gamma co-expression construct, or a functional fragment thereof; or a nucleic acid sequence having at least 95%, 96%, 97%, 98% or 99% sequence identity to the sequence shown in SEQ ID NO: 15; or encoding a first polypeptide having at least 95%, 96%, 97%, 98% or 99% sequence identity to the sequence shown in SEQ ID NO: 10, or encoding a functional fragment thereof, and a second polypeptide having at least 95%, 96%, 97%, 98% or 99% sequence identity to the sequence shown in SEQ ID NO: 13, or a functional fragment thereof, wherein the microglia-specific promoter is
a) a TMEM119 promoter or a promoter having at least 95%, 96%, 97%, 98%, 99% or 100% sequence identity to the sequence shown in SEQ ID NO:3, SEQ ID NO:23 or SEQ ID NO:24, or a functional fragment thereof,
b) a P2RY12 promoter or a promoter having at least 95%, 96%, 97%, 98%, 99% or 100% sequence identity to the sequence shown in SEQ ID NO: 2, SEQ ID NO:21 or SEQ ID NO:22, or a functional fragment thereof; or
c) an OLFML3 promoter or a promoter having at least 95%, 96%, 97%, 98%, 99% or 100% sequence identity to the sequence shown in SEQ ID NO:4 or SEQ ID NO:25, or a functional fragment thereof.

In a particular embodiment, the invention relates to a viral vector encoding a GM-CSF-INF-gamma co-expression construct, or a functional fragment thereof; or a nucleic acid sequence having at least 95%, 96%, 97%, 98% or 99% sequence identity to the sequence shown in SEQ ID NO: 15; or encoding a first polypeptide having at least 95%, 96%, 97%, 98% or 99% sequence identity to the sequence shown in SEQ ID NO: 10, or encoding a functional fragment thereof, and a second polypeptide having at least 95%, 96%, 97%, 98% or 99% sequence identity to the sequence shown in SEQ ID NO: 13, or a functional fragment thereof, wherein the first promoter is a myelo-specific promoter and wherein the second promoter is a microglia-specific promoter, or vice versa.

The second promoter may be any promoter known in the art. However, in certain embodiments, a GM-CSF-INF-gamma co-expression construct or a functional fragment or mutant variant thereof as disclosed above may be expressed from a fusion promoter comprising a myelo-specific promoter and a microglia-specific promoter. That is, any of the myelo-specific promoters disclosed above may be combined with any of the microglia-specific promoters disclosed above, in any order.

In certain embodiments, a GM-CSF-INF-gamma co-expression construct or a functional fragment or mutant variant thereof as disclosed above may be expressed from a fusion promoter comprising miR223, a functional fragment thereof or a promoter with miR223 functionality, and a microglia-specific promoter.

That is, in a particular embodiment, the invention relates to a viral vector encoding a GM-CSF-INF-gamma co-expression construct; or a nucleic acid sequence having at least 95%, 96%, 97%, 98% or 99% sequence identity to the sequence shown in SEQ ID NO: 15; or encoding a first polypeptide having at least 95%, 96%, 97%, 98% or 99% sequence identity to the sequence shown in SEQ ID NO: 10, or encoding a functional fragment thereof, and a second polypeptide having at least 95%, 96%, 97%, 98% or 99% sequence identity to the sequence shown in SEQ ID NO: 13, or a functional fragment thereof, wherein the first promoter is a miR233 promoter, or a promoter having at least 95%, 96%, 97%, 98%, 99% or 100% sequence identity to the sequence shown in SEQ ID NO: 1, or a functional fragment thereof; and wherein the first promoter is operably linked to
i) a TMEM119 promoter, or a promoter having at least 95%, 96%, 97%, 98%, 99% or 100% sequence identity to the sequence shown in SEQ ID NO:3, SEQ ID NO:23 or SEQ ID NO:24, or a functional fragment thereof;
ii) a P2RY12 promoter, or a promoter having at least 95%, 96%, 97%, 98%, 99% or 100% sequence identity to the sequence shown in SEQ ID NO: 2, SEQ ID NO:21 or SEQ ID NO:22, or a functional fragment thereof;
iii) an OLFML3 promoter, or a promoter having at least 95%, 96%, 97%, 98%, 99% or 100% sequence identity to the sequence shown in SEQ ID NO:4 or SEQ ID NO:25, or a functional fragment thereof;
iv) an ITGAM promoter, or a promoter having at least 95%, 96%, 97%, 98%, 99% or 100% sequence identity to the sequence shown in SEQ ID NO:6, or a functional fragment thereof; and/or
v) an AIF1 promoter or a promoter having at least 95%, 96%, 97%, 98%, 99% or 100% sequence identity to the sequence shown in SEQ ID NO:5, or a functional fragment thereof.

In certain embodiments, the invention relates to a viral vector encoding a GM-CSF-INF-gamma co-expression construct, or a functional fragment thereof; or a nucleic acid sequence having at least 95%, 96%, 97%, 98% or 99% sequence identity to the sequence shown in SEQ ID NO: 15; or encoding a first polypeptide having at least 95%, 96%, 97%, 98% or 99% sequence identity to the sequence shown in SEQ ID NO: 10, or encoding a functional fragment thereof, and a second polypeptide having at least 95%, 96%, 97%, 98% or 99% sequence identity to the sequence shown in SEQ ID NO: 13, or a functional fragment thereof, wherein the promoter is a miR233 promoter, or a promoter having at least 95%, 96%, 97%, 98%, 99% or 100% sequence identity to the sequence shown in SEQ ID NO: 1, or a functional fragment thereof.

In certain embodiments, the invention relates to a viral vector encoding a GM-CSF-INF-gamma co-expression construct, or a functional fragment thereof; or a nucleic acid sequence having at least 95%, 96%, 97%, 98% or 99% sequence identity to the sequence shown in SEQ ID NO: 15; or encoding a first polypeptide having at least 95%, 96%, 97%, 98% or 99% sequence identity to the sequence shown in SEQ ID NO: 10, or encoding a functional fragment thereof, and a second polypeptide having at least 95%, 96%, 97%, 98% or 99% sequence identity to the sequence shown in SEQ ID NO: 13, or a functional fragment thereof, wherein the first promoter is an ITGAM promoter, or a promoter having at least 95%, 96%, 97%, 98%, 99% or 100% sequence identity to the sequence shown in SEQ ID NO:6, or a functional fragment thereof.

In certain embodiments, the invention relates to a viral vector encoding a GM-CSF-INF-gamma co-expression construct, or a functional fragment thereof; or a nucleic acid sequence having at least 95%, 96%, 97%, 98% or 99% sequence identity to the sequence shown in SEQ ID NO: 15; or encoding a first polypeptide having at least 95%, 96%, 97%, 98% or 99% sequence identity to the sequence shown in SEQ ID NO: 10, or encoding a functional fragment thereof, and a second polypeptide having at least 95%, 96%, 97%, 98% or 99% sequence identity to the sequence shown in SEQ ID NO: 13, or a functional fragment thereof, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof; and (b) a P2RY12 promoter, or a promoter having at least 95%, 96%, 97%, 98%, 99% or 100% sequence identity to the sequence shown in SEQ ID NO: 2, SEQ ID NO:21 or SEQ ID NO:22, or a functional fragment thereof. In certain embodiments, the fusion promoter comprises a nucleotide sequence having at least 90%, at least 95%, at least 96%, at least 97%, at least 98%, at least 99% or 100% sequence identity with SEQ ID NO:26 or SEQ ID NO:27.

In certain embodiments, the invention relates to a viral vector encoding a GM-CSF-INF-gamma co-expression construct, or a functional fragment thereof; or a nucleic acid sequence having at least 95%, 96%, 97%, 98% or 99% sequence identity to the sequence shown in SEQ ID NO: 15; or encoding a first polypeptide having at least 95%, 96%, 97%, 98% or 99% sequence identity to the sequence shown in SEQ ID NO: 10, or encoding a functional fragment thereof, and a second polypeptide having at least 95%, 96%, 97%, 98% or 99% sequence identity to the sequence shown in SEQ ID NO: 13, or a functional fragment thereof, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof; and (b) a TMEM119 promoter, or a promoter having at least 95%, 96%, 97%, 98%, 99% or 100% sequence identity to the sequence shown in SEQ ID NO:3, SEQ ID NO:23 or SEQ ID NO:24, or a functional fragment thereof. In certain embodiments, the fusion promoter comprises a nucleotide sequence having at least 90%, at least 95%, at least 96%, at least 97%, at least 98%, at least 99% or 100% sequence identity with SEQ ID NO:28.

In certain embodiments, the invention relates to a viral vector encoding a GM-CSF-INF-gamma co-expression construct, or a nucleic acid sequence having at least 95%, 96%, 97%, 98% or 99% sequence identity to the sequence shown in SEQ ID NO: 15; or encoding a first polypeptide having at least 95%, 96%, 97%, 98% or 99% sequence identity to the sequence shown in SEQ ID NO: 10, or encoding a functional fragment thereof, and a second polypeptide having at least 95%, 96%, 97%, 98% or 99% sequence identity to the sequence shown in SEQ ID NO: 13, or a functional fragment thereof, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof; and (b) an OLFML3 promoter, or a promoter having at least 95%, 96%, 97%, 98%, 99% or 100% sequence identity to the sequence shown in SEQ ID NO:4 or SEQ ID NO:25, or a functional fragment thereof. In certain embodiments, the fusion promoter comprises a nucleotide sequence having at least 90%, at least 95%, at least 96%, at least 97%, at least 98%, at least 99% or 100% sequence identity with SEQ ID NO:29.

In a particular embodiment, the invention relates to a viral vector comprising a transgene under control of one or more promoters, wherein the transgene encodes
a) G-CSF and IFN-gamma, or functional fragments thereof; or
b) or a first polypeptide having at least 95%, 96%, 97%, 98% or 99% sequence identity to the sequence shown in SEQ ID NO: 10, or a functional fragment thereof, and a second polypeptide having at least 95%, 96%, 97%, 98% or 99% sequence identity to the sequence shown in SEQ ID NO: 14, or a functional fragment thereof; or
c) a polypeptide having at least 95%, 96%, 97%, 98% or 99% sequence identity to the sequence shown in SEQ ID NO: 16.

That is, in certain embodiments, the invention relates to a viral vector encoding a G-CSF-INF-gamma co-expression construct. The co-expression construct may encode G-CSF or any functional fragment or variant thereof as defined above. The co-expression construct may further encode INF-gamma or any functional fragment or variant thereof as defined above. G-CSF and INF-gamma, and the functional fragments or variants thereof, may be expressed as separate polypeptides from the viral vector of the invention. In certain embodiments, G-CSF and INF-gamma may be expressed as a fusion protein.

Also provided herein are nucleic acid sequences encoding said co-expression construct.

An exemplary G-CSF-INF-gamma co-expression construct may comprise the following nucleic acid sequence:

(SEQ ID NO: 16)
TCCTTCCAGCCATGTTTAAATATACAAGTTATATCTTGGCTTTTCAGCTC
TGCATCGTTTTGGGTTCTCTTGGCTGTTACTGCCAGGACCCATATGTAAA
AGAAGCAGAAAACCTTAAGAAATATTTTAATGCCGGTCATTCAGATGTAG
CGGATAATGGAACTCTTTTCTTAGGCATTTTGAAGAATTGGAAAGAGGAG
AGTGACAGAAAAATAATGCAGAGCCAAATTGTCTCCTTTTACTTCAAACT
TTTTAAGAACTTTAAGGATGACCAGAGCATCCAAAAGAGTGTGGAGACCA
TCAAGGAAGACATGAATGTCAAGTTTTTCAATAGCAACAAAAAGAAACGA
GATGACTTCGAAAAGCTGACTAATTATTCGGTAACTGACTTGAATGTCCA
ACGCAAAGCAATACATGAACTCATCCAAGTGATGGCTGAACTGTCGCCAG
CAGCGAAAACAGGGAAGCGAAAAAGGAGTCAGATGCTGTTTCGAGGTCGA
AGAGCATCCCAGTAAGATATCCCCTCTCCCTCCCCCCCCCCTAACGTTAC
TGGCCGAAGCCGCTTGGAATAAGGCCGGTGTGCGTTTGTCTATATGTTAT
TTTCCACCATATTGCCGTCTTTTGGCAATGTGAGGGCCCGGAAACCTGGC
CCTGTCTTCTTGACGAGCATTCCTAGGGGTCTTTCCCCTCTCGCCAAAGG
AATGCAAGGTCTGTTGAATGTCGTGAAGGAAGCAGTTCCTCTGGAAGCTT
CTTGAAGACAAACAACGTCTGTAGCGACCCTTTGCAGGCAGCGGAACCCC
CCACCTGGCGACAGGTGCCTCTGCGGCCAAAAGCCACGTGTATAAGATAC
ACCTGCAAAGGCGGCACAACCCCAGTGCCACGTTGTGAGTTGGATAGTTG
TGGAAAGAGTCAAATGGCTCTCCTCAAGCGTATTCAACAAGGGGCTGAAG
GATGCCCAGAAGATACCCCATTGTATGGGATCTGATCTGGGGCCTCGGTG
CACATGCTTTACATGTGTTTAGTCGAGGTTAAAAAAACGTCTAGGCCCCC
CGAACCACGGGGACGTGGTTTTCCTTTGAAAAACACGATGATAAATGGCC
GGCCCCGCCACCCAGAGCCCCATGAAGCTGATGGCCCTGCAGCTGCTGCT
GTGGCACAGCGCCCTGTGGACCGTGCAGGAGGCCACCCCCCTCGGCCCCG
CCAGCAGCCTGCCCCAGAGCTTCCTGCTGAAGTGCCTCGAACAAGTGCGC
AAGATACAAGGCGACGGCGCCGCCCTGCAGGAGAAGCTCGTGAGCGAGTG
CGCCACCTACAAGCTGTGCCACCCCGAGGAGCTGGTGCTGCTGGGCCACA
GCCTCGGCATCCCCTGGGCCCCCCTGAGCAGCTGCCCCAGCCAAGCCCTG
CAGCTGGCCGGCTGCCTGAGCCAGCTGCACAGCGGCCTGTTCCTGTACCA
AGGCTTACTACAGGCCCTCGAAGGCATCAGCCCCGAGCTGGGCCCCACCC
TCGACACCCTGCAGCTGGACGTGGCCGACTTCGCCACCACCATCTGGCAG
CAGATGGAGGAGCTGGGCATGGCCCCCGCCCTGCAGCCCACCCAAGGCGC
CATGCCCGCCTTCGCCAGCGCCTTCCAGCGCCGCGCCGGGGGCGTGCTGG
TGGCCAGCCACCTGCAGAGCTTCCTCGAAGTGAGCTACCGCGTGCTGCGC
CACCTCGCCCAGCCC TGA

In a particular embodiment, the invention relates to a viral vector encoding a G-CSF-INF-gamma co-expression construct, or a functional fragment thereof; or a nucleic acid sequence having at least 95%, 96%, 97%, 98% or 99% sequence identity to the sequence shown in SEQ ID NO: 16, or a functional fragment thereof, or encoding a first polypeptide comprising having at least 95%, 96%, 97%, 98% or 99% sequence identity to the sequence shown in SEQ ID NO: 10, or a functional fragment thereof, and encoding a second polypeptide having at least 95%, 96%, 97%, 98% or 99% sequence identity to the sequence shown in SEQ ID NO: 14, or a functional fragment thereof, wherein the one or more promoters comprises:
a) a myelo-specific promoter, or a functional fragment thereof, and/or
b) a microglia-specific promoter, or a functional fragment thereof, and/or
c) a fusion promoter comprising or consisting of
   i) a first promoter, wherein said first promoter is a myelo-specific promoter or a microglia-specific promoter, or a functional fragment thereof; and
   ii) a second promoter.

That is, in certain embodiments, a G-CSF-INF-gamma co-expression construct or a functional fragment or mutant variant thereof as disclosed above may be expressed from a myelo-specific promoter or from a functional fragment thereof. In other embodiments, a G-CSF-INF-gamma co-expression construct or a functional fragment or mutant variant thereof as disclosed above may be expressed from a microglia-specific promoter or from a functional fragment thereof. In other embodiments, a G-CSF-INF-gamma co-expression construct or a functional fragment or mutant variant thereof as disclosed above may be expressed from a fusion promoter, preferably wherein the fusion promoter comprises a myelo-specific or a microglia-specific promoter or functional fragments thereof.

That is, in a particular embodiment, the invention relates to a viral vector encoding a G-CSF-INF-gamma co-expression construct, or a functional fragment thereof, or a nucleic acid sequence having at least 95%, 96%, 97%, 98% or 99% sequence identity to the sequence shown in SEQ ID NO: 16, or a functional fragment thereof, or encoding a first polypeptide having at least 95%, 96%, 97%, 98% or 99% sequence identity to the sequence shown in SEQ ID NO: 10, or a functional fragment thereof, and a second polypeptide having at least 95%, 96%, 97%, 98% or 99% sequence identity to the sequence shown in SEQ ID NO: 14, or a functional fragment thereof, wherein the myelo-specific promoter is
a) a miR233 promoter, or a functional fragment thereof, or a promoter having at least 95%, 96%, 97%, 98%, 99% or 100% sequence identity to the sequence shown in SEQ ID NO: 1, or a functional fragment thereof;
b) an ITGAM promoter, or a functional fragment thereof, or a promoter having at least 95%, 96%, 97%, 98%, 99% or 100% sequence identity to the sequence shown in SEQ ID NO:6, or a functional fragment thereof, or
c) an AIF1 promoter, or a functional fragment thereof; or a promoter having at least 95%, 96%, 97%, 98%, 99% or 100% sequence identity to the sequence shown in SEQ ID NO:5, or a functional fragment thereof.

In a particular embodiment, the invention relates to a viral vector encoding a G-CSF-INF-gamma co-expression construct, or a functional fragment thereof; or a nucleic acid sequence having at least 95%, 96%, 97%, 98% or 99% sequence identity to the sequence shown in SEQ ID NO: 16, or a functional fragment thereof; or encoding a first polypeptide having at least 95%, 96%, 97%, 98% or 99% sequence identity to the sequence shown in SEQ ID NO: 10, or a functional fragment thereof, and a second polypeptide having at least 95%, 96%, 97%, 98% or 99% sequence identity to the sequence shown in SEQ ID NO: 14, or a functional fragment thereof, wherein the microglia-specific promoter is
a) a TMEM119 promoter or a promoter having at least 95%, 96%, 97%, 98%, 99% or 100% sequence identity to the sequence shown in SEQ ID NO:3, SEQ ID NO:23 or SEQ ID NO:24, or a functional fragment thereof,
b) a P2RY12 promoter or a promoter having at least 95%, 96%, 97%, 98%, 99% or 100% sequence identity to the sequence shown in SEQ ID NO: 2, SEQ ID NO:21 or SEQ ID NO:22, or a functional fragment thereof; or
c) an OLFML3 promoter or a promoter having at least 95%, 96%, 97%, 98%, 99% or 100% sequence identity to the sequence shown in SEQ ID NO:4 or SEQ ID NO:25, or a functional fragment thereof.

In a particular embodiment, the invention relates to a viral vector encoding a G-CSF-INF-gamma co-expression construct, or a functional fragment thereof; or a nucleic acid sequence having at least 95%, 96%, 97%, 98% or 99% sequence identity to the sequence shown in SEQ ID NO: 16, or a functional fragment thereof; or encoding a first polypeptide having at least 95%, 96%, 97%, 98% or 99% sequence identity to the sequence shown in SEQ ID NO: 10, or a functional fragment thereof, and a second polypeptide having at least 95%, 96%, 97%, 98% or 99% sequence identity to the sequence shown in SEQ ID NO: 14, or a functional fragment thereof, wherein the first promoter is a myelo-specific promoter and wherein the second promoter is a microglia-specific promoter, or vice versa.

The second promoter may be any promoter known in the art. However, in certain embodiments, a G-CSF-INF-gamma co-expression construct or a functional fragment or mutant variant thereof as disclosed above may be expressed from a fusion promoter comprising a myelo-specific promoter and a microglia-specific promoter. That is, any of the myelo-specific promoters disclosed above may be combined with any of the microglia-specific promoters disclosed above, in any order.

In certain embodiments, a G-CSF-INF-gamma co-expression construct or a functional fragment or mutant variant thereof as disclosed above may be expressed from a fusion promoter comprising miR223, a functional fragment thereof or a promoter with miR223 functionality, and a microglia-specific promoter.

That is, in a particular embodiment, the invention relates to a viral vector encoding a G-CSF-INF-gamma co-expression construct, or a functional fragment thereof; or a nucleic acid sequence having at least 95%, 96%, 97%, 98% or 99% sequence identity to the sequence shown in SEQ ID NO: 16, or a functional fragment thereof; or encoding a first polypeptide having at least 95%, 96%, 97%, 98% or 99% sequence identity to the sequence shown in SEQ ID NO: 10, or a functional fragment thereof, and a second polypeptide having at least 95%, 96%, 97%, 98% or 99% sequence identity to the sequence shown in SEQ ID NO: 14, or a functional fragment thereof, wherein the first promoter is a miR233 promoter, or a promoter having at least 95%, 96%, 97%, 98%, 99% or 100% sequence identity to the sequence shown in SEQ ID NO: 1, or a functional fragment thereof; and wherein the first promoter is operably linked to
i) a TMEM119 promoter, or a promoter having at least 95%, 96%, 97%, 98%, 99% or 100% sequence identity to the sequence shown in SEQ ID NO:3, SEQ ID NO:23 or SEQ ID NO:24, or a functional fragment thereof;
ii) a P2RY12 promoter, or a promoter having at least 95%, 96%, 97%, 98%, 99% or 100% sequence identity to the sequence shown in SEQ ID NO: 2, SEQ ID NO:21 or SEQ ID NO:22, or a functional fragment thereof;
iii) an OLFML3 promoter, or a promoter having at least 95%, 96%, 97%, 98%, 99% or 100% sequence identity to the sequence shown in SEQ ID NO:4 or SEQ ID NO:25, or a functional fragment thereof;
iv) an ITGAM promoter, or a promoter having at least 95%, 96%, 97%, 98%, 99% or 100% sequence identity to the sequence shown in SEQ ID NO:6, or a functional fragment thereof; and/or
v) an AIF1 promoter or a promoter having at least 95%, 96%, 97%, 98%, 99% or 100% sequence identity to the sequence shown in SEQ ID NO:5, or a functional fragment thereof.

In certain embodiments, the invention relates to a viral vector encoding a G-CSF-INF-gamma co-expression construct, or a functional fragment thereof; or a nucleic acid sequence having at least 95%, 96%, 97%, 98% or 99% sequence identity to the sequence shown in SEQ ID NO: 16, or a functional fragment thereof; or encoding a first polypeptide having at least 95%, 96%, 97%, 98% or 99% sequence identity to the sequence shown in SEQ ID NO: 10, or a functional fragment thereof, and a second polypeptide having at least 95%, 96%, 97%, 98% or 99% sequence identity to the sequence shown in SEQ ID NO: 14, or a functional fragment thereof, wherein the promoter is a miR233 promoter, or a promoter having at least 95%, 96%, 97%, 98%, 99% or 100% sequence identity to the sequence shown in SEQ ID NO: 1, or a functional fragment thereof.

In certain embodiments, the invention relates to a viral vector encoding a G-CSF-INF-gamma co-expression construct, or a functional fragment thereof; or a nucleic acid sequence having at least 95%, 96%, 97%, 98% or 99% sequence identity to the sequence shown in SEQ ID NO: 16, or a functional fragment thereof; or encoding a first polypeptide having at least 95%, 96%, 97%, 98% or 99% sequence identity to the sequence shown in SEQ ID NO: 10, or a functional fragment thereof, and a second polypeptide having at least 95%, 96%, 97%, 98% or 99% sequence identity to the sequence shown in SEQ ID NO: 14, or a functional fragment thereof, wherein the first promoter is an ITGAM promoter, or a promoter having at least 95%, 96%, 97%, 98%, 99% or 100% sequence identity to the sequence shown in SEQ ID NO:6, or a functional fragment thereof.

In certain embodiments, the invention relates to a viral vector encoding a G-CSF-INF-gamma co-expression construct, or a functional fragment thereof; or a nucleic acid sequence having at least 95%, 96%, 97%, 98% or 99% sequence identity to the sequence shown in SEQ ID NO: 16, or a functional fragment thereof; or encoding a first polypeptide having at least 95%, 96%, 97%, 98% or 99% sequence identity to the sequence shown in SEQ ID NO: 10, or a functional fragment thereof, and a second polypeptide having at least 95%, 96%, 97%, 98% or 99% sequence identity to the sequence shown in SEQ ID NO: 14, or a functional fragment thereof, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof; and (b) a P2RY12 promoter, or a promoter having at least 95%, 96%, 97%, 98%, 99% or 100% sequence identity to the sequence shown in SEQ ID NO: 2, SEQ ID NO:21 or SEQ ID NO:22, or a functional fragment thereof. In certain embodiments, the fusion promoter comprises a nucleotide sequence having at least 90%, at least 95%, at least 96%, at least 97%, at least 98%, at least 99% or 100% sequence identity with SEQ ID NO:26 or SEQ ID NO:27.

In certain embodiments, the invention relates to a viral vector encoding a G-CSF-INF-gamma co-expression construct, or a functional fragment thereof; or a nucleic acid sequence having at least 95%, 96%, 97%, 98% or 99% sequence identity to the sequence shown in SEQ ID NO: 16, or a functional fragment thereof; or encoding a first polypeptide having at least 95%, 96%, 97%, 98% or 99% sequence identity to the sequence shown in SEQ ID NO: 10, or a functional fragment thereof, and a second polypeptide having at least 95%, 96%, 97%, 98% or 99% sequence identity to the sequence shown in SEQ ID NO: 14, or a functional fragment thereof, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof; and (b) a TMEM119 promoter, or a promoter having at least 95%, 96%, 97%, 98%, 99% or 100% sequence identity to the sequence shown in SEQ ID NO:3, SEQ ID NO:23 or SEQ ID NO:24, or a functional fragment thereof. In certain embodiments, the fusion promoter comprises a nucleotide sequence having at least 90%, at least 95%, at least 96%, at least 97%, at least 98%, at least 99% or 100% sequence identity with SEQ ID NO:28.

In certain embodiments, the invention relates to a viral vector encoding a G-CSF-INF-gamma co-expression construct, or a functional fragment thereof; or a nucleic acid sequence having at least 95%, 96%, 97%, 98% or 99% sequence identity to the sequence shown in SEQ ID NO: 16, or a functional fragment thereof; or encoding a first polypeptide having at least 95%, 96%, 97%, 98% or 99% sequence identity to the sequence shown in SEQ ID NO: 10, or a functional fragment thereof, and a second polypeptide having at least 95%, 96%, 97%, 98% or 99% sequence identity to the sequence shown in SEQ ID NO: 14, or a functional fragment thereof, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof; and (b) an OLFML3 promoter, or a promoter having at least 95%, 96%, 97%, 98%, 99% or 100% sequence identity to the sequence shown in SEQ ID NO:4 or SEQ ID NO:25, or a functional fragment thereof. In certain embodiments, the fusion promoter comprises a nucleotide sequence having at least 90%, at least 95%, at least 96%, at least 97%, at least 98%, at least 99% or 100% sequence identity with SEQ ID NO:29.

In a particular embodiment, the invention relates to a viral vector comprising a transgene under control of one or more promoters, wherein the transgene encodes
a) IL-2, or a functional fragment thereof; or
b) a polypeptide having at least 95%, 96%, 97%, 98%, 99% or 100% sequence identity to the sequence shown in SEQ ID NO: 17, or a functional fragment thereof.

That is, in certain embodiments, the invention relates to a viral vector encoding Interleukin-2 (IL-2). The term “IL-2” refers to the protein sequence of SEQ ID NO: 17, and/or to any sequence with a sequence identity of >95% homology thereof. Also provided herein are nucleic acid sequences encoding said proteins.

(SEQ ID NO: 17)
MYRMQLLSCIALSLALVTNSAPTSSSTKKTQLQLEHLLLDLQMILNGINN
YKNPKLTRMLTFKFYMPKKATELKHLQCLEEELKPLEEVLNLAQSKNFHL
RPRDLISNINVIVLELKGSETTFMCEYADETATIVEFLNRWITFCQSIIS
TLT

Interleukin-2 (IL-2) is an interleukin, a type of cytokine signaling molecule in the immune system. It is a 15.5-16 kDa protein that regulates the activities of white blood cells (leukocytes, often lymphocytes) that are responsible for immunity. IL-2 is part of the body's natural response to microbial infection, and in discriminating between foreign (“non-self”) and “self”. IL-2 mediates its effects by binding to IL-2 receptors, which are expressed by lymphocytes. The major sources of IL-2 are activated CD4+ T cells and activated CD8+ T cells.

Aldesleukin is a form of recombinant interleukin-2. It is manufactured using recombinant DNA technology and is marketed as a protein therapeutic and branded as Proleukin. It has been approved by the Food and Drug Administration (FDA) and in several European countries for the treatment of cancers (malignant melanoma, renal cell cancer) in large intermittent doses and has been extensively used in continuous doses.

A functional fragment of IL-2 is a fragment of at least 50, at least 100 amino acids, at least 110, at least 120, at least 130 or at least 140 amino acids having at least 95% sequence identity with SEQ ID NO:17, wherein the fragment has IL-2 activity. Assays to determine whether a protein has IL-2 activity have been described in the art, for example by Leivestad et al., A simple and sensitive bioassay for the detection of IL-2 activity; J Immunol Methods. 1988 Nov. 10; 114 (1-2):95-9. doi: 10.1016/0022-1759(88)90159-7.

In a particular embodiment, the invention relates to a viral vector encoding IL-2, or a functional fragment thereof; or a polypeptide having at least 95%, 96%, 97%, 98% or 99% sequence identity to the sequence shown in SEQ ID NO: 17, or a functional fragment thereof, wherein the one or more promoters comprises:
a) a myelo-specific promoter, or a functional fragment thereof, and/or
b) a microglia-specific promoter, or a functional fragment thereof, and/or
c) a fusion promoter comprising or consisting of
   i) a first promoter, wherein said first promoter is a myelo-specific promoter or a microglia-specific promoter, or a functional fragment thereof; and
   ii) a second promoter.

That is, in certain embodiments, IL-2 or a functional fragment or mutant variant thereof as disclosed above may be expressed from a myelo-specific promoter or from a functional fragment thereof. In other embodiments, IL-2 or a functional fragment or mutant variant thereof as disclosed above may be expressed from a microglia-specific promoter or from a functional fragment thereof. In other embodiments, IL-2 or a functional fragment or mutant variant thereof as disclosed above may be expressed from a fusion promoter, preferably wherein the fusion promoter comprises a myelo-specific or a microglia-specific promoter or functional fragments thereof.

That is, in a particular embodiment, the invention relates to a viral vector encoding IL-2, or a functional fragment thereof, or a polypeptide having at least 95%, 96%, 97%, 98% or 99% sequence identity to the sequence shown in SEQ ID NO: 17, or a functional fragment thereof, wherein the myelo-specific promoter is
a) a miR233 promoter, or a functional fragment thereof; or a promoter having at least 95%, 96%, 97%, 98%, 99% or 100% sequence identity to the sequence shown in SEQ ID NO: 1, or a functional fragment thereof;
b) an ITGAM promoter, or a functional fragment thereof, or a promoter having at least 95%, 96%, 97%, 98%, 99% or 100% sequence identity to the sequence shown in SEQ ID NO:6, or a functional fragment thereof, or
c) an AIF1 promoter, or a functional fragment thereof; or a promoter having at least 95%, 96%, 97%, 98%, 99% or 100% sequence identity to the sequence shown in SEQ ID NO:5, or a functional fragment thereof.

In a particular embodiment, the invention relates to a viral vector encoding IL-2, or a functional fragment thereof; or a polypeptide having at least 95%, 96%, 97%, 98% or 99% sequence identity to the sequence shown in SEQ ID NO: 17, or a functional fragment thereof, wherein the microglia-specific promoter is
a) a TMEM119 promoter or a promoter having at least 95%, 96%, 97%, 98%, 99% or 100% sequence identity to the sequence shown in SEQ ID NO:3, SEQ ID NO:23 or SEQ ID NO:24, or a functional fragment thereof,
b) a P2RY12 promoter or a promoter having at least 95%, 96%, 97%, 98%, 99% or 100% sequence identity to the sequence shown in SEQ ID NO: 2, SEQ ID NO:21 or SEQ ID NO:22, or a functional fragment thereof; or
c) an OLFML3 promoter or a promoter having at least 95%, 96%, 97%, 98%, 99% or 100% sequence identity to the sequence shown in SEQ ID NO:4 or SEQ ID NO:25, or a functional fragment thereof.

In a particular embodiment, the invention relates to a viral vector encoding IL-2, or a functional fragment thereof; or a polypeptide having at least 95%, 96%, 97%, 98% or 99% sequence identity to the sequence shown in SEQ ID NO: 17, or a functional fragment thereof, wherein the first promoter is a myelo-specific promoter and wherein the second promoter is a microglia-specific promoter, or vice versa.

The second promoter may be any promoter known in the art. However, in certain embodiments, IL-2 or a functional fragment or mutant variant thereof as disclosed above may be expressed from a fusion promoter comprising a myelo-specific promoter and a microglia-specific promoter. That is, any of the myelo-specific promoters disclosed above may be combined with any of the microglia-specific promoters disclosed above, in any order.

In certain embodiments, IL-2 or a functional fragment or mutant variant thereof as disclosed above may be expressed from a fusion promoter comprising miR223, a functional fragment thereof or a promoter with miR223 functionality, and a microglia-specific promoter.

That is, in a particular embodiment, the invention relates to a viral vector encoding IL-2, or a functional fragment thereof, or a polypeptide having at least 95%, 96%, 97%, 98% or 99% sequence identity to the sequence shown in SEQ ID NO: 17, or a functional fragment thereof, wherein the first promoter is a miR233 promoter, or a promoter having at least 95%, 96%, 97%, 98%, 99% or 100% sequence identity to the sequence shown in SEQ ID NO: 1, or a functional fragment thereof, and wherein the first promoter is operably linked to
i) a TMEM119 promoter, or a promoter having at least 95%, 96%, 97%, 98%, 99% or 100% sequence identity to the sequence shown in SEQ ID NO:3, SEQ ID NO:23 or SEQ ID NO:24, or a functional fragment thereof,
ii) a P2RY12 promoter, or a promoter having at least 95%, 96%, 97%, 98%, 99% or 100% sequence identity to the sequence shown in SEQ ID NO: 2, SEQ ID NO:21 or SEQ ID NO:22, or a functional fragment thereof;
iii) an OLFML3 promoter, or a promoter having at least 95%, 96%, 97%, 98%, 99% or 100% sequence identity to the sequence shown in SEQ ID NO:4 or SEQ ID NO:25, or a functional fragment thereof;
iv) an ITGAM promoter, or a promoter having at least 95%, 96%, 97%, 98%, 99% or 100% sequence identity to the sequence shown in SEQ ID NO:6, or a functional fragment thereof; and/or
v) an AIF1 promoter or a promoter having at least 95%, 96%, 97%, 98%, 99% or 100% sequence identity to the sequence shown in SEQ ID NO:5, or a functional fragment thereof.

In certain embodiments, the invention relates to a viral vector encoding IL-2, or a functional fragment thereof; or a polypeptide having at least 95%, 96%, 97%, 98% or 99% sequence identity to the sequence shown in SEQ ID NO: 17, or a functional fragment thereof, wherein the promoter is a miR233 promoter, or a promoter having at least 95%, 96%, 97%, 98%, 99% or 100% sequence identity to the sequence shown in SEQ ID NO: 1, or a functional fragment thereof.

In certain embodiments, the invention relates to a viral vector encoding IL-2, or a functional fragment thereof; or a polypeptide having at least 95%, 96%, 97%, 98% or 99% sequence identity to the sequence shown in SEQ ID NO: 17, or a functional fragment thereof, wherein the first promoter is an ITGAM promoter, or a promoter having at least 95%, 96%, 97%, 98%, 99% or 100% sequence identity to the sequence shown in SEQ ID NO:6, or a functional fragment thereof.

In certain embodiments, the invention relates to a viral vector encoding IL-2, or a functional fragment thereof; or a polypeptide having at least 95%, 96%, 97%, 98% or 99% sequence identity to the sequence shown in SEQ ID NO: 17, or a functional fragment thereof, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof, and (b) a P2RY12 promoter, or a promoter having at least 95%, 96%, 97%, 98%, 99% or 100% sequence identity to the sequence shown in SEQ ID NO:2, SEQ ID NO:21 or SEQ ID NO:22, or a functional fragment thereof. In certain embodiments, the fusion promoter comprises a nucleotide sequence having at least 90%, at least 95%, at least 96%, at least 97%, at least 98%, at least 99% or 100% sequence identity with SEQ ID NO:26 or SEQ ID NO:27.

In certain embodiments, the invention relates to a viral vector encoding IL-2, or a functional fragment thereof; or a polypeptide having at least 95%, 96%, 97%, 98% or 99% sequence identity to the sequence shown in SEQ ID NO: 17, or a functional fragment thereof, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof; and (b) a TMEM119 promoter, or a promoter having at least 95%, 96%, 97%, 98%, 99% or 100% sequence identity to the sequence shown in SEQ ID NO:3, SEQ ID NO:23 or SEQ ID NO:24, or a functional fragment thereof. In certain embodiments, the fusion promoter comprises a nucleotide sequence having at least 90%, at least 95%, at least 96%, at least 97%, at least 98%, at least 99% or 100% sequence identity with SEQ ID NO:28.

In certain embodiments, the invention relates to a viral vector encoding IL-2, or a functional fragment thereof; or a polypeptide having at least 95%, 96%, 97%, 98% or 99% sequence identity to the sequence shown in SEQ ID NO: 17, or a functional fragment thereof, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof; and (b) an OLFML3 promoter, or a promoter having at least 95%, 96%, 97%, 98%, 99% or 100% sequence identity to the sequence shown in SEQ ID NO:4 or SEQ ID NO:25, or a functional fragment thereof. In certain embodiments, the fusion promoter comprises a nucleotide sequence having at least 90%, at least 95%, at least 96%, at least 97%, at least 98%, at least 99% or 100% sequence identity with SEQ ID NO:29.

In a particular embodiment, the invention relates to a viral vector comprising a transgene under control of one or more promoters, wherein the transgene encodes
a) IL-15, or a functional fragment thereof; or
b) a polypeptide having at least 95%, 96%, 97%, 98%, 99% or 100% sequence identity to the sequence shown in SEQ ID NO: 18, or a functional fragment thereof.

That is, in certain embodiments, the invention relates to a viral vector encoding Interleukin-15 (IL-15). The term “IL-15” refers to the protein sequence of SEQ ID NO: 18, and/or to any sequence with a sequence identity of >95% homology thereof. Also provided herein are nucleic acid sequences encoding said proteins.

(SEQ ID NO: 18)
MRISKPHLRSISIQCYLCLLLNSHFLTEAGIHVFILGCFSAGLPKTEANW
VNVISDLKKIEDLIQSMHIDATLYTESDVHPSCKVTAMKCFLLELQVISL
ESGDASIHDTVENLIILANNSLSSNGNVTESGCKECEELEEKNIKEFLQS
FVHIVQMFINTS

Interleukin-15 (IL-15) is a cytokine with structural similarity to Interleukin-2 (IL-2). Like IL-2, IL-15 binds to and signals through a complex composed of IL-2/IL-15 receptor beta chain (CD122) and the common gamma chain (gamma-C, CD132). IL-15 is secreted by mononuclear phagocytes (and some other cells) following infection by virus(es). This cytokine induces the proliferation of natural killer cells, i.e. cells of the innate immune system whose principal role is to kill virally infected cells. IL-15 regulates the activation and proliferation of T and natural killer (NK) cells. Survival signals that maintain memory T cells in the absence of antigen are provided by IL-15. This cytokine is also implicated in NK cell development. In rodent lymphocytes, IL-15 prevents apoptosis by inducing BCL2L1/BCL-x(L), an inhibitor of the apoptosis pathway. In humans with celiac disease IL-15 similarly suppresses apoptosis in T-lymphocytes by inducing Bcl-2 and/or Bcl-xL.

IL-15 has been shown to enhance the anti-tumor immunity of CD8+ T cells in pre-clinical models. A phase I clinical trial to evaluate the safety, dosing, and anti-tumor efficacy of IL-15 in patients with metastatic melanoma and renal cell carcinoma (kidney cancer) has begun to enroll patients at the National Institutes of Health.

A functional fragment of IL-15 is a fragment of at least 50, at least 100 amino acids, at least 110, at least 120, at least 130 or at least 140 amino acids having at least 95% sequence identity with SEQ ID NO:18, wherein the fragment has IL-15 activity. Assays to determine whether a protein has IL-15 activity have been described in the art, for example by Hu et al., Discovery of a novel IL-15 based protein with improved developability and efficacy for cancer immunotherapy; Sci Rep. 2018 May 16; 8(1):7675. doi: 10.1038/s41598-018-25987-4.

In a particular embodiment, the invention relates to a viral vector encoding IL-15, or a functional fragment thereof; or a polypeptide having at least 95%, 96%, 97%, 98% or 99% sequence identity to the sequence shown in SEQ ID NO: 18, or a functional fragment thereof, wherein the one or more promoters comprises:
a) a myelo-specific promoter, or a functional fragment thereof; and/or
b) a microglia-specific promoter, or a functional fragment thereof; and/or
c) a fusion promoter comprising or consisting of
   i) a first promoter, wherein said first promoter is a myelo-specific promoter or a microglia-specific promoter, or a functional fragment thereof; and
   ii) a second promoter.

That is, in certain embodiments, IL-15 or a functional fragment or mutant variant thereof as disclosed above may be expressed from a myelo-specific promoter or from a functional fragment thereof. In other embodiments, IL-15 or a functional fragment or mutant variant thereof as disclosed above may be expressed from a microglia-specific promoter or from a functional fragment thereof. In other embodiments, IL-15 or a functional fragment or mutant variant thereof as disclosed above may be expressed from a fusion promoter, preferably wherein the fusion promoter comprises a myelo-specific or a microglia-specific promoter or functional fragments thereof.

That is, in a particular embodiment, the invention relates to a viral vector encoding IL-15, or a functional fragment thereof, or a polypeptide having at least 95%, 96%, 97%, 98% or 99% sequence identity to the sequence shown in SEQ ID NO: 18, or a functional fragment thereof, wherein the myelo-specific promoter is
a) a miR233 promoter, or a functional fragment thereof, or a promoter having at least 95%, 96%, 97%, 98%, 99% or 100% sequence identity to the sequence shown in SEQ ID NO: 1, or a functional fragment thereof;
b) an ITGAM promoter, or a functional fragment thereof, or a promoter having at least 95%, 96%, 97%, 98%, 99% or 100% sequence identity to the sequence shown in SEQ ID NO:6, or a functional fragment thereof, or
c) an AIF1 promoter, or a functional fragment thereof; or a promoter having at least 95%, 96%, 97%, 98%, 99% or 100% sequence identity to the sequence shown in SEQ ID NO:5, or a functional fragment thereof.

In a particular embodiment, the invention relates to a viral vector encoding IL-15, or a functional fragment thereof, or a polypeptide having at least 95%, 96%, 97%, 98% or 99% sequence identity to the sequence shown in SEQ ID NO: 18, or a functional fragment thereof, wherein the microglia-specific promoter is
a) a TMEM119 promoter or a promoter having at least 95%, 96%, 97%, 98%, 99% or 100% sequence identity to the sequence shown in SEQ ID NO:3, SEQ ID NO:23 or SEQ ID NO:24, or a functional fragment thereof,
b) a P2RY12 promoter or a promoter having at least 95%, 96%, 97%, 98%, 99% or 100% sequence identity to the sequence shown in SEQ ID NO: 2, SEQ ID NO:21 or SEQ ID NO:22, or a functional fragment thereof; or
c) an OLFML3 promoter or a promoter having at least 95%, 96%, 97%, 98%, 99% or 100% sequence identity to the sequence shown in SEQ ID NO:4 or SEQ ID NO:25, or a functional fragment thereof.

In a particular embodiment, the invention relates to a viral vector encoding IL-15, or a functional fragment thereof; or a polypeptide having at least 95%, 96%, 97%, 98% or 99% sequence identity to the sequence shown in SEQ ID NO: 18, or a functional fragment thereof, wherein the first promoter is a myelo-specific promoter and wherein the second promoter is a microglia-specific promoter, or vice versa.

The second promoter may be any promoter known in the art. However, in certain embodiments, IL-15 or a functional fragment or mutant variant thereof as disclosed above may be expressed from a fusion promoter comprising a myelo-specific promoter and a microglia-specific promoter. That is, any of the myelo-specific promoters disclosed above may be combined with any of the microglia-specific promoters disclosed above, in any order.

In certain embodiments, IL-15 or a functional fragment or mutant variant thereof as disclosed above may be expressed from a fusion promoter comprising miR223, a functional fragment thereof or a promoter with miR223 functionality, and a microglia-specific promoter.

That is, in a particular embodiment, the invention relates to a viral vector encoding IL-15, or a functional fragment thereof; or a polypeptide having at least 95%, 96%, 97%, 98% or 99% sequence identity to the sequence shown in SEQ ID NO: 18, or a functional fragment thereof, wherein the first promoter is a miR233 promoter, or a promoter having at least 95%, 96%, 97%, 98%, 99% or 100% sequence identity to the sequence shown in SEQ ID NO: 1, or a functional fragment thereof; and wherein the first promoter is operably linked to
i) a TMEM119 promoter, or a promoter having at least 95%, 96%, 97%, 98%, 99% or 100% sequence identity to the sequence shown in SEQ ID NO:3, SEQ ID NO:23 or SEQ ID NO:24, or a functional fragment thereof;
ii) a P2RY12 promoter, or a promoter having at least 95%, 96%, 97%, 98%, 99% or 100% sequence identity to the sequence shown in SEQ ID NO: 2, SEQ ID NO:21 or SEQ ID NO:22, or a functional fragment thereof;
iii) an OLFML3 promoter, or a promoter having at least 95%, 96%, 97%, 98%, 99% or 100% sequence identity to the sequence shown in SEQ ID NO:4 or SEQ ID NO:25, or a functional fragment thereof;
iv) an ITGAM promoter, or a promoter having at least 95%, 96%, 97%, 98%, 99% or 100% sequence identity to the sequence shown in SEQ ID NO:6, or a functional fragment thereof; and/or
v) an AIF1 promoter or a promoter having at least 95%, 96%, 97%, 98%, 99% or 100% sequence identity to the sequence shown in SEQ ID NO:5, or a functional fragment thereof.

In certain embodiments, the invention relates to a viral vector encoding IL-15, or a functional fragment thereof; or a polypeptide having at least 95%, 96%, 97%, 98% or 99% sequence identity to the sequence shown in SEQ ID NO: 18, or a functional fragment thereof, wherein the promoter is a miR233 promoter, or a promoter having at least 95%, 96%, 97%, 98%, 99% or 100% sequence identity to the sequence shown in SEQ ID NO: 1, or a functional fragment thereof.

In certain embodiments, the invention relates to a viral vector encoding IL-15, or a functional fragment thereof; or a polypeptide having at least 95%, 96%, 97%, 98% or 99% sequence identity to the sequence shown in SEQ ID NO: 18, or a functional fragment thereof, wherein the first promoter is an ITGAM promoter, or a promoter having at least 95%, 96%, 97%, 98%, 99% or 100% sequence identity to the sequence shown in SEQ ID NO:6, or a functional fragment thereof.

In certain embodiments, the invention relates to a viral vector encoding IL-15, or a functional fragment thereof; or a polypeptide having at least 95%, 96%, 97%, 98% or 99% sequence identity to the sequence shown in SEQ ID NO: 18, or a functional fragment thereof, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof; and (b) a P2RY12 promoter, or a promoter having at least 95%, 96%, 97%, 98%, 99% or 100% sequence identity to the sequence shown in SEQ ID NO: 2, SEQ ID NO:21 or SEQ ID NO:22, or a functional fragment thereof. In certain embodiments, the fusion promoter comprises a nucleotide sequence having at least 90%, at least 95%, at least 96%, at least 97%, at least 98%, at least 99% or 100% sequence identity with SEQ ID NO:26 or SEQ ID NO:27.

In certain embodiments, the invention relates to a viral vector encoding IL-15, or a functional fragment thereof; or a polypeptide having at least 95%, 96%, 97%, 98% or 99% sequence identity to the sequence shown in SEQ ID NO: 18, or a functional fragment thereof, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof; and (b) a TMEM119 promoter, or a promoter having at least 95%, 96%, 97%, 98%, 99% or 100% sequence identity to the sequence shown in SEQ ID NO:3, SEQ ID NO:23 or SEQ ID NO:24, or a functional fragment thereof. In certain embodiments, the fusion promoter comprises a nucleotide sequence having at least 90%, at least 95%, at least 96%, at least 97%, at least 98%, at least 99% or 100% sequence identity with SEQ ID NO:28.

In certain embodiments, the invention relates to a viral vector encoding IL-15, or a functional fragment thereof; or a polypeptide having at least 95%, 96%, 97%, 98% or 99% sequence identity to the sequence shown in SEQ ID NO: 18, or a functional fragment thereof, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof; and (b) an OLFML3 promoter, or a promoter having at least 95%, 96%, 97%, 98%, 99% or 100% sequence identity to the sequence shown in SEQ ID NO:4 or SEQ ID NO:25, or a functional fragment thereof. In certain embodiments, the fusion promoter comprises a nucleotide sequence having at least 90%, at least 95%, at least 96%, at least 97%, at least 98%, at least 99% or 100% sequence identity with SEQ ID NO:29.

In a particular embodiment, the invention relates to a viral vector comprising a transgene under control of one or more promoters, wherein the transgene encodes
a) IL-21, or a functional fragment thereof, or
b) a polypeptide having at least 95%, 96%, 97%, 98%, 99% or 100% sequence identity to the sequence shown in SEQ ID NO: 19, or a functional fragment thereof.

That is, in certain embodiments, the invention relates to a viral vector encoding Interleukin-21 (IL-21). The term “IL-21” refers to the protein sequence of SEQ ID NO: 19, and/or to any sequence with a sequence identity of >95% homology thereof. Also provided herein are nucleic acid sequences encoding said proteins.

(SEQ ID NO: 19)
MRSSPGNMERIVICLMVIFLGTLVHKSSSQGQDRHMIRMRQLIDIVDQLK
NYVNDLVPEFLPAPEDVETNCEWSAFSCFQKAQLKSANTGNNERIINVSI
KKLKRKPPSTNAGRRQKHRLTCPSCDSYEKKPPKEFLERFKSLLQKMIHQ
HLSSRTHGSEDS

Interleukin-21 (IL-21) is a cytokine that has potent regulatory effects on cells of the immune system, including natural killer (NK) cells and cytotoxic T cells that can destroy virally infected or cancerous cells. This cytokine induces cell division/proliferation in its target cells.

A role for IL-21 in modulating the differentiation programming of human T cells was reported, where it was shown to enrich for a population of central memory-type CTL with a unique CD28+ CD127hi CD45RO+ phenotype with IL-2 producing capacity. Tumor-reactive antigen-specific CTL generated by priming in the presence of IL-21 led to a stable, ‘helper-independent’ phenotype. IL-21 is also noted to have anti-tumour effects through continued and increased CD8+ cell response to achieve enduring tumor immunity.

IL-21 was approved for Phase 1 clinical trials in metastatic melanoma (MM) and renal cell carcinoma (RCC) patients. It was shown to be safe for administration with flu-like symptoms as side effects. Dose-limiting toxicities included low lymphocyte, neutrophil, and thrombocyte count as well as hepatotoxicity. According to the Response Evaluation Criteria in Solid Tumors (RECIST) response scale, 2 out of 47 MM patients and 4 out of 19 RCC patients showed complete and partial responses, respectively. In addition, there was an increase of perforin, granzyme B, IFN-gamma, and CXCR3 mRNA in peripheral NK cells and CD8+ T cells. This suggested that IL-21 enhances the CD8+ effector functions thus leading to anti-tumor response. IL-21 proceeded to Phase 2 clinical trials where it was administered alone or coupled with drugs as sorafinib and rituximab.

A functional fragment of IL-21 is a fragment of at least 50, at least 100 amino acids, at least 110, at least 120, at least 130 or at least 140 amino acids having at least 95% sequence identity with SEQ ID NO:19, wherein the fragment has IL-21 activity. Assays to determine whether a protein has IL-21 activity have been described in the art, for example by Maurer et al., Generation and characterization of human anti-human IL-21 neutralizing monoclonal antibodies; MAbs. 2012 January-February; 4(1): 69-83.

In a particular embodiment, the invention relates to a viral vector encoding IL-21, or a functional fragment thereof; or a polypeptide having at least 95%, 96%, 97%, 98% or 99% sequence identity to the sequence shown in SEQ ID NO: 19, or a functional fragment thereof, wherein the one or more promoters comprises:
a) a myelo-specific promoter, or a functional fragment thereof; and/or
b) a microglia-specific promoter, or a functional fragment thereof; and/or
c) a fusion promoter comprising or consisting of
   i) a first promoter, wherein said first promoter is a myelo-specific promoter or a microglia-specific promoter, or a functional fragment thereof; and
   ii) a second promoter.

That is, in certain embodiments, IL-21 or a functional fragment or mutant variant thereof as disclosed above may be expressed from a myelo-specific promoter or from a functional fragment thereof. In other embodiments, IL-21 or a functional fragment or mutant variant thereof as disclosed above may be expressed from a microglia-specific promoter or from a functional fragment thereof. In other embodiments, IL-21 or a functional fragment or mutant variant thereof as disclosed above may be expressed from a fusion promoter, preferably wherein the fusion promoter comprises a myelo-specific or a microglia-specific promoter or functional fragments thereof.

That is, in a particular embodiment, the invention relates to a viral vector encoding IL-21, or a functional fragment thereof, or a polypeptide having at least 95%, 96%, 97%, 98% or 99% sequence identity to the sequence shown in SEQ ID NO: 19, or a functional fragment thereof, wherein the myelo-specific promoter is
a) a miR233 promoter, or a functional fragment thereof, or a promoter having at least 95%, 96%, 97%, 98%, 99% or 100% sequence identity to the sequence shown in SEQ ID NO: 1, or a functional fragment thereof;
b) an ITGAM promoter, or a functional fragment thereof, or a promoter having at least 95%, 96%, 97%, 98%, 99% or 100% sequence identity to the sequence shown in SEQ ID NO:6, or a functional fragment thereof, or
c) an AIF1 promoter, or a functional fragment thereof; or a promoter having at least 95%, 96%, 97%, 98%, 99% or 100% sequence identity to the sequence shown in SEQ ID NO:5, or a functional fragment thereof.

In a particular embodiment, the invention relates to a viral vector encoding IL-21, or a functional fragment thereof, or a polypeptide having at least 95%, 96%, 97%, 98% or 99% sequence identity to the sequence shown in SEQ ID NO: 19, or a functional fragment thereof, wherein the microglia-specific promoter is
a) a TMEM119 promoter or a promoter having at least 95%, 96%, 97%, 98%, 99% or 100% sequence identity to the sequence shown in SEQ ID NO:3, SEQ ID NO:23 or SEQ ID NO:24, or a functional fragment thereof;
b) a P2RY12 promoter or a promoter having at least 95%, 96%, 97%, 98%, 99% or 100% sequence identity to the sequence shown in SEQ ID NO: 2, SEQ ID NO:21 or SEQ ID NO:22, or a functional fragment thereof; or
c) an OLFML3 promoter or a promoter having at least 95%, 96%, 97%, 98%, 99% or 100% sequence identity to the sequence shown in SEQ ID NO:4 or SEQ ID NO:25, or a functional fragment thereof.

In a particular embodiment, the invention relates to a viral vector encoding IL-21, or a functional fragment thereof; or a polypeptide having at least 95%, 96%, 97%, 98% or 99% sequence identity to the sequence shown in SEQ ID NO: 19, or a functional fragment thereof, wherein the first promoter is a myelo-specific promoter and wherein the second promoter is a microglia-specific promoter, or vice versa.

The second promoter may be any promoter known in the art. However, in certain embodiments, IL-21 or a functional fragment or mutant variant thereof as disclosed above may be expressed from a fusion promoter comprising a myelo-specific promoter and a microglia-specific promoter. That is, any of the myelo-specific promoters disclosed above may be combined with any of the microglia-specific promoters disclosed above, in any order.

In certain embodiments, IL-21 or a functional fragment or mutant variant thereof as disclosed above may be expressed from a fusion promoter comprising miR223, a functional fragment thereof or a promoter with miR223 functionality, and a microglia-specific promoter.

That is, in a particular embodiment, the invention relates to a viral vector encoding IL-21, or a functional fragment thereof; or a polypeptide having at least 95%, 96%, 97%, 98% or 99% sequence identity to the sequence shown in SEQ ID NO: 19, or a functional fragment thereof, wherein the first promoter is a miR233 promoter, or a promoter having at least 95%, 96%, 97%, 98%, 99% or 100% sequence identity to the sequence shown in SEQ ID NO: 1, or a functional fragment thereof; and wherein the first promoter is operably linked to
i) a TMEM119 promoter, or a promoter having at least 95%, 96%, 97%, 98%, 99% or 100% sequence identity to the sequence shown in SEQ ID NO:3, SEQ ID NO:23 or SEQ ID NO:24, or a functional fragment thereof;
ii) a P2RY12 promoter, or a promoter having at least 95%, 96%, 97%, 98%, 99% or 100% sequence identity to the sequence shown in SEQ ID NO: 2, SEQ ID NO:21 or SEQ ID NO:22, or a functional fragment thereof;
iii) an OLFML3 promoter, or a promoter having at least 95%, 96%, 97%, 98%, 99% or 100% sequence identity to the sequence shown in SEQ ID NO:4 or SEQ ID NO:25, or a functional fragment thereof;
iv) an ITGAM promoter, or a promoter having at least 95%, 96%, 97%, 98%, 99% or 100% sequence identity to the sequence shown in SEQ ID NO:6, or a functional fragment thereof; and/or
v) an AIF1 promoter or a promoter having at least 95%, 96%, 97%, 98%, 99% or 100% sequence identity to the sequence shown in SEQ ID NO:5, or a functional fragment thereof.

In certain embodiments, the invention relates to a viral vector encoding IL-21, or a functional fragment thereof; or a polypeptide having at least 95%, 96%, 97%, 98% or 99% sequence identity to the sequence shown in SEQ ID NO: 19, or a functional fragment thereof, wherein the promoter is a miR233 promoter, or a promoter having at least 95%, 96%, 97%, 98%, 99% or 100% sequence identity to the sequence shown in SEQ ID NO: 1, or a functional fragment thereof.

In certain embodiments, the invention relates to a viral vector encoding IL-21, or a functional fragment thereof; or a polypeptide having at least 95%, 96%, 97%, 98% or 99% sequence identity to the sequence shown in SEQ ID NO: 19, or a functional fragment thereof, wherein the first promoter is an ITGAM promoter, or a promoter having at least 95%, 96%, 97%, 98%, 99% or 100% sequence identity to the sequence shown in SEQ ID NO:6, or a functional fragment thereof.

In certain embodiments, the invention relates to a viral vector encoding IL-21, or a functional fragment thereof; or a polypeptide having at least 95%, 96%, 97%, 98% or 99% sequence identity to the sequence shown in SEQ ID NO: 19, or a functional fragment thereof, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof; and (b) a P2RY12 promoter, or a promoter having at least 95%, 96%, 97%, 98%, 99% or 100% sequence identity to the sequence shown in SEQ ID NO: 2, SEQ ID NO:21 or SEQ ID NO:22, or a functional fragment thereof. In certain embodiments, the fusion promoter comprises a nucleotide sequence having at least 90%, at least 95%, at least 96%, at least 97%, at least 98%, at least 99% or 100% sequence identity with SEQ ID NO:26 or SEQ ID NO:27.

In certain embodiments, the invention relates to a viral vector encoding IL-21, or a functional fragment thereof, or a polypeptide having at least 95%, 96%, 97%, 98% or 99% sequence identity to the sequence shown in SEQ ID NO: 19, or a functional fragment thereof, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof; and (b) a TMEM119 promoter, or a promoter having at least 95%, 96%, 97%, 98%, 99% or 100% sequence identity to the sequence shown in SEQ ID NO:3, SEQ ID NO:23 or SEQ ID NO:24, or a functional fragment thereof. In certain embodiments, the fusion promoter comprises a nucleotide sequence having at least 90%, at least 95%, at least 96%, at least 97%, at least 98%, at least 99% or 100% sequence identity with SEQ ID NO:28.

In certain embodiments, the invention relates to a viral vector encoding IL-21, or a functional fragment thereof; or a polypeptide having at least 95%, 96%, 97%, 98% or 99% sequence identity to the sequence shown in SEQ ID NO: 19, or a functional fragment thereof, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof; and (b) an OLFML3 promoter, or a promoter having at least 95%, 96%, 97%, 98%, 99% or 100% sequence identity to the sequence shown in SEQ ID NO:4 or SEQ ID NO:25, or a functional fragment thereof. In certain embodiments, the fusion promoter comprises a nucleotide sequence having at least 90%, at least 95%, at least 96%, at least 97%, at least 98%, at least 99% or 100% sequence identity with SEQ ID NO:29.

In a particular embodiment, the invention relates to a viral vector comprising a transgene under control of one or more promoters, wherein the transgene encodes
a) IFN-alpha, or a functional fragment thereof, or
b) a polypeptide having at least 95%, 96%, 97%, 98%, 99% or 100% sequence identity to the sequence shown in SEQ ID NO: 20, or a functional fragment thereof.

That is, in certain embodiments, the invention relates to a viral vector encoding Interferon-alpha (IFN-alpha). The term “IFN-alpha” refers to the protein sequence of SEQ ID NO: 20, and/or to any sequence with a sequence identity of >95% homology thereof. Also provided herein are nucleic acid sequences encoding said proteins.

(SEQ ID NO: 20)
MALTFALLVALLVLSCKSSCSVGCDLPQTHSLGSRRTLMLLAQMRKISLF
SCLKDRHDFGFPQEEFGNQFQKAETIPVLHEMIQQIFNLFSTKDSSAAWD
ETLLDKFYTELYQQLNDLEACVIQGVGVTETPLMKEDSILAVRKYFQRIT
LYLKEKKYSPCAWEVVRAEIMRSFSLSTNLQESLRSKE

Human interferon alpha-2 (IFNα2) is a cytokine belonging to the family of type I IFNs. IFNα2 is a protein secreted by cells infected by a virus and acting on other cells to inhibit viral infection.

If given orally, IFNα2 is degraded by digestive enzymes and is no longer active. Thus, IFNα2 is mainly administrated by injection essentially subcutaneous or intramuscular. Once in the blood, IFNα2 is rapidly eliminated by the kidney. Due to the short life of IFNα2 in the organism, several injections per week are required. Peginterferon alpha-2a and Peginterferon alpha-2b (polyethylene glycol linked to IFNα2) are long-lasting IFNα2 formulations, which enable a single injection per week.

Recombinant IFNα2 (α2a and α2b) has demonstrated efficiency in the treatment of patients diagnosed with some viral infections (such as chronic viral hepatitis B and hepatitis C) or some kinds of cancer (melanoma, renal cell carcinoma and various hematological malignancies).

A functional fragment of IFN-alpha is a fragment of at least 50, at least 100 amino acids, at least 110, at least 120, at least 130 or at least 140 amino acids having at least 95% sequence identity with SEQ ID NO:20, wherein the fragment has IFN-alpha activity. Assays to determine whether a protein has IFN-alpha activity have been described in the art, for example by Moll et al., The differential activity of interferon-α subtypes is consistent among distinct target genes and cell types; Cytokine. 2011 January; 53(1): 52-59.

In a particular embodiment, the invention relates to a viral vector encoding IFN-alpha, or a functional fragment thereof or a polypeptide having at least 95%, 96%, 97%, 98% or 99% sequence identity to the sequence shown in SEQ ID NO: 20, or a functional fragment thereof, wherein the one or more promoters comprises:
a) a myelo-specific promoter, or a functional fragment thereof, and/or
b) a microglia-specific promoter, or a functional fragment thereof, and/or
c) a fusion promoter comprising or consisting of
   i) a first promoter, wherein said first promoter is a myelo-specific promoter or a microglia-specific promoter, or a functional fragment thereof; and
   ii) a second promoter.

That is, in certain embodiments, IFN-alpha or a functional fragment or mutant variant thereof as disclosed above may be expressed from a myelo-specific promoter or from a functional fragment thereof. In other embodiments, IFN-alpha or a functional fragment or mutant variant thereof as disclosed above may be expressed from a microglia-specific promoter or from a functional fragment thereof. In other embodiments, IFN-alpha or a functional fragment or mutant variant thereof as disclosed above may be expressed from a fusion promoter, preferably wherein the fusion promoter comprises a myelo-specific or a microglia-specific promoter or functional fragments thereof.

That is, in a particular embodiment, the invention relates to a viral vector encoding IFN-alpha, or a functional fragment thereof, or a polypeptide having at least 95%, 96%, 97%, 98% or 99% sequence identity to the sequence shown in SEQ ID NO: 20, or a functional fragment thereof, wherein the myelo-specific promoter is
a) a miR233 promoter, or a functional fragment thereof, or a promoter having at least 95%, 96%, 97%, 98%, 99% or 100% sequence identity to the sequence shown in SEQ ID NO: 1, or a functional fragment thereof;
b) an ITGAM promoter, or a functional fragment thereof, or a promoter having at least 95%, 96%, 97%, 98%, 99% or 100% sequence identity to the sequence shown in SEQ ID NO:6, or a functional fragment thereof, or.
c) an AIF1 promoter, or a functional fragment thereof; or a promoter having at least 95%, 96%, 97%, 98%, 99% or 100% sequence identity to the sequence shown in SEQ ID NO:5, or a functional fragment thereof.

In a particular embodiment, the invention relates to a viral vector encoding IFN-alpha, or a functional fragment thereof, or a polypeptide having at least 95%, 96%, 97%, 98% or 99% sequence identity to the sequence shown in SEQ ID NO: 20, or a functional fragment thereof, wherein the microglia-specific promoter is
a) a TMEM119 promoter or a promoter having at least 95%, 96%, 97%, 98%, 99% or 100% sequence identity to the sequence shown in SEQ ID NO:3, SEQ ID NO:23 or SEQ ID NO:24, or a functional fragment thereof;
b) a P2RY12 promoter or a promoter having at least 95%, 96%, 97%, 98%, 99% or 100% sequence identity to the sequence shown in SEQ ID NO: 2, SEQ ID NO:21 or SEQ ID NO:22, or a functional fragment thereof; or
c) an OLFML3 promoter or a promoter having at least 95%, 96%, 97%, 98%, 99% or 100% sequence identity to the sequence shown in SEQ ID NO:4 or SEQ ID NO:25, or a functional fragment thereof.

In a particular embodiment, the invention relates to a viral vector encoding IFN-alpha, or a functional fragment thereof; or a polypeptide having at least 95%, 96%, 97%, 98% or 99% sequence identity to the sequence shown in SEQ ID NO: 20, or a functional fragment thereof, wherein the first promoter is a myelo-specific promoter and wherein the second promoter is a microglia-specific promoter, or vice versa.

The second promoter may be any promoter known in the art. However, in certain embodiments, IFN-alpha or a functional fragment or mutant variant thereof as disclosed above may be expressed from a fusion promoter comprising a myelo-specific promoter and a microglia-specific promoter. That is, any of the myelo-specific promoters disclosed above may be combined with any of the microglia-specific promoters disclosed above, in any order.

In certain embodiments, IFN-alpha or a functional fragment or mutant variant thereof as disclosed above may be expressed from a fusion promoter comprising miR223, a functional fragment thereof or a promoter with miR223 functionality, and a microglia-specific promoter.

That is, in a particular embodiment, the invention relates to a viral vector encoding IFN-alpha, or a functional fragment thereof; or a polypeptide having at least 95%, 96%, 97%, 98% or 99% sequence identity to the sequence shown in SEQ ID NO: 20, or a functional fragment thereof, wherein the first promoter is a miR233 promoter, or a promoter having at least 95%, 96%, 97%, 98%, 99% or 100% sequence identity to the sequence shown in SEQ ID NO: 1, or a functional fragment thereof; and wherein the first promoter is operably linked to
i) a TMEM119 promoter, or a promoter having at least 95%, 96%, 97%, 98%, 99% or 100% sequence identity to the sequence shown in SEQ ID NO:3, SEQ ID NO:23 or SEQ ID NO:24, or a functional fragment thereof;
ii) a P2RY12 promoter, or a promoter having at least 95%, 96%, 97%, 98%, 99% or 100% sequence identity to the sequence shown in SEQ ID NO:2, SEQ ID NO:21 or SEQ ID NO:22, or a functional fragment thereof;
iii) an OLFML3 promoter, or a promoter having at least 95%, 96%, 97%, 98%, 99% or 100% sequence identity to the sequence shown in SEQ ID NO:4 or SEQ ID NO:25, or a functional fragment thereof;
iv) an ITGAM promoter, or a promoter having at least 95%, 96%, 97%, 98%, 99% or 100% sequence identity to the sequence shown in SEQ ID NO:6, or a functional fragment thereof; and/or
v) an AIF1 promoter or a promoter having at least 95%, 96%, 97%, 98%, 99% or 100% sequence identity to the sequence shown in SEQ ID NO:5, or a functional fragment thereof.

In certain embodiments, the invention relates to a viral vector encoding IFN-alpha, or a functional fragment thereof; or a polypeptide having at least 95%, 96%, 97%, 98% or 99% sequence identity to the sequence shown in SEQ ID NO: 20, or a functional fragment thereof, wherein the promoter is a miR233 promoter, or a promoter having at least 95%, 96%, 97%, 98%, 99% or 100% sequence identity to the sequence shown in SEQ ID NO: 1, or a functional fragment thereof.

In certain embodiments, the invention relates to a viral vector encoding IFN-alpha, or a functional fragment thereof; or a polypeptide having at least 95%, 96%, 97%, 98% or 99% sequence identity to the sequence shown in SEQ ID NO: 20, or a functional fragment thereof, wherein the first promoter is an ITGAM promoter, or a promoter having at least 95%, 96%, 97%, 98%, 99% or 100% sequence identity to the sequence shown in SEQ ID NO:6, or a functional fragment thereof.

In certain embodiments, the invention relates to a viral vector encoding IFN-alpha, or a functional fragment thereof; or a polypeptide having at least 95%, 96%, 97%, 98% or 99% sequence identity to the sequence shown in SEQ ID NO: 20, or a functional fragment thereof, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof; and (b) a P2RY12 promoter, or a promoter having at least 95%, 96%, 97%, 98%, 99% or 100% sequence identity to the sequence shown in SEQ ID NO: 2, SEQ ID NO:21 or SEQ ID NO:22, or a functional fragment thereof. In certain embodiments, the fusion promoter comprises a nucleotide sequence having at least 90%, at least 95%, at least 96%, at least 97%, at least 98%, at least 99% or 100% sequence identity with SEQ ID NO:26 or SEQ ID NO:27.

In certain embodiments, the invention relates to a viral vector encoding IFN-alpha, or a functional fragment thereof; or a polypeptide having at least 95%, 96%, 97%, 98% or 99% sequence identity to the sequence shown in SEQ ID NO: 20, or a functional fragment thereof, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof; and (b) a TMEM119 promoter, or a promoter having at least 95%, 96%, 97%, 98%, 99% or 100% sequence identity to the sequence shown in SEQ ID NO:3, SEQ ID NO:23 or SEQ ID NO:24, or a functional fragment thereof. In certain embodiments, the fusion promoter comprises a nucleotide sequence having at least 90%, at least 95%, at least 96%, at least 97%, at least 98%, at least 99% or 100% sequence identity with SEQ ID NO:28.

In certain embodiments, the invention relates to a viral vector encoding IFN-alpha, or a functional fragment thereof; or a polypeptide having at least 95%, 96%, 97%, 98% or 99% sequence identity to the sequence shown in SEQ ID NO: 20, or a functional fragment thereof, wherein the promoter is a fusion promoter comprising (a) a miR233 promoter, or a promoter having at least 95%, 96%, 97%, 98%, 99% or 100% sequence identity to the sequence shown in SEQ ID NO: 1, or a functional fragment thereof; and (b) an OLFML3 promoter, or a promoter having at least 95%, 96%, 97%, 98%, 99% or 100% sequence identity to the sequence shown in SEQ ID NO:4 or SEQ ID NO:25, or a functional fragment thereof. In certain embodiments, the fusion promoter comprises a nucleotide sequence having at least 90%, at least 95%, at least 96%, at least 97%, at least 98%, at least 99% or 100% sequence identity with SEQ ID NO:29.

In a particular embodiment, the invention relates to the viral vector according to the invention, wherein the one or more promoters comprise:
a) a myelo-specific promoter, or a functional fragment thereof; and/or
b) a microglia-specific promoter, or a functional fragment thereof; and/or
c) a fusion promoter comprising or consisting of
   i) a first promoter, wherein said first promoter is a myelo-specific promoter or a microglia-specific promoter, or a functional fragment thereof; and
   ii) a second promoter.

That is, any of the transgenes disclosed above, or the functional fragments or variants thereof, may be operably linked to one or more promoters. In certain embodiments, the transgenes disclosed above, or the functional fragments or variants thereof, may be operably linked to a myelo-specific promoter or a functional fragment thereof. In certain embodiments, the transgenes disclosed above, or the functional fragments or variants thereof, may be operably linked to a microglia-specific promoter or a functional fragment thereof. In certain embodiments, the transgenes disclosed above, or the functional fragments or variants thereof, may be operably linked to a fusion promoter comprising a myelo-specific or microglia-specific promoter or functional fragments thereof, and a second promoter.

In a particular embodiment, the invention relates to the viral vector according to the invention, wherein the myelo-specific promoter is
a) a miR233 promoter, or a functional fragment thereof, or a promoter having at least 95%, 96%, 97%, 98%, 99% or 100% sequence identity to the sequence shown in SEQ ID NO: 1, or a functional fragment thereof;
b) an ITGAM promoter, or a functional fragment thereof, or a promoter having at least 95%, 96%, 97%, 98%, 99% or 100% sequence identity to the sequence shown in SEQ ID NO:6, or a functional fragment thereof, or
c) an AIF1 promoter, or a functional fragment thereof; or a promoter having at least 95%, 96%, 97%, 98%, 99% or 100% sequence identity to the sequence shown in SEQ ID NO:5, or a functional fragment thereof.

The term “myelo-specific promoter” as used herein refers to any promoter that can drive expression in a myeloid cell. The skilled person is aware of methods to identify whether a promoter can drive expression in a myeloid cell. For example, a myeloid cell, such as the monocytic cell line THP-1, may be transduced with a viral vector encoding a fluorescent marker under control of the promoter in question. If expression of the fluorescent marker can be detected in the myeloid cell upon integration of the viral vector into the genome of the myeloid cell, the promoter is determined to be a myelo-specific promoter. Myelo-specific promoters within the meaning of the present invention include, without limitation, the miR223 promoter, the AIF1 promoter and the ITGAM promoter.

In a particular embodiment, the invention relates to viral vector according to the invention, wherein the microglia-specific promoter is
a) a TMEM119 promoter or a promoter having at least 95%, 96%, 97%, 98%, 99% or 100% sequence identity to the sequence shown in SEQ ID NO:3, SEQ ID NO:23 or SEQ ID NO:24, or a functional fragment thereof;
b) a P2RY12 promoter or a promoter having at least 95%, 96%, 97%, 98%, 99% or 100% sequence identity to the sequence shown in SEQ ID NO: 2, SEQ ID NO:21 or SEQ ID NO:22, or a functional fragment thereof; or
c) an OLFML3 promoter or a promoter having at least 95%, 96%, 97%, 98%, 99% or 100% sequence identity to the sequence shown in SEQ ID NO:4 or SEQ ID NO:25, or a functional fragment thereof.

The term “microglia-specific promoter” as used herein refers to any promoter that can drive expression in microglia. The skilled person is aware of methods to identify whether a promoter can drive expression in microglia. For example, microglia, such as an immortalized microglia cell line, may be transduced with a viral vector encoding a fluorescent marker under control of the promoter in question. If expression of the fluorescent marker can be detected in microglia upon integration of the viral vector into the genome of the microglia, the promoter is determined to be a microglia-specific promoter. Microglia-specific promoters within the meaning of the present invention include, without limitation, the P2RY12 promoter, the TMEM119 promoter, the OLFML3 promoter, the ITGAM promoter and the AIF1 promoter.

In certain embodiments, the invention relates to the viral vector according to the invention, wherein the first promoter is a myelo-specific promoter and wherein the second promoter is a microglia-specific promoter, or vice versa.

That is, the fusion promoter may preferably comprise a myelo-specific promoter and a microglia specific promoter. In certain embodiments, the microglia-specific promoter is fused to the 5′ end of the myelo-specific promoter. In certain embodiments, the microglia-specific promoter is fused to the 3′ end of the myelo-specific promoter.

In a particular embodiment, the invention relates to the viral vector according to the invention, wherein the first promoter is a miR233 promoter, or a promoter having at least 95%, 96%, 97%, 98%, 99% or 100% sequence identity to the sequence shown in SEQ ID NO: 1, or a functional fragment thereof; and wherein the first promoter is operably linked to
i) a TMEM119 promoter, or a promoter having at least 95%, 96%, 97%, 98%, 99% or 100% sequence identity to the sequence shown in SEQ ID NO:3, SEQ ID NO:23 or SEQ ID NO:24, or a functional fragment thereof;
ii) a P2RY12 promoter, or a promoter having at least 95%, 96%, 97%, 98%, 99% or 100% sequence identity to the sequence shown in SEQ ID NO: 2, SEQ ID NO:21 or SEQ ID NO:23, or a functional fragment thereof;
iii) an OLFML3 promoter, or a promoter having at least 95%, 96%, 97%, 98%, 99% or 100% sequence identity to the sequence shown in SEQ ID NO:4 or SEQ ID NO:25, or a functional fragment thereof
iv) an ITGAM promoter, or a promoter having at least 95%, 96%, 97%, 98%, 99% or 100% sequence identity to the sequence shown in SEQ ID NO:6, or a functional fragment thereof; and/or
v) an AIF1 promoter or a promoter having at least 95%, 96%, 97%, 98%, 99% or 100% sequence identity to the sequence shown in SEQ ID NO:5, or a functional fragment thereof.

In a particular embodiment, the invention relates to the viral vector according to the invention, wherein the first promoter is a miR233 promoter, or a promoter having at least 95%, 96%, 97%, 98%, 99% or 100% sequence identity to the sequence shown in SEQ ID NO: 1, or a functional fragment thereof; and wherein the first promoter is operably linked to a TMEM119 promoter, or a promoter having at least 95%, 96%, 97%, 98%, 99% or 100% sequence identity to the sequence shown in SEQ ID NO: 3, SEQ ID NO:23 or SEQ ID NO:24, or a functional fragment thereof. In a certain embodiment, the viral vector according to the invention comprised a promoter having at least 95%, 96%, 97%, 98%, 99% or 100% sequence identity to the sequence shown in SEQ ID NO: 28.

In a particular embodiment, the invention relates to the viral vector according to the invention, wherein the viral vector comprises at least one transcriptional regulatory element, and wherein said at least one transcriptional regulatory element is arranged such that it inhibits or activates a transcriptional activity of the promoter.

In a particular embodiment, the invention relates to the viral vector according to the invention, wherein the at least one transcriptional regulatory element comprises a binding site for a transcriptional activator or repressor, in particular wherein the transcriptional activator or repressor comprises:
i) an antibiotic-binding domain, in particular a tetracycline/doxycycline-binding domain, a macrolide-binding domain or a pristinamycin-binding domain;
ii) a hormone-binding domain, in particular a RU486-binding domain or an abscisic acid-binding domain;
iii) a steroid-binding domain, in particular an ecdysone-binding domain;
iv) a dimerizer system, in particular a rapamycin-based of rapalog-based dimerizer system.

In a particular embodiment, the viral vector according to the invention, wherein the viral vector encodes a riboswitch, and wherein the riboswitch controls translation of an mRNA encoding the therapeutic protein or the combination of therapeutic proteins.

That is, the viral vectors encoding any one of the transgenes disclosed above, or a functional fragment or variant thereof, may comprise regulatory elements that allow controlling expression of the transgene. Preferably, the regulatory elements are any of the regulatory elements disclosed elsewhere herein.

In a particular embodiment, the invention relates to the viral vector according to the invention, wherein the viral vector is
a) a retroviral vector, in particular a lentiviral vector, more particularly a lentiviral SIN vector; or
b) a foamy viral vector; or
c) a viral vector selected from the group consisting of: an adenoviral vector, an adeno-associated viral vector, a herpes viral vector, a parvoviral vector, a coronaviral vector, and an alpha-retroviral vector.

The viral vector according to the invention may be any type of viral vector that allows delivering a transgene to a mammalian cell or, preferably, to a human cell.

In certain embodiments, the viral vector is a retroviral vector. As used herein, the term “retrovirus” refers to a virus, consisting of an outer envelope glycoprotein shell of viral origin including, but not limited to vesicular stomatitis virus (VSV) glycoprotein (VSVG), with membrane fusion activity, enclosing viral RNA, as well as viral proteins necessary for reverse transcription of its genomic RNA into a linear double-stranded DNA copy, and for subsequently covalent integration of its genomic DNA into a host genome.

Retroviruses are a common tool for gene delivery (Miller, 2000, Nature. 357: 455-460). Once the virus is integrated into the host genome, it is referred to as a “provirus.” The provirus serves as a template for RNA polymerase II and directs the expression of RNA molecules which encode the structural proteins and enzymes needed to produce new viral particles. Illustrative retroviruses include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV) and lentivirus.

As used herein, the term “lentivirus” refers to a group (or genus) of complex retroviruses. Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, HIV based vector backbones (i.e., HIV cis-acting sequence elements) are preferred.

The term “vector” is used herein to refer to a nucleic acid molecule, capable transferring or transporting another nucleic acid molecule. The transferred nucleic acid is generally linked to, i.e., inserted into, the vector nucleic acid molecule. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses.

Within the present invention, viral vectors are used to transduce target cells. The term “transduction” relates to the generation of conditions, which aim and allow for bringing a viral vector into physical contact with the target cell, followed by introduction of viral nucleic acids into the target cell, and in case of retroviruses its reverse transcription to DNA, and the integration into the genome of the target cell.

The term “lentiviral vector” may be used to refer to lentiviral infectious particles, consisting of an viral envelope glycoprotein decorated biological membrane or just biological membranes without viral envelope protein shell with membrane fusion potential, enclosing a lentiviral capsid structure formed by lentiviral protein, with the capsid structure enclosing lentiviral RNA and lentiviral proteins necessary for reverse transcription and stable integration into the genome of a target cell.

Lentiviral vectors enable delivery of the nucleic acid molecule encoding a therapeutic polypeptide into dividing and/or non-dividing cells. Lentiviral vectors can be used for in vitro transduction as well as for in vivo injection, whereas AAV infectious particles can be used for the delivery of DNA into non-dividing cells by in vivo injection.

Preferably, the viral vector according to the invention is a self-inactivating lentiviral vector. “Self-inactivating” (SIN) vectors are replication-defective vectors, e.g., viral or lentiviral vectors, in which the right (3′) LTR enhancer-promoter region, known as the U3 region, has been modified (e.g., by deletion and/or substitution) to prevent viral transcription beyond the first round of viral replication. Consequently, the vectors are capable of infecting and then integrating into the host genome only once, and cannot be passed further. This is because the right (3′) LTR U3 region is used as a template for the left (5′) LTR U3 region during viral replication and, thus, the viral transcript cannot be made without the U3 enhancer-promoter. If the viral transcript is not made, it cannot be processed or packaged into virions, hence the life cycle of the virus ends.

In certain embodiments, the viral vector may be a foamy viral vector. The term “foamy viral vector”, as used herein, refers to a viral vector that employs foamy virus derived parts. Methods to develop a viral vector are known to the skilled person (e.g., Mergia, A, and M Heinkelein, 2003, Current topics in microbiology and immunology vol. 277: 131-59).

In certain embodiment, the viral vector is selected from the group consisting of: an adenoviral vector, an adeno-associated viral vector, a herpes viral vector, a parvoviral vector, a coronaviral vector, and an alpha-retroviral vector.

The term “Adenoviral vector”, as used herein, refers to a viral vector or plasmid containing structural and functional genetic elements, or portions thereof, that are primarily derived from an Adenovirus.

The term “Adenovirus”, as used herein, refers to members of the family Adenoviridae. Adenoviridae typically are medium-sized (90-100 nm), non-enveloped (without an outer lipid bilayer) viruses with an icosahedral nucleocapsid containing a double stranded DNA genome. Methods to obtain adenoviral vectors are known to the skilled person (see, e.g., Kamen, A., and Henry, O., 2004, The Journal of Gene Medicine: A cross-disciplinary journal for research on the science of gene transfer and its clinical applications, 6(S1), S184-S192; Volpers, C. and Kochanek, S., 2004, The Journal of Gene Medicine: A cross-disciplinary journal for research on the science of gene transfer and its clinical applications, 6(S1), S164-S171).

The term “herpes viral vector”, as used herein, refers to a viral vector or plasmid containing structural and functional genetic elements, or portions thereof, that are primarily derived from a herpes virus. The term “herpes virus”, as used herein, refers to any virus from the genus Simplexvirus. Methods to obtain herpes viral vectors are known to the person skilled in the art (see, e.g., Logvinoff, Carine, and Alberto L. Epstein, 2001, Human gene therapy 12.2: 161-167).

The term “alpha-retroviral vector”, as used herein, refers to a viral vector or plasmid containing structural and functional genetic elements, or portions thereof, that are primarily derived from an alpha-retrovirus. The term “alpha-retrovirus”, as used herein, refers to any virus from the genus Alpharetrovirus. Methods to obtain alpha-retroviral vectors are known to the person skilled in the art (see, e.g., Garoff, Henrik, and Kejun Li, 1998, Gene Therapy. 61-69).

In certain embodiments, the viral vector may be an adeno-associated viral (AAV) vector. There are currently two classes of recombinant AAVs (rAAVs) in use: single-stranded AAV (ssAAV) and self-complementary AAV (scAAV). ssAAVs are packaged as either sense (plus-stranded) or anti-sense (minus-stranded) genomes.

That is, in certain embodiments, the viral vector is a DNA-based viral vector. In such embodiments, viral DNA may be directly integrated into the genome of the target cell without reverse transcription of the viral DNA.

In a particular embodiment, the invention relates to a fusion promoter comprising
a) a miR223 promoter or a promoter having at least 95%, 96%, 97%, 98%, 99% or 100% sequence identity to the sequence shown in SEQ ID NO: 1, or a functional fragment thereof; and
b) a microglia-specific promoter, or a functional fragment thereof;
wherein the miR223 promoter or the promoter having at least at least 95%, 96%, 97%, 98%, 99% or 100% sequence identity to the sequence shown in SEQ ID NO: 1, or the functional fragment thereof, is operably linked to the microglia-specific promoter, or the functional fragment thereof.

The promoter miR223 shows great potential for use in cell and gene therapy applications targeting HSCs or keratinocytes due to its resistance to methylation upon cell differentiation. In certain embodiments, the promoter miR223, or functional fragments or variants thereof, may be fused to a second promoter, preferably a microglia-specific promoter.

Thus, in a particular embodiment, the invention relates to the fusion promoter according to the invention, wherein the microglia-specific promoter is
a) a TMEM119 promoter or a promoter having at least 95%, 96%, 97%, 98%, 99% or 100% sequence identity to the sequence shown in SEQ ID NO:3, SEQ ID NO:23 or SEQ ID NO:24, or a functional fragment thereof
b) a P2RY12 promoter or a promoter having at least 95%, 96%, 97%, 98%, 99% or 100% sequence identity to the sequence shown in SEQ ID NO: 2, SEQ ID NO: 21 or SEQ ID NO: 22, or a functional fragment thereof
c) an OLFML3 promoter or a promoter having at least 95%, 96%, 97%, 98%, 99% or 100% sequence identity to the sequence shown in SEQ ID NO:4 or SEQ ID NO:25, or a functional fragment thereof,
d) an ITGAM promoter or a promoter having at least 95%, 96%, 97%, 98%, 99% or 100% sequence identity to the sequence shown in SEQ ID NO:6, or a functional fragment thereof; or
e) an AIF1 promoter or a promoter having at least 95%, 96%, 97%, 98%, 99% or 100% sequence identity to the sequence shown in SEQ ID NO:5, or a functional fragment thereof.

In certain embodiments, the fusion promoter comprises the miR223 promoter and the P2RY12 promoter. That is, in certain embodiments, the invention relates to a fusion promoter comprising
a) a miR223 promoter or a promoter having at least 95%, 96%, 97%, 98%, 99% or 100% sequence identity to the sequence shown in SEQ ID NO: 1, or a functional fragment thereof; and
b) a P2RY12 promoter or a promoter having at least 95%, 96%, 97%, 98%, 99% or 100% sequence identity to the sequence shown in SEQ ID NO: 2, SEQ ID NO:21 or SEQ ID NO:22, or a functional fragment thereof.

In a particular embodiment, the miR223-P2RY12 fusion promoter according to the invention comprises the nucleotide sequence as set forth in SEQ ID NO:26 or SEQ ID NO:27. In a particular embodiment, the miR223-P2RY12 fusion promoter according to the invention comprises a nucleotide sequence having at least 90%, at least 95%, at least 96%, at least 97%, at least 98%, at least 99% sequence identity with the nucleotide sequence as set forth in SEQ ID NO:26 or SEQ ID NO:27.

In certain embodiments, the fusion promoter comprises the miR223 promoter and the TMEM119 promoter. That is, in certain embodiments, the invention relates to a fusion promoter comprising
a) a miR223 promoter or a promoter having at least 95%, 96%, 97%, 98%, 99% or 100% sequence identity to the sequence shown in SEQ ID NO: 1, or a functional fragment thereof; and
b) a TMEM119 promoter or a promoter having at least 95%, 96%, 97%, 98%, 99% or 100% sequence identity to the sequence shown in SEQ ID NO:3, SEQ ID NO:23 or SEQ ID NO:24, or a functional fragment thereof.

In a particular embodiment, the miR223-TMEM119 fusion promoter according to the invention comprises the nucleotide sequence as set forth in SEQ ID NO:28. In a particular embodiment, the miR223-TMEM119 fusion promoter according to the invention comprises a nucleotide sequence having at least 90%, at least 95%, at least 96%, at least 97%, at least 98%, at least 99% sequence identity with the nucleotide sequence as set forth in SEQ ID NO:28.

In certain embodiments, the fusion promoter comprises the miR223 promoter and the OLFML3 promoter. That is, in certain embodiments, the invention relates to a fusion promoter comprising
a) a miR223 promoter or a promoter having at least 95%, 96%, 97%, 98%, 99% or 100% sequence identity to the sequence shown in SEQ ID NO: 1, or a functional fragment thereof; and
b) an OLFML3 promoter or a promoter having at least 95%, 96%, 97%, 98%, 99% or 100% sequence identity to the sequence shown in SEQ ID NO: 8 or SEQ ID NO:9, or a functional fragment thereof.

In a particular embodiment, the miR223-OLFML3 fusion promoter according to the invention comprises the nucleotide sequence as set forth in SEQ ID NO:29. In a particular embodiment, the miR223-OLFML3 fusion promoter according to the invention comprises a nucleotide sequence having at least 90%, at least 95%, at least 96%, at least 97%, at least 98%, at least 99% sequence identity with the nucleotide sequence as set forth in SEQ ID NO:29.

In certain embodiments, the fusion promoter comprises the miR223 promoter and the AIF1 promoter. That is, in certain embodiments, the invention relates to a fusion promoter comprising
a) a miR223 promoter or a promoter having at least 95%, 96%, 97%, 98%, 99% or 100% sequence identity to the sequence shown in SEQ ID NO: 1, or a functional fragment thereof; and
b) an AIF1 promoter or a promoter having at least 95%, 96%, 97%, 98%, 99% or 100% sequence identity to the sequence shown in SEQ ID NOS, or a functional fragment thereof.

In certain embodiments, the fusion promoter comprises the miR223 promoter and the ITGAM promoter. That is, in certain embodiments, the invention relates to a fusion promoter comprising
a) a miR223 promoter or a promoter having at least 95%, 96%, 97%, 98%, 99% or 100% sequence identity to the sequence shown in SEQ ID NO: 1, or a functional fragment thereof; and
b) an ITGAM promoter or a promoter having at least 95%, 96%, 97%, 98%, 99% or 100% sequence identity to the sequence shown in SEQ ID NO:6, or a functional fragment thereof.

In a particular embodiment, the invention relates to the fusion promoter according to the invention, wherein the fusion promoter comprises at least one transcriptional regulatory element, wherein said at least one transcriptional regulatory element is arranged such that it inhibits or activates a transcriptional activity of the promoter.

In a particular embodiment, the invention relates to the fusion promoter according to the invention, wherein the at least one transcriptional regulatory element comprises a binding site for a transcriptional activator or repressor, in particular wherein the transcriptional activator or repressor comprises:
i) an antibiotic-binding domain, in particular a tetracycline/doxycycline-binding domain, a macrolide-binding domain or a pristinamycin-binding domain;
ii) a hormone-binding domain, in particular a RU486-binding domain or an abscisic acid-binding domain;
iii) a steroid-binding domain, in particular an ecdysone-binding domain;
iv) a dimerizer system, in particular a rapamycin-based of rapalog-based dimerizer system.

In a particular embodiment, the invention relates to the fusion promoter according to the invention, wherein the viral vector encodes a riboswitch, and wherein the riboswitch controls translation of an mRNA encoding the therapeutic protein or the combination of therapeutic proteins.

That is, the fusion promoter of the invention may comprise regulatory elements that allow controlling expression of a transgene in a more precise manner. Preferably, the regulatory elements are any of the regulatory elements disclosed elsewhere herein.

In a particular embodiment, the invention relates to the fusion promoter according to the invention, wherein the fusion promoter
a) comprises any one of the sequences set forth in SEQ ID NO: 26-29: or
b) comprises a sequence having 90%, 91%, 92%, 93%, 94% or 95% sequence identity with any one of the sequence set forth in SEQ ID NO:26-29, wherein the promoter drives expression in microglia and/or myeloid cells.

In a particular embodiment, the invention relates to the fusion promoter according to the invention, wherein the fusion promoter
a) comprises the sequence set forth in SEQ ID NO:28: or
b) comprises a sequence having 90%, 91%, 92%, 93% or 95% sequence identity with the sequence set forth in SEQ ID NO:28, wherein the promoter drives expression in microglia and/or myeloid cells.

In a particular embodiment, the invention relates to a host cell comprising the viral vector according to the invention.

That is, the present invention further relates to a host cell comprising the viral vector according to the invention. In certain embodiments, a host cell may be a cell that is used to produce the viral vector according to the invention. For example, the host cell may be a HEK293T cell. In certain embodiment, a host cell may be a cell (e.g. a HSC) that was infected with infectious viral particles or a progeny cell thereof (e.g. a macrophage) comprising viral nucleic acids irrespective of its virus producing capabilities.

A host cell is also said to comprise a viral vector according to the invention, if the host cell has been transfected with plasmids encoding genetic elements for the production of viral vector and the plasmids have integrated into the genome of the host cell in “stable produced cell”. Thus, a viral vector does not necessarily have to be in a circular form to be comprised in a host cell.

In a particular embodiment, the invention relates to the host cell according to the invention, wherein the host cell is a hematopoietic stem cell, preferably a hematopoietic stem cell of a CD34-positive enriched cell population, or wherein the host cell is a myeloid cell. That is, in certain embodiments, the host cell may be a transduced hematopoietic stem cell, preferably a hematopoietic stem cell of a CD34-positive enriched cell population, or a transduced myeloid cell. In particular, a host cell that is used for the treatment and/or prevention of any of the diseases and/or disorders disclosed herein is preferably a transduced hematopoietic stem cell, preferably a hematopoietic stem cell of a CD34-positive enriched cell population, or a transduced myeloid cell.

In certain embodiments, the host cell may be a hematopoietic stem cell. That is, in certain embodiments, the invention relates to a hematopoietic stem cell that has been transduced with any of the viral vectors disclosed herein.

The term “Haematopoietic stem cells” identical to the term “hematopoietic stem cell” or “HSC” or “HSPC” relates to any cell population obtained upon, but not limited to, bone marrow aspiration, apheresis upon stem cell mobilization, or obtained from (umbilical) cord blood, and/or to any cell population in which CD34-positive or CD133-positive cells were enriched by any method, but not limited to CD34-positive and/or CD133-positive cell labelling and enrichment, or by depletion of lineage-positive cells by any method known-in-the-art.

As used herein, “CD34-positive enriched” indicates that the population comprises a higher number and/or higher percentage of CD34-positive cells than is found in the cell populations before the enrichment step. Various methods for CD34-positive cell enrichment are known to the person skilled in the art (see, e.g., Baldwin, K. et. al., 2015, Stem cells, 33(5), 1532-1542; Wojciechowski, Joel C et al., 2008, British journal of haematology vol. 140, 6 673-81; Gori, J. L. et. al., 2012, Blood, The Journal of the American Society of Hematology, 120(13), e35-e44; Kilic, P. et al., 2019, Cells Tissues Organs, 207(1), 15-20.)

In a preferred embodiment, the host cell is a cell in an enriched population of CD34-positive bone marrow cells. In a more preferred embodiment, the host cell is a hematopoietic stem and progenitor cell in an enriched population of CD34-positive bone marrow cells. In a most preferred embodiment, the host cell is a hematopoietic stem cell in an enriched population of CD34-positive bone marrow cells.

In other embodiments, the host cell may be a myeloid cell. That is, in certain embodiments, the invention relates to a granulocyte (neutrophils, eosinophils, and basophils), a monocyte, a macrophage, a Kupffer cell or a mast cell that has been transduced with any of the viral vectors disclosed herein. In certain embodiments, the host cell is a macrophage. In certain embodiments, the host cell is a monocyte. In additional embodiments, the host cell is a microglia.

The skilled person is aware of methods to enrich and/or identify the above-disclosed cell types and to transduce them with viral vectors.

In a particular embodiment, the invention relates to a pharmaceutical composition comprising the viral vector according to the invention and/or the host cell according to the invention.

That is, in certain embodiments, the invention relates to a pharmaceutical composition comprising any one of the viral vectors disclosed herein and/or any one of the host cells disclosed herein.

In certain embodiments, the pharmaceutical composition comprises a viral vector according to the invention. In such embodiments, the pharmaceutical composition is preferably used to transduce a target cell, such as a hematopoietic stem cell, ex vivo. Alternatively, the pharmaceutical composition may be directly administered to a subject in need such that the viral vector comprised in the pharmaceutical composition transduces a target cell in vivo. The skilled person is aware of viral vectors that are suitable for targeting a specific population of target cells in vivo. The skilled person is further aware of ways to formulate a viral vector in a pharmaceutical composition.

In other embodiments, the pharmaceutical composition comprises a host cell comprising a viral vector according to the invention. Such host cells may be obtained by transducing a host cell with any one of the vectors according to the invention. Pharmaceutical compositions comprising a transduced host cell may be administered to a subject in need. The skilled person is aware of methods to formulate a transduced host cell in a pharmaceutical composition.

The term “pharmaceutical composition” as used herein means compositions which result from the combination of individual components which are themselves pharmaceutically acceptable. For example, where intravenous or intrathecal administration is foreseen, the components are suitable or acceptable (in both quality and quantity) for intravenous or intrathecal administration. The skilled person is aware of pharmaceutically acceptable components that are suitable for formulating viral vectors and host cells, respectively.

In certain embodiments, the invention relates to a pharmaceutical composition comprising the viral vector according to the invention and/or the host cell according to the invention and at least one additional therapeutic agent.

The term “therapeutic agent”, as used herein, refers a compound or a composition of matter that upon administration to a subject in a therapeutically effective amount, provides a therapeutic benefit to the subject. A therapeutic agent may be any type of drug, medicine, pharmaceutical, hormone, antibiotic, protein, gene, growth factor and/or bioactive material used for treating, controlling, or preventing diseases or medical conditions.

In some embodiments, the pharmaceutical composition of the invention (and any additional therapeutic agent) is formulated, dosed, and administered in a fashion consistent with good medical practice. Factors for consideration in this context include the particular disorder being treated, the particular subject being treated, the clinical condition of the subject, the cause of the disorder, the site of delivery of the agent, the method of administration, the scheduling of administration, and other factors known to medical practitioners.

The viral vector according to the invention and/or the host cell according to the invention need not be, but is optionally formulated in the pharmaceutical composition with one or more further therapeutic agents currently used to prevent or treat the disorders in question.

Vectors of the invention can be administered to a subject parenterally, preferably intravascularly (including intravenously) and intrathecally. When administered parenterally, it is preferred that the vectors be given in a pharmaceutical vehicle suitable for injection such as a sterile aqueous solution or dispersion. Following administration, the subject is monitored to detect changes in gene expression. Dose and duration of treatment is determined individually depending on the condition or disease to be treated. A wide variety of conditions or diseases can be treated based on the gene expression produced by administration of the gene of interest in the vector of the present invention. The dosage of vector delivered using the method of the invention will vary depending on the desired response by the host and the vector used.

Within the present invention, it is envisioned that the viral vector, the host cells or the pharmaceutical composition according to the invention is administered into the bloodstream or into the liquor cerebrospinalis (or in brain tissue) of a subject. As used herein, “introducing” host cells “into the subject's bloodstream” shall include, without limitation, introducing such cells into one of the subject's veins or arteries via injection. Such administering can also be performed, for example, once, a plurality of times, and/or over one or more extended periods. A single injection is preferred, but repeated injections overtime (e.g., quarterly, half-yearly or yearly) may be necessary in some instances. Such administering is also preferably performed using an admixture of host cells and a pharmaceutical acceptable carrier. Pharmaceutically acceptable carriers are well known to those skilled in the art and include, but are not limited to, 0.01-0.1 M and preferably 0.05 M phosphate buffer or 0.8% saline. Additionally, such pharmaceutically acceptable carriers can be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions and suspensions, including saline and buffered media. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as Ringer's dextrose, those based on Ringer's dextrose, and the like. Fluids used commonly for i.v. administration are found, for example, in Remington: The Science and Practice of Pharmacy, 20th Ed., p. 808, Lippincott Williams & Wilkins (2000). Preservatives and other additives may also be present, such as, for example, antimicrobials, antioxidants, chelating agents, inert gases, and the like.

It is preferred herein that the viral vector, the host cell or the pharmaceutical composition according to the invention is administered into the bloodstream of a subject. However, the viral vector, the host cell or the pharmaceutical composition according to the invention may also be administered directly to a target tissue. That is, in certain embodiments, the viral vector, the host cell or the pharmaceutical composition according to the invention may be injected directly into the brain. Alternatively, the viral vector, the host cell or the pharmaceutical composition according to the invention may be administered by direct CNS injection, injection into the CSF, intrathecal injection and/or intravascular administration.

Alternatively, the viral vector, the host cell or the pharmaceutical composition according to the invention may be administered directly into a tumor.

In a particular embodiment, the invention relates to a viral vector according to the invention, the host cell according to the invention or the pharmaceutical composition according to the invention for use in medicine.

That is, the viral vector, the host cell or the pharmaceutical composition according to the invention may be used for the treatment of a subject in need. The term “treatment” as used herein includes preventative (e.g., prophylactic), curative or palliative treatment and “treating” as used herein also includes preventative, curative and palliative treatment. The term “subject” as used herein relates to animals, preferably mammals, and, more preferably, humans.

In a particular embodiment, the invention relates to the viral vector according to the invention, the host cell according to the invention or the pharmaceutical composition according to the invention for use in the treatment of a disease or disorder which has its origin or a manifestation in the brain or is brain-based.

Targeting brain cells for therapeutic treatments is challenging due to the selective permeability of the blood-brain-barrier. Within the present invention, the inventors target diseases or disorders of the brain by cell and gene therapy. For that, hematopoietic stem cells or a population of cells comprising hematopoietic stem cells may be transformed with any one of the viral vectors disclosed herein and administered to a subject suffering of a disease or disorder in the brain. Hematopoietic stem cells can circulate in the blood stream and are able to cross the blood brain barrier, especially during temporary leakage of the blood-brain-barrier upon treatment related irradiation or chemotherapy by e.g. busulfan administration. Once inside the brain, hematopoietic stem cells can differentiate into macrophages that show characteristics of microglia and can replace microglia in the brain (Speicher et al., Generating microglia from human pluripotent stem cells: novel in vitro models for the study of neurodegeneration; Molecular Neurodegeneration; 14, Article number 46 (2016)). The viral vectors of the present invention are particularly suited for targeting the brain since they have been demonstrated to be active both in macrophages and in microglial cells.

While it is preferred to administer hematopoietic stem cells comprising the viral vector according to the invention into the bloodstream of a subject in need, the viral vector, the host cell or the pharmaceutical composition according to the invention may also be administered directly into the brain (intracranial).

In a particular embodiment, the invention relates to the viral vector according to the invention, the host cell according to the invention or the pharmaceutical composition according to the invention for use in the prevention and/or treatment of a PGRN-associated disease or disorder, in particular wherein the viral vector encodes PGRN, or a functional fragment thereof.

Different diseases and disorders have been reported to be caused by abnormal expression of progranulin. In particular, mutations in the PGRN gene have been reported as the cause of various neurodegenerative diseases or disorders. Thus, the viral vectors according to the invention may be used to restore the progranulin levels in the brains of subjects suffering from a PGRN-associated disease or disorder. For that, it is preferred that the transgene encoded in the viral vector is the PGRN gene or a polynucleotide encoding a polypeptide having PGRN functionality and at least 95%, 96%, 97%, 98% or 99% sequence identity to the sequences shown in SEQ ID NO: 7, SEQ ID NO: 8 or SEQ ID NO: 9.

In a particular embodiment, the invention relates to the viral vector, the host cell or the pharmaceutical composition for use according to the invention, wherein the PGRN-associated disease or disorder is a neurodegenerative disease or disorder.

That is, the PGRN-associated disease may be a neurodegenerative disease or disorder. Within the present invention, the neurodegenerative disease or disorder is preferably a neurodegenerative disease or disorder which is associated with abnormal PGRN expression.

In a particular embodiment, the invention relates to the viral vector, the host cell or the pharmaceutical composition for use according to the invention, wherein the neurodegenerative disease or disorder is a frontotemporal degenerative disease or disorder. In a particular embodiment, the invention relates to the viral vector, the host cell or the pharmaceutical composition for use according to the invention, wherein the degenerative disease or disorder is selected from the group consisting of: Alzheimer's disease, amyotrophic lateral sclerosis, neuronal ceroid lipofuscinosis and Parkinson's disease.

In a particular embodiment, the invention relates to the viral vector, the host cell or the pharmaceutical composition for use according to the invention, wherein the frontotemporal degenerative disease or disorder is frontotemporal dementia. Preferably, the frontotemporal degenerative disease or disorder is frontotemporal dementia that is caused by a mutation in the PGRN gene.

In a particular embodiment, the invention relates to a viral vector according to the invention, the host cell according to the invention or the pharmaceutical composition according to the invention for use in the treatment of cancer, lymphoma and/or sarcoma in particular wherein the viral vector encodes at least one of IL-12, IFN-gamma, G-CSF, GM-CSF, IL-2, IL-15, IL-21 and/or IFN-alpha; or functional fragments thereof.

That is, the viral vectors of the invention may be used in the treatment of cancer. It has been demonstrated herein that the promoters of the invention are active in different myeloid cells as well as in microglia. Accordingly, the viral vectors according to the invention or host cells comprising the viral vector according to the invention may be used in the treatment of cancer in the brain, as well as in other parts of the body.

For example, hematopoietic stem cells comprising a viral vector according to the invention may be administered to a subject suffering from cancer. The hematopoietic stem cells may differentiate into myeloid cells and migrate to the site of the tumor to elicit an immune response against the tumor. The myeloid cell may comprise a transgene encoding one of the cytokines discloses herein to increase the immune response against the tumor. Alternatively or in addition, the transgene may encode an antigen-binding protein that directs the myeloid cell to the tumor to elicit a more pronounced immune response against the tumor.

In a particular embodiment, the invention relates to the viral vector, the host cell or the pharmaceutical composition for use according to the invention, wherein the cancer, lymphoma and/or sarcoma is a brain tumor or a brain metastasis.

That is, the viral vector or the host cell according to the invention may be used to treat tumors in the brain. The brain tumor may be a primary or secondary brain tumor. As described above, hematopoietic stem cells comprising the viral vector according to the invention may migrate to the brain and differentiate into macrophages that show characteristics of microglia and can replace microglia in the brain. Inside the brain, these microglia and microglia-like cells can secrete cytokines, such as IL-12, IFN-gamma, G-CSF, GM-CSF, IL-2, IL-15, IL-21, IFN-alpha or combinations or fusion variants thereof to trigger an immune response in the brain against the tumor.

In a particular embodiment, the invention relates to the viral vector, the host cell or the pharmaceutical composition for use according to the invention, wherein the brain tumor is selected from the group consisting of: glioblastoma, glioma, ganglioneuroblastoma, astrocytoma, oligodendroglioma, PNET (primitive neuroectodermal tumor), medulloblastoma, CNS lymphoma, meningioma, retinoblastoma and neuroblastoma.

In a particular embodiment, the invention relates to the viral vector, the host cell or the pharmaceutical composition for use according to the invention, wherein the brain tumor is a metastatic tumor originating from any form of breast cancer, lung cancer, colon cancer, testicular cancer, renal carcinomas, melanoma, ovary carcinomas, prostate carcinoma, neuroendocrine tumors or any other solid tumor or any sarcoma, or any hematologic tumor, comprising all forms of leukemia and lymphomas.

The term “cancer”, as used herein, refers to a disease characterized by dysregulated cell proliferation and/or growth. The term comprises benign and malignant cancerous diseases, such as tumors, and may refer to an invasive or non-invasive cancer. The term comprises all types of cancers, including carcinomas, sarcomas, lymphomas, leukemias, germ cell tumors, and blastomas.

In a particular embodiment, the invention relates to the viral vector, the host cell or the pharmaceutical composition for use according to the invention, wherein the viral vector, the host cell or the pharmaceutical composition is administered in conjunction with a therapy that reduces the integrity of the blood-brain-barrier, in particular wherein the therapy that reduces the integrity of the blood-brain-barrier is a bone marrow conditioning therapy, a CNS conditioning therapy, and/or a blood-brain-barrier conditioning therapy.

As disclosed above, the invention may be used in the prevention and/or treatment of a disease or disorder which has its origin or a manifestation in the brain or is brain-based. For that, it is envisioned that hematopoietic stem cells comprising the viral vector of the invention are administered to a subject in need. Once administered to the subject, the hematopoietic stem cells can migrate into the brain and differentiate into microglia-like macrophages, or into microglia.

Alternatively, AAV-based viral vectors according to the invention, or pharmaceutical composition comprising AAV-based viral vectors according to the invention may be applied directly into the brain compartments for in vivo infection of cells in need.

To replace microglia in the brain more efficiently with the transduced cells of the invention, it is preferred that endogenous microglia are depleted before the administration of transduced cells. Various treatment regimens that reduce the integrity of the blood-brain-barrier have been reported to result in the depletion of microglia. For example, Capotondo et al. have demonstrated that brain conditioning is instrumental for successful microglia reconstitution following hematopoietic stem cell transplantation (Proc Natl Acad Sci USA. 2012 Sep. 11; 109(37): 15018-15023).

In a particular embodiment, the invention relates to the viral vector, the host cell or the pharmaceutical composition for use according to the invention, wherein the bone marrow conditioning therapy comprises the use of cytotoxic agents, alkylating agents, Busulphan, Treosulfan, Etoposide, Lomustin, radiotherapy, targeted radiotherapy (e.g. Yttrium-90 labeled anti-CD45 antibody, or Yttrium-90 labeled anti-CD66 antibody), ACK2 (anti-c-kit antibody), CD117 antibody-drug-conjugates, CD45-SAP, colony-stimulating factor 1 (CSF1) specific agents, PLX3397, BLZ9445, PLX5622, RG7155, PLX647, Ki20227, GW2580, IL-34 and/or desatinib.

In a particular embodiment, the invention relates to the viral vector, the host cell or the pharmaceutical composition for use according to the invention, wherein the CNS conditioning therapy comprises the use of Busulphan.

In a particular embodiment, the invention relates to the viral vector, the host cell or the pharmaceutical composition for use according to the invention, wherein the blood-brain-barrier conditioning therapy comprises radiotherapy or targeted radiotherapy.

In a particular embodiment, the invention relates to the viral vector, the host cell or the pharmaceutical composition for use according to the invention, wherein the viral vector, the host cell or the pharmaceutical composition is administered after the therapy that reduces the integrity of the blood-brain-barrier, in particular wherein the viral vector, the host cell or the pharmaceutical composition is administered not earlier than half a day after the therapy that reduces the integrity of the blood-brain-barrier.

That is, the viral vector, the host cell or the pharmaceutical composition according to the invention, may be administered to the subject in need 0.5, 1, 2, 3, 4, 5, 6, 7, 8, 9, 10, 11, 12, 13, 14 or 15 days after the therapy that reduced the integrity of the blood-brain-barrier.

While the viral vectors of the invention are particularly well suited for the treatment of brain-based diseases or disorders due to the activity of their promoters in myeloid cells and microglia, it is important to understand that the viral vectors may also be used to target tumors in the CNS or any other part of the body. In principle, the viral vectors of the invention may be used to treat cancer in any organ or tissue that is accessible for myeloid cells, such as macrophages or monocytes.

In a particular embodiment, the invention relates to the viral vector according to the invention, the host cell according to the invention or the pharmaceutical composition according to the invention for use in the treatment of autoimmune diseases.

That is, the viral vector, the host cell or the pharmaceutical composition according to the invention may also be used in the treatment of autoimmune diseases.

The term “autoimmune disease” as used herein is defined as a disorder that results from an autoimmune response. An autoimmune disease is the result of an inappropriate and excessive response to a self-antigen. Examples of autoimmune diseases include but are not limited to, Addison's disease, alopecia areata, ankylosing spondylitis, autoimmune bullous diseases other than pemphigus vulgaris, autoimmune hepatitis, autoimmune parotitis, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, all types of vasculitis, vitiligo, myxedema, pernicious anemia, ulcerative colitis, among others.

Transgenes that may be used for the treatment of autoimmune diseases include IL-1, IL-1R antagonist, IL-2, IL-4, IL-10, TGFbeta, FOXP3, T-bet, GATA-3, CD36 family (CD36-L1, CD36-L2) binding CD1b, CD1c, CD1D, and T cell receptor recognition of MHC-related protein number one (MR1).

In a particular embodiment, the invention relates to the viral vector according to the invention, the host cell according to the invention or the pharmaceutical composition according to the invention for use in the treatment of autoinflammatory diseases.

The term “autoinflammatory disease” as used herein should be understood to encompass any autoinflammatory disease. Non-limiting examples of an autoinflammatory disease which may be treated with the viral vector, the host cell or the pharmaceutical composition of the invention are hypocomplementemic and normocomplementemic urticarial vasculitis, pericarditis, myositis, anti-synthetase syndrome, scleritis, macrophage activation syndrome, Beret's Syndrome, PAPA Syndrome, Blau's Syndrome, gout, adult and juvenile Still's disease, cryropyrinopathy, Muckle-Wells syndrome, familial cold-induced auto-inflammatory syndrome, neonatal onset multisystemic inflammatory disease, familial Mediterranean fever, chronic infantile neurologic, cutaneous and articular syndrome, systemic juvenile idiopathic arthritis, Hyper IgD syndrome, Schnitzler's syndrome, and TNF receptor-associated periodic syndrome (TRAPS).

Transgenes that may be used for the treatment of autoinflammatory diseases include IL-1Receptor-antagonist, IL-1beta.

In a particular embodiment, the invention relates to the viral vector according to the invention, the host cell according to the invention or the pharmaceutical composition according to the invention for use in the treatment of allergic diseases.

The term “allergic disease” as used herein refers to any symptoms, tissue damage, or loss of tissue function resulting from allergy and includes, without limitation, diseases such as atopic dermatitis, urticaria, contact dermatitis, allergic conjunctivitis, allergic rhinitis, allergic asthma, anaphylaxis, food allergy and hay fever.

Transgenes that may be used for the treatment of allergic diseases include genes encoding proteins, comprising antibodies and other receptor-binding proteins against any part of any IgE, comprising Fc, Fab, including variable and hypervariable region of Fab; or any receptor of cells implied to mediate allergic reactions, including mast cells, eosinophils, B cells, and T cells. Furthermore soluble potentially neutralizing binding proteins and peptides or antibodies should be induced by genes against IL-1, IL-4, IL-33 and any other cytokine, comprising all forms of interleukines and chemokines, implicated in allergic diseases.

In a particular embodiment, the invention relates to the viral vector according to the invention, the host cell according to the invention or the pharmaceutical composition according to the invention for use in hematopoietic and solid organ transplantation.

That is, the viral vector, the host cell or the pharmaceutical composition according to the invention may be administered to a subject in need before hematopoietic or solid organ transplantation. Transgenes that may be used in hematopoietic and solid organ transplantation include IL-1, IL-1R antagonist, IL-2, IL-4, IL-10, TGFbeta, FOXP3, T-bet, GATA-3, CD36 family (CD36-L1, CD36-L2) binding CD1b, CD1c, CD1D, and T cell receptor recognition of MHC-related protein number one (MR1).

In a particular embodiment, the invention relates to a method for treating a disease or disorder which has its origin or a manifestation in the brain or is brain based in a subject in need, the method comprising the steps of:
a) genetically modifying a hematopoietic stem cell and/or a population of enriched CD34-positive bone marrow cells, the modification step comprising a step of contacting the hematopoietic stem cell and/or the population of enriched CD34-positive bone marrow cells with the viral vector according to the invention; or genetically modifying a myeloid cell and/or a population of enriched myeloid cells, the modification step comprising a step of contacting the myeloid cell and/or the population of enriched myeloid cells with the viral vector according to the invention;
b) administering the genetically modified cells from step (a) intravenously to the subject in need; and
c) treating a disease or disorder which has its origin or a manifestation in the brain or is brain based in the subject in need.

That is, the invention further relates to methods for treating diseases or disorders in the brain. As mentioned above, host cells comprising the viral vector according to the invention may migrate into the brain of a subject suffering from a brain-based disease or disorder and replace microglia.

For that, a cell, such as a hematopoietic stem cell or a myeloid cell may be transduced ex vivo with a viral vector according to the invention. A population of transduced cells may then be administered to a subject in need. In certain embodiment, a transduced hematopoietic stem cell is administered to the subject in need. This may be advantageous, since stem cells have a higher potential to cross the blood-brain-barrier than other cell types. However, the host cell may also be a myeloid cell, such as a monocyte and/or macrophage. However, monocytes and/or macrophages are preferably used in subjects with a compromised blood-brain-barrier.

In a particular embodiment, the invention relates to the method according to the invention, wherein the hematopoietic stem cell and/or the population of enriched CD34-positive bone marrow cells, or the myeloid cell and/or the population of enriched myeloid cells have been obtained from the subject in need or from a foreign donor.

That is, in certain embodiments, the method of the invention comprises the use of autologous cells. The skilled person is aware of methods to enrich certain cell types from the blood of a subject. Consequently, a certain type of blood cell may be enriched from the blood of a subject in need, transduced with a viral vector according to the invention, and administered back to the subject in need. Autologous cells have the advantage that they reduce the risk of immunogenic reactions.

In other embodiments, the cell that is administered to the subject in need may originate from a foreign donor. The skilled person is aware of methods to identify compatible donors or to manipulate the cells and/or the subject in need thereof such that the risk of an immunogenic reaction is reduced.

In a particular embodiment, the invention relates to a method for treating a disease or disorder which has its origin or a manifestation in the brain or is brain based in a subject in need, the method comprising the steps of:
a) mobilizing hematopoietic stem cells in the subject in need;
b) administering the viral vector according to the invention intravenously to the subject in need subsequent to the mobilization of hematopoietic stem cells in step (a); and
c) treating a disease or disorder which has its origin or a manifestation in the brain or is brain based in the subject in need.

That is, the viral vector of the invention or a pharmaceutical composition comprising the viral vector according to the invention may also be directly administered to a subject in need. Preferably, the subject is pre-treated with an agent that results in the mobilization of hematopoietic stem cells in said subject, such that the mobilized hematopoietic stem cells can be infected with the viral vector according to the invention in vivo. Agent that are commonly used to stimulate the mobilization of hematopoietic stem cells from the bone marrow are G-CSF and Plerixafor. However, other agents that stimulate the mobilization of hematopoietic stem cells from the bone marrow are known in the art and may be used as part of the claimed method.

In certain embodiments, the in vivo transduced hematopoietic stem cells may migrate to the brain where they differentiate into microglia or microglia-like cells. In such embodiments, the method may be used for the prevention and/or treatment of brain-based diseases and disorders. The microglia-like cells may express one or more transgenes that are required for the prevention and/or treatment of the brain-based disease or disorder. For example, the microglia-like cells may express PGRN when used for the prevention and/or treatment of one of the PGRN-associated diseases or disorders disclosed herein. Alternatively, the microglia-like cells may express one of the cytokines disclosed herein when used in the treatment of brain tumors.

In a particular embodiment, the invention relates to the method according to the invention, wherein the mobilization of hematopoietic stem cells in the subject in need comprises the administration of G-CSF and/or Plerixafor. Plerixafor (INN and USAN, trade name Mozobil) is an immunostimulant used to mobilize hematopoietic stem cells in cancer patients into the bloodstream.

In a particular embodiment, the invention relates to a method according to the invention, wherein the disease or disorder which has its origin or a manifestation in the brain or is brain based is a PGRN-associated disease or disorder, in particular wherein the PGRN-associated disease or disorder is a neurodegenerative disease or disorder, in particular wherein the neurodegenerative disease or disorder is a frontotemporal degenerative disease or a neurodegenerative disorder, in particular wherein the frontotemporal degenerative disease or neurodegenerative disorder is selected from the group consisting of Alzheimer's disease, amyotrophic lateral sclerosis, neuronal ceroid lipofuscinosis, and Parkinson's disease, in particular wherein the viral vector encodes PGRN, or a functional fragment thereof.

That is, viral vectors encoding progranulin or a polypeptide having PGRN functionality and at least 95%, 96%, 97%, 98% or 99% sequence identity to the sequence shown in SEQ ID NO: 7, SEQ ID NO: 8 or SEQ ID NO: 9 may be used in the treatment of any of the PGRN-associated neurodegenerative diseases disclosed herein.

In a particular embodiment, the invention relates to the method according to the invention, wherein the disease or disorder which has its origin, or a manifestation, in the brain or is brain based is a brain tumor, in particular wherein the brain tumor is selected from the group consisting of: glioma, glioblastoma, ganglioneuroblastoma, astrocytoma, oligodendroglioma, PNET (primitive neuroectodermal tumor), medulloblastoma, CNS lymphoma, and neuroblastoma; or wherein the brain tumor is a metastatic tumor originating from any form of breast cancer, lung cancer, colon cancer, testicular cancer, renal carcinomas, melanoma, prostate cancer, or any other solid tumor or any sarcoma, or any hematologic tumor, comprising all forms of leukemia and lymphomas, in particular wherein the viral vector encodes IL-12, IFN-gamma, GM-CSF, G-CSF, 11-2, IL-15, IL-21 and/or IFN-alpha, or functional fragments thereof.

In a particular embodiment, the invention relates to the method according to the invention, wherein the method comprises an additional step of temporarily reducing the integrity of the blood-brain-barrier, in particular wherein reducing the integrity of the blood-brain-barrier comprises a bone marrow conditioning therapy, a CNS conditioning therapy, and/or a blood-brain-barrier conditioning therapy.

In a particular embodiment, the invention relates to the method according to the invention, wherein the therapy that reduces the integrity of the blood-brain-barrier is performed prior to the administration of the genetically modified cells to the subject in need, in particular wherein the time interval between the therapy that reduces the integrity of the blood-brain-barrier and the administration of the genetically modified cells is carried out after the therapy that reduces the integrity of the blood-brain-barrier.

The therapy for reducing the integrity of the blood brain barrier may be any one of the therapies disclosed herein. In certain embodiments, the therapy for reducing the integrity of the blood brain barrier may be administered 0.5, 1, 2, 3, 4, 5, 6, 7, 8, 9, 10, 11, 12, 13, 14 or 15 days before the administration of the viral vector, the host cell or the pharmaceutical composition according to the invention.

As mentioned above, the viral vector, the host cell or the pharmaceutical composition according to the invention may also be used to treat cancer in other parts of the body. That is, in a particular embodiment, the invention relates to a method for treating cancer in a subject in need, the method comprising the steps of:
a) mobilizing hematopoietic stem cells in the subject in need;
b) administering the viral vector according to the invention intravenously to the subject in need subsequent to the mobilization of hematopoietic stem cells in step (a); and
c) treating cancer in the subject in need.

For the treatment of cancer, it is preferred that the viral vector encodes at least one of IL-12, IFN-gamma, GM-CSF, G-CSF, 11-2, IL-15, IL-21 and/or IFN-alpha, or functional fragments thereof.

In a particular embodiment, the invention relates to a method for expressing a transgene in the brain and/or CNS of a subject, the method comprising the steps of:
a) genetically modifying a hematopoietic stem cell and/or a population of enriched CD34-positive bone marrow cells, the modification step comprising a step of contacting the hematopoietic stem cell and/or the population of enriched CD34-positive bone marrow cells with the viral vector according to the invention; or genetically modifying a myeloid cell and/or a population of enriched myeloid cells, the modification step comprising a step of contacting the myeloid cell and/or the population of enriched myeloid cells with the viral vector according to the invention;
b) administering the genetically modified cells from step (a) intravenously or intrathecally to the subject in need; and
c) expressing the transgene encoded by the viral vector in the brain and/or CNS of the subject.

That is, the method may be used for expressing a transgene in the brain or in the central nervous system of a subject in need. For this, a population of cells may be transduced with the viral vector according to the invention ex vivo. In certain embodiments, the population of cells may be a population of hematopoietic stem cells or a population of enriched CD34-positive bone marrow cells. Preferably, the population of enriched CD34-positive bone marrow cells comprises hematopoietic stems cells and/or hematopoietic progenitor cells. In certain embodiments, the population of cells may be an enriched population of myeloid cells. The myeloid cell may be any myeloid cell disclosed herein. In certain embodiments the myeloid cell may be a macrophage.

The term “population of cells” is used to denote a plurality of cells. For example a population of hematopoietic stem cells refers to a plurality of stem cells. A population of hematopoietic stem cells may consist exclusively of hematopoietic stem cells. However, a “population of hematopoietic stem cells”, as used herein, is preferably understood to be a population of cells comprising hematopoietic stem cells. That is, the “population of hematopoietic stem cells” may comprise other cell types, in particular CD34-positive cell types. The skilled person is aware of methods to enrich hematopoietic stem cells from a mixture of cells, for example from blood or bone marrow. For example, hematopoietic stem cells may be enriched based on the expression of the cell surface marker CD34, resulting in a population of enriched CD-34-positive bone marrow cells. A population of enriched CD-34-positive bone marrow cells may be a population of cells wherein at least 70%, at least 80%, at least 90% or at least 95% of all cells in the population express the cell surface marker CD34.

An enriched population of myeloid cells is a population of cells wherein at least 70%, at least 80%, at least 90% or at least 95% of all cells in the population are myeloid cells. The skilled person is aware of combinations of cell surface markers that may be used to enrich a specific type or specific types of myeloid cells by flow cytometry.

The population of cells may be transduced with any of the viral vectors disclosed herein. The transduction step may take place ex vivo. The skilled person is aware of methods to transduce a cell with a viral vector.

In a particular embodiment, the invention relates to the method according to the invention, wherein the hematopoietic stem cell and/or the population of enriched CD34-positive bone marrow cells; or wherein the myeloid cell and/or the population of enriched myeloid cells has been obtained from the subject or from a foreign donor.

That is, the population of cells may comprise autologous or allogeneic cells as described above.

In a particular embodiment, the invention relates to a method for expressing a transgene in the brain and/or CNS of a subject, the method comprising the steps of:
a) mobilizing hematopoietic stem cells in the subject;
b) administering the viral vector according to the invention intravenously to the subject in need subsequent to the mobilization of hematopoietic stem cells in step (a); and
c) expressing the transgene encoded in the viral vector in the brain and/or CNS of the subject.

That is, in certain embodiments, the transgene may be delivered to a subject in need in vivo. That is, the viral vector according to the invention may be administered directly to a subject, preferably after the subject received a stem cell mobilization therapy. Accordingly, in a particular embodiment, the invention relates to the method according to the invention, wherein the mobilization of hematopoietic stem cells in the subject comprises the administration of G-CSF or Plerixafor.

In a particular embodiment, the invention relates to the method according to the invention, wherein the method comprises an additional step of temporarily reducing the integrity of the blood-brain-barrier, in particular wherein reducing the integrity of the blood-brain-barrier comprises a bone marrow conditioning therapy, a CNS conditioning therapy, and/or a blood-brain-barrier conditioning therapy.

That is, migration of the transduced hematopoietic stem cells to the brain may be more efficient if microglia have been depleted in the subject in need before the viral vector is administered. Methods and compounds to deplete microglia in a subject are known in the art and have been disclosed herein.

In a particular embodiment, the invention relates to the method according to the invention, wherein the therapy that reduces the integrity of the blood-brain-barrier is performed prior to the administration of the genetically modified cells to the subject in need, in particular wherein the time interval between the therapy that reduces the integrity of the blood-brain-barrier and the administration of the genetically modified cells is carried out after the therapy that reduces the integrity of the blood-brain-barrier.

That is, the therapy for reducing the integrity of the blood brain barrier may be administered 1, 2, 3, 4, 5, 6, 7, 8, 9, 10, 11, 12, 13, 14 or 15 days before the administration of the viral vector according to the invention or the pharmaceutical composition comprising the viral vector according to the invention.

In a particular embodiment, the invention relates to a method for treating a disease or disorder which has its origin or a manifestation in the brain or is brain based in a subject in need, the method comprising the steps of:
a) administering the viral vector according to the invention into the brain of the subject in need or intrathecally; and
b) treating a disease or disorder which has its origin or a manifestation in the brain or is brain based in the subject in need.

That is, the viral vectors according to the invention or a pharmaceutical composition comprising the viral vectors according to the invention may be administered directly into the brain or into the spinal canal (intrathecally). In such embodiments, it is preferred that the viral vector is an AAV-based viral vector. Accordingly, in a particular embodiment, the invention relates to the viral vector according to the invention, wherein the viral vector is an AAV-based viral vector.

It is to be understood that the method may be used for the treatment or prevention of any brain-based disease or disorder disclosed herein, in particular neurodegenerative diseases and disorders and cancer. The term “intrathecally” as used herein means administered into or within the fluid-filled spaces between the thin layers of tissue that cover the brain and spinal cord.

The present invention provides novel retroviral vectors for use in human therapy, particularly for use in in the treatment of a disease or disorder which has its origin in the brain or is brain based, particularly a PGRN-associated neurodegenerative disease or disorder including frontotemporal degenerative disease or disorder such as Alzheimer's disease, amyotrophic lateral sclerosis, and Parkinson's disease. The invention also provides retroviral vectors for use in the treatment of brain tumors, particularly brain tumors selected from the group consisting of glioblastoma, glioma, ganglioneuroblastoma, astrocytoma, oligodendroglioma, PNET (primitive neuroectodermal), medulloblastoma, CNS lymphoma, and neuroblastoma, or any other CNS tumor and further in the treatment of brain metastasis, originating from any forms of breast, lung, colon, testicular, renal carcinomas and melanoma, or any other solid tumor, and any hematologic tumor, comprising all forms of leukemia and lymphomas.

In particular, the present invention provides a retroviral gene therapy vector, particularly a lentiviral gene therapy vector, comprising a nucleotide sequence encoding a therapeutic transgene, particularly a PGRN cDNA, under control of a tissue specific promoter, which vector can be used for the transduction of haematopoietic stem cells (HSC). The specific vector architecture according to the present invention results in exclusive expression of the therapeutic transgene in HSC-derived monocytes/macrophages, dendritic cells, and microglia-like and microglia cells in the brain and leads to moderate levels of gene expression to avoid hippocampal toxicity and neurodegeneration, affecting neurons and glia cells as seen with alternative constructs.

The vector according to the invention comprises the safety-feature of a myelo-/microglia-specific promoter for phagocyte-specific expression, preferably but not restricted to the miR223 gene promoter, or to a fusion promoter construct comprising the miR223 promoter, to drive transgene expression, particularly expression of a PGRN cDNA.

Accordingly, in a specific embodiment, the invention relates to the introduction of a PGRN-encoding expression cassette, comprising a myelo-/microglia-specific promoter, preferably but not restricted to the miR223 promoter, into HSC by a lentiviral self-inactivating (SIN) gene therapy vector.

In another specific embodiment, the invention relates to the introduction of a PGRN-encoding expression cassette, comprising a myelo-/microglia-specific promoter selected from the group consisting of a TMEM119 promoter, a P2RY12 promoter, an OLFML3 promoter, an AIF1 promoter and an ITGAM promoter.

In still another specific embodiment, the invention relates to the introduction of a PGRN-encoding expression cassette, comprising a myelo-/microglia-specific promoter, preferably but not restricted to a miR223 fusion promoter, particularly a fusion promoter, wherein the miR223 promoter or a functional part thereof is fused with all or functional part of a promoter selected from the group consisting of a TMEM119 promoter, a P2RY12 promoter, an OLFML3 promoter, an AIF1 promoter and a ITGAM promoter.

In one aspect, the invention relates to the use of a TMEM119 promoter construct, a P2RY12 promoter construct, an OLFML3 promoter construct, or fusion constructs consisting of miR223 fused to TMEM119, miR223 fused to P2RY12, or of miR223 fused to OLFML3 promoter, to drive PGRN expression in HSC-derived monocytes/macrophages, dendritic cells, as well as in microglia-like cells or microglia upon migration of macrophages into the brain.

Transduction of the HSC is followed by administration of the ex vivo treated HSC to the patient. In a specific embodiment, the ex vivo treated HSC are administered intravenously.

For HSC transplantation, the bone marrow of the patient will be conditioned with a suitable conditioning compound or treatment, particularly with busulphan, treosulfan, radiotherapy, or biological agents that may deplete endogenous brain microglia, but preferably with busulphan. This will allow HSC-derived transgenic monocytes/macrophages or dendritic cells, to enter the brain, to reach a considerable level of chimerism of HSC-derived monocytes/macrophages, microglia-like macrophages, dendritic cells, and/or microglia cells in the brain, and thereby deliver sufficient amounts of PGRN or other transgenes into the brain.

In a specific embodiment of the invention, the patients are pre-treated with busulphan, treosulfan, radiotherapy, or biologics such as monoclonal antibody-based or small-molecule-based inhibitors of colony-stimulating factor 1 (CSF1) and CSF1 receptor (CSF1R) inhibitors such as PLX3397, BLZ9445, PLX5622, RG7155, PLX647, Ki20227, GW2580 or the CSF1R-ligand IL-34, desatinib and any combination thereof, within a window of between the past 5 to the past 20 days before administration, but particularly within the last 8 days or the last 15 days before introduction.

The retroviral vectors according to the invention can also be used for targeting bone-marrow derived macrophages and microglia involved in brain tumors and metastasis. The specific vector architecture according to the present invention comprising the myelo-/microglia-specific promoters is the fundament for the successful expression of proteins in bone marrow derived monocytes/macrophages, dendritic cells, microglia-like cells and microglia, in order to reversing or slowing tumor progression.

In particular, the present invention relates to the use of the retroviral vector constructs according to the invention and as described herein for the treatment of patients suffering from a brain tumor, particularly from a brain tumor selected from the group consisting of glioblastoma, glioma, ganglioneuroblastoma, astrocytoma, oligodendroglioma, PNET (primitive neuroectodermal), medulloblastoma, CNS lymphoma, and neuroblastoma, or any other CNS tumor.

In another specific embodiment, the present invention relates to the use of the retroviral vector constructs according to the invention and as described herein for the treatment of patients suffering from brain metastasis, originating from any forms of breast, lung, colon, testicular, renal carcinomas and melanoma, or any other solid tumor, and any hematologic tumor, comprising all forms of leukemia and lymphomas.

In particular, the present invention provides the following embodiments:
1. A retroviral vector molecule comprising a nucleic acid molecule encoding a therapeutic polypeptide or a combination of therapeutic polypeptides under control of a myelo-/microglia-specific promoter, or a combination of myelospecific and microglia-specific promoters, in particular a fusion-promoter, which drives expression of the therapeutic polypeptide or the combination of therapeutic polypeptides in HSC-derived myeloid cells, HSC-derived blood monocytes/macrophages, dendritic cells and in brain microglia or microglia-like cells upon migration of macrophages into the brain.
2. The retroviral vector of embodiment 1, wherein the microglia-specific promoter is a promoter or a promoter fragment, which has the promoter functionality of a promoter selected from the group consisting of a TMEM119 promoter, a P2RY12 promoter, an OLFML3 promoter, an AIF1 promoter and an ITGAM promoter.
3. The retroviral vector of embodiment 1, wherein the myelo-/microglia-specific promoter is a promoter or a promoter fragment, which has the promoter functionality of a promoter selected from
   (a) an AIF1 promoter or an ITGAM promoter; or
   (b) a fusion promoter comprising a promoter or a promoter fragment, which has the promoter functionality of a miR223 promoter and a promoter or a promoter fragment, which has the promoter functionality of a promoter selected from the group consisting of a TMEM119 promoter, a P2RY12 promoter, an OLFML3 promoter
4. The retroviral vector of embodiment 3, wherein the promoter or promoter fragment with miR233 promoter functionality has at least 95%, 96%, 97%, 98%, 99%, 100% sequence identity to the sequence shown in SEQ ID NO: 1, or is a fragment thereof of at least 200 nucleotides in length, which promoter or fragment still has the promoter functionality of the miR223 promoter.
5. The retroviral vector of any one of embodiments 1 to 3, wherein the promoter or promoter fragment with P2RY12 promoter functionality has at least 95%, 96%, 97%, 98%, 99%, 100% sequence identity to the sequence shown in SEQ ID NO: 2, or is a fragment thereof of at least 200 nucleotides in length, which promoter or fragment still has the promoter functionality of the P2RY12 promoter.
6. The retroviral vector of any one of embodiments 1 to 3, wherein the promoter or promoter fragment with TMEM119 promoter functionality has at least 95%, 96%, 97%, 98%, 99%, 100% sequence identity to the sequence shown in SEQ ID NO: 3, or is a fragment thereof of at least 200 nucleotides in length, which promoter or fragment still has the promoter functionality of the TMEM119 promoter.
7. The retroviral vector of any one of embodiments 1 to 3, wherein the promoter or promoter fragment with OLFML3 promoter functionality has at least 95%, 96%, 97%, 98%, 99%, 100% sequence identity to the sequence shown in SEQ ID NO: 4, or is a fragment thereof of at least 200 nucleotides in length, which promoter or fragment still has the promoter functionality of the OLFML3 promoter.
8. The retroviral vector of any one of embodiments 1 to 3, wherein the promoter or promoter fragment with AIF1 promoter functionality has at least 95%, 96%, 97%, 98%, 99%, 100% sequence identity to the sequence shown in SEQ ID NO: 5, or is a fragment thereof of at least 200 nucleotides in length, which promoter or fragment still has the promoter functionality of the AIF1 promoter.
9. The retroviral vector of any one of embodiments 1 to 3, wherein the promoter or promoter fragment with ITGAM promoter functionality has at least 95%, 96%, 97%, 98%, 99%, 100% sequence identity to the sequence shown in SEQ ID NO: 6, or is a fragment thereof of at least 200 nucleotides in length, which promoter or fragment still has the promoter functionality of the ITGAM promoter.
10. The retroviral vector of any one of embodiments 3 to 9, wherein the tissue-specific promoter is a miR223 promoter fusion promoter.
11. The retroviral vector of any one of embodiments 1 to 10, wherein the therapeutic polypeptide is PGRN or a functional fragment thereof
12. The retroviral vector of embodiment 11, wherein the therapeutic polypeptide has at least 95%, 96%, 97%, 98%, 99%, 100% sequence identity to the sequence shown in SEQ ID NO: 7, SEQ ID NO: 8 or SEQ ID NO: 9, or is a fragment thereof of at least 50 amino acids in length, which polypeptide still provides PGRN functionality.
13. The retroviral vector of embodiment 12, wherein the partial sequence of the PGRN polypeptide is at least 40 amino acids in length.
14. The retroviral vector of any one of embodiments 1 to 10, wherein the therapeutic polypeptide is selected from the group consisting of FasL/Fas, Trail/TRAIL-R, Lymphotoxin beta, decoyreceptors 1-3, TNF-alpha, TNF-alphaR, IFN-gamma, IFN-gamma Receptor, IL-1-IL31, IL1R-IL31Receptor, IL-10, IL-12, IL-23, CXCL-10, PD-1L, PD-1, PD-2L, PD-2, Granzyme B, Granulysine, nitric oxide synthase, DNA methyltransferase 3b (DNMT3b), Jumonji domain-containing protein 1A (JMJD1A), histone deacetylase 3 (HDAC3), and HDAC 9, CSF1 receptor (CSF1R) or the CSD1R-ligand IL-34, all Chemokines, Chemokine Receptors, VEGF, VEGF-Receptors, antagonists to metalloproteinases (e.g. MMP-9), CD40/CD40L, tumor specific ligands and receptors such as EGFR, Annexin1, FGFR-1, Her2, St6galnac5, MMP1-28 and their counterpart TIMPS1-4 (tissue inhibitors of metalloproteinases), Melanotransferrin, alpha4-beta1 Integrin and its ligand endothelial cell VCAM-1, E-cadherin, Alpha-v-beta3 integrin, Alpha-v-beta5 integrin, Alpha-v-beta6 integrin, Alpha-v-beta8 integrin, single-nucleotide variant neoantigens, INDEL frameshift neoantigens, splice variant antigens, fusion protein neoantigens, endogenous retroelement antigens, tumor-specific antigens, in particular tumor-specific antigens by cancer, in particular CCND1, BRCA, CEA, cancer-related antigen 72-4 (CA 72-4), cancer-related antigen 19-9 (CA 19-9), WT1 and NY-ESO-1), soluble and membrane bound.
15. The retroviral vector of embodiment 14, wherein the therapeutic polypeptide is Interferon gamma (IFNgamma) or a functional fragment thereof
16. The retroviral vector of embodiments 14, wherein the therapeutic polypeptide is P-Selectin, MSH, GM-CSF, IL-12, TNF-alpha or Granzyme B
17. The retroviral vector of embodiment 15, wherein the therapeutic polypeptide has at least 95%, 96%, 97%, 98%, 99%, 100% sequence identity to the sequence shown in SEQ ID NO: 10, or is a fragment thereof of at least 50 amino acids in length, which polypeptide still provides IFNgamma functionality.
18. The retroviral vector of any one of embodiments 1 to 17, wherein the retroviral vector is a lentiviral vector, particularly a lentiviral SIN vector.
19. The retroviral vector of any one of embodiments 1 to 17, wherein the retroviral vector is a foamy viral vector.
20. The viral vector of any one of embodiments 1 to 17, wherein the viral vector is an adenoviral and a herpes viral vector, or wherein the viral vector is an alpha-retroviral vector.
21. A retroviral vector according to any one of embodiments 1 to 20 for use in therapy.
22. The retroviral vector according to any one of embodiments 1 to 20 for use in the treatment of a disease or disorder which has its origin in the brain or is brain or nervous system based.
23. The retroviral vector according to any one of embodiments 1 to 13 and 18 to 232 for use in the treatment of a PGRN-associated disease or disorder.
24. The retroviral vector of embodiment 23, wherein the PGRN-associated disease or disorder is a neurodegenerative disease or disorder.
25. The retroviral vector of embodiment 24, wherein the neurodegenerative disease or disorder is frontotemporal degenerative disease or disorder.
26. The retroviral vector of embodiment 25, wherein the frontotemporal degenerative disease or disorder is selected from the group consisting of Alzheimer's disease, amyotrophic lateral sclerosis, and Parkinson's disease.
27. The retroviral vector according to any one of embodiments 1 to 10 and 14 to 22 for use in the treatment of brain tumors.
28. The retroviral vector according to embodiment 27 for use in the treatment of brain tumors selected from the group consisting of glioblastoma, glioma, ganglioneuroblastoma, astrocytoma, oligodendroglioma, PNET (primitive neuroectodermal), medulloblastoma, CNS lymphoma, and neuroblastoma, or any other CNS tumor.
29. The retroviral vector according to embodiment 27 for use in the treatment of brain metastasis, originating from any forms of breast, lung, colon, testicular, renal carcinomas and melanoma, or any other solid tumor, and any hematologic tumor, comprising all forms of leukemia and lymphomas.
30. A method for treating a disease or disorder which has its origin in the brain or is brain based comprising ex vivo genetic modification of haematopoietic stem cells and/or a population of enriched CD34-positive bone marrow cells of patients suffering from such a disease or disorder by retroviral transduction with a retroviral vector according to any one of embodiments 1 to 22 and administration of the modified cells to the patients.
31. The method of embodiment 30, wherein the retroviral vector is a vector according to any one of embodiments 1 to 13 and 18 to 22 and wherein the patients suffer from a PGRN-associated disease or disorder.
32. The method of embodiment 31, wherein the PGRN-associated disease or disorder is a neurodegenerative disease or disorder.
33. The method of embodiment 32, wherein the neurodegenerative disease or disorder is frontotemporal degenerative disease or disorder.
34. The method of embodiment 33, wherein the frontotemporal degenerative disease or disorder is selected from the group consisting of Alzheimer's disease, amyotrophic lateral sclerosis, and Parkinson's disease.
35. The method of embodiment 30, wherein the retroviral vector is a vector according to any one of embodiments 1 to 10 and 14 to 21 and wherein the patients suffer from a brain tumor.
36. The method of embodiment 35, wherein the brain tumor is selected from the group consisting of glioma, glioblastoma, ganglioneuroblastoma, astrocytoma, oligodendroglioma, PNET (primitive neuroectodermal), medulloblastoma, CNS lymphoma, and neuroblastoma, or any other CNS tumor.
37. The method of embodiment 35, wherein the patients suffer from brain metastasis, originating from any forms of breast, lung, colon, testicular, renal carcinomas and melanoma, or any other solid tumor, and any hematologic tumor, comprising all forms of leukemia and lymphomas.
38. The method of any one of embodiments 30 to 37, wherein the patients were pre-treated with Busulphan, Treosulfan, radiotherapy, biologics such as monoclonal antibody-based or small-molecule-based inhibitors of colony-stimulating factor 1 (CSF1) and CSF1 receptor (CSF1R) inhibitors such as PLX3397, BLZ9445, PLX5622, RG7155, PLX647, Ki20227, GW2580, or the CSF1R-ligand IL-34, desatinib, and any combination thereof, within a window of between the past 8 to the past 15 days before administration.

### BRIEF DESCRIPTION OF THE DRAWINGS

FIG. 1: SIN-lentiviral construct vUS6 comprising the transgene GRN under control of the promoter miR223 (SEQ ID NO:1). 2A: self cleaving peptide; GFP: green fluorescent protein; nls: nuclear localization sequence; PRE4: modified Woodchuck Hepatitis Virus (WHP) Posttranscriptional Regulatory Element.

FIG. 2: FACS analysis with HEK293T cells after transduction. Left: Untransduced cells. Right: Cells transduced with the lentiviral construct shown in FIG. 1 (vUS6) (Titer: 6.04×10⁶ TU/mL; MOI=2).

FIG. 3: FACS analysis with THP1 cells after transduction. Left: Untransduced cells. Right: Cells transduced with the lentiviral construct shown in FIG. 1 (vUS6) (Titer: 6.04×10⁶ TU/mL; MOI=2).

FIG. 4: FACS analysis with human microglia after transduction. Left: Untransduced cells. Right: Cells transduced with the lentiviral construct shown in FIG. 1 (vUS6) (Titer: 6.04×10⁶ TU/mL; MOI=2).

FIG. 5: Progranulin release by human microglia (GRN−/−) measured by ELISA. Left bar: untransduced cells. Right bar: Cells transduced with the lentiviral construct shown in FIG. 1 (vUS6).

FIG. 6: SIN-lentiviral construct comprising the transgene IL-12 under control of the promoter miR223 (SEQ ID NO:1). IRES: internal ribosome entry site; PRE4: modified Woodchuck Hepatitis Virus (WHP) Posttranscriptional Regulatory Element.

FIG. 7: FACS analysis with THP1 cells after transduction. Left: Untransduced cells. Right: Cells transduced with the lentiviral construct shown in FIG. 6 (Titer: 3.24×10⁶ TU/mL; MOI=2).

FIG. 8: FACS analysis with human microglia after transduction. Left: Untransduced cells. Right: Cells transduced with the lentiviral construct shown in FIG. 6 (Titer: 3.24×10⁶ TU/mL; MOI=2).

FIGS. 9A-9D: SIN-lentiviral constructs comprising the transgene GRN under control of the promoters (FIG. 9A) miR223-TMEM119 (vUS7; SEQ ID NO:28); (FIG. 9B) ITGAM (vUS8; SEQ ID NO:6); (FIG. 9C) miR223_P2RY12 (vUS11; SEQ ID NO: 26); and (FIG. 9D) miR223_OLFML3 (vUS12; SEQ ID NO:29). 2A: self cleaving peptide; GFP: green fluorescent protein; nls: nuclear localization sequence; PRE4: modified Woodchuck Hepatitis Virus (WHP) Posttranscriptional Regulatory Element.

FIGS. 10A-10F: FACS analysis with THP-1 cells transduced with different vectors upon differentiation to macrophages. (FIG. 10A) non-transduced control; (FIG. 10B) vUS6-LV_miR223_GRN (FIG. 1); (FIG. 10C) vUS7-LV_miR223-TMEM119 GRN (FIG. 9A); (FIG. 10D) vUS8-LV_ITGAM_GRN (FIG. 9B); (FIG. 10E) vUS11-LV_miR223-P2RY12 (FIG. 9C); (FIG. 10F) vUS12-LV_miR223-OLFML3 (FIG. 9D.

FIGS. 11A-11B: (FIG. 11A) Percentage of GFP positive cells (transduction rate) in differentiated THP-1 cells. (FIG. 11B) Mean fluorescent intensity of differentiated THP-1 cells.

FIGS. 12A-12F: FACS analysis of human microglia cell line (GRN −/−) transduced with different vectors. (FIG. 12A) non-transduced control; (FIG. 12B) vUS6-LV_miR223_GRN (FIG. 1); (FIG. 12C) vUS7-LV_miR223-TMEM119_GRN (FIG. 9A); (FIG. 12D) vUS8-LV_ITGAM_GRN (FIG. 9B); (FIG. 12E) vUS11-LV_miR223-P2RY12 (FIG. 9C); (FIG. 12F) vUS12-LV_miR223-OLFML3 (FIG. 9D.

FIGS. 13A-13B: (FIG. 13A) Percentage of GFP positive cells (transduction rate) in human microglia cell line (GRN −/−). (FIG. 13B) Mean fluorescent intensity of human microglia cell line (GRN −/−).

FIGS. 14A-14C: Restoration of GRN secretion in human microglia cell line (GRN −/−). (FIG. 14A) Amount of Granulin in supernatant. (FIG. 14B) Mean fluorescence intensity normalized by vector copy number. (FIG. 14C) Amount of Granulin normalized by (transduction rate×vector copy number)

FIGS. 15A-15F: Human CD34+ bone marrow cells were lentivirally transduced with vectors encoding GRN-2A-GFP as transgene, followed by differentiation to monocytes and FACS analysis in monocytes (day 12). (FIG. 15A) non-transduced control; (FIG. 15B) vUS6-LV_miR223_GRN (FIG. 1); (FIG. 15C) vUS7-LV_miR223-TMEM119 GRN (FIG. 9A); (FIG. 15D) vUS8-LV_ITGAM_GRN (FIG. 9B); (FIG. 15E) vUS11-LV_miR223-P2RY12 (FIG. 9C); (FIG. 15F) vUS12-LV_miR223-OLFML3 (FIG. 9D.

FIG. 16: Percentage of GFP positive monocytes.

FIGS. 17A-17D: Analysis of the activity of candidate promoters in human CD34+ cells upon differentiation into monocytes (day 7). (FIG. 17A) non-transduced control; (FIG. 17B) vUS6-LV_miR223_GRN (FIG. 1): (left) transduction in the presence of Amphotericin B, (right) transduction in the presence of Lentiboost; (FIG. 17C) vUS7-LV_miR223-TMEM119_GRN (FIG. 9A): (left) transduction in the presence of Amphotericin B, (right) transduction in the presence of Lentiboost; (FIG. 17D) vUS8-LV_ITGAM_GRN (FIG. 9B): (left) transduction in the presence of Amphotericin B, (right) transduction in the presence of Lentiboost.

FIG. 18: Vector copy number of human CD34+ cells upon differentiation into monocytes (day 12). (Top) Transduction in the presence of Amphotericin B, (bottom) transduction in the presence of Lentiboost.

FIG. 19: Summary of FIG. 18.

FIG. 20: Transduction efficiency (% GFP-positive cells divided by vector copy number (VCN)) in myeloid cells obtained from human CD34+ cells.

FIG. 21: miR223 promoter activity in monocytes, macrophages and iPSC-derived homologs to haematopoietic stem cells.

### EXAMPLES

### Example 1: Production of Gene Modified Haematopoietic Stem Cells (HSC) Appropriate for the Treatment of Patients Suffering from Frontotemporal Dementia Due to GRN Gene Mutation

HSC obtained by leukapheresis upon HSC mobilization, are transduced with a lentiviral self-inactivating (SIN) vector, comprising human PGRN (progranulin) encoding cDNA according to SEQ ID NO: 7 under control of the miR223 promoter according to SEQ ID NO: 1. Preferentially, the genetically modified autologous HSC population is cryopreserved after genetic manipulation.

### Example 2: Production of Gene Modified CD34+ Cells Appropriate for the Treatment of Patients Suffering from Frontotemporal Dementia Due to GRN Gene Mutation

A CD34+ cell population, obtained by leukapheresis upon HSC mobilization followed by CD34+ cell isolation, is transduced with a lentiviral SIN vector encoding PGRN according to SEQ ID NO: 7 under control of a miR223 fusion promoter construct consisting of the TMEM119 promoter contained in SEQ ID NO: 3 fused to the miR223 promoter according to SEQ ID NO: 1.

### Example 3: Production of Gene Modified CD34+ Enriched Bone Marrow Cells Appropriate for the Treatment of Patients Suffering from Frontotemporal Dementia Due to GRN Gene Mutation

CD34+ enriched bone marrow cells are transduced with a lentiviral vector encoding PGRN according to SEQ ID NO:8 under control of the ITGAM promotor according to SEQ ID NO: 6.

### Example 4: Production of Gene Modified CD34+ Enriched Bone Marrow Cells Appropriate for the Treatment of Patients Suffering from Frontotemporal Dementia Due to GRN Gene Mutation

CD34+ bone marrow cells are transduced with a foamy viral vector encoding PGRN according to SEQ ID NO: 8 under control of the ITGAM promotor according to SEQ ID NO: 6.

### Example 5: Production of Gene Modified Blood Derived Monocytes Appropriate for the Treatment of Patients Suffering from Glioblastoma

Blood derived monocytes of a patient suffering from glioblastoma are transduced with a lentiviral vector encoding interferon-gamma under control of the ITGAM promoter according to SEQ ID NO: 6.

### Example 6: Production of Gene Modified Blood Derived Monocytes Appropriate for the Treatment of Patients Suffering from Renal Carcinoma and Brain Metastases

Blood derived monocytes of a patient suffering from renal carcinoma and optionally suffering from brain metastases, are transduced with a lentiviral vector encoding interferon-gamma under control of the miR223 promoter according to SEQ ID NO: 1.

### Example 7: Experiments Using Lentiviral-SIN Vectors for Phagocyte-Specific Transgene Expression

### Testing of Transduction of Cell Lines

The developed lentiviral SIN-vectors enable phagocyte-specific transgene expression and may therefore be used for applications in human diseases in which phagocytes are the disease-causing cells, or in which phagocytes can provide therapeutic elements to cure or treat these diseases. This is the case for disorders which have their origin in the brain, such as neurodegenerative disease, or brain cancer or metastasis, as well as for disorders outside of the brain, such as immunodeficiency or cancer.

Transduction with a Vector Comprising a GRN Transgene:

Following cell lines were analyzed:
1. HEK293Tcells (Human embryonic kidney cells) as positive control;
2. THP1 cells (Human phagocyte cell line) as model for phagocyte correction;
3. Immortalised human microglia cell line (Im-hMicro), in which the GRN gene was knocked-out (GRN−/−), as disease model.

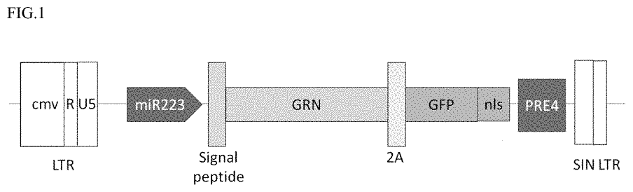

For transduction of cell lines, a lentiviral self-inactivating (SIN) vector was used, comprising the miR223 promoter as internal promoter, and hGRN encoding cDNA as transgene, as well as an EGFP-reporter linked to GRN by the 2A sequence (FIG. 1). Transduction experiments were conducted at MOI 2 (based on a titer of 6.04*TU/mL) in DMEM medium, supplemented with 10% FBS without addition of cytokines or transduction enhancers. GFP expression was measured by FACS 48 h after transduction and correlates with the expression of the GRN transgene.

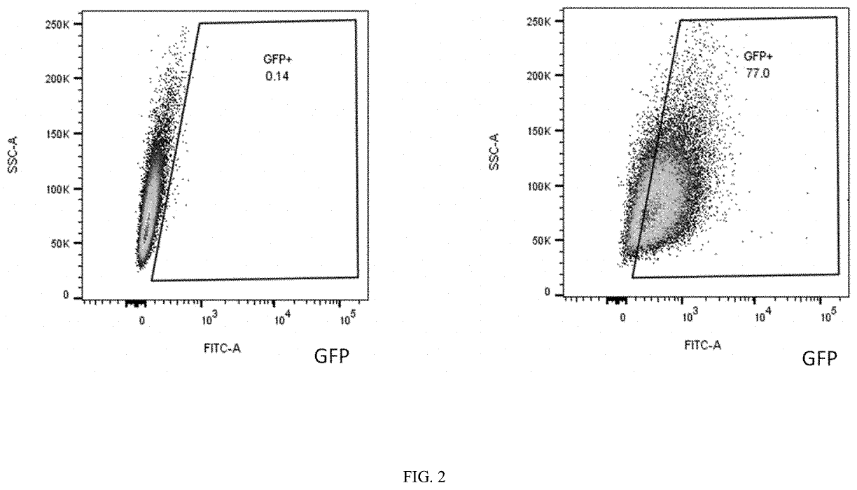

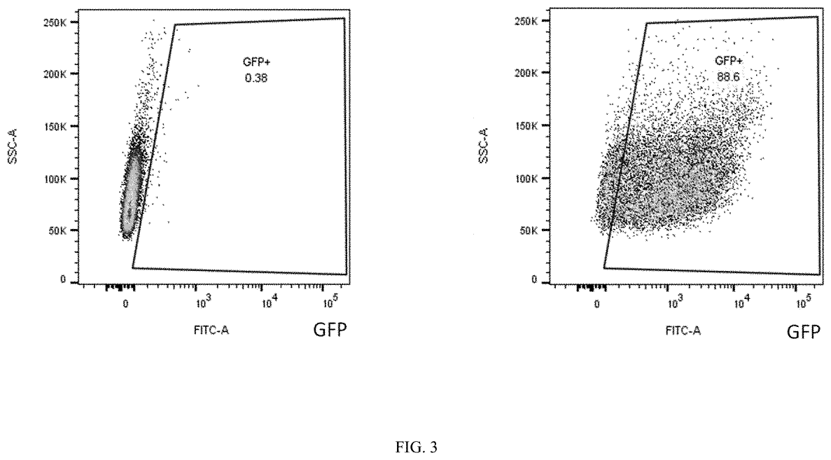

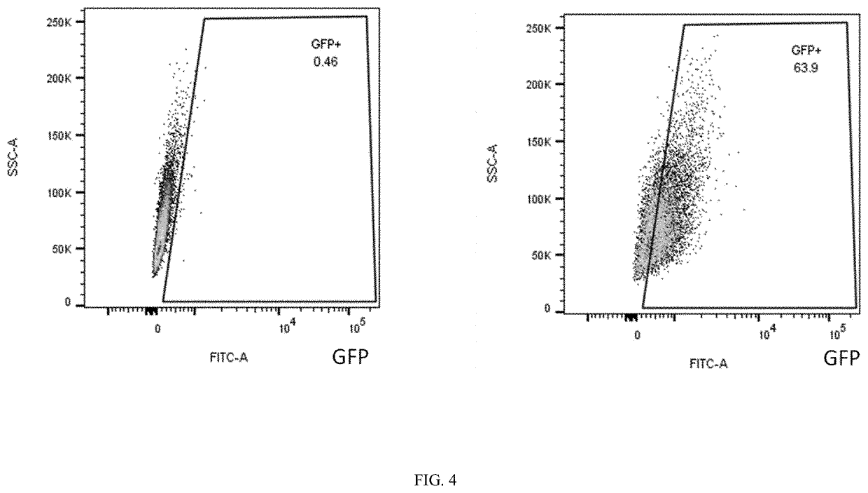

In the vector construct, miR223 promoter activity led to the translation of one mRNA species encoding both granulin and GFP, which were co-translated into two separate proteins in a molecular ratio of 1:1, of which GFP was detected by flow cytometry analysis. In all three cell lines, transduction with the LV-miR223-GRN vector led to high expression levels of GFP: HEK293T cells, 77% GFP-positive cells (FIG. 2); THP1 cells, 88.6% GFP-positive cells (FIG. 3), immortalized human microglia GRN −/− cell line, 63.9% GFP-positive cells (FIG. 4). Accordingly, it has been successfully demonstrated that the promoter miR223 drives expression of transgenes in phagocytic cells as well as in microglia.

Additionally, in the immortalized human microglia GRN −/− cell line, an ELISA assay measuring the release of the progranulin protein into the cell culture supernatant was performed 10 days after transduction, showing reconstitution of progranulin protein production and release from microglia cells (FIG. 5). For the ELISA, the culture medium containing DMEM supplemented with 10% FBS, was exchanged for 24 h before analyses for DMEM medium without FBS. Untransduced cells were compared to transduced cells.

Transduction with IL-12 Transgene Vector:

Following cell lines were analyzed:
1. THP1 cells (Human phagocyte cell line) as model for phagocyte correction; and
2. Immortalised human microglia cell line (Im-hMicro), in which the GRN gene was knocked-out (GRN−/−) as model for microglia correction.

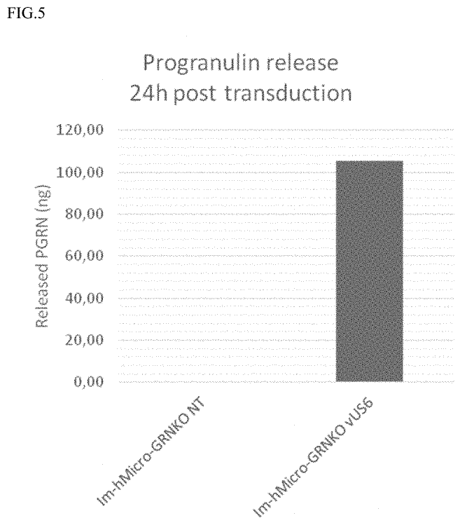

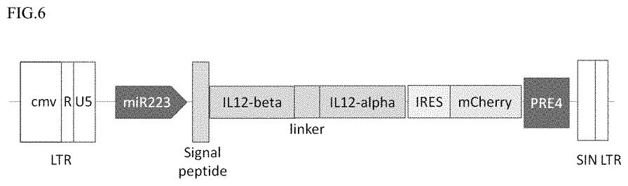

For transduction of cell lines, a lentiviral self-inactivating (SIN) vector was used, comprising the miR223 promoter as internal promoter, and human IL12-beta and IL12-alpha encoding cDNA subunits fused by a protein linker to one protein with IL-12 activity, as well as an mCherry-reporter linked to IL12 subunits preceded by an internal ribosomal entry site (IRES) (FIG. 6). Experiments were conducted at MOI 2 based on a titer of 3.24*10⁶ TU/mL, in DMEM medium, supplemented with 10% FBS without addition of cytokines or transduction enhancers. mCherry expression was measured by FACS 48 h after transduction and correlates with the expression of the IL12 transgene.

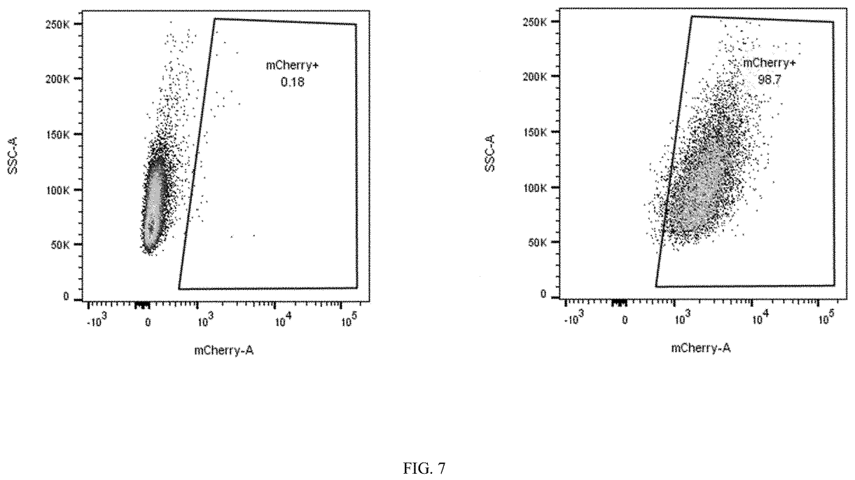

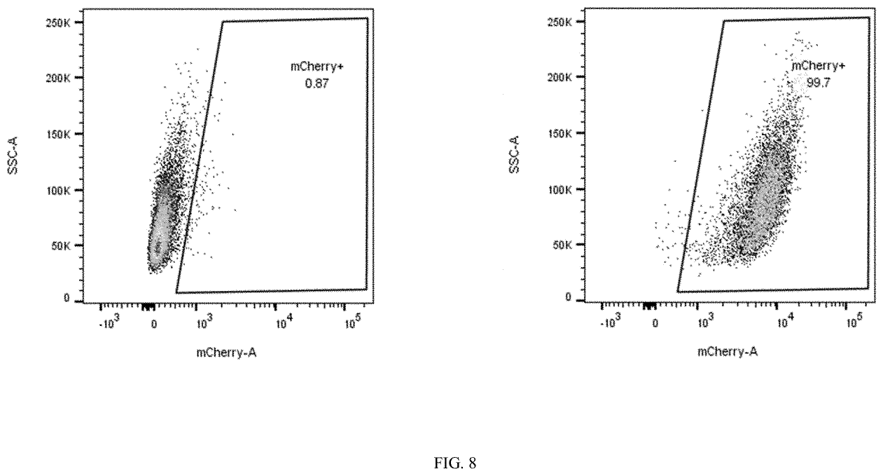

In both cell lines, transduction with the LV-miR223-IL12 vector led to high expression levels of mCherry: THP1 cells, 98.7% mCherry-positive cells (FIG. 7), immortalized human microglia GRN −/− cell line, 99.7% GFP-positive cells (FIG. 8). Accordingly, it has been successfully demonstrated that the promoter miR223 drives expression of transgenes in phagocytic cells as well as in microglia.

### Example 8: Analysis of Additional Promoters in a Phagocytic Cell Line

It has been demonstrated in Example 7 that the promoter miR223 can drive expression in human phagocytic cells as well as in human microglia. In this Example, the activity of additional promoters was tested in phagocytic cells.

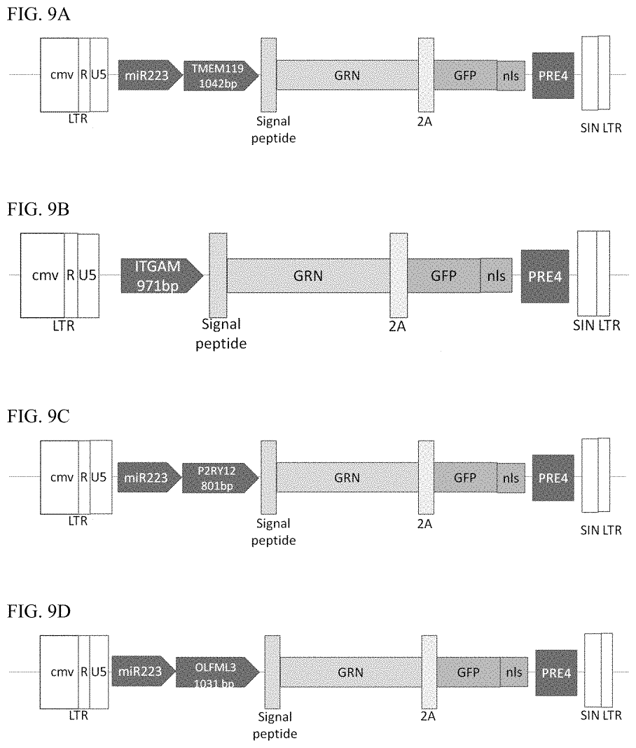

The following lentiviral constructs were tested:
LV-miR223-hGRN-2A-EGFP-NLS-WPRE (FIG. 1)
LV-miR223-TMEM119-hGRN-2A-EGFP-NLS-WPRE (FIG. 9A)
LV-ITGAM-hGRN-2A-EGFP-NLS-WPRE (FIG. 9B)
LV-miR223-P2RY12-hGRN-2A-EGFP-NLS-WPRE (FIG. 9C)
LV-miR223-OLFML3-hGRN-2A-EGFP-NLS-WPRE (FIG. 9D)

THP-1 cells were seeded in a 96-well plate in a density of 40.000 cells per well. Transduction was carried out right after seeding by adding the appropriate amount of virus (MOI 2) and resuspending the cells in the well. For differentiation of untransduced or transduced THP-1 cells, cells were cultured in differentiation medium (RPMI 10% FBS, 1× GlutaMAX, 1× PenStrep, 10 ng/mL PMA) and incubated for 72 hours. Adherent cells were detached with StemPro Accutase and cells were washed with PBS. For analysis, Fc receptor was blocked by FcR blocking reagent at a dilution of 1:20, cells were strained with LIVE/DEAD Fixable Violet dye (1:1000) and PE-Cy7-CD11b (1:200) followed by a analysis in a LSR II Fortessa flow cytometer. Only single and viable cells (negative for LIVE/DEAD Fixable Violet staining) were analyzed. Differentiation to macrophages was followed by quantification of PE-Cy7-CD11b staining increasing upon differentiation to macrophages. GRN/GFP co-expression was assayed by quantification of GFP fluorescent signal intensity. All experiments were performed in duplicates.

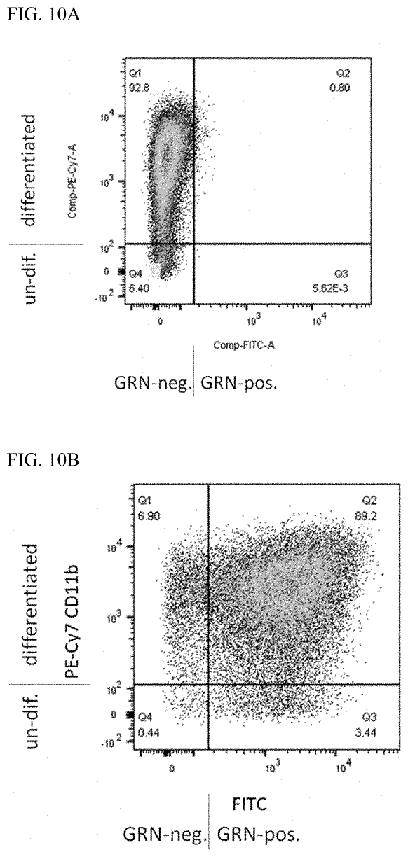

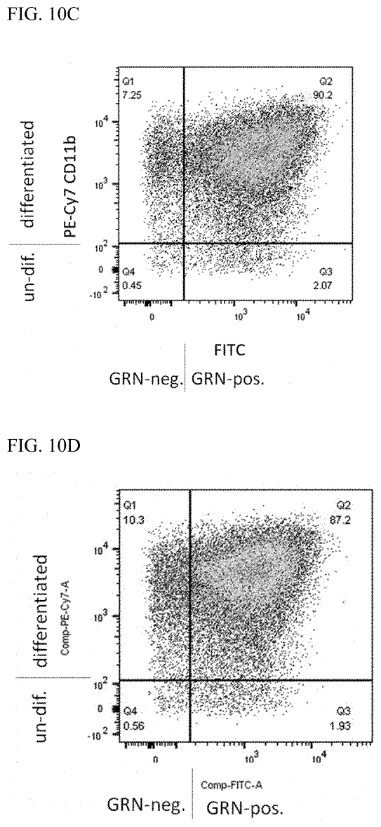

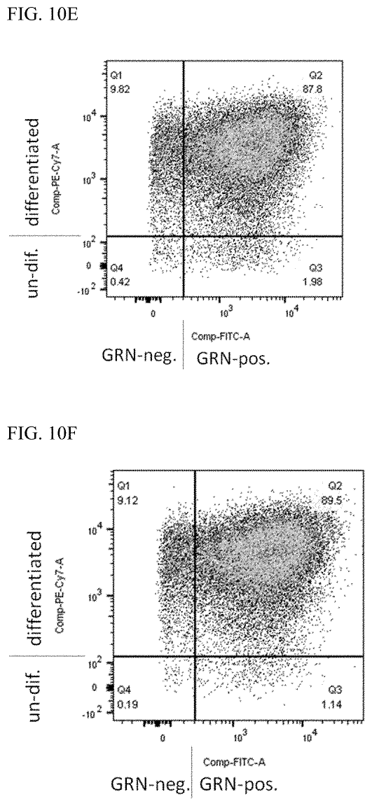

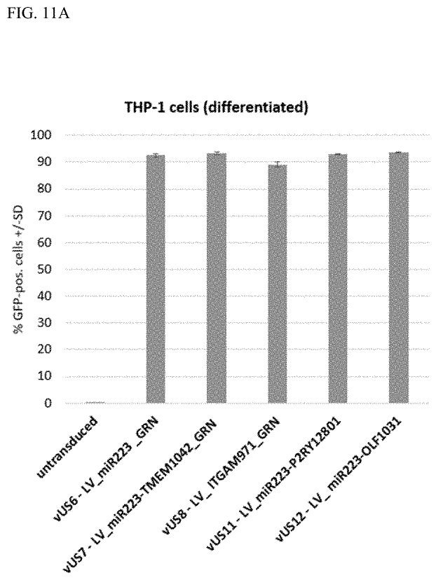

All tested promoter variants resulted in high transgene expression in THP-1 cells. The results of this assay are summarized in FIGS. 10A-10F and FIGS. 11A-11B.

### Example 9: Analysis of Additional Promoters in Microglia

Activity of the lentiviral constructs described in Example 8 was also tested in microglia

For that, immortalized human microglia were seeded in a 96-well plate in a density of 40.000 cells per well. Transduction was carried out right after seeding by adding the appropriate amount of virus (MOI 2) and resuspending the cells in the well. Adherent cells were detached with TrypLE Express and cells were washed with PBS. For analysis, cells were strained with LIVE/DEAD Fixable Violet dye (1:1000) followed by a analysis in a LSR II Fortessa flow cytometer. Only single and viable cells (negative for LIVE/DEAD Fixable Violet staining) were analyzed. GRN/GFP co-expression was assayed by quantification of GFP fluorescent signal intensity.

For quantification of granulin secretion upon gene therapy treatment of granulin-deficient human microglia, cells were seeded at a density of 150.000 cells per well in a 24-well plate in 500 μL of medium. 24 hours after seeding, the culture medium was removed and replaced by 500 μL of fresh, antibiotics-free medium. Conditioned medium was collected after 24 hours of culture, debris were removed by centrifugation at 17,000 g for 10 minutes and samples were stored at −20° C. until processing. For quantification of granulin protein concentration, supernatant samples were concentrated with Amicon Ultra-0.5 3K centrifugal filter devices and the concentration of PGRN was determined with Progranulin (human) ELISA Kit (Adipogen, cat. #AG-45A-0018YEK-KI01) following the manufacturer's protocol. Results in ng represent total ng of PGRN released by 150.000 cells into 500 μL of medium within 24 hours. All experiments were performed in duplicates.

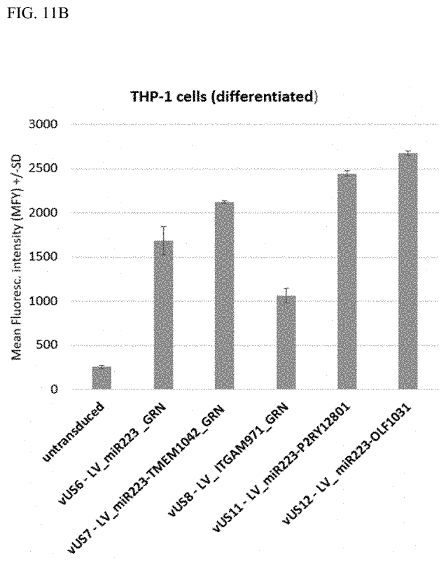

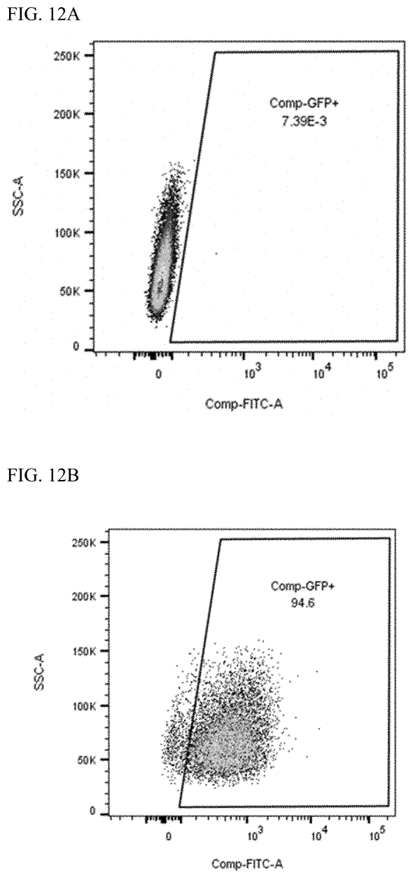

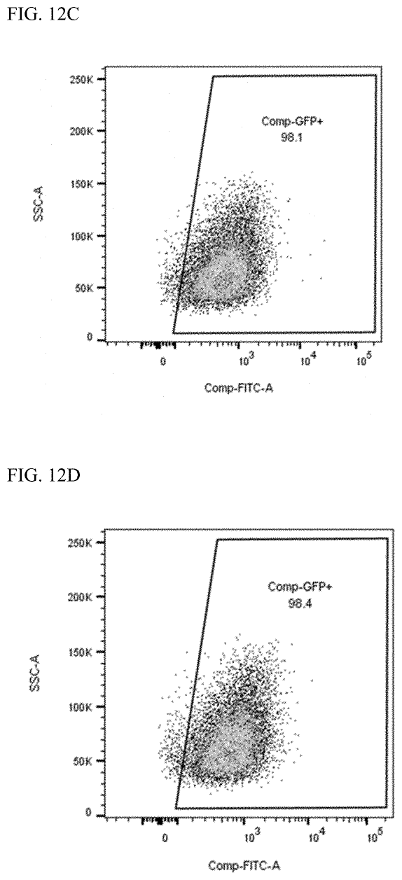

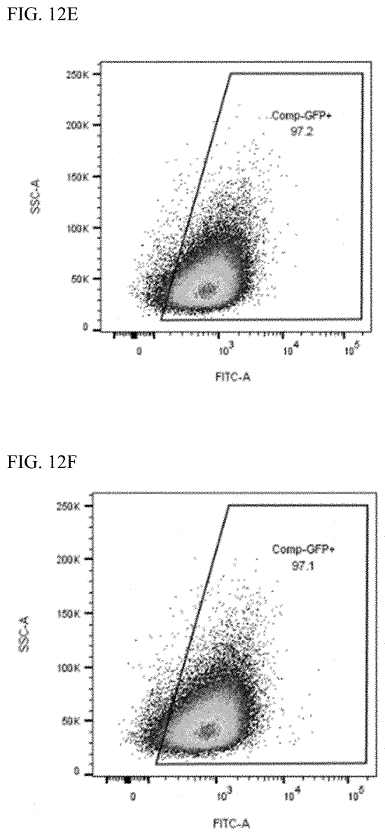

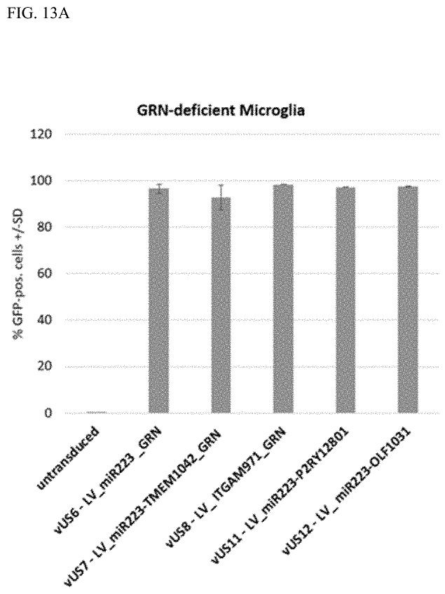

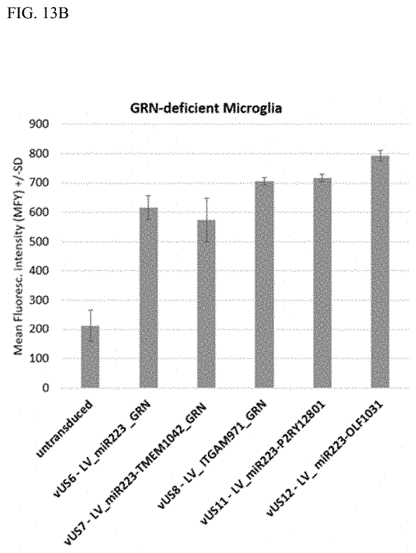

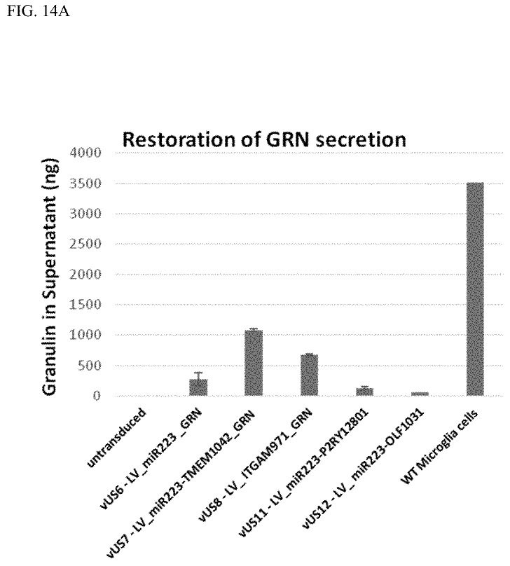

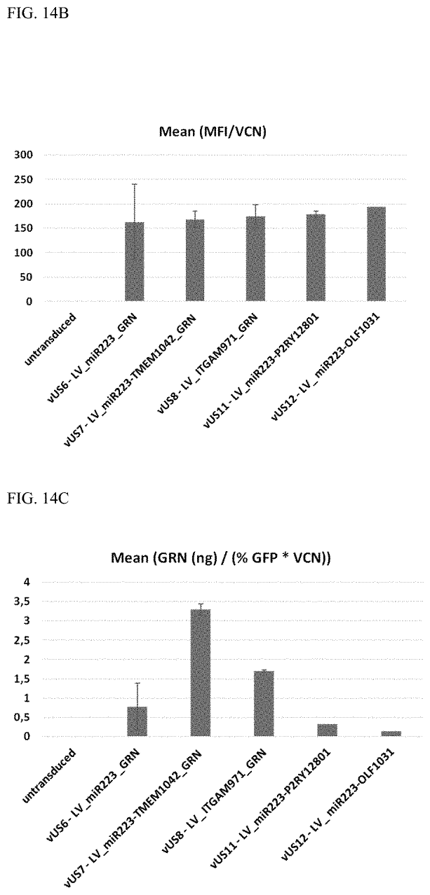

All tested promoter variants resulted in high transgene expression in microglia. The results of this assay are summarized in FIGS. 12A-12F and FIGS. 13A-13B. Restoration of granulin secretion in GRN−/− cells is shown in FIGS. 14A-14C.

### Example 10: Transduction of Human CD34+ Bone Marrow Cells Followed by Differentiation to Monocytes

Commercially available human CD34+ bone marrow cells were thawed and taken into culture (day 0) in X-VIVO 20, HSA 1%, SCF 300 ng/mL, Flt3-L 300 ng/mL, TPO 100 ng/mL. On day 1 and day 2, cells were transduced with MOI3 in the presence of protamine sulfate 4 μg/mL and amphotericin B 1 μg/mL. Cells were re-plated in expansion medium (X-VIVO 20, HSA 1%, PenStrep 1×, SCF 100 ng/mL, Flt3-L 100 ng/mL, TPO 100 ng/mL) on day 3. On day 5, medium was changed to pro-myelocytes expansion medium (IMDM, HEPES 5 mM, GlutaMax 2 mM, FBS 10%, PenStrep 0.5×, SCF 100 ng/mL, IL-3 100 ng/mL). And on day 8 to pro-myelocytes differentiation medium (IMDM, HEPES 5 mM, GlutaMax 2 mM, FBS 10%, PenStrep 0.5×, SCF 50 ng/mL, IL-3 20 ng/mL, GM-CSF 20 ng/mL, M-CSF 100 ng/mL). Cells were analyzed on day 12 for lineage markers and for marker gene expression by flow cytometry analysis.

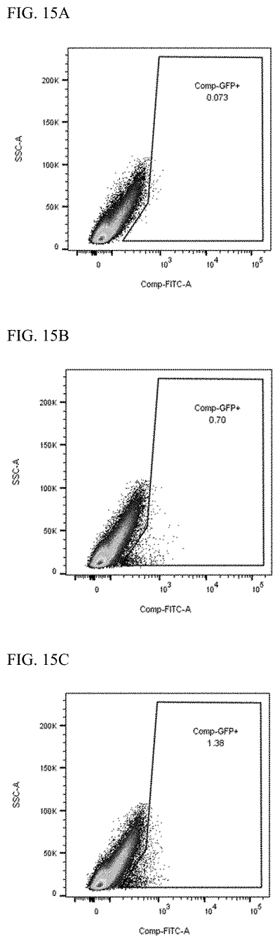

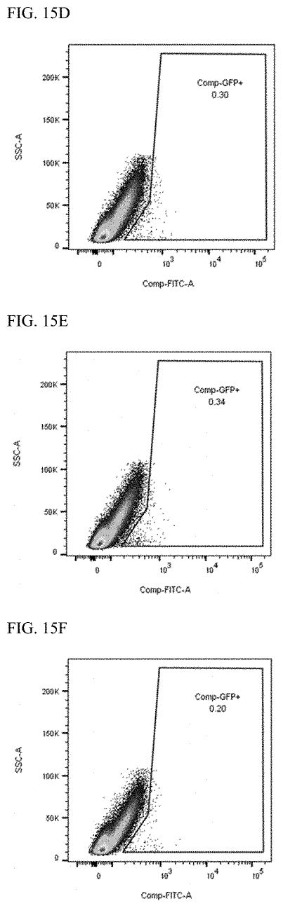

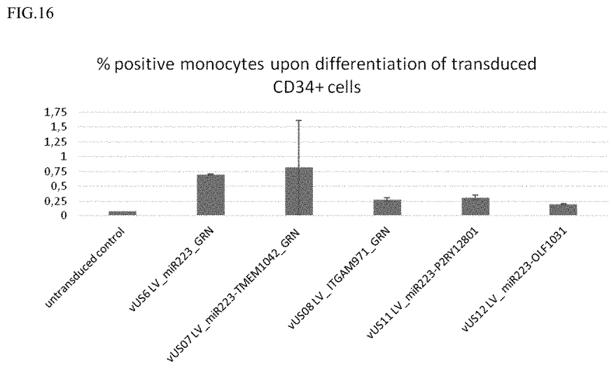

Analysis of differentiated cells for CD11b and CD14 expression revealed more than 80% differentiated monocytes. Within the monocytic cell populations, GFP marker gene expression co-expressed with granulin was analyzed (see FIGS. 15A-15F and 16).

### Example 11: Analysis of Additional Promoters in CD34+ Cells

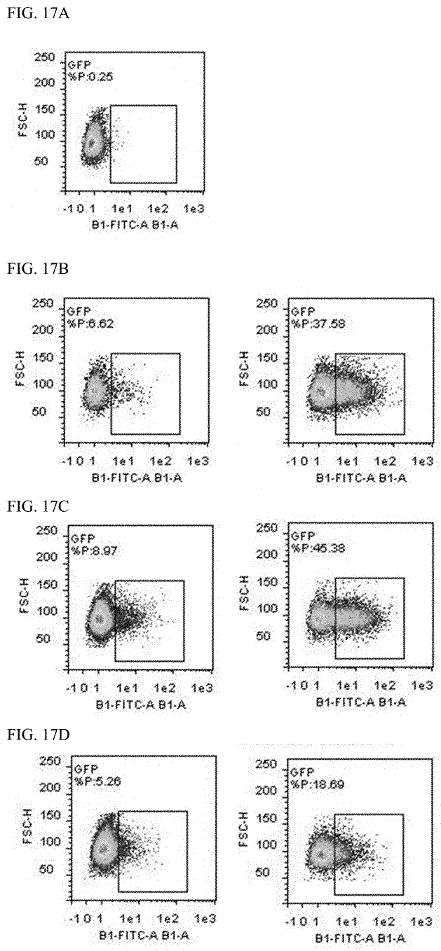

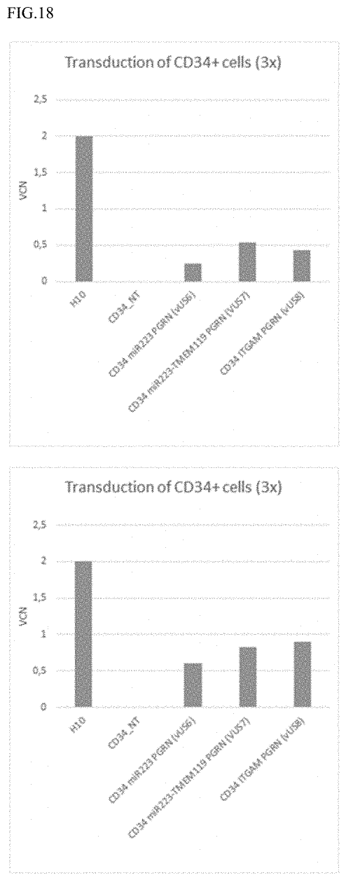

Human CD34+ cells were thawed (day 0) and taken into culture (in X-VIVO 20, HSA 1%, SCF 300 ng/mL, Flt3-L 300 ng/mL, TPO 100 ng/mL). On day 1 and 2, cells were consecutively transduced three times with viral infectious particles (in presence of transduction enhancers (Amphotericin B or Lentiboost)). From end of day 2 to day 5, cells were cultured n pro-myelocytes expansion medium (IMDM, FBS 10%, PenStrep 0.5×, SCF 100 ng/mL, IL-3 100 ng/mL). On day 5, cells were re-plated in pro-myelocytes differentiation medium (IMDM, FBS 20%, PenStrep 0.5×, SCF 100 ng/mL, IL-3 100 ng/mL, G-CSF 20 ng/mL). GFP marker expression was quantified by FACS analysis on day 7 (FIGS. 17A-17D); VCNs were quantified on days 12 of culture (FIG. 18).

### Example 12: Vector Copy Number Determination in Transduced CD34+ Cells

Cells at day 12 of the differentiation protocol were pelleted and the cell pellets were stored at −20° C. until processing.
Genomic DNA was isolated from the cell pellets using the QIAamp® DNA Blood Mini Kit (Qiagen, Hilden, Germany) following the manufacturer's protocol.
VCN was determined by qPCR with the delta-delta Ct method, using FOXP2 as reference gene and WPRE for the integrated vector gene.
Clone H10 carrying 2 γ integrations was used as a reference.

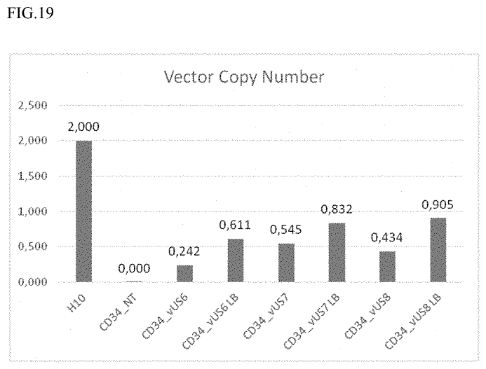

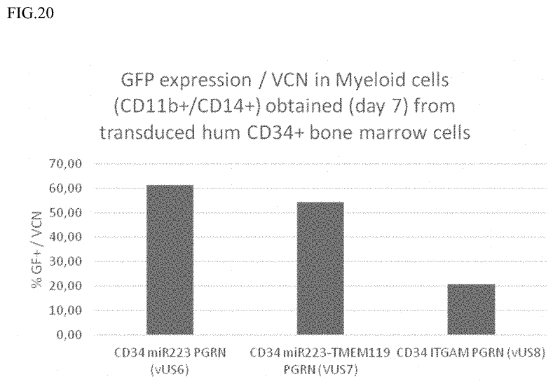

The results are summarized in FIGS. 19 and 20.

### Example 13: Transgene Expression in HSPCs and Myeloid Cells

p47phox-deficient iPSCs were transduced with lentiviral vectors encoding p47phox under control of the miR223 promoter or under control of the constitutively active SFFV promoter. Cells were differentiated to embryoid bodies and further to monocytes and macrophages. iPSCs (CD133+). CD34-positive cells as iPSC-derived homolog to haematopoietic stem cells in embryoid bodies, monocytes (CD14+) and macrophages (CD206) were analyzed for transgenic p47phox expression.

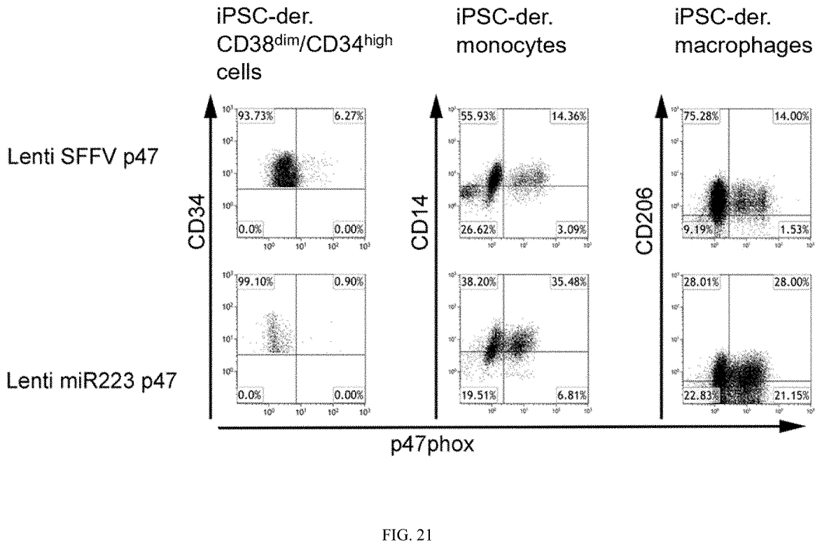

FIG. 21 shows that the miR223 promoter is active in monocytes and macrophages, but inactive in iPSC-derived homologs to haematopoietic stem cells.

1. A viral vector comprising a nucleic acid molecule encoding a therapeutic polypeptide or a combination of therapeutic polypeptides under control of a promoter or promoter fragment, wherein the promoter or promoter fragment drives expression of the therapeutic protein or the combination of therapeutic proteins in myeloid cells and microglia, and wherein the promoter or promoter fragment is inactive in progenitor and/or stem cells.

2. The viral vector according to claim 1, wherein the promoter is
a) a miR223 promoter or a promoter having at least 95%, 96%, 97%, 98%, 99% or 100% sequence identity to the sequence shown in SEQ ID NO: 1, or a functional fragment thereof; or
b) an ITGAM promoter or a promoter having at least 95%, 96%, 97%, 98%, 99% or 100% sequence identity to the sequence shown in SEQ ID NO: 6, or a functional fragment thereof; or
c) an AIF1 promoter or a promoter having at least 95%, 96%, 97%, 98%, 99% or 100% sequence identity to the sequence shown in SEQ ID NO: 5, or a functional fragment thereof; or
d) a TMEM119 promoter, or a promoter having at least 95%, 96%, 97%, 98%, 99% or 100% sequence identity to the sequence shown in SEQ ID NO: 3, SEQ ID NO:23 or SEQ ID NO:24, or a functional fragment thereof, or
e) a P2RY12 promoter, or a promoter having at least 95%, 96%, 97%, 98%, 99% or 100% sequence identity to the sequence shown in SEQ ID NO: 2, SEQ ID NO: 21 or SEQ ID NO: 22, or a functional fragment thereof, or
f) an OLFML3 promoter, or a promoter having at least 95%, 96%, 97%, 98%, 99% or 100% sequence identity to the sequence shown in SEQ ID NO: 4 or SEQ ID NO:25, or a functional fragment thereof; or
g) a fusion promoter comprising a miR233 promoter, or a promoter having at least 95%, 96%, 97%, 98%, 99% or 100% sequence identity to the sequence shown in SEQ ID NO: 1, or a functional fragment thereof, operably linked to
i) a TMEM119 promoter, or a promoter having at least 95%, 96%, 97%, 98%, 99% or 100% sequence identity to the sequence shown in SEQ ID NO: 3, SEQ ID NO:23 or SEQ ID NO:24, or a functional fragment thereof; and/or
ii) a P2RY12 promoter, or a promoter having at least 95%, 96%, 97%, 98%, 99% or 100% sequence identity to the sequence shown in SEQ ID NO: 2, SEQ ID NO: 21 or SEQ ID NO:22, or a functional fragment thereof; and/or
iii) an OLFML3 promoter, or a promoter having at least 95%, 96%, 97%, 98%, 99% or 100% sequence identity to the sequence shown in SEQ ID NO: 4 or SEQ ID NO:25, or a functional fragment thereof; and/or
iv) an ITGAM promoter, or a promoter having at least 95%, 96%, 97%, 98%, 99% or 100% sequence identity to the sequence shown in SEQ ID NO:6, or a functional fragment thereof; and/or
v) an AIF1 promoter or a promoter having at least 95%, 96%, 97%, 98%, 99% or 100% sequence identity to the sequence shown in SEQ ID NO:5, or a functional fragment thereof.

3. The viral vector according to claim 1 or 2, wherein the viral vector comprises at least one transcriptional regulatory element, wherein said at least one transcriptional regulatory element is arranged such that it inhibits or activates a transcriptional activity of the promoter.

4. The viral vector according to claim 3, wherein the at least one transcriptional regulatory element comprises a binding site for a transcriptional activator or repressor, in particular wherein the transcriptional activator or repressor comprises:
i) an antibiotic-binding domain, in particular a tetracycline/doxycycline-binding domain, a macrolide-binding domain or a pristinamycin-binding domain;
ii) a hormone-binding domain, in particular a RU486-binding domain or an abscisic acid-binding domain;
iii) a steroid-binding domain, in particular an ecdysone-binding domain; or
iv) a dimerizer system, in particular a rapamycin-based of rapalog-based dimerizer system.

5. The viral vector according to any one of claims 1 to 4, wherein the viral vector encodes a riboswitch, wherein the riboswitch controls translation of an mRNA encoding the therapeutic protein or the combination of therapeutic proteins.

6. The viral vector according to any one of claims 1 to 5, wherein the therapeutic polypeptide is
i) a polypeptide that restores a cellular function and/or elicits a cellular response in a cell; or
ii) a polypeptide that enables and/or increases target specificity of a cell.

7. The viral vector according to claim 6, wherein the polypeptide that restores a cellular function and/or elicits a cellular response in a cell comprises at least a fragment of one or more polypeptides selected from the group consisting of: PGRN, Presenilin1, Presenilin 2, IL-2, IL-12, IL-15, IL-21, IFN-alpha, IFN-alpha Receptor, IFN gamma, IFN-gamma Receptor, FasL/Fas, CD11b, selectins, such as L-Selectin or P-Selectin, PSGL (P-Selectin Ligand), TRAIL, TRAIL-R, Lymphotoxin beta (LT-β), LT-βR, decoyreceptors 1-3, TNF-alpha, TNF-alphaR, MSH, G-CSF, GM-CSF, IL-1, IL-6, IL-7, IL-8, IL31, IL1R, IL31R, IL-10, IL-23, CXCR3 ligands such as CXCL9 and CXCL-10, PD-1, PD-1L, PD-2 (PDC2), PD-2L, Granzyme B, Granulysine, CD11b, TIGIT, CD 112, CD 155, nitric oxide synthase, DNA methyltransferase 3b (DNMT3b), Jumonji domain-containing protein 1A (JMJD1A), somatostatine, histone deacetylases (HDAC) such as HDAC3 or HDAC 9, CSF1 receptor (CSF1R), IL-34, TAM, all chemokines and chemokine receptors, all cytokines and cytokine receptors.

8. The viral vector according to claim 6, wherein the polypeptide that enables and/or increases target specificity of a cell enables and/or increases specificity to a tumor antigen, in particular wherein the tumor antigen is VEGF, a VEGF-Receptor, an antagonists to a metalloproteinase (e.g. MMP-9), CD40/CD40L, EGFR, Annexin1, FGFR-1, Her2, St6galnac5, MMP1-28, TIMPS1-4, Melanotransferrin, alpha4-beta1 Integrin, VCAM-1, E-cadherin, Alpha-v-beta3 integrin, Alpha-v-beta5 integrin, Alpha-v-beta6 integrin, Alpha-v-beta8 integrin, CCND1, BRCA, CEA, cancer-related antigen 72-4 (CA 72-4), cancer-related antigen 19-9 (CA 19-9), WT1, CD 11b, L-Selectin, NY-ESO-1, or a fragment thereof.

9. A viral vector comprising a transgene under control of one or more promoters, wherein the transgene encodes
a) PGRN, or a functional fragment thereof; or
b) a polypeptide having at least 95%, 96%, 97%, 98%, 99% or 100% sequence identity to the sequence shown in SEQ ID NO: 7, SEQ ID NO: 8 or SEQ ID NO: 9, or a functional fragment thereof.

10. A viral vector comprising a transgene under control of one or more promoters, wherein the transgene encodes
a) IL-12, or a functional fragment thereof; or
b) a polypeptide having at least 95%, 96%, 97%, 98%, 99% or 100% sequence identity to the sequence shown in SEQ ID NO: 11, or a functional fragment thereof; and/or a polypeptide having at least 95%, 96%, 97%, 98%, 99% or 100% sequence identity to the sequence shown in SEQ ID NO: 12, or a functional fragment thereof.

11. A viral vector comprising a transgene under control of one or more promoters, wherein the transgene encodes
a) IFN-gamma, or a functional fragment thereof; or
b) a polypeptide having at least 95%, 96%, 97%, 98%, 99% or 100% sequence identity to the sequence shown in SEQ ID NO: 10, or a functional fragment thereof.

12. A viral vector comprising a transgene under control of one or more promoters, wherein the transgene encodes
a) GM-CSF, or a functional fragment thereof; or
b) a polypeptide having at least 95%, 96%, 97%, 98%, 99% or 100% sequence identity to the sequence shown in SEQ ID NO: 13, or a functional fragment thereof.

13. A viral vector comprising a transgene under control of one or more promoters, wherein the transgene encodes
a) G-CSF, or a functional fragment thereof; or
b) a polypeptide having at least 95%, 96%, 97%, 98%, 99% or 100% sequence identity to the sequence shown in SEQ ID NO: 14, or a functional fragment thereof.

14. A viral vector comprising a transgene under control of one or more promoters, wherein the transgene encodes
a) GM-CSF and IFN-gamma, or functional fragments thereof; or
b) a polypeptide having at least 95%, 96%, 97%, 98%, 99% or 100% sequence identity to the sequence shown in SEQ ID NO: 15, or a functional fragment thereof.

15. A viral vector comprising a transgene under control of one or more promoters, wherein the transgene encodes
a) G-CSF and IFN-gamma, or functional fragments thereof; or
b) a polypeptide having at least 95%, 96%, 97%, 98%, 99% or 100% sequence identity to the sequence shown in SEQ ID NO: 16, or a functional fragment thereof.

16. A viral vector comprising a transgene under control of one or more promoters, wherein the transgene encodes
a) IL-2, or a functional fragment thereof; or
b) a polypeptide having at least 95%, 96%, 97%, 98%, 99% or 100% sequence identity to the sequence shown in SEQ ID NO: 17, or a functional fragment thereof.

17. A viral vector comprising a transgene under control of one or more promoters, wherein the transgene encodes
a) IL-15, or a functional fragment thereof; or
b) a polypeptide having at least 95%, 96%, 97%, 98%, 99% or 100% sequence identity to the sequence shown in SEQ ID NO: 18, or a functional fragment thereof.

18. A viral vector comprising a transgene under control of one or more promoters, wherein the transgene encodes
a) IL-21, or a functional fragment thereof; or
b) a polypeptide having at least 95%, 96%, 97%, 98%, 99% or 100% sequence identity to the sequence shown in SEQ ID NO: 19, or a functional fragment thereof.

19. A viral vector comprising a transgene under control of one or more promoters, wherein the transgene encodes
a) IFN-alpha, or a functional fragment thereof; or
b) a polypeptide having at least 95%, 96%, 97%, 98%, 99% or 100% sequence identity to the sequence shown in SEQ ID NO: 20, or a functional fragment thereof.

20. The viral vector according to any one of claims 9 to 19, wherein the one or more promoters comprise:
a) a myelo-specific promoter, or a functional fragment thereof; and/or
b) a microglia-specific promoter, or a functional fragment thereof; and/or
c) a fusion promoter comprising or consisting of
i) a first promoter, wherein said first promoter is a myelo-specific promoter or a microglia-specific promoter, or a functional fragment thereof; and
ii) a second promoter.

21. The viral vector according to claim 20, wherein the myelo-specific promoter is
a) a miR233 promoter, or a functional fragment thereof; or a promoter having at least 95%, 96%, 97%, 98%, 99% or 100% sequence identity to the sequence shown in SEQ ID NO: 1, or a functional fragment thereof;
b) an AIF1 promoter, or a functional fragment thereof; or a promoter having at least 95%, 96%, 97%, 98%, 99% or 100% sequence identity to the sequence shown in SEQ ID NO:5, or a functional fragment thereof; or
c) an ITGAM promoter, or a functional fragment thereof; or a promoter having at least 95%, 96%, 97%, 98%, 99% or 100% sequence identity to the sequence shown in SEQ ID NO:6, or a functional fragment thereof.

22. The viral vector according to claim 20 or 21, wherein the microglia-specific promoter is
a) a TMEM119 promoter or a promoter having at least 95%, 96%, 97%, 98%, 99% or 100% sequence identity to the sequence shown in SEQ ID NO:3, SEQ ID NO:23 or SEQ ID NO:24, or a functional fragment thereof; or
b) a P2RY12 promoter or a promoter having at least 95%, 96%, 97%, 98%, 99% or 100% sequence identity to the sequence shown in SEQ ID NO: 2, SEQ ID NO:21 or SEQ ID NO:22, or a functional fragment thereof;
c) an OLFML3 promoter or a promoter having at least 95%, 96%, 97%, 98%, 99% or 100% sequence identity to the sequence shown in SEQ ID NO:4 or SEQ ID NO:25, or a functional fragment thereof.

23. The viral vector according to any one of claims 20 to 22, wherein the first promoter is a myelo-specific promoter and wherein the second promoter is a microglia-specific promoter, or vice versa.

24. The viral vector according to any one of claims 20 to 23, wherein the first promoter is a miR233 promoter, or a promoter having at least 95%, 96%, 97%, 98%, 99% or 100% sequence identity to the sequence shown in SEQ ID NO: 1, or a functional fragment thereof; and wherein the first promoter is operably linked to
i) a TMEM119 promoter, or a promoter having at least 95%, 96%, 97%, 98%, 99% or 100% sequence identity to the sequence shown in SEQ ID NO: 5, SEQ ID NO:6 or SEQ ID NO:7, or a functional fragment thereof;
ii) a P2RY12 promoter, or a promoter having at least 95%, 96%, 97%, 98%, 99% or 100% sequence identity to the sequence shown in SEQ ID NO: 2, SEQ ID NO: 3 or SEQ ID NO: 4, or a functional fragment thereof;
iii) an OLFML3 promoter, or a promoter having at least 95%, 96%, 97%, 98%, 99% or 100% sequence identity to the sequence shown in SEQ ID NO: 8 or SEQ ID NO:9, or a functional fragment thereof
iv) an ITGAM promoter, or a promoter having at least 95%, 96%, 97%, 98%, 99% or 100% sequence identity to the sequence shown in SEQ ID NO: 11, or a functional fragment thereof; and/or
v) an AIF1 promoter or a promoter having at least 95%, 96%, 97%, 98%, 99% or 100% sequence identity to the sequence shown in SEQ ID NO: 10, or a functional fragment thereof.

25. The viral vector according to any one of claims 9 to 24, wherein the viral vector comprises at least one transcriptional regulatory element, and wherein said at least one transcriptional regulatory element is arranged such that it inhibits or activates a transcriptional activity of the promoter.

26. The viral vector according to claim 25, wherein the at least one transcriptional regulatory element comprises a binding site for a transcriptional activator or repressor, in particular wherein the transcriptional activator or repressor comprises:
i) an antibiotic-binding domain, in particular a tetracycline/doxycycline-binding domain, a macrolide-binding domain or a pristinamycin-binding domain;
ii) a hormone-binding domain, in particular a RU486-binding domain or an abscisic acid-binding domain;
iii) a steroid-binding domain, in particular an ecdysone-binding domain;
iv) a dimerizer system, in particular a rapamycin-based of rapalog-based dimerizer system.

27. The viral vector according to any one of claims 9 to 26, wherein the viral vector encodes a riboswitch, and wherein the riboswitch controls translation of an mRNA encoding the therapeutic protein or the combination of therapeutic proteins.

28. The viral vector according to any one of claims 1 to 27, wherein the viral vector is
a) a retroviral vector, in particular a lentiviral vector, more particularly a lentiviral SIN vector; or
b) a foamy viral vector; or
c) a viral vector selected from the group consisting of: an adenoviral vector, an adeno-associated viral vector, a herpes viral vector, a parvoviral vector, a coronaviral vector, and an alpha-retroviral vector.

29. A fusion promoter comprising
a) a miR223 promoter or a promoter having at least 95%, 96%, 97%, 98%, 99% or 100% sequence identity to the sequence shown in SEQ ID NO: 1, or a functional fragment thereof; and
b) a microglia-specific promoter, or a functional fragment thereof;
wherein the miR223 promoter or the promoter having at least at least 95%, 96%, 97%, 98%, 99% or 100% sequence identity to the sequence shown in SEQ ID NO: 1, or the functional fragment thereof, is operably linked to the microglia-specific promoter, or the functional fragment thereof.

30. The fusion promoter according to claim 29, wherein the microglia-specific promoter is
a) a TMEM119 promoter or a promoter having at least 95%, 96%, 97%, 98%, 99% or 100% sequence identity to the sequence shown in SEQ ID NO:3, SEQ ID NO:23 or SEQ ID NO:24, or a functional fragment thereof;
b) a P2RY12 promoter or a promoter having at least 95%, 96%, 97%, 98%, 99% or 100% sequence identity to the sequence shown in SEQ ID NO: 2, SEQ ID NO: 21 or SEQ ID NO: 22, or a functional fragment thereof;
c) an OLFML3 promoter or a promoter having at least 95%, 96%, 97%, 98%, 99% or 100% sequence identity to the sequence shown in SEQ ID NO:4 or SEQ ID NO:25, or a functional fragment thereof,
d) an AIF1 promoter or a promoter having at least 95%, 96%, 97%, 98%, 99% or 100% sequence identity to the sequence shown in SEQ ID NO:5, or a functional fragment thereof; or
e) an ITGAM promoter or a promoter having at least 95%, 96%, 97%, 98%, 99% or 100% sequence identity to the sequence shown in SEQ ID NO:6, or a functional fragment thereof.

31. The fusion promoter according to claim 29 or 30, wherein the fusion promoter comprises at least one transcriptional regulatory element, wherein said at least one transcriptional regulatory element is arranged such that it inhibits or activates a transcriptional activity of the promoter.

32. The fusion promoter according to claim 31, wherein the at least one transcriptional regulatory element comprises a binding site for a transcriptional activator or repressor, in particular wherein the transcriptional activator or repressor comprises:
i) an antibiotic-binding domain, in particular a tetracycline/doxycycline-binding domain, a macrolide-binding domain or a pristinamycin-binding domain;
ii) a hormone-binding domain, in particular a RU486-binding domain or an abscisic acid-binding domain;
iii) a steroid-binding domain, in particular an ecdysone-binding domain;
iv) a dimerizer system, in particular a rapamycin-based of rapalog-based dimerizer system.

33. The fusion promoter according to any one of claims 29 to 32, wherein the fusion promoter
a) comprises any one of the sequences set forth in SEQ ID NO:26-29: or
b) comprises a sequence having 90%, 91%, 92%, 93%, 94% or 95% sequence identity with any one of the sequence set forth in SEQ ID NO: 26-29, wherein the promoter drives expression in microglia and/or myeloid cells.

34. A host cell comprising the viral vector according to any one of claims 1 to 28.

35. The host cell according to claim 34, wherein the host cell is a hematopoietic stem cell, preferably a hematopoietic stem cell of a CD34-positive enriched cell population, or wherein the host cell is a myeloid cell.

36. A pharmaceutical composition comprising the viral vector according to any one of claims 1 to 28 and/or the host cell according to claim 34 or 35.

37. The viral vector according to any one of claims 1 to 28, the host cell according to claim 32 or 33 or the pharmaceutical composition according to claim 36 for use in medicine.

38. The viral vector according to any one of claims 1 to 28, the host cell according to claim 34 or 35 or the pharmaceutical composition according to claim 36 for use in the treatment of a disease or disorder which has its origin or a manifestation in the brain or is brain-based.

39. The viral vector according to any one of claims 9 or 20 to 28, the host cell according to claim 34 or 35 or the pharmaceutical composition according to claim 36 for use in the prevention and/or treatment of a PGRN-associated disease or disorder, in particular wherein the viral vector encodes PGRN, or a functional fragment thereof.

40. The viral vector, the host cell or the pharmaceutical composition for use according to claim 39, wherein the PGRN-associated disease or disorder is a neurodegenerative disease or disorder.

41. The viral vector, the host cell or the pharmaceutical composition for use according to claim 40, wherein the neurodegenerative disease or disorder is a degenerative disease or disorder.

42. The viral vector, the host cell or the pharmaceutical composition for use according to claim 41, wherein the degenerative disease or disorder is selected from the group consisting of: Alzheimer's disease, amyotrophic lateral sclerosis, neuronal ceroid lipofuscinosis and Parkinson's disease.

43. The viral vector according to any one of claims 10 to 28, the host cell according to claim 34 or 35 or the pharmaceutical composition according to claim 36 for use in the treatment of cancer, lymphoma and/or sarcoma in particular wherein the viral vector encodes at least one of IL-12, IFN-gamma, G-CSF, GM-CSF, IL-2, IL-15, IL-21 and/or IFN-alpha; or functional fragments thereof.

44. The viral vector, the host cell or the pharmaceutical composition for use according to claim 43, wherein the cancer, lymphoma and/or sarcoma is a brain tumor or a brain metastasis.

45. The viral vector, the host cell or the pharmaceutical composition for use according to claim 44, wherein the brain tumor is selected from the group consisting of: glioblastoma, glioma, ganglioneuroblastoma, astrocytoma, oligodendroglioma, PNET (primitive neuroectodermal tumor), medulloblastoma, CNS lymphoma, meningioma, retinoblastoma and neuroblastoma.

46. The viral vector, the host cell or the pharmaceutical composition for use according to claim 44, wherein the brain tumor is a metastatic tumor originating from any form of breast cancer, lung cancer, colon cancer, testicular cancer, renal carcinomas, melanoma, ovary carcinomas, prostate carcinoma, neuroendocrine tumors or any other solid tumor or any sarcoma, or any hematologic tumor, comprising all forms of leukemia and lymphomas.

47. The viral vector, the host cell or the pharmaceutical composition for use according to any one of claims 37 to 46, wherein the viral vector, the host cell or the pharmaceutical composition is administered in conjunction with a therapy that reduces the integrity of the blood-brain-barrier, in particular wherein the therapy that reduces the integrity of the blood-brain-barrier is a bone marrow conditioning therapy, a CNS conditioning therapy, and/or a blood-brain-barrier conditioning therapy.

48. The viral vector, the host cell or the pharmaceutical composition for use according to claim 47, wherein the bone marrow conditioning therapy comprises the use of cytotoxic agents, alkylating agents, Busulphan, Treosulfan, Etoposide, Lomustin, radiotherapy, targeted radiotherapy (e.g. Yttrium-90 labeled anti-CD45 antibody, or Yttrium-90 labeled anti-CD66 antibody), ACK2 (anti-c-kit antibody), CD117 antibody-drug-conjugates, CD45-SAP, colony-stimulating factor 1 (CSF1) specific agents, PLX3397, BLZ9445, PLX5622, RG7155, PLX647, Ki20227, GW2580, IL-34 and/or desatinib.

49. The viral vector, the host cell or the pharmaceutical composition for use according to claim 47 or 48, wherein the CNS conditioning therapy comprises the use of Busulphan.

50. The viral vector, the host cell or the pharmaceutical composition for use according to any one of claims 47 to 49, wherein the blood-brain-barrier conditioning therapy comprises radiotherapy or targeted radiotherapy.

51. The viral vector, the host cell or the pharmaceutical composition for use according to any one of claims 47 to 50, wherein the viral vector, the host cell or the pharmaceutical composition is administered after the therapy that reduces the integrity of the blood-brain-barrier, in particular wherein the viral vector, the host cell or the pharmaceutical composition is administered not earlier than half a day after the therapy that reduces the integrity of the blood-brain-barrier.

52. The viral vector according to any one of claims 1 to 28, the host cell according to claim 34 or 35 or the pharmaceutical composition according to claim 36 for use in the treatment of autoimmune diseases.

53. The viral vector according to any one of claims 1 to 28, the host cell according to claim 34 or 35 or the pharmaceutical composition according to claim 36 for use in the treatment of autoinflammatory diseases.

54. The viral vector according to any one of claims 1 to 28, the host cell according to claim 34 or 35 or the pharmaceutical composition according to claim 36 for use in the treatment of allergic diseases.

55. The viral vector according to any one of claims 1 to 28, the host cell according to claim 34 or 35 or the pharmaceutical composition according to claim 36 for use in hematopoietic and solid organ transplantation.

56. A method for treating a disease or disorder which has its origin or a manifestation in the brain or is brain based in a subject in need, the method comprising the steps of:
a) genetically modifying a hematopoietic stem cell and/or a population of enriched CD34-positive bone marrow cells, the modification step comprising a step of contacting the hematopoietic stem cell and/or the population of enriched CD34-positive bone marrow cells with the viral vector according to any one of claims 1 to 28; or genetically modifying a myeloid cell and/or a population of enriched myeloid cells, the modification step comprising a step of contacting the myeloid cell and/or the population of enriched myeloid cells with the viral vector according to any one of claims 1 to 28;
b) administering the genetically modified cells from step (a) intravenously to the subject in need; and
c) treating a disease or disorder which has its origin or a manifestation in the brain or is brain based in the subject in need.

57. The method according to claim 56, wherein the hematopoietic stem cell and/or the population of enriched CD34-positive bone marrow cells, or the myeloid cell and/or the population of enriched myeloid cells have been obtained from the subject in need or from a foreign donor.

58. A method for treating a disease or disorder which has its origin or a manifestation in the brain or is brain based in a subject in need, the method comprising the steps of:
a) mobilizing hematopoietic stem cells in the subject in need;
b) administering the viral vector according to any one of claims 1 to 28 intravenously to the subject in need subsequent to the mobilization of hematopoietic stem cells in step (a); and
c) treating a disease or disorder which has its origin or a manifestation in the brain or is brain based in the subject in need.

59. The method according to claim 58, wherein the mobilization of hematopoietic stem cells in the subject in need comprises the administration of G-CSF and/or Plerixafor.

60. The method according to any one of claims 56 to 59, wherein the disease or disorder which has its origin or a manifestation in the brain or is brain based is a PGRN-associated disease or disorder, in particular wherein the PGRN-associated disease or disorder is a neurodegenerative disease or disorder, in particular wherein the neurodegenerative disease or disorder is a degenerative disease or a neurodegenerative disorder, in particular wherein the degenerative disease or neurodegenerative disorder is selected from the group consisting of Alzheimer's disease, amyotrophic lateral sclerosis, neuronal ceroid lipofuscinosis, and Parkinson's disease, in particular wherein the viral vector encodes PGRN, or a functional fragment thereof.

61. The method according to any one of claims 56 to 59, wherein the disease or disorder which has its origin, or a manifestation, in the brain or is brain based is a brain tumor, in particular wherein the brain tumor is selected from the group consisting of: glioma, glioblastoma, ganglioneuroblastoma, astrocytoma, oligodendroglioma, PNET (primitive neuroectodermal tumor), medulloblastoma, CNS lymphoma, and neuroblastoma; or wherein the brain tumor is a metastatic tumor originating from any form of breast cancer, lung cancer, colon cancer, testicular cancer, renal carcinomas, melanoma, prostate cancer, or any other solid tumor or any sarcoma, or any hematologic tumor, comprising all forms of leukemia and lymphomas, in particular wherein the viral vector encodes IL-12, IFN-gamma, GM-CSF, G-CSF, 11-2, IL-15, IL-21 and/or IFN-alpha, or functional fragments thereof.

62. The method according to any one of claims 56 to 61, wherein the method comprises an additional step of reducing the integrity of the blood-brain-barrier, in particular wherein reducing the integrity of the blood-brain-barrier comprises a bone marrow conditioning therapy, a CNS conditioning therapy, and/or a blood-brain-barrier conditioning therapy.

63. The method according to claim 62, wherein the therapy that reduces the integrity of the blood-brain-barrier is performed prior to the administration of the genetically modified cells to the subject in need, in particular wherein the time interval between the therapy that reduces the integrity of the blood-brain-barrier and the administration of the genetically modified cells is carried out after the therapy that reduces the integrity of the blood-brain-barrier.

64. A method for treating cancer in a subject in need, the method comprising the steps of:
a) mobilizing hematopoietic stem cells in the subject in need;
b) administering the viral vector according to the invention intravenously to the subject in need subsequent to the mobilization of hematopoietic stem cells in step (a); and
c) treating cancer in the subject in need.

65. The method according to claim 64, wherein the mobilization of hematopoietic stem cells in the subject in need comprises the administration of G-CSF and/or Plerixafor.

66. A method for expressing a transgene in the brain and/or CNS of a subject, the method comprising the steps of:
a) genetically modifying a hematopoietic stem cell and/or a population of enriched CD34-positive bone marrow cells, the modification step comprising a step of contacting the hematopoietic stem cell and/or the population of enriched CD34-positive bone marrow cells with the viral vector according to any one of claims 1 to 26; or genetically modifying a myeloid cell and/or a population of enriched myeloid cells, the modification step comprising a step of contacting the myeloid cell and/or the population of enriched myeloid cells with the viral vector according to any one of claims 1 to 28;
b) administering the genetically modified cells from step (a) intravenously to the subject in need; and
c) expressing the transgene encoded by the viral vector in the brain and/or CNS of the subject.

67. The method according to claim 66, wherein the hematopoietic stem cell and/or the population of enriched CD34-positive bone marrow cells; or wherein the myeloid cell and/or the population of enriched myeloid cells has been obtained from the subject or from a foreign donor.

68. A method for expressing a transgene in the brain and/or CNS of a subject, the method comprising the steps of:
a) mobilizing hematopoietic stem cells in the subject;
b) administering the viral vector according to any one of claims 1 to 28 intravenously to the subject in need subsequent to the mobilization of hematopoietic stem cells in step (a); and
c) expressing the transgene encoded in the viral vector in the brain and/or CNS of the subject.

69. The method according to claim 68, wherein the mobilization of hematopoietic stem cells in the subject comprises the administration of G-CSF or Plerixafor.

70. The method according to any one of claims 66 to 69, wherein the method comprises an additional step of reducing the integrity of the blood-brain-barrier, in particular wherein reducing the integrity of the blood-brain-barrier comprises a bone marrow conditioning therapy, a CNS conditioning therapy, and/or a blood-brain-barrier conditioning therapy.

71. The method according to claim 70, wherein the therapy that reduces the integrity of the blood-brain-barrier is performed prior to the administration of the genetically modified cells to the subject in need, in particular wherein the time interval between the therapy that reduces the integrity of the blood-brain-barrier and the administration of the genetically modified cells is carried out after the therapy that reduces the integrity of the blood-brain-barrier.

72. A method for treating a disease or disorder which has its origin or a manifestation in the brain or is brain based in a subject in need, the method comprising the steps of:
a) administering the viral vector according to any one of claims 1 to 28 into the brain of the subject in need or intrathecally; and
b) treating a disease or disorder which has its origin or a manifestation in the brain or is brain based in the subject in need.

73. The viral vector according to claim 72, wherein the viral vector is an AAV-based viral vector.
